(19) United States
(12) Patent Application Publication
(10) Pub. No.: US 2012/0065187 A1
(43) Pub. Date: Mar. 15, 2012

(54) HETEROCYCLIC INHIBITORS OF HISTAMINE RECEPTORS FOR THE TREATMENT OF DISEASE

(75) Inventors: Allen J. Borchardt, San Diego, CA (US); Clay Beauregard, Fort Worth, TX (US); Travis Cook, Carlsbad, CA (US); Robert L. Davis, Tipard, OR (US); Daniel A. Gamache, Arlington, TX (US); John M. Yanni, Burleson, TX (US)

(73) Assignees: ALCON RESEARCH, LTD, Fort Worth, TX (US); KALYPSYS, INC., Del Mar, CA (US)

(21) Appl. No.: 13/301,131

(22) Filed: Nov. 21, 2011

Related U.S. Application Data
(63) Continuation of application No. 12/556,866, filed on Sep. 10, 2009.
(60) Provisional application No. 61/095,826, filed on Sep. 10, 2008.
(60) Provisional application No. 61/231,749, filed on Aug. 6, 2009.

(51) Int. Cl. A61K 31/4985; A61K 31/55; A61K 31/551; A61K 31/5415; A61P 11/06; A61P 37/00; A61P 37/08; A61P 27/02; A61P 17/04; A61P 17/00; C07D 487/04; A61P 29/00

(52) U.S. Cl. 514/210.21; 544/346; 514/250; 544/251; 540/575; 514/214.02; 514/217.05; 514/218; 514/225.2; 514/226.2

(57) ABSTRACT

The present invention relates to compounds and methods which may be useful as inhibitors of H₁R and/or H₄R for the treatment or prevention of inflammatory, autoimmune, allergic, and ocular diseases.

This application claims the benefit of U.S. Provisional Application Nos. 61/095,826, filed Sep. 10, 2008, and No. 61/231,749, filed Aug. 6, 2009, the disclosures of which are hereby incorporated by reference as if written herein in their entireties.

Disclosed herein are new heterocyclic compounds and compositions and their application as pharmaceuticals for the treatment of disease. Methods of inhibition of histamine receptor activity in a human or animal subject are also provided for the treatment of allergic diseases, inflammation, asthma, rhinitis, chronic obstructive pulmonary disease, conjunctivitis, rheumatoid arthritis, and general and localized pruritis.

Histamine, a low molecular weight biogenic amine, is a potent chemical mediator of normal and pathological physiology. Histamine functions as a secreted signal in immune and inflammatory responses, as well as a neurotransmitter. The functions of histamine are mediated through 4 distinct cell surface receptors (H₁R, H₂R, H₃R and H₄R). Histamine receptors vary in expression, signaling, function and histamine affinity, and therefore have different potential therapeutic applications (Zhang M, Thurmond R L, and Dunford P J Pharmacology & Therapeutics. 2007).

All 4 histamine receptors are G protein-coupled receptors (GPCRs). Upon histamine or other agonist binding, they activate distinct signaling pathways through different heterotrimeric G proteins. The H₁R couples to the G_{q} family of G proteins, whose primary signaling cascade induces second messenger calcium mobilization from intracellular stores, followed by multiple downstream effects. H₁R can also increase cyclic GMP (cGMP) production and activate NFκB, a potent, positive transcriptional regulator of inflammation. The H₂R couples to the Gₛ family of G proteins and increases cyclic AMP (cAMP) formation by stimulating adenylate cyclase, although it can also induce calcium mobilization in some cell types. The H₃R mediates its function through G_{i/o} proteins and decreases cAMP formation by inhibiting adenylate cyclase. Like other G_{i/o}-coupled receptors, H₃R also activates the mitogen-activated protein/extracellular-signal regulated protein (MAP/ERK) kinase pathway. H₄R has also been demonstrated to couple to G_{i/o} proteins, with canonical inhibition of cAMP formation and MAP kinase activation. However, H₄R also couples to calcium mobilization in certain cell types. In fact, H₄R signaling in mast cells is primarily through calcium mobilization with little to no impact on cAMP formation.

The H₁R is expressed in many cell types, including endothelial cells, most smooth muscle cells, cardiac muscle, central nervous system (CNS) neurons, and lymphocytes. H₁R signaling causes smooth muscle contraction (including bronchoconstriction), vasodilation, and increased vascular permeability, hallmarks of allergic and other immediate hypersensitivity reactions. In the CNS, H₁R activation is associated with wakefulness. Its activation is also associated with pruritus and nociception in skin and mucosal tissues. For many years, the anti-allergic and anti-inflammatory activities of H₁R antagonists have been utilized to treat acute and chronic allergic disorders and other histamine-mediated pathologies, such as itch and hives.

The H₂R is expressed similarly to the H₁R, and can also be found in gastric parietal cells and neutrophils. H₂R is best known for its central role in gastric acid secretion but has also been reported to be involved in increased vascular permeability and airway mucus production. Antagonists of H2R are widely used in treating peptic ulcers and gastroesophageal reflux disease. These drugs are also used extensively to reduce the risk of gastrointestinal (GI) bleeding associated with severe upper GI ulcers and GI stress in the inpatient setting.

The H₃R is primarily found in the CNS and peripheral nerves innervating cardiac, bronchial, and GI tissue. H₃R signaling regulates the release of multiple neurotransmitters, such as acetylcholine, dopamine, serotonin, and histamine itself (where it acts as a CNS autoreceptor). In the CNS, H₃R participates in the processes of cognition, memory, sleep, and feeding behaviors. H₃R antagonists may be used potentially for treating cognition disorders (such as Alzheimer's disease), sleep and wakefulness disorders, attention disorders, and metabolic disorders (especially related to obesity).

Existence of the H₄R was predicted in the early 1990s, but its cloning by multiple groups was not reported until 2000. In contrast to the other histamine receptors, the H₄R has a distinctly selective expression profile in bone marrow and on certain types of hematopoietic cells. H₄R signaling modulates the function of mast cells, eosinophils, dendritic cells, and subsets of T cells. The H₄R appears to control multiple behaviors of these cells, such as activation, migration, and cytokine and chemokine production (Zhang M, Thurmond R L, and Dunford P J Pharmacology & Therapeutics. 2007).

Of the 4 known histamine receptors, H₁R, H₂R and H₄R have been shown clearly to affect inflammation and other immune responses and are proposed therapeutic targets for treating immune and inflammatory disorders (Jutel et al., 2002; Akdis & Simons, 2006). The H₁R was the first described histamine receptor, and ligands targeting this receptor were initially developed in the 1930s and in widespread use by the 1940s. Common H₁R antagonist drugs currently approved for use include systemic agents such as diphenhydramine (Benadryl, also used topically), cetirizine (Zyrtec), fexofenadine (Allegra), loratadine (Claritin) and desloratadine (Clarinex), and topical agents such as olopatadine (Patanol, Pataday, Patanase), ketotifen, azelastine (Optivar, Astelin) and epinastine (Elestat). Traditional uses have included allergic diseases and reactions such as asthma, rhinitis, and other chronic obstructive pulmonary disorders, ocular disorders such as allergic conjunctivitis, and pruritis of varying etiologies.

However, H₁ receptor antagonists have certain deficiencies as therapeutic agents in the treatment of diseases where histamine is an important mediator. First, their effects are often only moderate and reduce allergic symptoms by only 40 to 50%. In particular, H₁ receptor antagonists, especially systemic agents, have little to no effect in relieving nasal congestion. In allergic asthma, despite the fact that histamine levels rapidly increase in the airways and in plasma (correlating with disease severity), H₁ receptor antagonists have largely failed as a therapeutic strategy, though some effect is seen with administration during the priming phase as opposed to the challenge phase (Thurmond R L et al., Nat Rev Drug Discov, 2008, 7:41-53). Additionally, although the efficacy of H₁ receptor antagonists against pruritus in acute urticarias, associated with hives and insect stings, and in chronic idiopathic urticaria is well proven, H₁R antagonists are mostly ineffective in the treatment of atopic dermatitis-associated pruritus, with the only modest benefits derived from some first-generation compounds likely a consequence of their sedative properties (Sharpe, G. R. & Shuster, S. Br. I Dermatol. 1993, 129:575-9). Finally, sedation caused by H₁R antagonists that cross the blood-brain barrier, among other side effects, limits the utility of many H₁R antagonists in diseases for which they would otherwise be efficacious. These deficiencies render H₁R antagonists amenable to replacement by or supplementation with other agents.

Consequently, attention has focused on the more recently discovered H₄ receptor as a therapeutic target. Given the ability of H₄R to modulate the cellular function of eosinophils, mast cells, dendritic cells and T cells (M. Zhang et al., Pharmacol Ther 2007), it is natural to speculate that the H₄R may be involved in various inflammatory diseases, and that H₄R antagonists would have therapeutic potential (Jutel et al., 2006). Indeed, both in vitro and in vivo evidence has been demonstrated for the utility of H₄R antagonists as anti-inflammatory agents in inflammatory bowel disease (IBD) (Sander L E et al., Gut 2006; 55:498-504). The finding that H₄ receptor antagonists inhibit histamine-induced migration of mast cells and eosinophils in vitro and in vivo, both of which are important effector cells in the allergic response, raises the possibility that this class of compounds could reduce the allergic hyper-responsiveness developed upon repeated exposure to antigens, which is characterized by an increase in the number of mast cells and other inflammatory cells in the nasal and bronchial mucosa (Fung-Leung W P et al., Curr Opin Inves Drugs, 2004 5:11 1174-1182). In contrast to some of the H₁R antagonists, H₄R antagonists given during the allergen challenge phase of a mouse model of asthma are equally effective to those given during sensitization (Thurmond R L et al., Nat Rev Drug Discov, 2008, 7:41-53). In two recent mouse studies, a selective H₄R agonist was shown to induce itch, whereas these responses, and those of histamine, were blocked by pretreatment with H₄R antagonists. Similarly, histamine or H₄ receptor agonist-induced itch was markedly attenuated in H4 receptor-deficient animals (Dunford, P. J. et al., J. Allergy Clin. Immunol, 2007, 119:176-183). The presence of the H₄R in nasal tissue was first discovered by Nakaya et al. (Nakaya, M. et al., Ann Otol Rhinol Laryngol, 2004, 113: 552-557). In addition, a more recent finding showed that there is a significant increase in the level of H₄R in human nasal polyp tissue taken from patients with chronic rhinosinusitis (infection of the nose and nasal cavities) when compared to normal nasal mucosa. Jóküti et al. suggest that the administration of H₄R antagonists might be a new way to treat nasal polyps and chronic rhinosinusitis. The administration of H₄R antagonists may prevent the accumulation of eosinophils as a result of impaired cell chemotaxis toward polypous tissue (Jóküti, A. et al., Cell Biol Int, 2007, 31: 1367). Although scientific data on the role of the H₄R in rhinitis is limited, at present, it is the only indication for which an H₄R inverse agonist (CZC-13788) is reported to be in preclinical development (Hale, R. A. et al., Drug News Perspect, 2007, 20: 593-600).

Current research efforts include both a focus on H₄R selective agents and an alternate path toward dual H₁R/H₄R agents. Johnson & Johnson have developed a well-characterized H₄R antagonist, JNJ-7777120, which is 1000-fold selective over H₁, H₂, and H₃ receptors, and equipotent across human and several nonhuman species. An exemplary H₁R/H₄R dual agent has yet to publish as of the time of this writing, and the ideal proportion of H₁R versus H₄R antagonism is a nascent topic of debate. Nevertheless, the concept of dual activity via a single agent is well-precedented, and the design of multiply active ligands is a current topic in pharmaceutical discovery (Morphy R and Rankovic Z, J Med. Chem. 2005; 48(21):6523-43). Additional reports have shown potential for H₄R antagonists, or potentially, H₁R/H₄R dual antagonists, in the treatment of metabolic disorders such as obesity (Jorgensen E et al., Neuroendocrinology. 2007; 86(3):210-4), vascular or cardiovascular diseases such as atherosclerosis (Tanihide A et al., TCM 2006: 16(8): 280-4), inflammation and pain (Coruzzi G et al., Eur J Pharmacol. 2007 Jun. 1; 563(1-3):240-4), rheumatoid arthritis (Grzybowska-Kowalczyk A et al., Inflamm Res. 2007 April; 56 Suppl 1:S59-60) and other inflammatory and autoimmune diseases including systemic lupus erythematosus (Zhang M, Thurmond R L, and Dunford P J Pharmacology & Therapeutics. 2007). What is clear is that a need still exists in the art for improved and varied antihistamines for the treatment of disease, and that compounds with H₄R and/or H₁R/H₄R antagonist activity may fill this need.

Histamine is reportedly implicated in allergic rhinitis by acting on three HR subtypes, the H₁R, H₃R and H₄R. For many years, the classical application of H₁R antagonists (antihistamines) has been the treatment of allergic rhinitis. H₁R antagonists relieve edema and vasoconstriction, both important symptoms of the disease, but these drugs do not affect the underlying inflammatory responses. After the discovery of the H₃R and H₄R subtypes, the traditional role for H₁R antagonists in rhinitis has been reappraised. It has been shown that the H₃R agonist (R)-a-methyl-histamine can induce the dilatation of nasal blood vessels and that this effect can be counteracted by the H₃R antagonist/H₄R agonist clobenpropit (Taylor-Clark, T., et al, Pulm Pharm Ther, 2008, 21: 455-460). Although a role for the H₄R cannot be ruled out, this H₃R antagonist-mediated mechanism in nasal decongestion has certainly caught the attention of scientists from Pfizer Inc. Recently, patient recruitment started for a Phase II clinical trial to test a H₃R antagonist (PF-03654746, unpublished structure) as a novel nasal decongestant in patients with seasonal allergic rhinitis. A dual target approach is being pursued by GSK that is currently recruiting patients to test a systemic H₁/H₃ antagonist (GSK835726, unpublished structure) for seasonal allergic rhinitis in a Phase I clinical trial. A second Phase I trial with another H₁/H₃ antagonist (GSK1004723, unpublished structure) for intranasal administration to treat rhinitis has recently been completed. With these compounds, the mode of action of the classical H₁R antagonist is combined with the potential clinical benefit of added nasal decongestion by H₃R blockade. The synergistic role of the H₁R and H₃R has been demonstrated in vivo in experiments performed at Schering-Plough. In view of the role of the H₄R in allergic rhinitis, other potential treatment paradigms may also be considered, such as combining H₁/H₄, H₃/H₄ or even H₁/H₃/H₄ antagonists/inverse agonist activity in the same molecule approach is being pursued by GSK that is currently recruiting patients to test a systemic H₁/H₃ antagonist (GSK835726, unpublished structure) for seasonal allergic rhinitis in a Phase I clinical trial. A second Phase I trial with another H₁/H₃ antagonist (GSK1004723, unpublished structure) for intranasal administration to treat rhinitis has recently been completed. With these compounds, the mode of action of the classical H₁R antagonist is combined with the potential clinical benefit of added nasal decongestion by H₃R blockade. The synergistic role of the H₁R and H₃R has been demonstrated in vivo in experiments performed at Schering-Plough (McLeod, R. et al., Am J Rhinol, 1999, 3: 391-399). In view of the role of the H₄R in allergic rhinitis, other potential treatment paradigms may also be considered, such as combining H₁/H₄, H₃/H₄ or even H₁/H₃/H₄ antagonists/inverse agonist activity in the same molecule.

Novel compounds and pharmaceutical compositions, certain of which have been found to inhibit the histamine type-1 receptor (H₁R) and/or the histamine type-4 receptor (H₄R) have been discovered, together with methods of synthesizing and using the compounds including methods for the treatment of histamine receptor-mediated diseases in a patient by administering the compounds.

Provided herein are compounds of structural Formula (I), or a salt thereof, wherein,

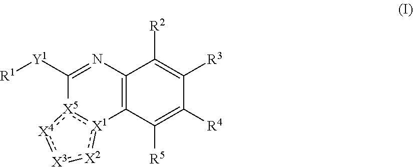

the ring comprising X¹-X⁵ is aromatic;

X¹ and X⁵ are independently selected from the group consisting of C, CH and N;

X² is selected from the group consisting of [C(R⁶)(R⁷)]ₙ, NR⁸, O and S;

X³ is selected from the group consisting of [C(R⁹)(R¹⁰)]ₘ, NR¹¹, O, and S;

X⁴ is selected from the group consisting of [C(R¹²)(R¹³)], NR¹⁴, O and S;

n and m are each an integer from 1 to 2;

Y¹ is selected from the group consisting of a bond, lower alkyl, lower alkoxy, OR¹⁵, NR¹⁶R¹⁷, and lower aminoalkyl;

R¹ is selected from the group consisting of:

null, when Y¹ is selected from the group consisting of OR¹⁵, and NR¹⁶R¹⁷; and

aryl, heterocycloalkyl, cycloalkyl, and heteroaryl, any of which may be optionally substituted, when Y¹ is a bond;

R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;

R⁶, R⁷, R⁹, R¹⁰, R¹², and R¹³ are independently selected from the group consisting of null, hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;

R⁸, R¹¹, and R¹⁴ are independently selected from the group consisting of null, hydrogen, alkyl, heteroalkyl, alkoxy, haloalkyl, perhaloalkyl, aminoalkyl, C-amido, carboxyl, acyl, hydroxy, aryl, arylalkyl, cycloalkyl, cycloalkylalkyl, heterocycloalkyl, heterocycloalkylalkyl, heteroaryl, and heteroarylalkyl, any of which may be optionally substituted;

R¹⁵ and R¹⁶ are independently selected from the group consisting of aminoalkyl, alkylaminoalkyl, aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted; and

R¹⁷ is independently selected from the group consisting of hydrogen, aminoalkyl, alkylaminoalkyl aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted.

Certain compounds disclosed herein may possess useful histamine receptor inhibitory activity, and may be used in the treatment or prophylaxis of a disease or condition in which H₁R and/or H₄R plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting H₁R and/or H₄R. Other embodiments provide methods for treating a H₁R- and/or H₄R-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of H₁R and/or H₄R.

In certain embodiments provided herein,

X¹ and X⁵ are independently selected from the group consisting of C and N;

X² is selected from the group consisting of [C(R⁶)(R⁷)]ₙ, NR⁸, and O;

X³ is selected from the group consisting of [C(R⁹)(R¹⁰)]ₘ, NR¹¹, and O;

X⁴ is selected from the group consisting of NR¹⁴, O, and S; and

Y¹ is selected from the group consisting of bond, OR¹⁵, and NR¹⁶R¹⁷; R¹ is selected from the group consisting of:

null, when Y¹ is selected from the group consisting of OR¹⁵ and NR¹⁶R¹⁷; and

optionally substituted heterocycloalkyl, when Y¹ is a bond.

In certain embodiments provided herein, R⁸, R¹¹, and R¹⁴ are independently selected from the group consisting of null, hydrogen, and C₁-C₃ alkyl.

In other embodiments provided herein,

Y¹ is bond;

X⁴ is NR¹⁴;

R¹ is heterocycloalkyl; and

R¹⁴ is null.

Provided herein are compounds of structural Formula (II), or a salt thereof, wherein,

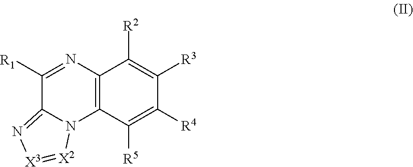

X² is selected from the group consisting of:

CH and N;

X³ is selected from the group consisting of:

CR⁹ and N;

with the proviso that at least one of X² and X³ is N;

R¹ is selected from the group consisting of heterocycloalkyl, which may be optionally substituted;

R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; and R⁹ is selected from the group consisting of hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;

with the provisos that

when X³ is CR⁹; and R⁹ is 2-furanyl; and R¹ is selected from the group consisting of piperazin-1-yl and 4-(2-hydroxyethyl)piperazin-1-yl; then R², R³, R⁴, and R⁵ are not all hydrogen; and

when X³ is N; then R¹ is selected from the group consisting of 4-methylpiperazin-1-yl, piperazin-1-yl, and 4-(hexahydropyrrolo[1,2-a]pyrazin-2(1H)-yl); and

when compounds have structural Formula (IIIa), wherein:

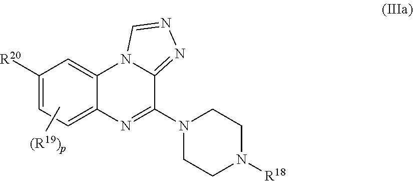

p is an integer from 0 to 3; and

R¹⁸ is selected from the group consisting of hydrogen and methyl; and

R²⁰ is selected from the group consisting of hydrogen and chlorine; and

R¹⁹ is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R¹⁹ are not all hydrogen; and

when compounds have structural Formula (IIIa), wherein:

p is an integer from 0 to 3; and

R¹⁸ is methyl; and

R²⁰ is nitro; and

R¹⁹ is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R¹⁹ are not all hydrogen; and

when compounds have structural Formula (IIIb), wherein:

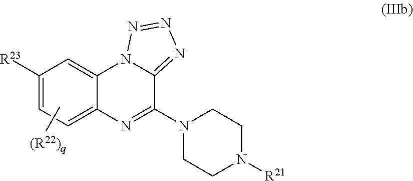

q is an integer from 0 to 3; and

R²¹ is methyl; and

R²³ is selected from the group consisting of hydrogen and methyl; and

R²² is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R²² are not all hydrogen; and

when compounds have structural Formula (IIIb), wherein:

R²¹ and R²³ are hydrogen; and

R²² is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R²² are not all hydrogen.

In certain embodiments provided herein,

X² is CH;

X³ is N; and

R¹ is selected from the group consisting of 4-methylpiperazin-1-yl and piperazin-1-yl.

In certain embodiments provided herein,

X² is N;

X³ is CR⁹; and

R⁹ is selected from the group consisting of hydrogen, lower alkyl, halogen, haloalkyl, perhaloalkyl, amino, carboxyl, cyano, nitro, aryl, cycloalkyl, heterocycloalkyl, any of which may be optionally substituted.

In other embodiments provided herein,

X² and X³ are N;

R¹ is selected from the group consisting of 4-methylpiperazin-1-yl and piperazin-1-yl; and

R⁴ is selected from the group consisting of halogen, haloalkyl, perhaloalkyl, and perhaloalkoxy.

Provided herein are compounds of structural Formula (IV), or a salt thereof, wherein,

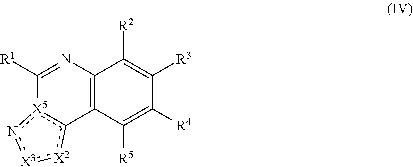

or a salt, wherein:

the 5-membered ring comprising X², X³, and X⁵ is aromatic;

X⁵ is selected from the group consisting of C and N;

X² is selected from the group consisting of:

N, when X⁵ is N; and

O and CR⁶, when X⁵ is C;

X³ is selected from the group consisting of CR⁹ and O, when X⁵ is C; and

CR⁹, when X⁵ is N;

R¹ is heterocycloalkyl, which may be optionally substituted;

R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; and

R⁶ and R⁹ are independently selected from the group consisting of hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;

with the provisos that

when X⁵ is N; then R¹ is selected from the group consisting of 4-methylpiperazin-1-yl, piperazin-1-yl and bicyclic heterocycloalkyl;

when X² is O; and X³ is CR⁹; and X⁵ is C; then R¹ cannot be 4-morpholino, 4-piperidinyl, or 4-phenylpiperidin-4-ol;

when X² is N; and X³ is CR⁹; and X⁵ is N; and R¹ is 4-methylpiperazin-1-yl; and R⁴ is hydrogen; then R², R³, R⁵, and R⁹ are not all hydrogen; and

when X² is N; and X³ is CR⁹; and X⁵ is N; and R¹ is piperazin-1-yl; and R⁴ is methyl; then R², R³, R⁵, and R⁹ are not all hydrogen; and

when X² is N; and X³ is CR⁹; and X⁵ is N; and R¹ is 4-methylpiperazin-1-yl; and R⁴ is methoxy; then R³ cannot be methoxy.

In certain embodiments provided herein, X⁵ is N.

In other embodiments provided herein,

X² is N;

X³ is CR⁹;

R⁴ is selected from the group consisting of halogen, haloalkyl, lower alkenyl, perhaloalkyl, and perhaloalkoxy; and

R⁹ is selected from the group consisting of hydrogen and lower alkyl.

In further embodiments provided herein, X⁵ is C.

In yet further embodiments provided herein,

X² is CR⁶; and

X³ is O.

In certain embodiments provided herein,

X² is O;

X³ is CR⁹; and

R¹ is selected from the group consisting of a 5-membered heterocycloalkyl and a 6-membered heterocycloalkyl containing at least two nitrogens.

In certain embodiments provided herein,

R², R³, R⁴, and R⁵ are independently selected from the group consisting of hydrogen, C₁-C₁₀ alkyl, C₁-C₁₀ alkenyl, alkoxy, halogen, haloalkyl, perhaloalkyl, perhaloalkoxy, cyano, and nitro; and

R⁹ is selected from the group consisting of hydrogen, C₁-C₁₀ alkyl, heteroalkyl, alkoxy, halogen, haloalkyl, perhaloalkyl, amino, carboxyl, cyano, nitro, aryl, heteroaryl, cycloalkyl, heterocycloalkyl, any of which may be optionally substituted.

In other embodiments provided herein,

R², R³, and R⁵ are independently selected from the group consisting of hydrogen, lower alkyl, lower alkenyl, halogen, perhaloalkyl, haloalkyl, and perhaloalkoxy; and

R⁴ is selected from the group consisting of lower alkyl, lower alkenyl, halogen, perhaloalkyl, haloalkyl, and perhaloalkoxy.

In further embodiments provided herein,

R², R³, and R⁵ are independently selected from the group consisting of hydrogen, lower alkyl, lower alkenyl, halogen, perhaloalkyl, haloalkyl, and perhaloalkoxy; and

R⁴ is selected from the group consisting of lower alkyl, lower alkenyl, bromine, fluorine, perhaloalkyl, haloalkyl, and perhaloalkoxy.

In certain embodiments provided herein,

R² is selected from the group consisting of lower alkyl, lower alkenyl, halogen, perhaloalkyl, haloalkyl, and perhaloalkoxy;

R³ and R⁵ are independently selected from the group consisting of hydrogen, lower alkyl, lower alkenyl, halogen, perhaloalkyl, haloalkyl, and perhaloalkoxy; and

R⁴ is selected from the group consisting of lower alkyl, lower alkenyl, halogen, perhaloalkyl, haloalkyl, and perhaloalkoxy.

In certain embodiments provided herein,

R² and R⁵ are independently selected from the group consisting of hydrogen, lower alkyl, lower alkenyl, halogen, perhaloalkyl, haloalkyl, and perhaloalkoxy;

R³ is selected from the group consisting of lower alkyl, lower alkenyl, halogen, perhaloalkyl, haloalkyl, and perhaloalkoxy; and

R⁴ is selected from the group consisting of lower alkyl, lower alkenyl, halogen, perhaloalkyl, haloalkyl, and perhaloalkoxy.

In other embodiments provided herein, R², R³, R⁴, and R⁵ are independently selected from the group consisting of hydrogen, lower alkyl, halogen, haloalkyl, perhaloalkyl, and perhaloalkoxy.

In further embodiments provided herein, R², R³, and R⁵ are independently selected from the group consisting of hydrogen, halogen, haloalkyl, lower alkyl, lower alkenyl, alkoxy, perhaloalkyl, and perhaloalkoxy.

In yet further embodiments provided herein, R², R³ and R⁵ are independently selected from the group consisting of hydrogen, halogen, haloalkyl, lower alkyl, perhaloalkyl, and perhaloalkoxy.

In other embodiments provided herein, R⁴ is selected from the group consisting of halogen, lower alkyl, lower alkenyl, perhaloalkoxy, and perhaloalkyl.

In certain embodiments provided herein, R⁴ is selected from the group consisting of halogen, C₁-C₃ alkyl, and perhaloalkyl.

In certain embodiments provided herein, wherein R⁴ is selected from the group consisting of methyl, halogen, and perhaloalkyl.

In other embodiments provided herein, wherein R⁴ is selected from the group consisting of methyl, bromine, chlorine, and perhaloalkyl

In further embodiments provided herein, R⁴ is selected from the group consisting of halogen and perhaloalkyl.

In yet further embodiments provided herein, R⁴ is selected from the group consisting of bromine, chlorine, and perhaloalkyl.

In certain embodiments provided herein, R⁴ is perhaloalkyl.

In other embodiments provided herein, R⁴ is halogen.

In other embodiments provided herein, R³ and R⁴ are halogen.

In further embodiments provided herein, R² and R³ are independently selected from the group consisting of hydrogen and halogen.

In yet further embodiments provided herein, R² and R³ are independently selected from the group consisting of hydrogen, chlorine, and fluorine.

In yet further embodiments provided herein, R² and R³ are hydrogen.

In certain embodiments provided herein, R³ is selected from the group consisting of hydrogen, C₁-C₃ alkyl, halogen, and perhaloalkyl.

In other embodiments provided herein, R³ is hydrogen.

In other embodiments provided herein, R³ is halogen.

In further embodiments provided herein, R² and R⁵ are independently selected from the group consisting of hydrogen, lower alkyl, halogen, and perhaloalkyl.

In certain embodiments provided herein, R² and R⁵ are independently selected from the group consisting of hydrogen and halogen.

In other embodiments provided herein, R⁵ is hydrogen.

In other embodiments provided herein, R² is halogen.

In further embodiments provided herein, R² is hydrogen.

In further embodiments provided herein,

R¹ is piperazin-1-yl;

R² is hydrogen; and

R⁴ is selected from the group consisting of halogen and perhaloalkyl.

In yet further embodiments provided herein,

R² is hydrogen;

R³ is halogen; and

R⁴ is methyl.

In yet further embodiments provided herein,

R² and R⁴ are halogen; and

R³ is hydrogen.

In yet further embodiments provided herein,

R² and R³ are hydrogen; and

R⁴ is perhaloalkyl.

In certain embodiments provided herein, R⁹ is selected from the group consisting of hydrogen and C₁-C₃ alkyl.

In other embodiments provided herein, R⁹ is selected from the group consisting of hydrogen and methyl.

In other embodiments provided herein,

R³ is hydrogen; and

R⁹ is methyl.

In certain embodiments provided herein, R⁶ is hydrogen.

In certain embodiments, R¹ is selected from the group consisting of 4-methylpiperazin-1-yl and piperazin-1-yl.

In other embodiments provided herein, R¹ is 4-methylpiperazin-1-yl.

In further embodiments provided herein, R¹ is piperazin-1-yl.

As used herein, the terms below have the meanings indicated.

When ranges of values are disclosed, and the notation “from n₁ . . . to n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon group having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl groups include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether group, wherein the term alkyl is as defined below. Examples of suitable alkyl ether groups include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl group containing from 1 to 20 carbon atoms. In certain embodiments, said alkyl group will comprise from 1 to 10 carbon atoms. In further embodiments, said alkyl group will comprise from 1 to 6 carbon atoms. Alkyl groups may be optionally substituted as defined herein. Examples of alkyl groups include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) group wherein the term alkyl is as defined above and wherein the sulfur may be singly or doubly oxidized. Examples of suitable alkyl thioether groups include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon group having one or more triple bonds and containing from 2 to 20 carbon atoms. In certain embodiments, said alkynyl group comprises from 2 to 6 carbon atoms. In further embodiments, said alkynyl group comprises from 2 to 4 carbon atoms. The term “alkynylene” refers to a carbon-carbon triple bond attached at two positions such as ethynylene (—C:::C—, —C≡C—). Examples of alkynyl groups include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl group derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent group C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′ group, with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl group having the meaning as defined above wherein one or more hydrogens are replaced with a halogen. Specifically embraced are monohaloalkyl, dihaloalkyl and polyhaloalkyl groups. A monohaloalkyl group, for one example, may have an iodo, bromo, chloro or fluoro atom within the group. Dihalo and polyhaloalkyl groups may have two or more of the same halo atoms or a combination of different halo groups. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon group, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 7 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, which may be optionally substituted as provided.

The term “lower heteroalkyl,” as used herein, alone or in combination, refers to a stable straight or branched chain, or cyclic hydrocarbon group, or combinations thereof, fully saturated or containing from 1 to 3 degrees of unsaturation, consisting of one to six atoms in which one to three may be heteroatoms selected from the group consisting of O, N, and S, and the remaining atoms are carbon. The nitrogen and sulfur atoms may optionally be oxidized and the nitrogen heteroatom may optionally be quaternized. The heteroatom(s) O, N and S may be placed at any interior or terminal position of the heteroalkyl group. Up to two heteroatoms may be consecutive, such as, for example, —CH₂—NH—OCH₃.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′ group with R and R′ as defined herein.

The term “thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “inhibition” (and by extension, “inhibitor”) as used herein encompasses all forms of functional protein (enzyme, kinase, receptor, channel, etc., for example) inhibition, including neutral antagonism, inverse agonism, competitive inhibition, and non-competitive inhibition (such as allosteric inhibition). Inhibition may be phrased in terms of an IC₅₀, defined below.

In certain embodiments, “H₁R inhibitor” is used herein to refer to a compound that exhibits an IC₅₀ with respect to the histamine type-1 receptor of no more than about 100 μM and more typically not more than about 50 μM, as measured in the in vitro histamine receptor cell-based assays described generally hereinbelow. Similarly, “H₃R inhibitor” is used herein to refer to a compound that exhibits an IC₅₀ with respect to the histamine type-3 receptor of no more than about 100 μM and more typically not more than about 50 μM, as measured in the in vitro histamine receptor cell-based assays described generally hereinbelow. Also similarly, “H₄R inhibitor” is used herein to refer to a compound that exhibits an IC₅₀ with respect to the histamine type-4 receptor of no more than about 100 μM and more typically not more than about 50 μM, as measured in the in vitro histamine receptor cell-based assays described generally hereinbelow. A “H₁/H₄ inhibitor” is used herein to refer to a compound that exhibits an IC₅₀ with respect to both the histamine type-1 receptor and the histamine type-4 receptor of no more than about 100 μM and more typically not more than about 50 μM, as measured in the in vitro histamine receptor cell-based assays described generally hereinbelow; the amount of inhibition need not be equivalent at each receptor, but should not be negligible. In certain embodiments, such as, for example, in the case of an in vitro ligand-binding assay protocol, “IC₅₀” is that concentration of inhibitor which is required to displace a natural ligand or reference standard to a half-maximal level. In other embodiments, such as, for example, in the case of certain cellular or in vivo protocols which have a functional readout, “IC₅₀” is that concentration of inhibitor which reduces the activity of a functional protein (e.g., H₁R and/or H₄R) to a half-maximal level. Certain compounds disclosed herein have been discovered to exhibit inhibitory activity against H₁R and/or H₄R. In certain embodiments, compounds will exhibit an IC₅₀ with respect to H₁R and/or H₄R of no more than about 10 μM; in further embodiments, compounds will exhibit an IC₅₀ with respect to H₁R and/or H₄R of no more than about 5 μM; in yet further embodiments, compounds will exhibit an IC₅₀ with respect to H₁R and/or H₄R of not more than about 1 μM; in yet further embodiments, compounds will exhibit an IC₅₀ with respect to H₁R and/or H₄R of not more than about 200 nM, as measured in the H₁R and/or H₄R assay described herein.

The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual, ocular, and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Examples of fillers or diluents for use in oral pharmaceutical formulations such as capsules and tablets include, without limitation, lactose, mannitol, xylitol, dextrose, sucrose, sorbitol, compressible sugar, microcrystalline cellulose (MCC), powdered cellulose, cornstarch, pregelatinized starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers such as polyethylene oxide, and hydroxypropyl methyl cellulose. Fillers may have complexed solvent molecules, such as in the case where the lactose used is lactose monohydrate. Fillers may also be proprietary, such in the case of the filler PROSOLV® (available from JRS Pharma). PROSOLV is a proprietary, optionally high-density, silicified microcrystalline cellulose composed of 98% microcrystalline cellulose and 2% colloidal silicon dioxide. Silicification of the microcrystalline cellulose is achieved by a patented process, resulting in an intimate association between the colloidal silicon dioxide and microcrystalline cellulose. ProSolv comes in different grades based on particle size, and is a white or almost white, fine or granular powder, practically insoluble in water, acetone, ethanol, toluene and dilute acids and in a 50 g/l solution of sodium hydroxide.

Examples of disintegrants for use in oral pharmaceutical formulations such as capsules and tablets include, without limitation, sodium starch glycolate, sodium carboxymethyl cellulose, calcium carboxymethyl cellulose, croscarmellose sodium, povidone, crospovidone (polyvinylpolypyrrolidone), methyl cellulose, microcrystalline cellulose, powdered cellulose, low-substituted hydroxy propyl cellulose, starch, pregelatinized starch, and sodium alginate.

Additionally, glidants and lubricants may be used in oral pharmaceutical formulations to ensure an even blend of excipients upon mixing. Examples of lubricants include, without limitation, calcium stearate, glyceryl monostearate, glyceryl palmitostearate, hydrogenated vegetable oil, light mineral oil, magnesium stearate, mineral oil, polyethylene glycol, sodium benzoate, sodium lauryl sulfate, sodium stearyl fumarate, stearic acid, talc, and zinc stearate. Examples of glidants include, without limitation, silicon dioxide (SiO₂), talc cornstarch, and poloxamers. Poloxamers (or LUTROL®, available from the BASF Corporation) are A-B-A block copolymers in which the A segment is a hydrophilic polyethylene glycol homopolymer and the B segment is hydrophobic polypropylene glycol homopolymer.

Examples of tablet binders include, without limitation, acacia, alginic acid, carbomer, carboxymethyl cellulose sodium, dextrin, ethylcellulose, gelatin, guar gum, hydrogenated vegetable oil, hydroxyethyl cellulose, hydroxypropyl cellulose, hydroxypropylmethyl cellulose, copolyvidone, methyl cellulose, liquid glucose, maltodextrin, polymethacrylates, povidone, pregelatinized starch, sodium alginate, starch, sucrose, tragacanth, and zein.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Topical ophthalmic, otic, and nasal formulations of the present invention may comprise excipients in addition to the active ingredient. Excipients commonly used in such formulations include, but are not limited to, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants. Any of a variety of excipients may be used in formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, guar gum, xanthan gum, carrageenin, agar and acacia, starch derivatives, such as starch acetate and hydroxypropyl starch, and also other synthetic products such as polyvinyl alcohol, polyvinylpyrrolidone, polyvinyl methyl ether, polyethylene oxide, preferably cross-linked polyacrylic acid and mixtures of those products. The concentration of the excipient is, typically, from 1 to 100,000 times the concentration of the active ingredient. In preferred embodiments, the excipients to be included in the formulations are typically selected on the basis of their inertness towards the active ingredient component of the formulations.

Relative to ophthalmic, otic, and nasal formulations, suitable tonicity-adjusting agents include, but are not limited to, mannitol, sodium chloride, glycerin, sorbitol and the like. Suitable buffering agents include, but are not limited to, phosphates, borates, acetates and the like. Suitable surfactants include, but are not limited to, ionic and nonionic surfactants (though nonionic surfactants are preferred), RLM 100, POE 20 cetylstearyl ethers such as Procol® CS20 and poloxamers such as Pluronic® F68.

The formulations set forth herein may comprise one or more preservatives. Examples of such preservatives include p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, amino alcohols such as AMP-95, or sorbic acid. In certain embodiments, the formulation may be self-preserved so that no preservation agent is required.

For ophthalmic, otic, or nasal administration, the formulation may be a solution, a suspension, or a gel. In preferred aspects, the formulations are for topical application to the eye, nose, or ear in aqueous solution in the form of drops. The term “aqueous” typically denotes an aqueous formulation wherein the formulation is >50%, more preferably >75% and in particular >90% by weight water. These drops may be delivered from a single dose ampoule which may preferably be sterile and thus render bacteriostatic components of the formulation unnecessary. Alternatively, the drops may be delivered from a multi-dose bottle which may preferably comprise a device which extracts any preservative from the formulation as it is delivered, such devices being known in the art.

For ophthalmic disorders, components of the invention may be delivered to the eye as a concentrated gel or a similar vehicle, or as dissolvable inserts that are placed beneath the eyelids.

The formulations of the present invention that are adapted for topical administration to the eye are preferably isotonic, or slightly hypotonic in order to combat any hypertonicity of tears caused by evaporation and/or disease. This may require a tonicity agent to bring the osmolality of the formulation to a level at or near 210-320 milliosmoles per kilogram (mOsm/kg). The formulations of the present invention generally have an osmolality in the range of 220-320 mOsm/kg, and preferably have an osmolality in the range of 235-300 mOsm/kg. The ophthalmic formulations will generally be formulated as sterile aqueous solutions.

In certain ophthalmic embodiments, the compositions of the present invention are formulated with one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; and carbomers, such as carbomer 934P, carbomer 941, carbomer 940 and carbomer 974P. Certain formulations of the present invention may be used with contact lenses or other ophthalmic products.

Preferred formulations are prepared using a buffering system that maintains the formulation at a pH of about 4.5 to a pH of about 8. A most preferred formulation pH is from 7 to 8.

In particular embodiments, a formulation of the present invention is administered once a day. However, the formulations may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. The formulations are administered at varying dosages, but typical dosages are one to two drops at each administration, or a comparable amount of a gel or other formulation. One of ordinary skill in the art would be familiar with determining a therapeutic regimen for a specific indication.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, galactomannan polymers (such as guar and derivatives thereof) and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Non-limiting examples of possible combination therapies include use of certain compounds of the invention with H₁R antagonists and/or H₃R antagonists. Specific, non-limiting examples of possible combination therapies include use of certain compounds of the invention with H₁R antagonists such as acrivastine, alcaftadine, antazoline, azelastine, bromazine, brompheniramine, cetirizine, chlorpheniramine, clemastine, desloratidine, diphenhydramine, diphenylpyraline, ebastine, emedastine, epinastine, fexofenadine, hydroxyzine, ketotifen, levocabastine, levocetirizine, loratidine, methdilazine, mizolastine, promethazine, olopatadine, and triprolidine.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating H₁R and/or H₄R-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of H₁R and/or H₄R-mediated disorders.

Specific diseases to be treated by the compounds, compositions, and methods disclosed herein include inflammation and related diseases, including autoimmune diseases. The compounds are useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. The compounds are also useful in treating osteoporosis and other related bone disorders. These compounds can also be used to treat gastrointestinal conditions such as reflux esophagitis, diarrhea, inflammatory bowel disease, Crohn's disease, gastritis, irritable bowel syndrome and ulcerative colitis. The compounds may also be used in the treatment of upper respiratory inflammation, such as, but not limited to, seasonal allergic rhinitis, non-seasonal allergic rhinitis, acute non-allergic rhinitis, chronic non-allergic rhinitis, Sampter's triad, non-allergic rhinitis with eosinophilia syndrome, nasal polyposis, atrophic rhinitis, hypertrophic rhinitis, membranous rhinitis, vasomotor rhinitis, rhinosinusitis, chronic rhinopharyngitis, rhinorrhea, occupational rhinitis, hormonal rhinitis, drug-induced rhinitis, gustatory rhinitis, as well as pulmonary inflammation, such as that associated with viral infections and cystic fibrosis. In addition, compounds disclosed herein are also useful in organ transplant patients either alone or in combination with conventional immunomodulators.

Moreover, compounds disclosed herein may be used in the treatment of tendonitis, bursitis, skin-related conditions such as psoriasis, allergic dermatitis, atopic dermatitis and other variants of eczema, allergic contact dermatitis, irritant contact dermatitis, seborrhoeic eczema, nummular eczematous dermatitis, autosensitization dermatitis, Lichen Simplex Chronicus, dyshidrotic dermatitis, neurodermatitis, stasis dermatitis, generalized ordinary urticaria, acute allergic urticaria, chronic allergic urticaria, autoimmune urticaria, chronic idiopathic urticaria, drug-induced urticaria, cholinergic urticaria, chronic cold urticaria, dermatographic urticaria, solar urticaria, urticaria pigmentosa, mastocytosis, acute or chronic pruritis associated with skin-localized or systemic diseases and disorders, such as pancreatitis, hepatitis, burns, sunburn, and vitiligo.

Further, the compounds disclosed herein can be used to treat respiratory diseases, including therapeutic methods of use in medicine for preventing and treating a respiratory disease or condition including: asthmatic conditions including allergen-induced asthma, exercise-induced asthma, pollution-induced asthma, cold-induced asthma, and viral-induced-asthma; chronic obstructive pulmonary diseases including chronic bronchitis with normal airflow, chronic bronchitis with airway obstruction (chronic obstructive bronchitis), emphysema, asthmatic bronchitis, and bullous disease; and other pulmonary diseases involving inflammation including bronchioectasis cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

The compounds disclosed herein are also useful in treating tissue damage in such diseases as vascular diseases, periarteritis nodosa, thyroiditis, sclerodoma, rheumatic fever, type I diabetes, neuromuscular junction disease including myasthenia gravis, white matter disease including multiple sclerosis, sarcoidosis, nephritis, nephrotic syndrome, Behcet's syndrome, polymyositis, gingivitis, periodontis, hypersensitivity, and swelling occurring after injury.

The compounds disclosed herein can be used in the treatment of otic diseases and otic allergic disorders, including eustachian tube itching.

The compounds disclosed herein can be used in the treatment of ophthalmic diseases, such as ophthalmic allergic disorders, including allergic conjunctivitis, vernal conjunctivitis, vernal keratoconjunctivitis, and giant papillary conjunctivitis, dry eye, glaucoma, glaucomatous retinopathy, diabetic retinopathy, retinal ganglion degeneration, ocular ischemia, retinitis, retinopathies, uveitis, ocular photophobia, and of inflammation and pain associated with acute injury to the eye tissue. The compounds can also be used to treat post-operative inflammation or pain as from ophthalmic surgery such as cataract surgery and refractive surgery. In preferred embodiments, the compounds of the present invention are used to treat an allergic eye disease selected from the group consisting of allergic conjunctivitis; vernal conjunctivitis; vernal keratoconjunctivitis; and giant papillary conjunctivitis.

Compounds disclosed herein are useful in treating patients with inflammatory pain such as reflex sympathetic dystrophy/causalgia (nerve injury), peripheral neuropathy (including diabetic neuropathy), and entrapment neuropathy (carpel tunnel syndrome). The compounds are also useful in the treatment of pain associated with acute herpes zoster (shingles), postherpetic neuralgia (PHN), and associated pain syndromes such as ocular pain. Pain indications include, but are not limited to, pain resulting from dermal injuries and pain-related disorders such as tactile allodynia and hyperalgesia. The pain may be somatogenic (either nociceptive or neuropathic), acute and/or chronic.

The present compounds may also be used in co-therapies, partially or completely, in place of other conventional anti-inflammatory therapies, such as together with steroids, NSAIDs, COX-2 selective inhibitors, 5-lipoxygenase inhibitors, LTB₄ antagonists and LTA₄ hydrolase inhibitors. The compounds disclosed herein may also be used to prevent tissue damage when therapeutically combined with antibacterial or antiviral agents.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein in their entireties. Where any inconsistencies arise, material literally disclosed herein controls.

### General Methods for Preparing Compounds

The following schemes can be used to practice the present invention.

The invention is further illustrated by the following examples, which may be made my methods known in the art and/or as shown below. Additionally, these compounds may be commercially available.

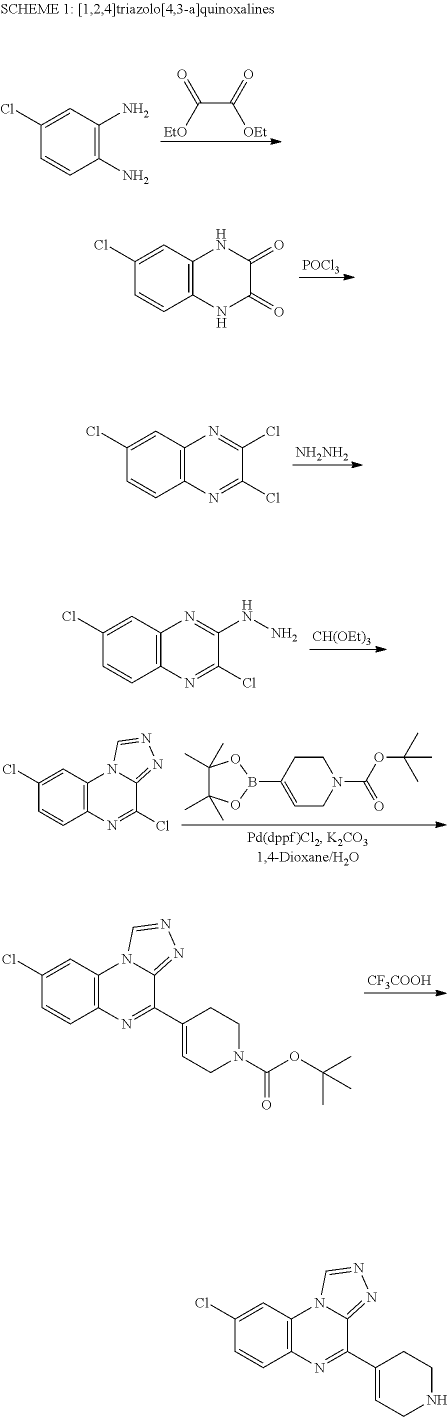

### EXAMPLE 1

### 8-chloro-4-(1,2,3,6-tetrahydropyridin-4-yl)-[1,2,4]triazolo[4,3-a]quinoxaline

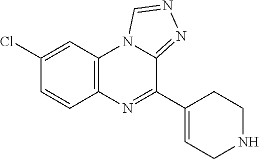

### Step 1

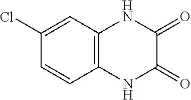

### 6-Chloroquinoxaline-2,3(1H,4H)-dione

A 100 mL round bottom flask was charged with 4-chlorobenzene-1,2-diamine (5.3 g, 37 mmol) and diethyl oxalate (31 mL). The resulting mixture was stirred overnight at reflux. Reaction progress was monitored by TLC (EtOAc/Petroleum ether=1:10). Work-up: the precipitate was collected by filtration, washed with EtOH (20 mL) and dried, to afford 7.0 g (96%) of the product as light yellow solid. ¹H NMR (300 MHz, DMSO-d₆) δ: 11.96 (br, 2H), 7.11 (m, 3H). MS m/z: 195 (M−H⁺).

### Step 2

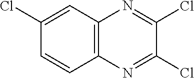

### 2,3,6-Trichloroquinoxaline

A 50 mL round bottom flask was charged with 6-chloroquinoxaline-2,3(1H,4H)-dione (7.0 g, 36 mmol) and phosphorus oxychloride (16 mL). The resulting mixture was stirred overnight at reflux. Reaction progress was monitored by TLC (EtOAc/Petroleum ether=1:10). Work-up: the reaction mixture was cooled to room temperature and cautiously poured over ice water. The solid was collected by filtration and re-dissolved in EtOAc (150 mL) then washed with brine (100 mL), dried over anhydrous Na₂SO₄, and concentrated in vacuo, to afford 7.4 g (89%) of the product as light yellow solid. ¹H NMR (300 MHz, DMSO-d₆) δ: 8.23 (d, J=2.4 Hz, 1H), 8.12 (d, J=8.7 Hz, 1H), 7.97 (dd, J=8.7, 2.4 Hz, 1H).

### Step 3

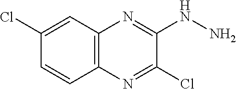

### 2,6-Dichloro-3-hydrazinylquinoxaline

A 250 mL round bottom flask was charged with 2,3,6-trichloroquinoxaline (4.6 g, 20 mmol) and EtOH (150 mL). To the above was added dropwise hydrazine hydrate (2.2 g, 44 mmol). The resulting solution was stirred overnight at room temperature. Reaction progress was monitored by TLC (EtOAc/Petroleum ether=1:2). Work-up: the resulting light yellow solid was collected by filtration, washed with water (50 mL) then ethyl acetate (50 mL), and dried, to give 1.5 g (34%) of the product as pink solid. ¹H NMR (300 MHz, DMSO-d₆) δ: 9.14 (br, 1H), 7.75 (d, J=8.7 Hz, 1H), 7.66 (s, 1H), 7.39 (d, J=8.7 Hz, 1H). MS m/z: 229 (M+H⁺).

### Step 4

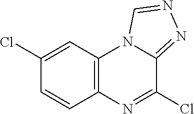

### 4,8-Dichloro-[1,2,4]triazolo[4,3-a]quinoxaline

A 50 mL round bottom flask was charged with 2,6-dichloro-3-hydrazinylquinoxaline (1.5 g, 6.6 mmol) and triethyl orthoformate (18 mL). The resulting mixture was stirred at 100° C. for 1 h. Reaction progress was monitored by TLC (EtOAc/Petroleum ether=1:2). Work-up: the resulting solid was collected by filtration, washed with MeOH (20 mL×2), and dried, to give 1.5 g (96%) of the product as light yellow powder. ¹H NMR (300 MHz, DMSO-d₆) δ: 10.20 (s, 1H), 8.70 (d, J=2.1 Hz, 1H), 8.06 (d, J=9.0 Hz, 1H), 7.78 (dd, J=9.0, 2.1 Hz, 1H). MS m/z: 239 (M+H⁺).

### Step 5

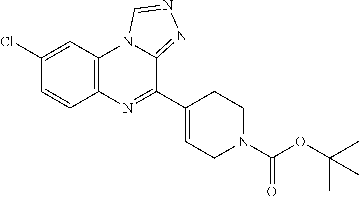

### tert-Butyl 4-(8-chloro-[1,2,4]triazolo[4,3-a]quinoxalin-4-yl)-5,6-dihydropyridine-1(2H)-carboxylate

A 250 mL 3-necked round bottom flask was charged with 4,8-dichloro-[1,2,4]triazolo[4,3-a]quinoxaline (Example 1, 1.5 g, 6.27 mmol), tert-butyl 4-(4,4,5,5-tetramethyl-1,3,2-dioxaborolan-2-yl)-5,6-dihydropyridine-1(2H)-carboxylate (2.1 g, 6.90 mmol), K₂CO₃ (2.6 g, 6.52 mmol), (1,1′-bis(diphenylphosphino)ferrocene)dichloropalladium(II) (0.51 g, 0.63 mmol), 1,4-dioxane (45 mL) and water (15 mL). The resulting mixture was stirred at 80° C. for 2 h under N₂ atmosphere. Reaction progress was monitored by TLC (EtOAc/Petroleum ether=1:1). Work-up: the reaction mixture was diluted with EtOAc (150 mL) and washed with brine (100 mL). The organic layer was dried over anhydrous Na₂SO₄ and concentrated in vacuo. The residue was further purified by flash column chromatography on silica gel with 10-40% EtOAc in petroleum ether, to afford 1.8 g (74%) of the product as light yellow crystals. ¹H NMR (300 MHz, DMSO-d₆) δ: 10.11 (s, 1H), 8.62 (d, J=2.1 Hz, 1H), 8.28 (br, 1H), 7.98 (d, J=9.0 Hz, 1H), 7.69 (dd, J=9.0, 2.1 Hz, 1H), 4.23 (d, J=9.6 Hz, 2H), 3.64-3.59 (m, 2H), 2.77 (br, 2H), 1.45 (s, 9H). MS m/z: 386 (M+H⁺).

### Step 6

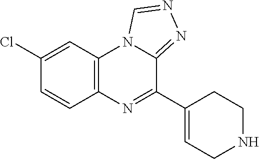

### 8-chloro-4-(1,2,3,6-tetrahydropyridin-4-yl)-[1,2,4]triazolo[4,3-a]quinoxaline

A 50 mL round bottom flask was charged with tert-butyl 4-(8-chloro-[1,2,4]triazolo[4,3-a]quinoxalin-4-yl)-5,6-dihydropyridine-1(2H)-carboxylate (1.05 g, 2.72 mmol) and CH₂Cl₂ (25 mL). To the above was added dropwise trifluoroacetic acid (2 mL) at 0° C. The resulting solution was stirred at room temperature for 4 h. Reaction progress was monitored by TLC (MeOH/CH₂Cl₂=1:10). Work-up: the reaction solution was concentrated under reduced pressure. The residue was purified by flash column chromatography on silica gel with a 1:10 MeOH/CH₂Cl₂, to afford 0.67 g (82%) of the product as light yellow crystals. ¹H NMR (300 MHz, DMSO-d₆) δ: 10.11 (s, 1H), 8.63 (d, J=2.1 Hz, 1H), 8.34 (t, J=3.3 Hz, 1H), 8.00 (d, J=8.7 Hz, 1H), 7.70 (dd, J=8.7, 2.4 Hz, 1H), 3.64 (m, 2H), 3.01 (m, 2H), 2.66 (m, 2H). MS m/z: 286 (M+H⁺).

### EXAMPLE 2

### 8-Chloro-4-(1-methyl-1,2,3,6-tetrahydropyridin-4-yl)-[1,2,4]triazolo[4,3-a]quinoxaline

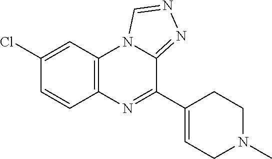

A 10 mL round bottom flask was charged with 8-chloro-4-(1,2,3,6-tetrahydropyridin-4-yl)-[1,2,4]triazolo[4,3-a]quinoxaline (140 mg, 0.489 mmol), HCHO (38%, 78 mg, 0.979 mmol), AcOH (35 mg, 0.587 mmol), CH₂Cl₂ (2 mL) and MeOH (2 mL). To the above was added NaB(OAc)₃H (160 mg, 0.734 mmol) in several batches. The resulting mixture was stirred at room temperature for 1.5 h. Reaction progress was monitored by TLC (MeOH/CH₂Cl₂=1:10). Work-up: the reaction solution was concentrated under reduced pressure. The residue was purified by flash column chromatography on silica gel with a 1:10 MeOH/CH₂Cl₂, to afford 75 mg (55%) of the product as light yellow solid. ¹H NMR (300 MHz, CDCl₃) δ: 9.24 (s, 1H), 8.46 (t, J=3.6 Hz, 1H), 8.02 (d, J=8.7 Hz, 1H), 7.92 (d, J=2.1 Hz, 1H), 7.60 (dd, J=8.7, 2.1 Hz, 1H), 3.63 (br, 2H), 3.04 (br, 4H), 2.66 (s, 3H). MS m/z: 300 (M+H⁺).

### EXAMPLE 3

### 8-Chloro-4-(piperidin-4-yl)-[1,2,4]triazolo[4,3-a]quinoxaline

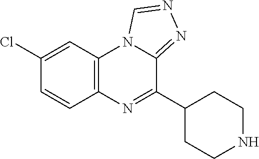

A 50 mL round bottom flask was charged with tert-butyl 4-(8-chloro-[1,2,4]triazolo[4,3-a]quinoxalin-4-yl)-5,6-dihydropyridine-1(2H)-carboxylate

(prepared as in Example 1, 80 mg, 0.21 mmol) and CH₂Cl₂ (4 mL). To the above was added dropwise trifluoroacetic acid (0.48 g, 4.14 mmol) at 0° C., followed by addition of triethylsilane (150 mg, 1.24 mmol). The resulting solution was stirred room temperature for 3 days. Reaction progress was monitored by TLC (MeOH/CH₂Cl₂=1:10). Work-up: the reaction solution was concentrated under reduced pressure. The residue was recrystallized from a 1:3 ethyl acetate/hexane, to afford 45 mg (53%) of the product as light yellow solid. ¹H NMR (300 MHz, CD₃OD) δ: 9.91 (d, J=0.9 Hz, 1H), 8.46 (t, J=2.1 Hz, 1H), 8.07 (d, J=8.7 Hz, 1H), 7.71 (m, 1H), 3.91 (m, 1H), 3.60 (m, 2H), 3.35-3.29 (m, 2H), 2.47-2.29 (m, 4H). MS m/z: 288 (M+H⁺).

### EXAMPLE 4

### 8-Chloro-4-(1-methylpiperidin-4-yl)-[1,2,4]triazolo[4,3-a]quinoxaline

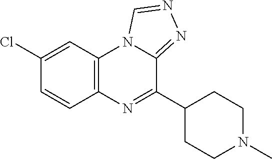

The title compound was prepared as described in Example 2, except that 8-chloro-4-(piperidin-4-yl)-[1,2,4]triazolo[4,3-a]quinoxaline was substituted for 8-chloro-4-(1,2,3,6-tetrahydropyridin-4-yl)-[1,2,4]triazolo[4,3-a]quinoxaline in step 1 of that route. ¹H NMR (300 MHz, CD₃OD) δ: 9.90 (s, 1H), 8.46 (d, J=2.1 Hz, 1H), 8.07 (d, J=8.4 Hz, 1H), 7.72 (dd, J=8.4, 2.1 Hz, 1H), 3.90 (m, 1H), 3.68 (m, 2H), 3.35 (m, 2H), 2.96 (s, 3H), 2.52-2.44 (m, 4H). MS m/z: 302 (M+H⁺).

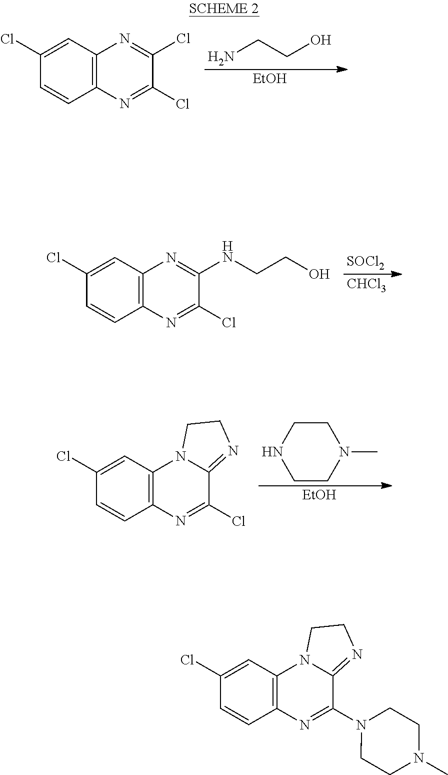

### EXAMPLE 5

### 8-Chloro-4-(4-methylpiperazin-1-yl)-1,2-dihydroimidazo[1,2-a]quinoxaline

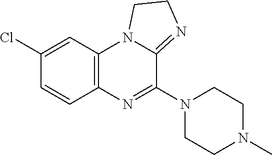

### Step 1

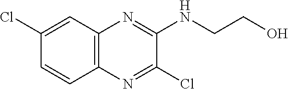

### 2-(3,7-Dichloroquinoxalin-2-ylamino)ethanol

A 250 mL 3-necked round bottom flask was charged with 2,3,6-trichloroquinoxaline (described in step 2 of Example 1, 4.46 g, 19.1 mmol) and EtOH (50 mL). To the above was added dropwise a solution of 2-aminoethanol (2.44 g, 40.1 mmol) in EtOH (20 mL) with the temperature maintained below 35° C. The resulting mixture was stirred at room temperature for 4 h and then cooled to 0° C. The precipitate was collected by filtration, washed with a 1:1 n-hexane/EtOAc and dried, to afford 4.0 g (81%) of the product.

### Step 2

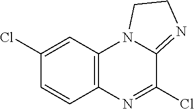

### 4,8-Dichloro-1,2-dihydroimidazo[1,2-a]quinoxaline

A 100 mL round bottom flask was charged with 2-(3,7-dichloroquinoxalin-2-ylamino)ethanol (4.0 g, 15.5 mmol), SOCl₂ (20 mL) and CHCl₃ (20 mL). The resulting solution was heated at reflux for 2 h then concentrated in vacuo. The residue was co-evaporated several times with CHCl₃ then EtOAc. The crude product thus obtained was washed with EtOAc to afford 2.4 g (65%) of the product.

### Step 3

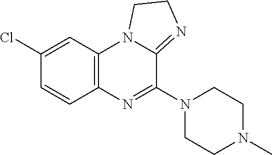

### 8-Chloro-4-(4-methylpiperazin-1-yl)-1,2-dihydroimidazo[1,2-a]quinoxaline

A 50 mL round bottom flask was charged with 4,8-dichloro-1,2-dihydroimidazo[1,2-a]quinoxaline (500 mg, 2.1 mmol), N-methylpiperazine (700 mg, 7.0 mmol) and EtOH (3 mL). The resulting solution was heated at reflux for 16 h then concentrated in vacuo. The residue was purified by flash column chromatography on silica gel to afford 500 mg (79%) of the product. ¹H NMR (300 MHz, CDCl₃) δ: 7.24 (d, J=8.4 Hz, 1H), 6.93 (dd, J=8.4, 2.4 Hz, 1H), 6.64 (d, J=2.4 Hz, 1H), 4.21-4.09 (m, 6H), 3.92 (m, 2H), 2.54 (m, 4H), 2.34 (s, 3H). MS m/z: 304 (M+H⁺).

### EXAMPLE 6

### 8-Chloro-4-(piperazin-1-yl)-1,2-dihydroimidazo[1,2-a]quinoxaline

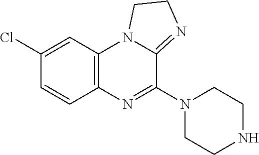

The title compound was prepared as described in Example 5, except that piperazine was substituted for N-methylpiperazine in step 3 of that route. ¹H NMR (300 MHz, CD₃OD/D₂O) δ: 7.82 (d, J=8.7 Hz, 1H), 7.62 (d, J=2.1 Hz, 1H), 7.56 (dd, J=8.7, 2.1 Hz, 1H), 4.81 (m, 2H), 4.32 (m, 2H), 3.79 (m, 4H), 3.47 (m, 4H). MS m/z: 290 (M+H⁺).

### EXAMPLE 7

### 8-Chloro-4-(4-methylpiperazin-1-yl)imidazo[1,2-a]quinoxaline

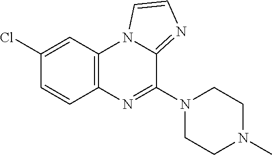

A 250 mL round bottom flask was charged with 8-chloro-4-(4-methylpiperazin-1-yl)-1,2-dihydroimidazo[1,2-a]quinoxaline (Example 5, 300 mg, 0.99 mmol), chloranil (1 g, 4 mmol) and xylene (100 mL). The resulting solution was heated at reflux for 16 h then cooled to room temperature. The reaction mixture was washed several times with diluted aqueous NaOH solution until the aqueous phase became colorless. The organic layer was concentrated under reduced pressure and the residue was purified by flash column chromatography on silica gel to afford 220 mg (74%) of the product. ¹H NMR (300 MHz, CDCl₃) δ: 7.90 (d, J=1.2 Hz, 1H), 7.66 (d, J=2.1 Hz, 1H), 7.62-7.58 (m, 2H), 7.34 (dd, J=8.7, 2.1 Hz, 1H), 4.42 (m, 4H), 2.61 (m, 4H), 2.37 (s, 3H). MS m/z: 302 (M+H⁺).

### EXAMPLE 8

### 8-Chloro-4-(piperazin-1-yl)imidazo[1,2-a]quinoxaline

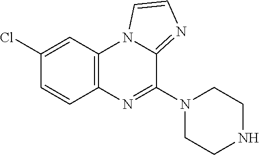

The title compound was prepared as described in Example 7, except that 8-chloro-4-(piperazin-1-yl)-1,2-dihydroimidazo[1,2-a]quinoxaline (Example 7) was substituted for 8-chloro-4-(4-methylpiperazin-1-yl)-1,2-dihydroimidazo[1,2-a]quinoxaline (Example 5) in step 1 of that route. ¹H NMR (300 MHz, D₂O) δ: 8.01 (d, J=1.2 Hz, 1H), 7.58 (m, 2H), 7.32 (d, J=9.0 Hz, 1H), 7.22 (d, J=1.8 Hz, 1H), 4.24 (m, 4H), 3.41 (m, 4H). MS m/z: 288 (M+H⁺).

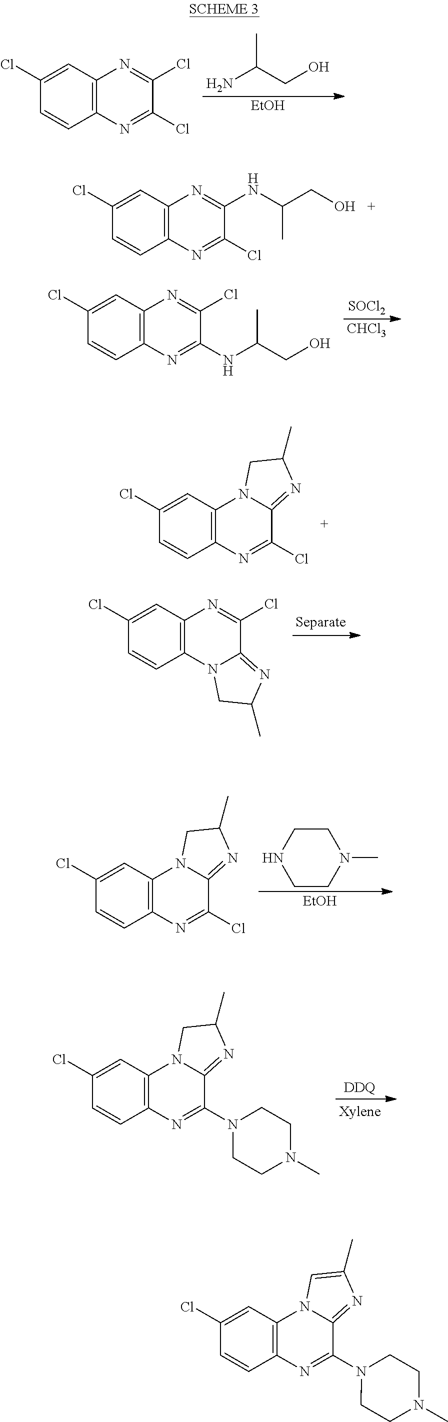

### EXAMPLE 9

### 8-Chloro-2-methyl-4-(4-methylpiperazin-1-yl)imidazo[1,2-a]quinoxaline

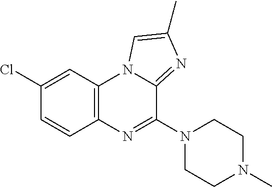

### Step 1

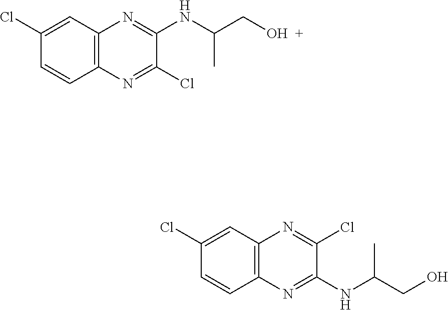

### Mixture of 2-(3,7-dichloroquinoxalin-2-ylamino)propan-1-ol and 2-(3,6-dichloroquinoxalin-2-ylamino)propan-1-ol

A 500 mL 3-necked round bottom flask was charged with 2,3,6-trichloroquinoxaline (described in step 2 of Example 1, 5.0 g, 21.4 mmol) and EtOH (100 mL). To the above was added dropwise a solution of 2-aminopropan-1-ol (3.7 mL, 47.5 mmol) in EtOH (50 mL). The resulting solution was heated at reflux for 4 h then concentrated under reduced pressure. The residue was purified by flash column chromatography on silica gel with 20% EtOAc in petroleum ether, to afford 2.5 g (54%) of the product as a mixture of two isomers.

### Step 2

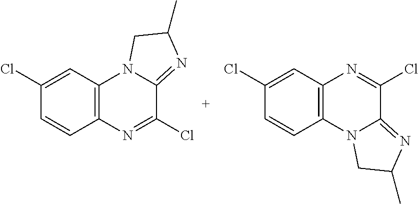

### 4,8-Dichloro-2-methyl-1,2-dihydroimidazo[1,2-a]quinoxaline and 4,7-dichloro-2-methyl-1,2-dihydroimidazo [1,2-a]quinoxaline

A 50 mL round bottom flask was charged with the mixture of 2-(3,7-dichloroquinoxalin-2-ylamino)propan-1-ol and 2-(3,6-dichloroquinoxalin-2-ylamino)propan-1-ol (1.8 g, 6.6 mmol), SOCl₂ (10 mL) and CHCl₃ (10 mL). The resulting solution was heated at reflux for 2 h then concentrated in vacuo. The residue was poured into saturated aqueous Na₂CO₃ and extracted with CH₂Cl₂. The combined organic layers were dried over Na₂SO₄ and concentrated in vacuo. The residue was purified by flash column chromatography on silica gel with 2% EtOAc in petroleum ether to afford 1.08 g (64%) of 4,8-dichloro-2-methyl-1,2-dihydroimidazo[1,2-a]quinoxaline (¹H NMR (300 MHz, CDCl₃) δ: 8.15 (d, J=8.7 Hz, 1H), 7.00 (dd, J=8.4, 2.1 Hz, 1H), 6.68 (d, J=2.4 Hz, 1H), 4.50 (m, 1H), 4.16 (m, 1H), 3.60 (m, 1H), 1.44 (d, J=6.6 Hz, 3H)), and 270 mg (0.16%) of 4,7-dichloro-2-methyl-1,2-dihydroimidazo[1,2-a]quinoxaline (¹H NMR (300 MHz, CDCl₃) δ: 7.57 (d, J=1.8 Hz, 1H), 7.33 (dd, J=8.4, 2.1 Hz, 1H), 6.65 (d, J=8.4 Hz, 1H), 4.52 (m, 1H), 4.20 (m, 1H), 3.62 (m, 1H), 1.45 (d, J=6.9 Hz, 3H)) as yellow solids.

### Step 3

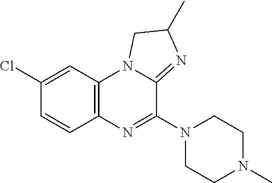

### 8-Chloro-2-methyl-4-(4-methylpiperazin-1-yl)-1,2-dihydroimidazo[1,2-a]quinoxaline

A 50 mL round bottom flask was charged with 4,8-dichloro-2-methyl-1,2-dihydroimidazo[1,2-a]quinoxaline (300 mg, 1.2 mmol), N-methylpiperazine (0.16 mL, 1.4 mmol), Et₃N (0.35 mL, 2.5 mmol) and anhydrous EtOH (20 mL). The resulting solution was heated at reflux for 2 h then concentrated in vacuo. The residue was dissolved in CH₂Cl₂, washed with brine, dried over MgSO₄, and concentrated in vacuo, to afford 360 mg (96%) of the product as yellow oil. ¹H NMR (300 MHz, DMSO-d₆) δ: 7.15 (d, J=8.7 Hz, 1H), 6.92 (dd, J=8.4, 2.4 Hz, 1H), 6.85 (d, J=2.1 Hz, 1H), 4.77 (m, 1H), 4.35 (m, 1H), 3.99 (m, 4H), 3.49 (m, 1H), 2.37 (m, 4H), 2.19 (s, 3H), 1.28 (d, J=6.0 Hz, 1H). MS m/z: 317 (M+H⁺).

### Step 4

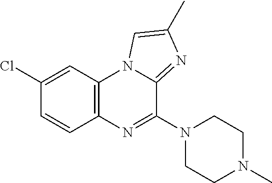

### 8-Chloro-2-methyl-4-(4-methylpiperazin-1-yl)imidazo[1,2-a]quinoxaline

A 50 mL round bottom flask was charged with 8-chloro-2-methyl-4-(4-methylpiperazin-1-yl)-1,2-dihydroimidazo[1,2-a]quinoxaline (360 mg, 1.13 mmol), 2,3-dichloro-5,6-dicyano-p-benzoquinone (515 mg, 2.26 mmol) and xylene (10 mL). The resulting solution was heated at reflux for 3 h then concentrated in vacuo. The residue was dissolved in 1 M aqueous NaOH (10 mL) and extracted with CH₂Cl₂. The organic layer was dried over anhydrous MgSO₄ and concentrated in vacuo. The residue was purified by flash column chromatography on silica gel with 3% MeOH in CH₂Cl₂, to afford 95 mg (26%) of the product as white solid. ¹H NMR (300 MHz, CDCl₃) δ: 7.60 (m, 3H), 7.31 (dd, J=8.7, 2.4 Hz, 1H), 4.40 (br, 4H), 2.62 (m, 4H), 2.46 (d, J=0.6 Hz, 3H), 2.38 (s, 3H). MS m/z: 315 (M+H⁺).

### EXAMPLE 10

### 8-Chloro-2-methyl-4-(piperazin-1-yl)imidazo[1,2-a]quinoxaline

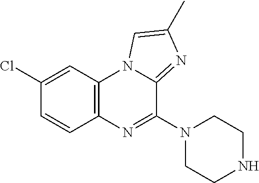

The title compound was prepared as described in Example 9, except that piperazine was substituted for N-methylpiperazine in step 3 of that route. ¹H NMR (300 MHz, CD₃OD) δ: 8.17 (s, 1H), 8.05 (d, J=2.1 Hz, 1H), 7.65 (d, J=8.7 Hz, 1H), 7.41 (dd, J=8.7, 2.4 Hz, 1H), 4.51 (t, J=5.4 Hz, 4H), 3.40 (t, J=5.4 Hz, 4H), 2.45 (s, 3H). MS m/z: 301 (M+H⁺).

### EXAMPLE 11

### 7-Chloro-2-methyl-4-(4-methylpiperazin-1-yl)imidazo[1,2-a]quinoxaline

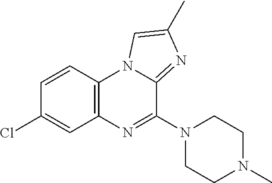

The title compound was prepared as described in Example 9, except that 4,7-dichloro-2-methyl-1,2-dihydroimidazo[1,2-a]quinoxaline was substituted for 4,8-dichloro-2-methyl-1,2-dihydroimidazo[1,2-a]quinoxaline in step 3 of that route. ¹H NMR (300 MHz, CD₃OD) δ: 8.06 (s, 1H), 7.85 (d, J=8.4 Hz, 1H), 7.59 (d, J=2.4 Hz, 1H), 7.26 (dd, J=9.0, 2.4 Hz, 1H), 4.33 (m, 4H), 2.62 (t, J=5.4 Hz, 4H), 2.43 (s, 3H), 2.35 (s, 3H). MS m/z: 315 (M+H⁺).

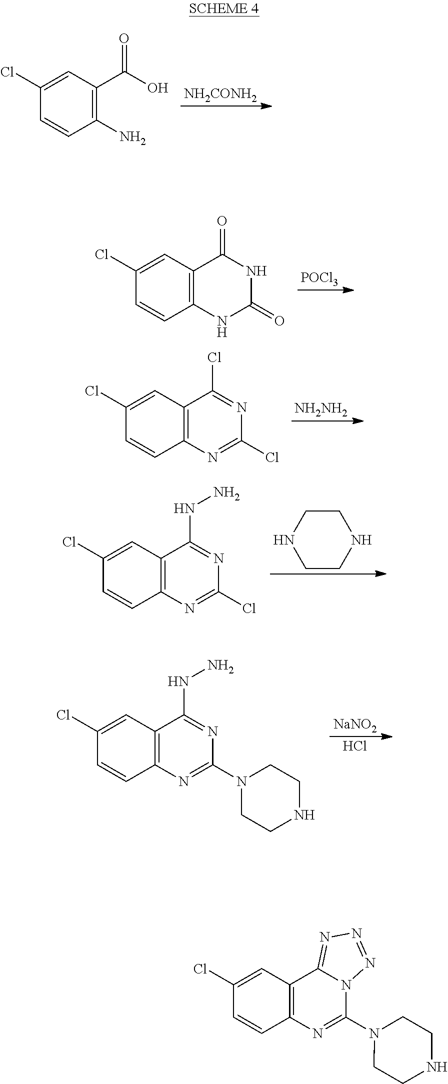

### EXAMPLE 12

### 9-Chloro-5-(piperazin-1-yl)tetrazolo[1,5-c]quinazoline

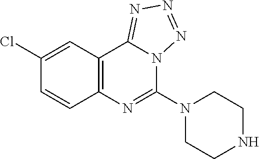

### Step 1

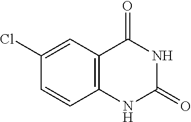

### 6-Chloroquinazoline-2,4(1H,3H)-dione

A 250 mL round bottom flask was charged with 2-amino-5-chlorobenzoic acid (17.2 g, 0.1 mol) and urea (30 g, 0.5 mol). The resulting mixture was heated to 200° C. for 3 h. Work up: the reaction mixture was washed by water and filtered. The solid was dried to give 18.5 g (94%) of the product. MS m/z: 196 (M+H⁺).

### Step 2

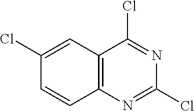

### 2,4,6-Trichloroquinazoline

The title compound was prepared as described in Example 1, except that 6-chloroquinazoline-2,4(1H,3H)-dione was substituted for 6-chloroquinoxaline-2,3(1H,4H)-dione in step 2 of that route. ¹H NMR (300 MHz, CDCl₃) δ: 8.24 (d, J=2.1 Hz, 1H), 7.99-7.90 (m, 2H).

### Step 3

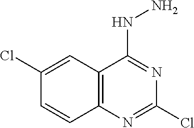

### 2,6-Dichloro-4-hydrazinylquinazoline

A 100 mL round bottom flask was charged with 2,4,6-trichloroquinazoline (1 g, 4.3 mmol) and ethanol (50 mL). To the above was added dropwise hydrazine hydrate (0.492 g, 9.8 mmol) at 0-5° C. The resulting mixture was stirred for 0.5 h below 10° C. then 2 h at room temperature. Reaction progress was monitored by TLC (EtOAc/Petroleum ether=1:4, Rf=0.3). Work-up: the resulting solid was collected by filtration, washed with ethanol and dried, to give 0.94 g (96%) of the product. ¹H NMR (300 MHz, DMSO-d₆) δ: 8.34 (s, 1H), 7.76 (m, 1H), 7.58 (m, 1H). MS m/z: 229 (M+H⁺).

### Step 4

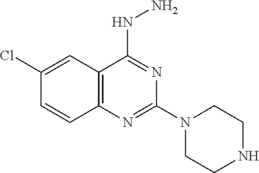

### 6-Chloro-4-hydrazinyl-2-(piperazin-1-yl)quinazoline

A 250 mL round bottom flask was charged with 2,6-dichloro-4-hydrazinylquinazoline (1 g, 4.4 mmol), piperazine (1.13 g, 13.1 mmol) and absolute ethanol (100 mL). The resulting mixture was heated at reflux for 8 h. Work-up: the reaction mixture was concentrated under reduced pressure. The resulting solid was collected by filtration, washed with ethanol and dried, to give 0.9 g (74%) of the product. MS m/z: 279 (M+H⁺).

### Step 5

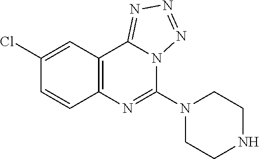

### 9-Chloro-5-(piperazin-1-yl)tetrazolo[1,5-c]quinazoline

A 250 mL round bottom flask was charged with 6-chloro-4-hydrazinyl-2-(piperazin-1-yl)quinazoline (1.6 g, 5.75 mmol) and 0.2 M HCl (80 mL). To the above was added dropwise a solution of NaNO₂ (0.6 g, 8.62 mmol) in water (2 mL) at 0-5° C. The resulting mixture was stirred at 5° C. for 1 h. Work up: the reaction mixture was washed with ethyl acetate (50 mL×3). The aqueous layer was basified to PH 8 by saturated aqueous Na₂CO₃. The precipitate was collected by filtration, washed with water and dried, to give 670 mg (40%) of the product. ¹H NMR (300 MHz, DMSO-d₆) δ: 8.36 (d, J=2.4 Hz, 1H), 7.84 (dd, J=9.0, 2.4 Hz, 1H), 7.72 (d, J=9.0 Hz, 1H), 3.98 (m, 4H), 2.92 (m, 4H). MS m/z: 290 (M+H⁺).

### EXAMPLE 13

### 9-chloro-5-(4-methylpiperazin-1-yl)tetrazolo[1,5-c]quinazoline

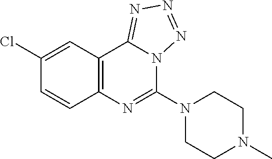

The title compound was prepared as described in Example 12, except that N-methylpiperazine was substituted for piperazine in step 4 of that route. MS m/z: 304 (M+H⁺).

### EXAMPLE 14

### 9-chloro-5-(4-methylpiperazin-1-yl)-[1,2,4]triazolo[4,3-c]quinazoline

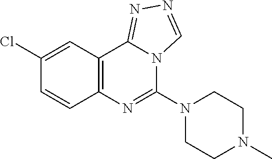

The title compound was prepared as described in Collection of Czechoslovak Chemical Communications (1984), 49(8), 1795-9, using 6-chloro-4-hydrazinyl-2-(4-methylpiperazin-1-yl)quinazoline described in step 3 of Example 12. MS m/z: 303 (M+H⁺).

### EXAMPLE 15

### 8-methyl-4-(4-methylpiperazin-1-yl)tetrazolo[1,5-a]quinoxaline

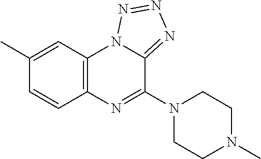

The title compound was obtained from a commercial source.

### EXAMPLE 16

### 7-Chloro-4-(piperazin-1-yl)tetrazolo[1,5-a]quinoxaline

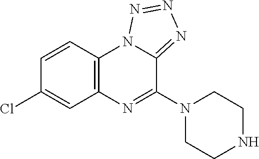

### Step 1

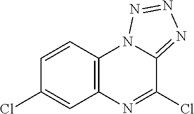

### 4,7-Dichlorotetrazolo[1,5-a]quinoxaline

A 100 mL round bottom flask was charged with 2,3,6-trichloroquinoxaline (described in step 2 of Example 1, 1.0 g, 4.27 mmol), NaN₃ (2.5 g, 38.46 mmol) and EtOH (50 mL). The resulting mixture was stirred at 60° C. overnight. Reaction progress was monitored by TLC (EtOAc/Petroleum ether=1:10). Work-up: the reaction mixture was concentrated under reduced pressure. The residue was mixed with water (30 mL) and extracted with EtOAc (50 and 20 mL). The combined organic layers were washed with brine (50 mL), dried over anhydrous Na₂SO₄, concentrated in vacuo, to afford 1.0 g (quantitative) of the product as yellow amorphous powder. ¹H NMR (300 MHz, CDCl₃) δ: 8.76 (d, J=2.1 Hz, 1H), 8.70 (d, J=9.0 Hz, 1H), 7.96 (dd, J=9.0, 2.1 Hz, 1H).

### Step 2

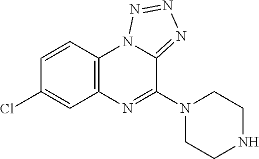

### 7-Chloro-4-(piperazin-1-yl)tetrazolo[1,5-a]quinoxaline

A 5 mL microwave reaction tube was charged with 4,7-dichlorotetrazolo[1,5-a]quinoxaline (0.27 g, 1.13 mmol), piperazine (0.15 g, 1.69 mmol), Cs₂CO₃ (1.14 g, 3.39 mmol) and DMF (4 mL). The resulting mixture was heated at 140° C. for 1 h in a Biotage microwave reactor. Work-up: the reaction mixture was diluted with EtOAc (30 mL) and washed with H₂O (30 mL). The organic layer was dried over anhydrous MgSO₄ and concentrated in vacuo. The residue was purified by flash column chromatography on silica gel with 5-10% MeOH in CH₂Cl₂ to provide 0.25 g of yellow solid. It was further purified by recrystallization from EtOAc, to afford 120 mg (37%) of the product as light yellow solid. ¹H NMR (300 MHz, CD₃OD) δ: 8.29 (d, J=8.7 Hz, 1H), 7.71 (d, J=2.4 Hz, 1H), 7.43 (dd, J=8.7, 2.4 Hz, 1H), 4.37 (br, 4H), 3.02 (m, 4H). MS m/z: 290 (M+H⁺).

### EXAMPLE 17

### 7-Chloro-4-(4-methylpiperazin-1-yl)tetrazolo[1,5-a]quinoxaline

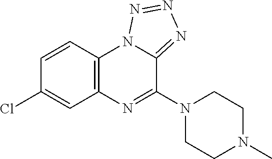

The title compound was prepared as described in Example 16, except that N-methylpiperazine was substituted for piperazine in step 2 of that route. ¹H NMR (300 MHz, CDCl₃) δ: 8.29 (d, J=8.7 Hz, 1H), 7.76 (d, J=2.1 Hz, 1H), 7.38 (dd, J=8.7, 2.1 Hz, 1H), 4.50 (br, 4H), 2.61 (t, J=5.1 Hz, 4H), 2.34 (s, 3H). MS m/z: 304 (M+H⁺).

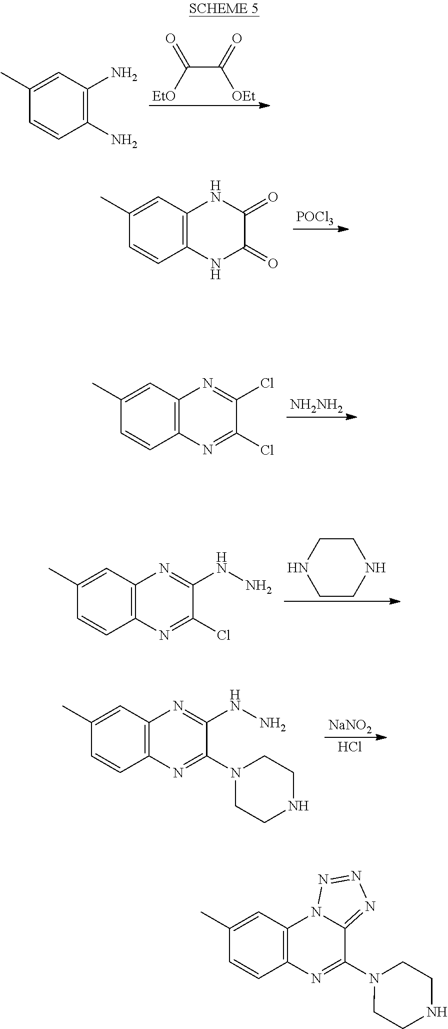

### EXAMPLE 18

### 8-Methyl-4-(piperazin-1-yl)tetrazolo[1,5-a]quinoxaline

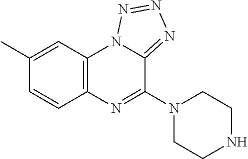

### Step 1

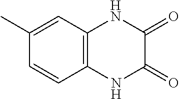

### 6-Methylquinoxaline-2,3(1H,4H)-dione

A 250 mL round bottom flask was charged with 4-methylbenzene-1,2-diamine (9.76 g, 0.08 mol) and diethyl oxalate (86 mL, 0.64 mol). The resulting mixture was heated at 140° C. overnight. Work-up: the reaction mixture was filtered and the solid was washed with ethanol and dried to give 13 g (92%) of the product. MS m/z: 175 (M+H⁺).

### Step 2

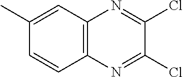

### 2,3-Dichloro-6-methylquinoxaline

The title compound was prepared as described in Example 1, except that 6-methylquinoxaline-2,3(1H,4H)-dione was substituted for 6-chloroquinoxaline-2,3(1H,4H)-dione in step 2 of that route. ¹H NMR (300 MHz, CDCl₃) δ: 7.92 (m, 1H), 7.79 (s, 1H), 7.54 (m, 1H), 2.59 (s, 3H).

### Step 3

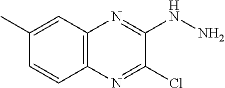

### 2-Chloro-3-hydrazinyl-6-methylquinoxaline

The title compound was prepared as described in Example 12, except that 2,3-dichloro-6-methylquinoxaline was substituted for 2,4,6-trichloroquinazoline in step 3 of that route. MS m/z: 209 (M+H⁺).

### Step 4

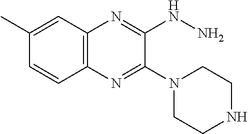

### 3-Hydrazinyl-6-methyl-2-(piperazin-1-yl)quinoxaline

The title compound was prepared as described in Example 12, except that 2-chloro-3-hydrazinyl-6-methylquinoxaline was substituted for 2,6-dichloro-4-hydrazinylquinazoline in step 4 of that route. MS m/z: 259 (M+H⁺).

### Step 5

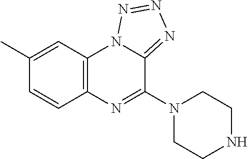

### 8-Methyl-4-(piperazin-1-yl)tetrazolo[1,5-a]quinoxaline

The title compound was prepared as described in Example 12, except that 3-hydrazinyl-6-methyl-2-(piperazin-1-yl)quinoxaline was substituted for 6-chloro-4-hydrazinyl-2-(piperazin-1-yl)quinazoline in step 5 of that route. ¹H NMR (300 MHz, CD₃OD) δ: 8.04 (s, 1H), 7.55 (d, J=8.7 Hz, 1H), 7.38 (m, 1H), 4.28 (m, 4H), 3.03 (m, 4H), 2.50 (s, 3H). MS m/z: 270 (M+H⁺).

### EXAMPLE 19

### 8-Chloro-4-(piperazin-1-yl)tetrazolo[1,5-a]quinoxaline

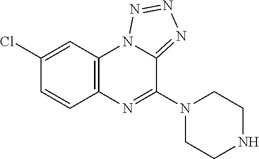

### Step 1

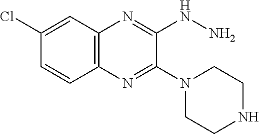

### 6-Chloro-3-hydrazinyl-2-(piperazin-1-yl)quinoxaline

The title compound was prepared as described in Example 12, except that 2,6-dichloro-3-hydrazinylquinoxaline (prepared in Example 1) was substituted for 2,6-dichloro-4-hydrazinylquinazoline in step 4 of that route. MS m/z: 279 (M+H⁺).

### Step 2

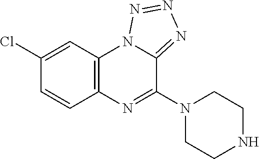

### 8-Chloro-4-(piperazin-1-yl)tetrazolo[1,5-a]quinoxaline

The title compound was prepared as described in Example 12, except that 6-chloro-3-hydrazinyl-2-(piperazin-1-yl)quinoxaline was substituted for 6-chloro-4-hydrazinyl-2-(piperazin-1-yl)quinazoline in step 5 of that route. ¹H NMR (300 MHz, CD₃OD) δ: 8.42 (d, J=2.4 Hz, 1H), 7.80 (d, J=9.0 Hz, 1H), 7.68 (dd, J=9.0, 2.4 Hz, 1H), 4.64 (m, 4H), 3.46 (m, 4H). MS m/z: 290 (M+H⁺).

### EXAMPLE 20

### 8-Chloro-4-(4-methylpiperazin-1-yl)tetrazolo[1,5-a]quinoxaline

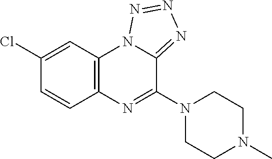

The title compound was prepared as described in Example 19, except that N-methylpiperazine was substituted for piperazine in step 1 of that route. ¹H NMR (300 MHz, CDCl₃) δ: 8.37 (d, J=2.7 Hz, 1H), 7.68 (d, J=8.7 Hz, 1H), 7.55 (dd, J=8.7, 2.4 Hz, 1H), 4.43 (br, 4H), 2.62 (m, 4H), 2.38 (s, 3H). MS m/z: 304 (M+H⁺).

### EXAMPLE 21

### 8-Methyl-4-(4-methylpiperazin-1-yl)-[1,2,4]triazolo[4,3-a]quinoxaline

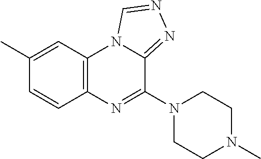

### Step 1

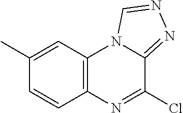

### 4-Chloro-8-methyl-[1,2,4]triazolo[4,3-a]quinoxaline

A 100 mL round bottom flask was charged with 2-chloro-3-hydrazinyl-6-methylquinoxaline (prepared in Example 18 step 1-3, 2.39 g, 11.4 mmol) and trimethyl orthoformate (40 mL). The resulting mixture was heated at reflux for 1.5 h. Work-up: the reaction mixture was filtered and the solid was washed with ethanol and dried to give 1.55 g (62%) of the product. MS m/z: 219 (M+H⁺).

### Step 2

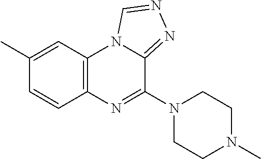

### 8-Methyl-4-(4-methylpiperazin-1-yl)-[1,2,4]triazolo[4,3-a]quinoxaline

The title compound was prepared as described in Example 12, except that 4-chloro-8-methyl-[1,2,4]triazolo[4,3-a]quinoxaline was substituted for 2,6-dichloro-4-hydrazinylquinazoline, and N-methylpiperazine for piperazine in step 4 of that route. ¹H NMR (300 MHz, CDCl₃) δ: 9.15 (s, 1H), 7.56 (m, 2H), 7.28 (m, 1H), 4.42 (br, 4H), 2.59 (m, 4H), 2.48 (s, 3H), 2.35 (s, 3H). MS m/z: 283 (M+H⁺).

### EXAMPLE 22

### 8-Methyl-4-(piperazin-1-yl)-[1,2,4]triazolo[4,3-a]quinoxaline

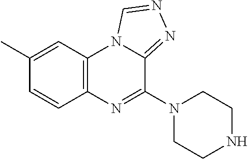

The title compound was prepared as described in Example 21, except that piperazine was substituted for N-methylpiperazine in step 2 of that route. ¹H NMR (300 MHz, CDCl₃) δ: 9.14 (s, 1H), 7.55 (m, 2H), 7.29 (m, 1H), 4.41 (br, 4H), 3.10 (m, 4H), 2.50 (s, 3H). MS m/z: 269 (M+H⁺).

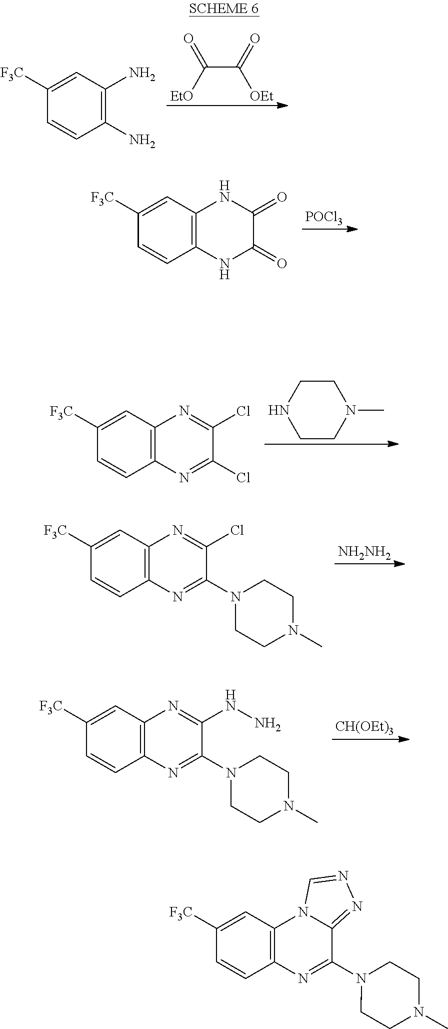

### EXAMPLE 23

### 4-(4-methylpiperazin-1-yl)-8-(trifluoromethyl)-[1,2,4]triazolo[4,3-a]quinoxaline

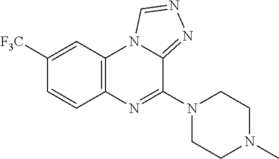

### Step 1

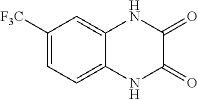

### 6-(Trifluoromethyl)-1,4-dihydroquinoxaline-2,3-dione

A 100 mL round bottom flask was charged with 4-(trifluoromethyl)benzene-1,2-diamine (5.3 g, 37 mmol) and diethyl oxalate (31 mL). The resulting mixture was stirred overnight at reflux. Reaction progress was monitored by TLC (EtOAc/Petroleum ether=1:10). Work-up: the precipitate was collected by filtration, washed with EtOH (20 mL) and dried, to afford 7.0 g (96%) of the product as light yellow solid.

### Step 2

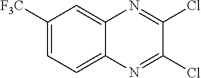

### 2,3-Dichloro-6-(trifluoromethyl)quinoxaline

A 100 mL round bottom flask was charged with 6-(trifluoromethyl)-1,4-dihydroquinoxaline-2,3-dione (7.0 g, 36 mmol) and phosphorus oxychloride (16 mL). The resulting mixture was stirred overnight at reflux. Reaction progress was monitored by TLC (EtOAc/Petroleum ether=1:10). Work-up: the reaction mixture was cooled to room temperature and cautiously poured into ice water. The solid was collected by filtration and re-dissolved in EtOAc (150 mL) then washed with brine (100 mL), dried over anhydrous Na₂SO₄, and concentrated in vacuo, to afford 7.4 g (89%) of the product as light yellow solid.

### Step 3

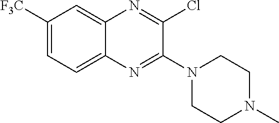

### 3-Chloro-2-(4-methylpiperazinyl)-6-(trifluoromethyl)quinoxaline

A 250 mL round bottom flask was charged with 2,3-dichloro-6-(trifluoromethyl)quinoxaline (4.6 g, 17.2 mmol) and EtOH (50 mL). To the above was added dropwise N-methylpiperazine (1.7 g, 17.2 mmol). The resulting solution was stirred overnight at room temperature. Reaction progress was monitored by TLC (EtOAc/Petroleum ether=1:2). Work-up: the reaction mixture was concentrated in vacuo. The residue was re-dissolved in EtOAc (50 mL) and washed with brine (20 mL). The organic layer was dried over anhydrous Na₂SO₄ and then concentrated in vacuo. The residue was further purified by flash column chromatography on silica gel with 10-20% EtOAc in petroleum ether, to afford 3.0 g (52%) of the product as white solid. MS m/z: 331 (M+H⁺).

### Step 4

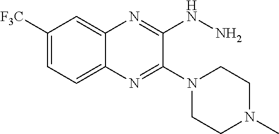

### 3-hydrazinyl-2-(4-methylpiperazin-1-yl)-6-(trifluoromethyl)quinoxaline

A 100 mL round bottom flask was charged with 3-chloro-2-(4-methylpiperazinyl)-6-(trifluoromethyl)quinoxaline (3.0 g, 9.1 mmol), hydrazine hydrate (9.0 g, 182 mmol) and EtOH (50 mL). The resulting solution was refluxed for 0.5 h. Work-up: the reaction mixture was concentrated in vacuo. The residue was re-dissolved in CH₂Cl₂ (50 mL) and washed with brine (20 mL). The organic layer was dried over anhydrous Na₂SO₄ and then concentrated in vacuo. The residue was further purified by flash column chromatography on silica gel with a 1:10 MeOH/CH₂Cl₂, to afford 1.5 g (50%) of the product as light yellow crystals. MS m/z: 327 (M+H⁺).

### Step 5

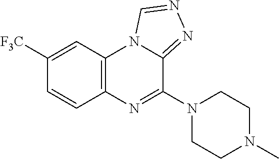

### 4-(4-methylpiperazin-1-yl)-8-(trifluoromethyl)-[1,2,4]triazolo[4,3-a]quinoxaline

A 100 mL round bottom flask was charged with 3-hydrazinyl-2-(4-methylpiperazin-1-yl)-6-(trifluoromethyl)quinoxaline (1.3 g, 3.9 mmol) and triethyl orthoformate (20 mL). The resulting mixture was stirred at 100° C. for 1 h. Reaction progress was monitored by TLC (EtOAc/Petroleum ether=2:1). Work-up: the reaction mixture was concentrated in vacuo. The residue was re-dissolved in EtOAc (50 mL) and washed with brine (20 mL). The organic layer was dried over anhydrous Na₂SO₄ and then concentrated in vacuo. The residue was further purified by flash column chromatography on silica gel with 10-40% EtOAc in petroleum ether, to afford 0.7 g (54%) of the product as white solid. ¹H NMR (300 MHz, CD₃OD) δ: 9.91 (s, 1H), 8.45 (s, 1H), 7.73 (m, 2H), 4.49 (m, 4H), 2.69 (m, 4H), 2.39 (s, 3H). MS m/z: 337 (M+H⁺).

### EXAMPLE 24

### 4-(piperazin-1-yl)-8-(trifluoromethyl)-[1,2,4]triazolo[4,3-a]quinoxaline

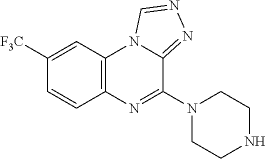

The title compound was prepared as described in Example 23, except that piperazine was substituted for N-methylpiperazine in step 3 of that route. ¹H NMR (300 MHz, CD₃OD) δ: 10.10 (s, 1H), 8.57 (s, 1H), 7.82 (m, 2H), 4.73 (m, 4H), 3.46 (m, 4H). MS m/z: 323 (M+H⁺).

### EXAMPLE 25

### 4-(4-methylpiperazin-1-yl)-7-(trifluoromethyl)-[1,2,4]triazolo[4,3-a]quinoxaline

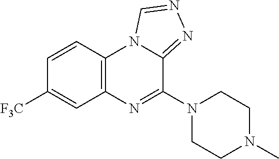

The title compound was prepared as described in Examples 50 and 21, except that 4-(trifluoromethyl)benzene-1,2-diamine was substituted for 4-methylbenzene-1,2-diamine as the starting material of that route. ¹H NMR (300 MHz, CD₃OD) δ: 9.81 (s, 1H), 8.20 (d, J=8.7 Hz, 1H), 7.53 (d, J=2.1 Hz, 1H), 7.56 (dd, J=8.7, 2.1 Hz, 1H), 4.46 (m, 4H), 2.67 (m, 4H), 2.37 (s, 3H). MS m/z: 337 (M+H⁺).

### EXAMPLE 26

### 4-(piperazin-1-yl)-7-(trifluoromethyl)-[1,2,4]triazolo[4,3-a]quinoxaline

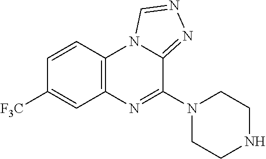

The title compound was prepared as described in Example 25, except that piperazine was substituted for N-methylpiperazine in the last step of that route. ¹H NMR (300 MHz, CD₃OD) δ: 9.87 (s, 1H), 8.25 (d, J=8.1 Hz, 1H), 7.94 (d, J=1.5 Hz, 1H), 7.64 (dd, J=8.7, 1.8 Hz, 1H), 4.60 (m, 4H), 2.67 (m, 4H). MS m/z: 323 (M+H⁺).

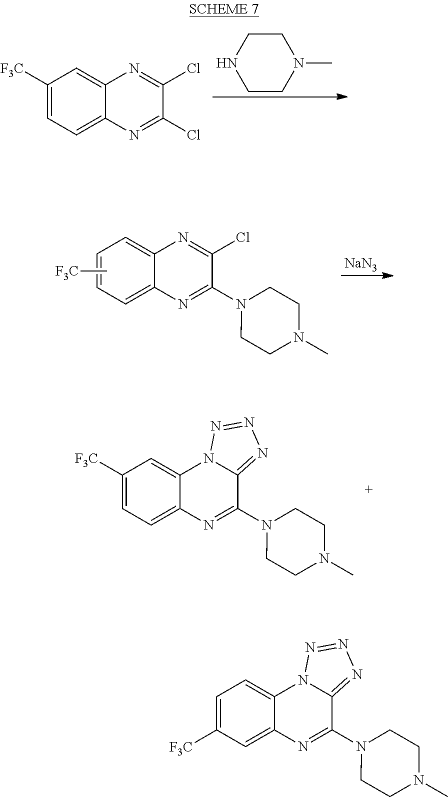

### EXAMPLE 27

### 4-(4-methylpiperazin-1-yl)-8-(trifluoromethyl)tetrazolo[1,5-a]quinoxaline

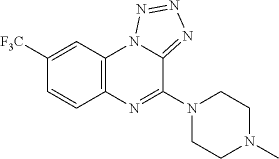

The title compound was prepared as described in Examples 23 and 16, except that 3-chloro-2-(4-methylpiperazinyl)-6-(trifluoromethyl)quinoxaline (prepared as described in Example 90 step 3) was substituted for 2,3,6-trichloroquinoxaline in step 1 of Example 16. ¹H NMR (300 MHz, CD₃OD) δ: 8.62 (s, 1H), 7.88 (m, 2H), 4.49-4.46 (m, 4H), 2.68 (t, J=5.1 Hz, 4H), 2.38 (s, 3H). MS m/z: 338 (M+H⁺).

### EXAMPLE 28

### 4-(4-methylpiperazin-1-yl)-7-(trifluoromethyl)tetrazolo[1,5-a]quinoxaline

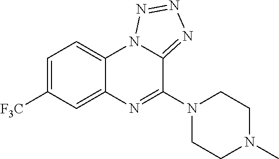

The title compound was prepared as described in Example 27, except that 2-chloro-3-(4-methylpiperazinyl)-6-(trifluoromethyl)quinoxaline was obtained in step 1 of that route. ¹H NMR (300 MHz, CD₃OD) δ: 8.35 (d, J=8.7 Hz, 1H), 7.86 (d, J=0.9 Hz, 1H), 7.63 (dd, J=8.7, 0.9 Hz, 1H), 4.42-4.38 (br, 4H), 2.67 (t, J=5.1 Hz, 4H), 2.39 (s, 3H). MS m/z: 338 (M+H⁺).

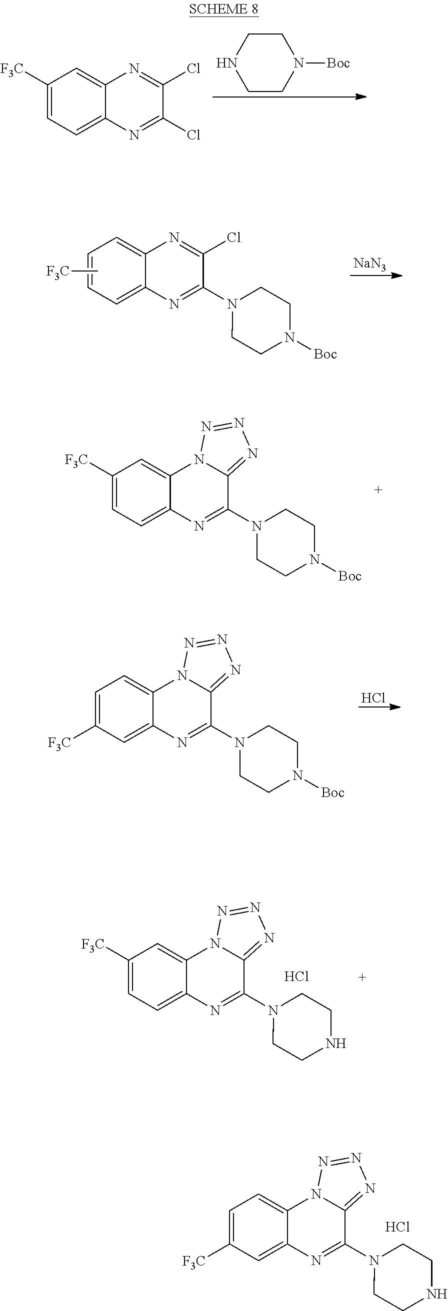

### EXAMPLE 29

### 4-(piperazin-1-yl)-8-(trifluoromethyl)tetrazolo[1,5-a]quinoxaline hydrochloride

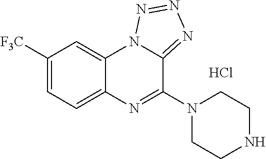

The title compound was prepared as described in Example 27, except that tert-butyl piperazinecarboxylate was substituted for N-methylpiperazine and tert-butyl 4-[3-chloro-6-(trifluoromethyl)quinoxalin-2-yl]piperazinecarboxylate was obtained in step 1 of that route. BOC group was then removed by methanolic HCl in EtOAc. ¹H NMR (300 MHz, CD₃OD) δ: 8.70 (d, J=2.1 Hz, 1H), 7.97 (d, J=9.0 Hz, 1H), 7.93 (dd, J=9.0, 2.1 Hz, 1H), 4.74-4.70 (br, 4H), 3.48 (t, J=5.1 Hz, 4H). MS m/z: 324 (M+H⁺).

### EXAMPLE 30

### 4-(piperazin-1-yl)-7-(trifluoromethyl)tetrazolo[1,5-a]quinoxaline hydrochloride

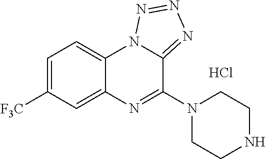

The title compound was prepared as described in Example 29, except that tert-butyl 4-[3-chloro-7-(trifluoromethyl)quinoxalin-2-yl]piperazinecarboxylate was obtained in step 1 of that route. ¹H NMR (300 MHz, CD₃OD) δ: 8.61 (d, J=8.4 Hz, 1H), 8.13 (d, J=1.8 Hz, 1H), 7.83 (dd, J=8.4, 1.8 Hz, 1H), 4.70 (t, J=5.1 Hz, 4H), 3.48 (t, J=5.1 Hz, 4H). MS m/z: 324 (M+H⁺).

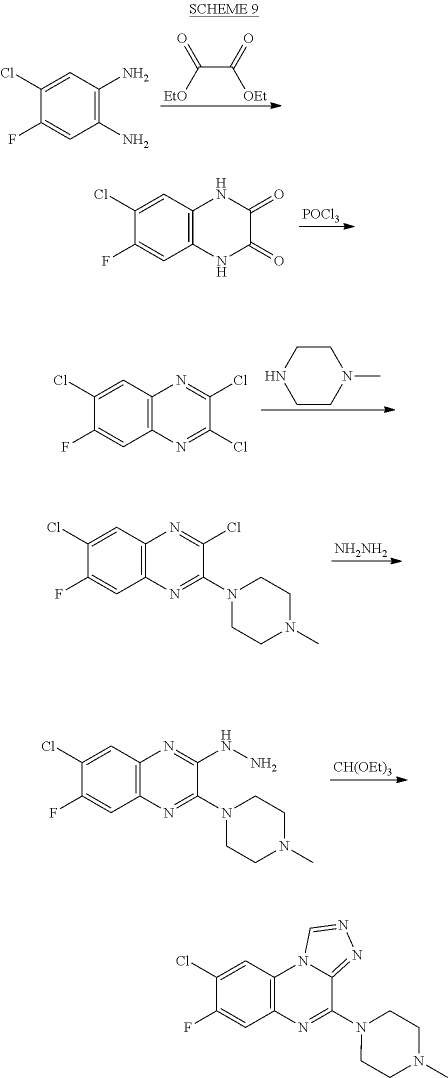

### EXAMPLE 31

### 8-chloro-7-fluoro-4-(4-methylpiperazin-1-yl)-[1,2,4]triazolo[4,3-a]quinoxaline

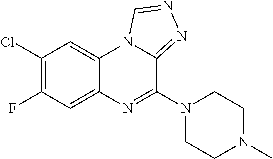

The title compound was prepared as described in Example 23, except that 4-chloro-5-fluorobenzene-1,2-diamine was substituted for 4-(trifluoromethyl)benzene-1,2-diamine in step 1 of that route. ¹H NMR (300 MHz, CD₃OD) δ: 9.80 (s, 1H), 8.36 (d, J=7.2 Hz, 1H), 7.53 (d, J=9.9 Hz, 1H), 5.45-3.28 (m, 8H), 2.97 (s, 3H). MS m/z: 321 (M+H⁺).

### EXAMPLE 32

### 8-chloro-7-fluoro-4-(piperazin-1-yl)-[1,2,4]triazolo[4,3-a]quinoxaline

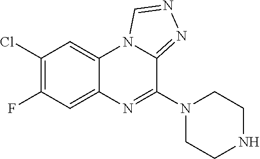

The title compound was prepared as described in Example 31, except that piperazine was substituted for N-methylpiperazine in step 3 of that route. ¹H NMR (300 MHz, CD₃OD) δ: 9.86 (s, 1H), 8.39 (d, J=7.2 Hz, 1H), 7.54 (d, J=9.9 Hz, 1H), 4.67 (t, J=5.1 Hz, 4H), 3.42 (t, J=5.1 Hz, 4H). MS m/z: 307 (M+H⁺).

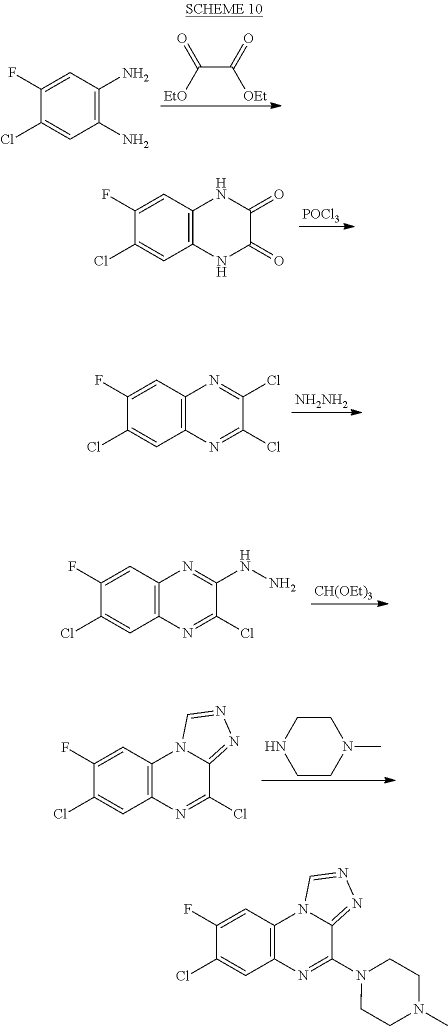

### EXAMPLE 33

### 7-chloro-8-fluoro-4-(4-methylpiperazin-1-yl)-[1,2,4]triazolo[4,3-a]quinoxaline

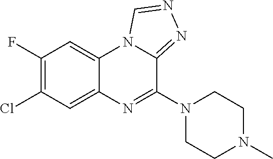

The title compound was prepared as described in Examples 50 and 21, except that 5-chloro-4-fluorobenzene-1,2-diamine was substituted for 4-methylbenzene-1,2-diamine as the starting material of that route. ¹H NMR (300 MHz, DMSO-d₆) δ: 9.93 (s, 1H), 8.38 (d, J=9.9 Hz, 1H), 7.72 (d, J=7.2 Hz, 1H), 4.30-4.27 (m, 4H), 3.34-3.31 (m, 4H), 2.23 (s, 3H). MS m/z: 321 (M+H⁺).

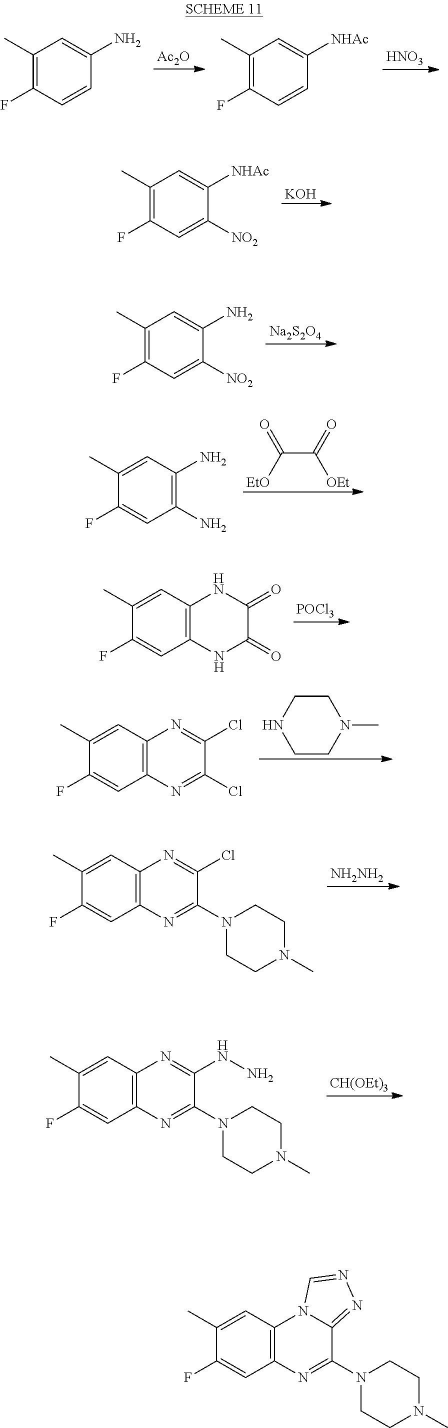

### EXAMPLE 34

### 7-fluoro-8-methyl-4-(4-methylpiperazin-1-yl)-[1,2,4]triazolo[4,3-a]quinoxaline

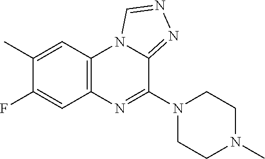

### Step 1

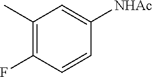

### N-(4-Fluoro-3-methylphenyl)acetamide

A 100 mL round bottom flask was charged with 4-fluoro-3-methylaniline (9.0 g, 0.072 mol) and acetyl acetate (32 mL). The resulting mixture was stirred 1 h at 0° C. Reaction progress was monitored by TLC (EtOAc/Petroleum ether=1:2). Work-up: the reaction solution was diluted with H₂O (100 mL) and neutralized with ammonia. The precipitate was collected by filtration, washed with H₂O, and dried under vacuum, to afford 12 g (quantitative yield) of product as white solids. MS m/z: 168 (M+H⁺).

### Step 2

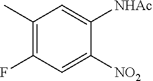

### N-(4-Fluoro-5-methyl-2-nitrophenyl)acetamide

A 100 mL round bottom flask was charged with N-(4-fluoro-3-methylphenyl)acetamide (10.5 g, 0.063 mol) and nitric acid (68%, 15 mL). To the solution was added dropwise fuming nitric acid (12 mL). The reaction solution was stirred 1 h at room temperature. Work-up: the reaction solution was diluted with H₂O (100 mL). The precipitate was collected by filtration, washed with H₂O, and dried under vacuum. It was further purified by column chromatography on silica gel with a 1:20 EtOAc/CH₂Cl₂, giving 8.47 g (64%) of the product as yellow solids. ¹H NMR (300 MHz, CDCl₃) δ: 10.28 (s, 1H), 8.65 (d, J=6.6 Hz, 1H), 7.87 (d, J=9.3 Hz, 1H), 2.36 (d, J=2.1 Hz, 3H), 2.28 (s, 3H).

### Step 3

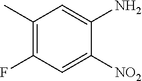

### 4-Fluoro-5-methyl-2-nitrophenylamine

A 250 mL round bottom flask was charged with N-(4-fluoro-5-methyl-2-nitrophenyl)acetamide (4.0 g, 0.019 mol), KOH (1.06 g, 0.019 mol), H₂O (30 mL) and MeOH (80 mL). The solution was kept in a 60° C. water-bath for 15 min. H₂O (30 mL) was added and the reaction mixture was kept in the bath for another 15 min before it was cooled in an ice-bath. The precipitates were collected by filtration, washed with cold water, and dried under vacuum, giving 3.15 g (98%) of the product as orange solids.

### Step 4

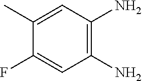

### 5-Fluoro-4-methylbenzene-1,2-diamine

A 250 mL round bottom flask was charged with 4-fluoro-5-methyl-2-nitrophenylamine (3.12 g, 0.018 mol), Na₂S₂O₄ (9.58 g, 0.055 mol), H₂O (45 mL) and EtOH (90 mL). The mixture was heated at reflux for 1 h. Work-up: the solvent was evaporated. The residue was suspended in triethylamine (15 mL) and ethyl acetate (300 mL), and then filtered. The filtrate was concentrated in vacuo, giving 2.1 g (82%) of the product as pale-red solids.

### Steps 5-9

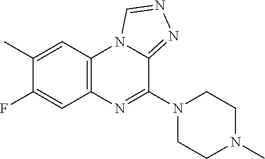

### 7-fluoro-8-methyl-4-(4-methylpiperazin-1-yl)-[1,2,4]-triazolo[4,3-a]quinoxaline

The title compound was prepared as described in Example 23, except that 5-fluoro-4-methylbenzene-1,2-diamine was substituted for 4-(trifluoromethyl)benzene-1,2-diamine in step 1 of that route. ¹H NMR (300 MHz, CD₃OD) δ: 9.68 (s, 1H), 7.93 (d, J=7.5 Hz, 1H), 7.25 (d, J=10.8 Hz, 1H), 4.38 (m, 4H), 2.64 (t, J=4.9 Hz, 4H), 2.39 (s, 3H), 2.37 (s, 3H). MS m/z: 301 (M+H⁺).

### EXAMPLE 35

### 7-fluoro-8-methyl-4-(4-methylpiperazin-1-yl)-[1,2,4]triazolo[4,3-a]quinoxaline

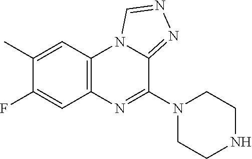

The title compound was prepared as described in Example 34, except that piperazine was substituted for N-methylpiperazine in step 7 of that route. ¹H NMR (300 MHz, D₂O) δ: 8.40 (s, 1H), 7.36 (d, J=8.1 Hz, 1H), 7.00 (d, J=10.5 Hz, 1H), 3.92 (t, J=5.1 Hz, 4H), 3.35 (t, J=5.1 Hz, 4H), 2.19 (s, 3H). MS m/z: 287 (M+H⁺).

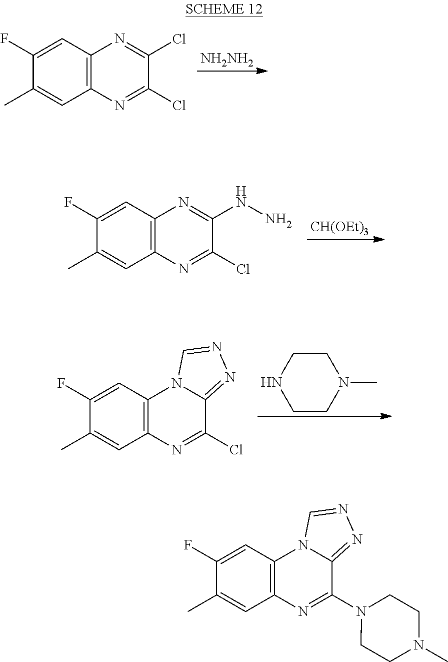

### EXAMPLE 36

### 8-fluoro-7-methyl-4-(4-methylpiperazin-1-yl)-[1,2,4]triazolo[4,3-a]quinoxaline

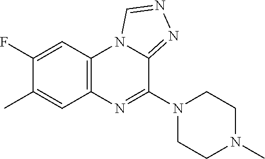

The title compound was prepared as described in Examples 50 and 21, except that 5-fluoro-4-methylbenzene-1,2-diamine (prepared in Example 34 step 1-4) was substituted for 4-methylbenzene-1,2-diamine as the starting material of that route. ¹H NMR (300 MHz, CDCl₃) δ: 9.05 (s, 1H), 7.62 (d, J=7.8 Hz, 1H), 7.37 (d, J=8.7 Hz, 1H), 4.42 (m, 4H), 2.60 (t, J=4.8 Hz, 4H), 2.37 (s, 6H). MS m/z: 301 (M+H⁺).

### EXAMPLE 37

### 7,8-difluoro-4-(4-methylpiperazin-1-yl)-[1,2,4]triazolo[4,3-a]quinoxaline

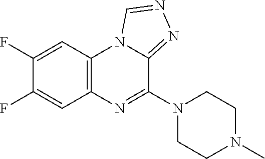

The title compound was prepared as described in Examples 50 and 21, except that 4,5-difluorobenzene-1,2-diamine was substituted for 4-methylbenzene-1,2-diamine as the starting material of that route. ¹H NMR (300 MHz, CD₃OD) δ: 9.67 (s, 1H), 8.08 (dd, J=10.5, 7.5 Hz, 1H), 7.45 (dd, J=11.4, 7.8, 1H), 4.38 (m, 4H), 2.63 (t, J=5.1 Hz, 4H), 2.36 (s, 3H). MS m/z: 305 (M+H⁺).

### EXAMPLE 38

### 7,8-difluoro-4-(piperazin-1-yl)-[1,2,4]triazolo[4,3-a]quinoxaline

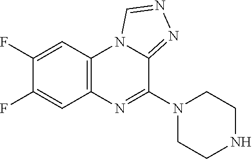

The title compound was prepared as described in Example 37, except that piperazine was substituted for N-methylpiperazine in the last step of that route. ¹H NMR (300 MHz, CD₃OD) δ: 9.68 (s, 1H), 8.09 (dd, J=10.5, 7.8 Hz, 1H), 7.46 (dd, J=11.7, 7.8 Hz, 1H), 4.35 (t, J=4.8 Hz, 4H), 2.99 (t, J=5.1 Hz, 4H). MS m/z: 291 (M+H⁺).

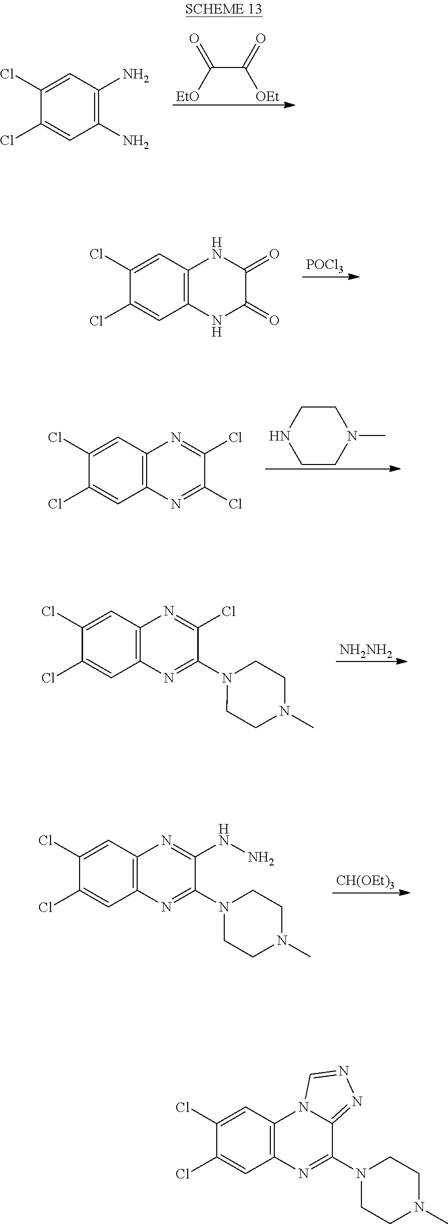

### EXAMPLE 39

### 7,8-dichloro-4-(4-methylpiperazin-1-yl)-[1,2,4]triazolo[4,3-a]quinoxaline

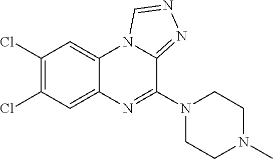

The title compound was prepared as described in Example 23, except that 4,5-dichlorobenzene-1,2-diamine was substituted for 4-(trifluoromethyl)benzene-1,2-diamine in step 1 of that route. ¹H NMR (300 MHz, CDCl₃) δ: 9.09 (s, 1H), 7.80 (s, 1H), 7.76 (s, 1H), 4.50-4.47 (m, 4H), 2.59 (t, J=5.1 Hz, 4H), 2.36 (s, 3H). MS m/z: 337 (M+H⁺).

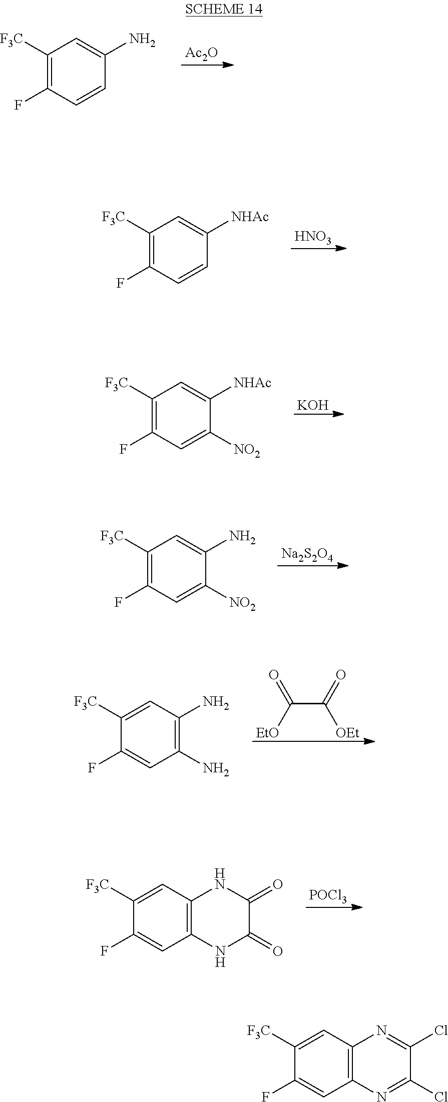

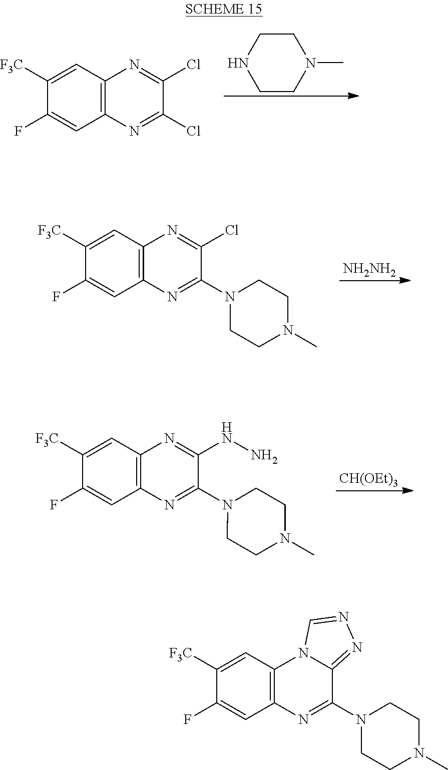

### EXAMPLE 40

### 8-fluoro-4-(4-methylpiperazin-1-yl)-7-(trifluoromethyl)-[1,2,4]triazolo[4,3-a]quinoxaline

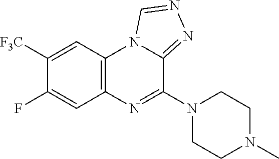

The title compound was prepared as described in Example 34, except that 4-fluoro-3-trifluoromethylaniline was substituted for 4-fluoro-3-methylaniline in step 1 of that route. ¹H NMR (300 MHz, CD₃OD) δ: 9.95 (s, 1H), 8.54 (d, J=6.0 Hz, 1H), 7.56 (d, J=12.0 Hz, 1H), 4.88-4.82 (m, 4H), 3.52-3.47 (m, 4H), 2.97 (s, 3H). MS m/z: 355 (M+H⁺).

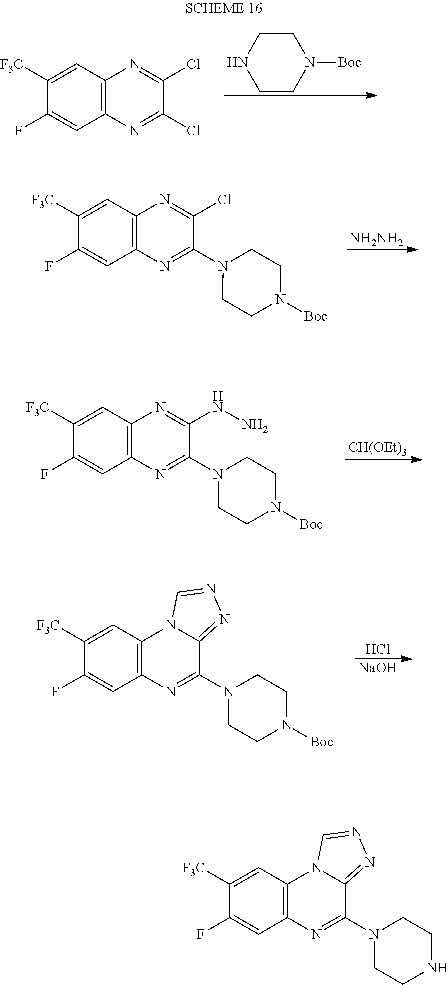

### EXAMPLE 41

### 7-Fluoro-4-(piperazin-1-yl)-8-(trifluoromethyl)-[1,2,4]triazolo[4,3-a]quinoxaline

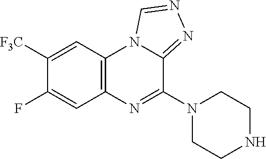

The title compound was prepared as described in Example 40, except that N—BOC piperazine was substituted for N-methylpiperazine. ¹H NMR (300 MHz, CD₃OD) δ: 9.86 (s, 1H), 8.44 (d, J=6.0 Hz, 1H), 7.41 (d, J=12.0 Hz, 1H), 4.47-4.43 (m, 4H), 3.02-2.99 (m, 4H). MS m/z: 341 (M+H⁺).

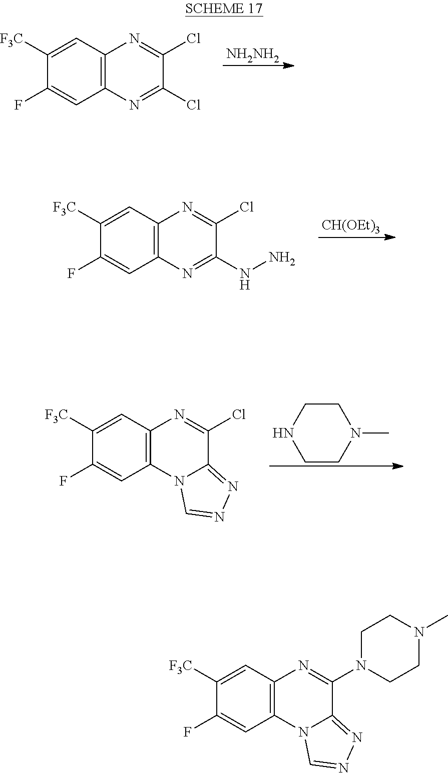

### EXAMPLE 42

### 8-fluoro-4-(4-methylpiperazin-1-yl)-7-(trifluoromethyl)-[1,2,4]triazolo[4,3-a]quinoxaline

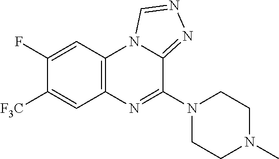

The title compound was prepared as described in Examples 50 and 21, except that 5-fluoro-4-trifluoromethylbenzene-1,2-diamine (prepared in Example 40 step 1-4) was substituted for 4-methylbenzene-1,2-diamine as the starting material of that route. ¹H NMR (300 MHz, CD₃OD) δ: 9.77 (s, 1H), 8.14 (d, J=12.0 Hz, 1H), 7.90 (d, J=9.0 Hz, 1H), 4.43-4.40 (m, 4H), 2.67-2.64 (m, 4H), 2.38 (s, 3H). MS m/z: 355 (M+H⁺).

### EXAMPLE 43

### 8-Fluoro-4-(piperazin-1-yl)-7-(trifluoromethyl)-[1,2,4]triazolo[4,3-a]quinoxaline

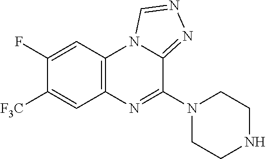

The title compound was prepared as described in Example 42, except that piperazine was substituted for N-methylpiperazine in the last step of that route. ¹H NMR (300 MHz, CD₃OD) δ: 9.75 (s, 1H), 8.11 (d, J=12.0 Hz, 1H), 7.86 (d, J=9.0 Hz, 1H), 4.39-4.36 (m, 4H), 3.00-2.96 (m, 4H). MS m/z: 341 (M+H⁺).

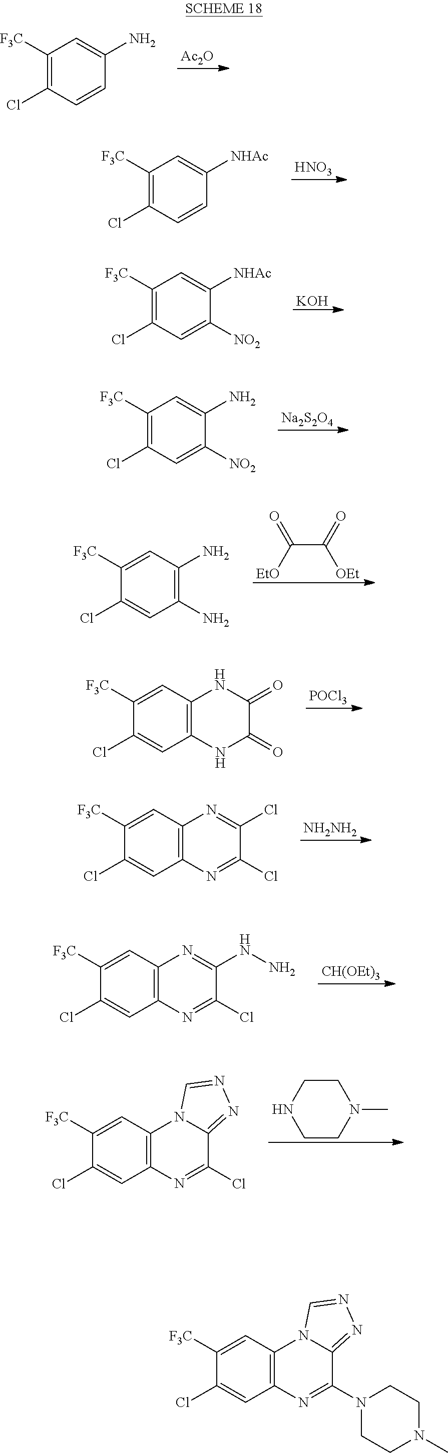

### EXAMPLE 44

### 7-Chloro-4-(4-methylpiperazin-1-yl)-8-(trifluoromethyl)-[1,2,4]triazolo[4,3-a]quinoxaline

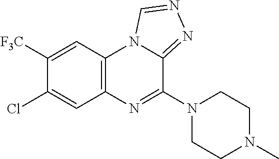

### Steps 1-4

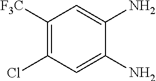

### 4-Chloro-5-(trifluoromethyl)benzene-1,2-diamine

The title compound was prepared as described in Example 34 step 1-4, except that 4-chloro-3-trifluoromethylaniline was substituted for 4-fluoro-3-methylaniline as the starting material of that route.

### Steps 5-9

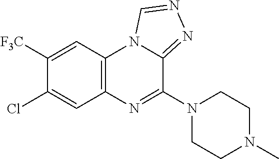

### 7-Chloro-4-(4-methylpiperazin-1-yl)-8-(trifluoromethyl)-[1,2,4]triazolo[4,3-a]quinoxaline

The title compound was prepared as described in Examples 50 and 21, except that 4-chloro-5-(trifluoromethyl)benzene-1,2-diamine was substituted for 4-methylbenzene-1,2-diamine as the starting material of that route. ¹H NMR (300 MHz, CD₃OD) δ: 9.81 (s, 1H), 8.39 (s, 1H), 7.95 (s, 1H), 4.46 (m, 4H), 2.64 (t, J=5.1 Hz, 4H), 2.37 (s, 3H). MS m/z: 371 (M+H⁺).

### EXAMPLE 45

### 7-Chloro-4-(piperazin-1-yl)-8-(trifluoromethyl)-[1,2,4]triazolo[4,3-a]quinoxaline

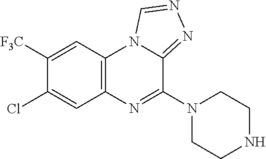

The title compound was prepared as described in Example 44, except that piperazine was substituted for N-methylpiperazine in the last step of that route. ¹H NMR (300 MHz, CD₃OD) δ: 10.12 (s, 1H), 8.73 (s, 1H), 7.96 (s, 1H), 4.58 (m, 4H), 3.28 (m, 4H). MS m/z: 357 (M+H⁺).

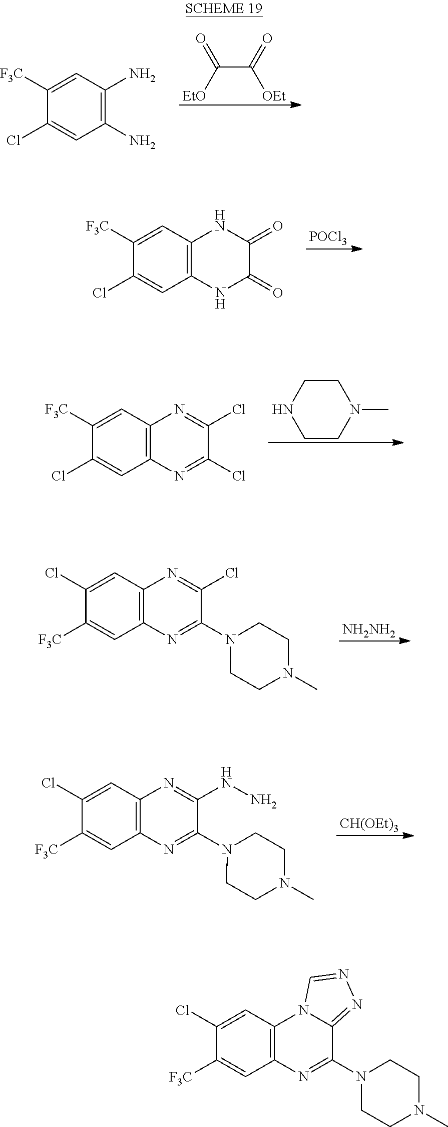

### EXAMPLE 46

### 8-Chloro-4-(4-methylpiperazin-1-yl)-7-(trifluoromethyl)-[1,2,4]triazolo[4,3-a]quinoxaline

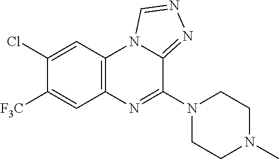

The title compound was prepared as described in Example 23, except that 4-chloro-5-(trifluoromethyl)benzene-1,2-diamine (prepared in Example 44 step 1-4) was substituted for 4-(trifluoromethyl)benzene-1,2-diamine as the starting material of that route. ¹H NMR (300 MHz, CD₃OD) δ: 9.89 (s, 1H), 8.49 (s, 1H), 7.71 (s, 1H), 4.50 (m, 4H), 2.64 (t, J=5.1 Hz, 4H), 2.37 (s, 3H). MS m/z: 371 (M+H⁺).

### EXAMPLE 47

### 8-Chloro-4-(piperazin-1-yl)-7-(trifluoromethyl)-[1,2,4]triazolo[4,3-a]quinoxaline

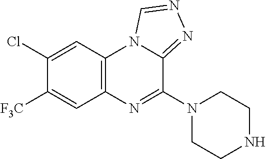

The title compound was prepared as described in Example 46, except that piperazine was substituted for N-methylpiperazine in step 3 of that route. ¹H NMR (300 MHz, CD₃OD) δ: 10.22 (s, 1H), 8.76 (s, 1H), 7.80 (s, 1H), 4.64 (m, 4H), 3.21 (m, 4H). MS m/z: 357 (M+H⁺).

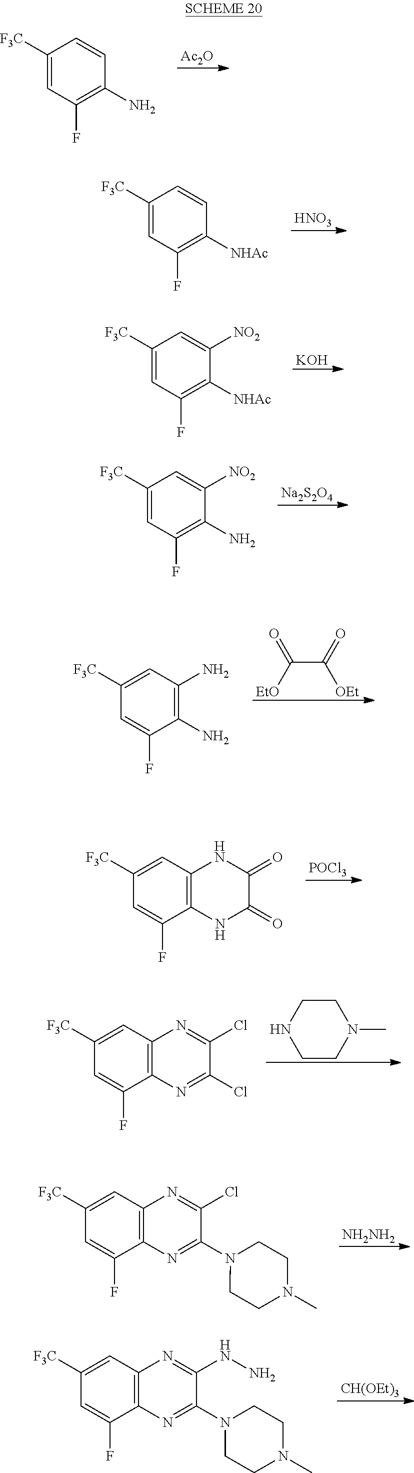

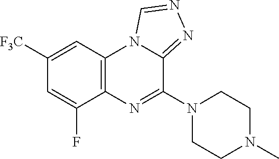

### EXAMPLE 48

### 6-Fluoro-4-(4-methylpiperazin-1-yl)-8-(trifluoromethyl)-[1,2,4]triazolo[4,3-a]quinoxaline

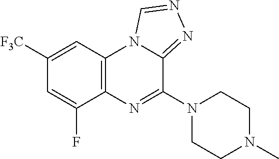

The title compound was prepared as described in Example 34, except that 2-fluoro-4-trifluoromethylaniline was substituted for 4-fluoro-3-methylaniline in step 1 of that route. ¹H NMR (300 MHz, CD₃OD) δ: 9.89 (s, 1H), 8.29 (s, 1H), 7.53 (d, J=9.3 Hz, 1H), 4.52 (m, 4H), 2.66 (d, J=4.8 Hz, 4H), 2.37 (s, 3H). MS m/z: 355 (M+H⁺).

### EXAMPLE 49

### 6-Fluoro-4-(piperazin-1-yl)-8-(trifluoromethyl)-[1,2,4]triazolo[4,3-a]quinoxaline

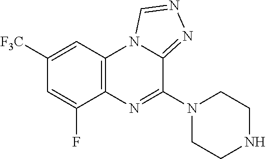

The title compound was prepared as described in Example 48, except that piperazine was substituted for N-methylpiperazine in step 7 of that route. ¹H NMR (300 MHz, CD₃OD) δ: 9.86 (s, 1H), 8.24 (s, 1H), 7.48 (d, J=9.9 Hz, 1H), 4.45 (m, 4H), 3.00 (m, 4H). MS m/z: 341 (M+H⁺).

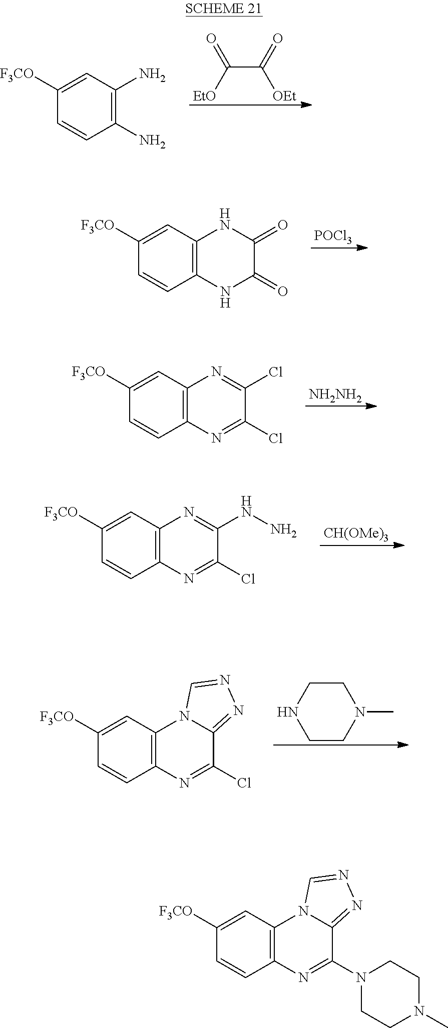

### EXAMPLE 50

### 4-(4-methylpiperazin-1-yl)-8-(trifluoromethoxy)-[1,2,4]triazolo[4,3-a]quinoxaline

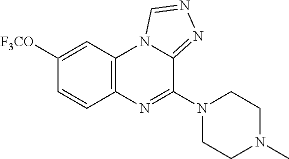

The title compound was prepared as described in Examples 50 and 21, except that 4-(trifluoromethoxy)benzene-1,2-diamine was substituted for 4-methylbenzene-1,2-diamine as the starting material of that route. ¹H NMR (300 MHz, DMSO-d₆) δ: 10.03 (s, 1H), 8.34 (d, J=1.2 Hz, 1H), 7.65 (d, J=9.0 Hz, 1H), 7.45 (dd, J=9.0, 1.2 Hz, 1H), 4.31 (br, 4H), 2.49-2.46 (m, 4H), 2.22 (s, 3H). MS m/z: 353 (M+H⁺).

### EXAMPLE 51

### 4-(piperazin-1-yl)-8-(trifluoromethoxy)-[1,2,4]triazolo[4,3-a]quinoxaline

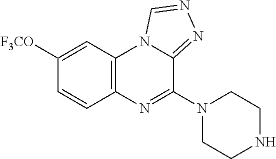

The HCl salt of the title compound was prepared as described in Example 50, except that piperazine was substituted for N-methylpiperazine in step 5 of that route. ¹H NMR (300 MHz, DMSO-d₆) δ: 10.01 (s, 1H), 8.32 (s, 1H), 7.63 (d, J=8.4 Hz, 1H), 7.43 (d, J=8.7 Hz, 1H), 4.25 (br, 4H), 2.84 (br, 4H). MS m/z: 339 (M+H⁺).

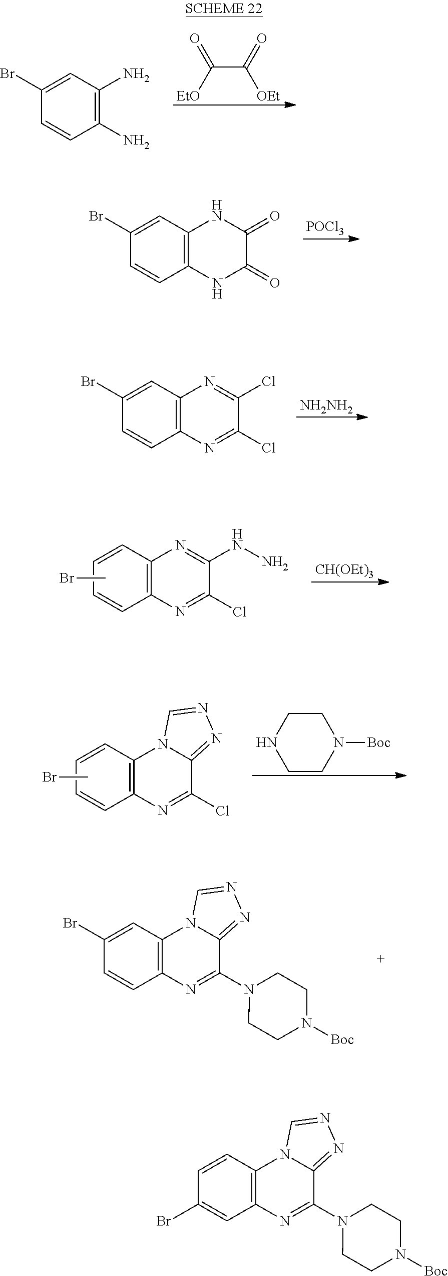

### EXAMPLE 52

### 8-bromo-4-(piperazin-1-yl)-[1,2,4]triazolo[4,3-a]quinoxaline

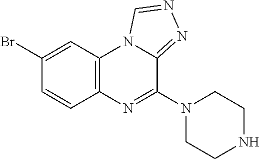

### Steps 1-5

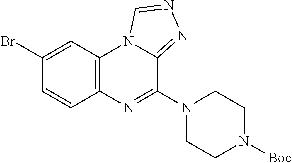

### tert-butyl 4-(8-bromo-[1,2,4]triazolo[4,3-a]quinoxalin-4-yl)piperazine-1-carboxylate

The title compound was prepared as described in Examples 50 and 21, except that 4-bromobenzene-1,2-diamine was substituted for 4-methylbenzene-1,2-diamine as the starting material, and N—BOC piperazine for N-methylpiperazine in the last step of that route. It was separated from the other regio-isomer by column chromatography on silica gel with a 1:1:2 EtOAc/CH₂Cl₂/petroleum ether. ¹H NMR (300 MHz, CDCl₃) δ: 9.14 (s, 1H), 7.88 (m, 1H), 7.56 (m, 2H), 4.42 (m, 4H), 3.63 (m, 4H), 1.50 (s, 9H).

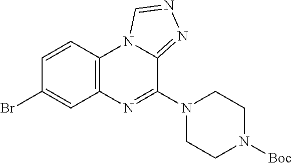

tert-butyl 4-(7-bromo-[1,2,4]triazolo[4,3-a]quinoxalin-4-yl)piperazine-1-carboxylate

The title compound was prepared as described in Examples 50 and 21, except that 4-bromobenzene-1,2-diamine was substituted for 4-methylbenzene-1,2-diamine as the starting material, and N—BOC piperazine for N-methylpiperazine in the last step of that route. It was separated from the other regio-isomer by column chromatography on silica gel with a 1:1:2 EtOAc/CH₂Cl₂/petroleum ether. ¹H NMR (300 MHz, CDCl₃) δ: 9.14 (s, 1H), 7.85 (d, J=2.1 Hz, 1H), 7.59 (d, J=8.7 Hz, 1H), 7.41 (dd, J=8.7, 2.1 Hz, 1H), 4.44 (m, 4H), 3.63 (t, J=5.1 Hz, 4H), 1.50 (s, 9H).

### Step 6

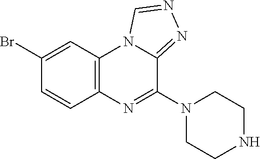

### 8-bromo-4-(piperazin-1-yl)-[1,2,4]triazolo[4,3-a]quinoxaline

A 50 mL round bottom flask was charged with tert-butyl 4-(8-bromo-10-hydro-1,2,4-triazolo[4,3-a]quinoxalin-4-yl)piperazinecarboxylate (0.13 g, 0.28 mmol), THF (15 mL) and concentrate HCl (0.5 mL). The reaction mixture was heated at reflux for 1 h. Work-up: the solid was collected by filtration, washed with THF, and dried under vacuum, giving 0.11 g (99%) of the product as white solids. ¹H NMR (300 MHz, D₂O) δ: 9.19 (s, 1H), 7.49 (d, J=1.8 Hz, 1H), 7.15 (dd, J=6.6, 2.1 Hz, 1H), 6.98 (d, J=5.7 Hz, 1H), 4.27 (t, J=5.1 Hz, 4H), 3.37 (t, J=5.1 Hz, 4H). MS m/z: 333 (M+H⁺).

### EXAMPLE 53

### 7-bromo-4-(piperazin-1-yl)-[1,2,4]triazolo[4,3-a]quinoxaline

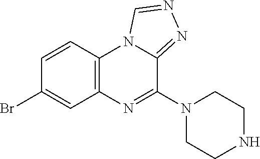

The HCl salt of the title compound was prepared as described in Example 52, except that tert-butyl 4-(7-bromo-10-hydro-1,2,4-triazolo[4,3-a]quinoxalin-4-yl)piperazinecarboxylate was substituted for tert-butyl 4-(8-bromo-10-hydro-1,2,4-triazolo[4,3-a]quinoxalin-4-yl)piperazinecarboxylate in the last step of that route. ¹H NMR (300 MHz, D₂O) δ: 9.30 (s, 1H), 7.26 (d, J=8.7 Hz, 1H), 7.22 (s, 1H), 7.04 (d, J=8.4 Hz, 1H), 4.29 (t, J=5.4 Hz, 4H), 3.36 (t, J=5.1 Hz, 4H). MS m/z: 333 (M+H⁺).

### EXAMPLE 54

### 8-bromo-4-(4-methylpiperazin-1-yl)-[1,2,4]triazolo[4,3-a]quinoxaline

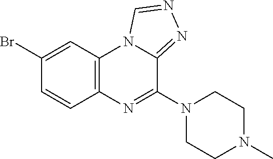

A 100 mL round bottom flask was charged with 8-bromo-4-piperazinyl-10-hydro-1,2,4-triazolo[4,3-a]quinoxaline HCl salt (1.30 g, 3.6 mmol), formaldehyde (40%, 6 mL), CH₂Cl₂ (20 mL), MeOH (20 mL) and NaBH₃(CN) (0.68 g, 0.011 mol). The resulting mixture was stirred at room temperature for 1 h. Work-up: the reaction mixture was diluted with H₂O (100 mL) and extracted with CH₂Cl₂ (50 mL×2). The combined organic layers was dried over anhydrous Na₂SO₄ and concentrated in vacuo. The residue was further purified by column chromatography on silica gel with 3% MeOH in CH₂Cl₂, giving 0.94 g (77%) of the product as white solids. ¹H NMR (300 MHz, CDCl₃) δ: 9.12 (s, 1H), 7.89 (m, 1H), 7.63 (m, 2H), 4.46 (m, 4H), 2.60 (t, J=5.1 Hz, 4H), 2.36 (s, 3H). MS m/z: 347 (M+H⁺).

### EXAMPLE 55

### 8-chloro-4-(4-methylpiperazin-1-yl)-[1,2,4]triazolo[4,3-a]quinoxaline

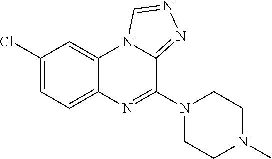

The title compound was obtained from commercial sources.

### EXAMPLE 56

### tert-butyl 4-(8-chloro-[1,2,4]triazolo[4,3-a]quinoxalin-4-yl)piperazine-1-carboxylate

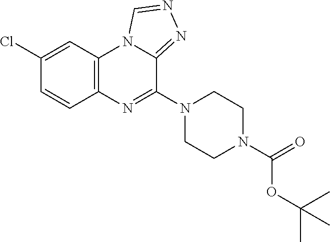

The title compound was prepared analogously to Example 54. MS m/z: 389 (M+H+).

### EXAMPLE 57

### 8-chloro-4-(piperazin-1-yl)-[1,2,4]triazolo[4,3-a]quinoxaline

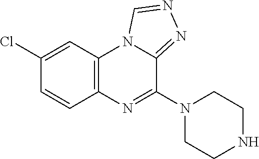

The title compound was prepared analogously to Example 54. MS m/z: 289 (M+H⁺).

### EXAMPLE 58

### 4-(4-methylpiperazin-1-yl)-[1,2,4]triazolo[4,3-a]quinoxaline

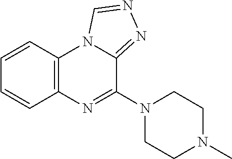

The title compound was prepared analogously to Example 54. MS m/z: 269 (M+H⁺).

### EXAMPLE 59

### 1-(8-chloro-[1,2,4]triazolo[4,3-a]quinoxalin-4-yl)pyrrolidin-3-amine

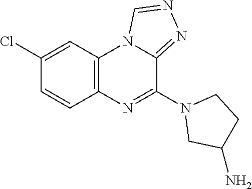

The title compound was prepared analogously to Example 54. MS m/z: 289 (M+H⁺).

### EXAMPLE 60

### 1-(8-chloro-[1,2,4]triazolo[4,3-a]quinoxalin-4-yl)-N-methylpyrrolidin-3-amine

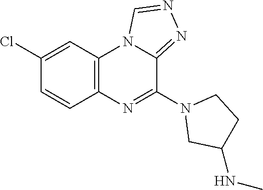

The title compound was prepared analogously to Example 54. MS m/z: 303 (M+H⁺).

### EXAMPLE 61

### 8-chloro-4-(tetrahydro-1H-pyrrolo[3,4-b]pyridin-6(2H,7H,7aH)-yl)-[1,2,4]triazolo[4,3-a]quinoxaline

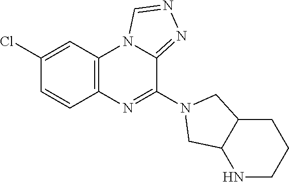

The title compound was prepared analogously to Example 54. MS m/z: 329 (M+H⁺).

### EXAMPLE 62

### 8-chloro-4-(5-methylhexahydropyrrolo[3,4-c]pyrrol-2(1H)-yl)-[1,2,4]triazolo[4,3-a]quinoxaline

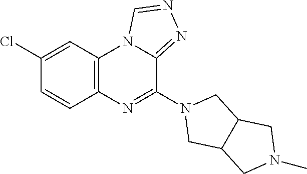

The title compound was prepared analogously to Example 54. MS m/z: 329 (M+H⁺).

### EXAMPLE 63

### 1-(8-chloro-[1,2,4]triazolo[4,3-a]quinoxalin-4-yl)azetidin-3-amine

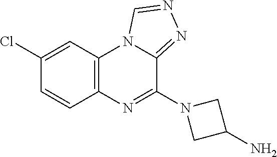

The title compound was prepared analogously to Example 54. MS m/z: 275 (M+H⁺).

### EXAMPLE 64

### 8-chloro-4-(4-cyclopropylpiperazin-1-yl)-[1,2,4]triazolo[4,3-a]quinoxaline

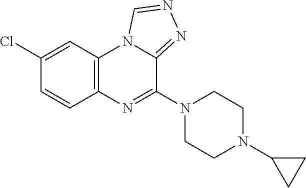

The title compound was prepared analogously to Example 54. MS m/z: 329 (M+H⁺).

### EXAMPLE 65

### 8-chloro-4-(hexahydropyrrolo[1,2-a]pyrazin-2(1H)-yl)-[1,2,4]triazolo[4,3-a]quinoxaline

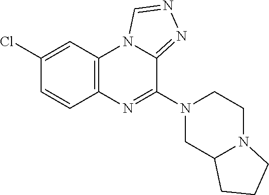

The title compound was prepared analogously to Example 54. MS m/z: 329 (M+H⁺).

### EXAMPLE 66

### 8-chloro-4-(1,4-diazepan-1-yl)-[1,2,4]triazolo[4,3-a]quinoxaline

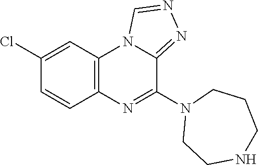

The title compound was prepared analogously to Example 54. MS m/z: 303 (M+H⁺).

### EXAMPLE 67

### 4-(2,5-diazabicyclo[2.2.1]heptan-2-yl)-8-chloro-[1,2,4]triazolo[4,3-a]quinoxaline

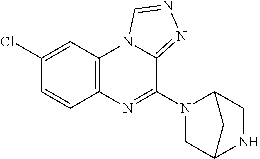

The title compound was prepared analogously to Example 54. MS m/z: 301 (M+H⁺).

### EXAMPLE 68

### 8-chloro-4-(4-methyl-1,4-diazepan-1-yl)-[1,2,4]triazolo[4,3-a]quinoxaline

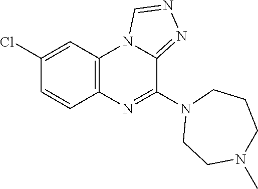

The title compound was prepared analogously to Example 54. MS m/z: 317 (M+H⁺).

### EXAMPLE 69

### 8-chloro-4-(hexahydropyrrolo[3,4-c]pyrrol-2(1H)-yl)-[1,2,4]triazolo[4,3-a]quinoxaline

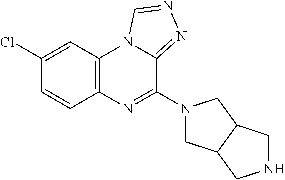

The title compound was prepared analogously to Example 54. MS m/z: 315 (M+H⁺).

### EXAMPLE 70

N¹-(8-chloro-[1,2,4]triazolo[4,3-a]quinoxalin-4-yl)ethane-1,2-diamine

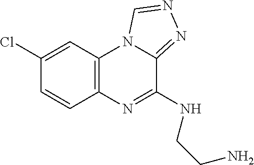

The title compound was prepared analogously to Example 54. MS m/z: 263 (M+H⁺).

### EXAMPLE 71

### 8-chloro-N-(2-morpholinoethyl)-[1,2,4]triazolo[4,3-a]quinoxalin-4-amine

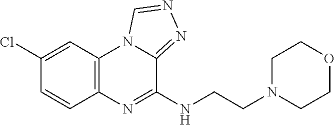

The title compound was prepared analogously to Example 54. MS m/z: 333 (M+H⁺).

### EXAMPLE 72

### 4-(azetidin-3-yloxy)-8-chloro-[1,2,4]triazolo[4,3-a]quinoxaline

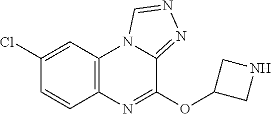

The title compound was prepared analogously to Example 54. MS m/z: 276 (M+H⁺).

### EXAMPLE 73

### 8-chloro-N-(piperidin-4-yl)-[1,2,4]triazolo[4,3-a]quinoxalin-4-amine

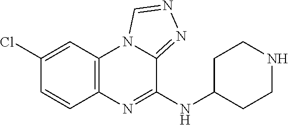

The title compound was prepared analogously to Example 54. MS m/z: 303 (M+H⁺).

### EXAMPLE 74

### 8-chloro-4-(piperidin-4-yloxy)-[1,2,4]triazolo[4,3-a]quinoxaline

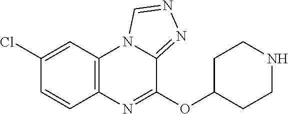

The title compound was prepared analogously to Example 54. MS m/z: 304 (M+H⁺).

### EXAMPLE 75

### 4-(azetidin-3-ylmethoxy)-8-chloro-[1,2,4]triazolo[4,3-a]quinoxaline

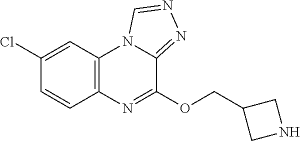

The title compound was prepared analogously to Example 54. MS m/z: 290 (M+H⁺).

### EXAMPLE 76

### (S)-8-chloro-4-((1-methylpyrrolidin-3-yl)methoxy)-[1,2,4]triazolo[4,3-a]quinoxaline

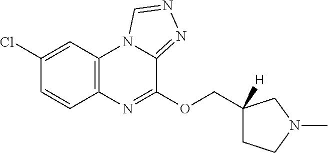

The title compound was prepared analogously to Example 54. MS m/z: 318 (M+H⁺).

### EXAMPLE 77

N¹-(8-chloro-[1,2,4]triazolo[4,3-a]quinoxalin-4-yl)-N¹,N²-dimethylethane-1,2-diamine

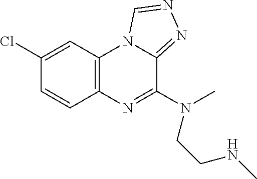

The title compound was prepared analogously to Example 54. MS m/z: 291 (M+H⁺).

### EXAMPLE 78

N¹-(8-chloro-[1,2,4]triazolo[4,3-a]quinoxalin-4-yl)-N¹,N²,N²-trimethylethane-1,2-diamine

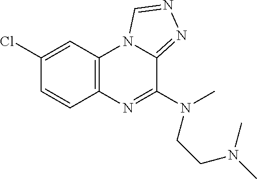

The title compound was prepared analogously to Example 54. MS m/z: 305 (M+H⁺).

### EXAMPLE 79

N¹-(8-chloro-[1,2,4]triazolo[4,3-a]quinoxalin-4-yl)-N-1-methylethane-1,2-diamine

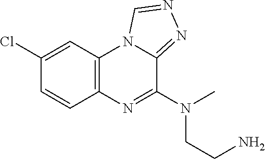

The title compound was prepared analogously to Example 54. MS m/z: 277 (M+H⁺).

### EXAMPLE 80

### 2-(8-chloro-[1,2,4]triazolo[4,3-a]quinoxalin-4-yloxy)-N-methylethanamine

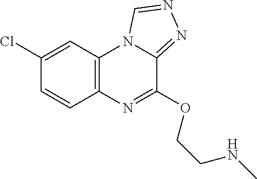

The title compound was prepared analogously to Example 54. MS M/Z: 278 (M+H⁺).

### EXAMPLE 81

### 1-(8-chloro-[1,2,4]triazolo[4,3-a]quinoxalin-4-yl)piperidin-4-amine

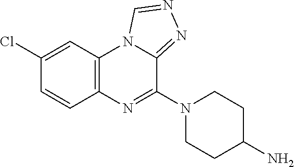

The title compound was prepared analogously to Example 54. MS M/Z: 303 (M+H⁺).

### EXAMPLE 82

### 8-chloro-4-(3,3-dimethylpiperazin-1-yl)-[1,2,4]triazolo[4,3-a]quinoxaline

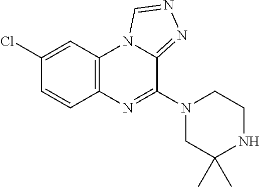

The title compound was prepared analogously to Example 54. MS M/Z: 317 (M⁺H⁺).

### EXAMPLE 83

### 8-chloro-4-((3S,5R)-3,5-dimethylpiperazin-1-yl)-[1,2,4]triazolo[4,3-a]quinoxaline

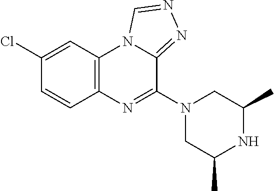

The title compound was prepared analogously to Example 54. MS M/Z: 317 (M+H⁺).

### EXAMPLE 84

### 8-chloro-1-methyl-4-(4-methylpiperazin-1-yl)-[1,2,4]triazolo[4,3-a]quinoxaline

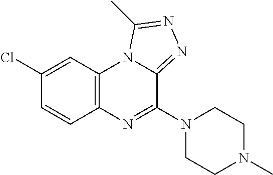

The title compound was prepared analogously to Example 54. MS M/Z: 317 (M+H⁺).

### EXAMPLE 85

### 8-chloro-1-methyl-4-(piperazin-1-yl)-[1,2,4]triazolo[4,3-a]quinoxaline

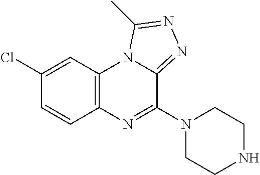

The title compound was prepared analogously to Example 54. MS M/Z: 303 (M+H⁺).

### EXAMPLE 86

### 8-chloro-1-ethyl-4-(4-methylpiperazin-1-yl)-[1,2,4]triazolo[4,3-a]quinoxaline

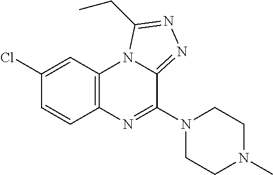

The title compound was prepared analogously to Example 54. MS M/Z: 331 (M+H⁺).

### EXAMPLE 87

### 8-chloro-1-isopropyl-4-(4-methylpiperazin-1-yl)-[1,2,4]triazolo[4,3-a]quinoxaline

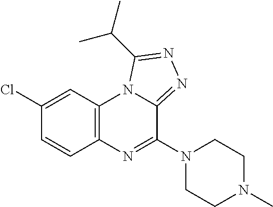

The title compound was prepared analogously to Example 54. MS M/Z: 345 (M+H⁺).

### EXAMPLE 88

### 4-(4-methylpiperazin-1-yl)-8-vinyl-[1,2,4]triazolo[4,3-a]quinoxaline

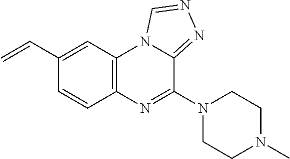

A 100 mL round bottom flask was charged with 8-bromo-4-(4-methylpiperazinyl)-10-hydro-1,2,4-triazolo[4,3-a]quinoxaline (Example 54, 0.86 g, 2.48 mmol), LiCl (0.21 g, 5.0 mmol), tri-n-butyl(vinyl)tin (0.94 g, 3.0 mmol), bis(triphenylphosphine)palladium(II) chloride (0.12 g, 0.2 mmol) and DMF (25 mL). The mixture was heated at 90° C. overnight. Work-up: the reaction solution was diluted with H₂O (100 mL) and extracted with EtOAc (100 mL×2). The combined organic layers was dried over anhydrous Na₂SO₄ and concentrated in vacuo. The residue was further purified by column chromatography on silica gel with 4% MeOH in CH₂Cl₂, giving 0.48 g (66%) of the product as white solids. ¹H NMR (300 MHz, CDCl₃) δ: 9.17 (s, 1H), 7.68-7.61 (m, 3H), 6.78 (dd, J=17.4, 11.1 Hz, 1H), 5.82 (d, J=17.4 Hz, 1H), 5.34 (d, J=11.1 Hz, 1H), 4.46 (m, 4H), 2.60 (t, J=5.1 Hz, 4H), 2.36 (s, 3H). MS m/z: 295 (M+H⁺).

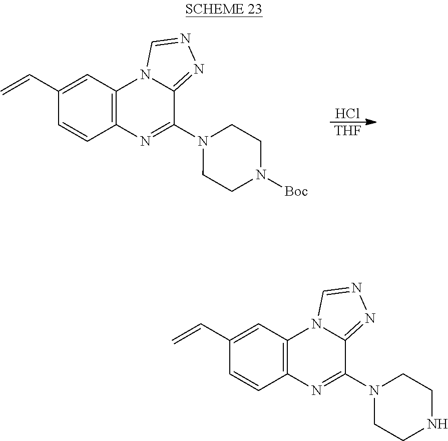

### EXAMPLE 89

### 4-(piperazin-1-yl)-8-vinyl-[1,2,4]triazolo[4,3-a]quinoxaline

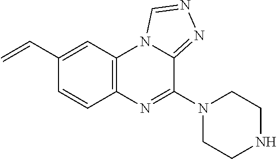

The HCl salt of the title compound was prepared as described in Example 52, except that tert-butyl 4-(8-vinyl-10-hydro-1,2,4-triazolo[4,3-a]quinoxalin-4-yl)piperazinecarboxylate (prepared as described in Example 88 from tert-butyl 4-(8-bromo-10-hydro-1,2,4-triazolo[4,3-a]quinoxalin-4-yl)piperazinecarboxylate) was substituted for tert-butyl 4-(8-bromo-10-hydro-1,2,4-triazolo[4,3-a]quinoxalin-4-yl)piperazinecarboxylate in step 6 of that route. ¹H NMR (300 MHz, D₂O) δ: 8.89 (s, 1H), 6.68 (m, 3H), 6.08 (dd, J=17.4, 10.8 Hz, 1H), 5.36 (d, J=17.4 Hz, 1H), 5.06 (d, J=10.8 Hz, 1H), 4.07 (t, J=5.1 Hz, 4H), 3.28 (t, J=5.1 Hz, 4H). MS m/z: 281 (M+H⁺).

### EXAMPLE 90

### 8-ethyl-4-(4-methylpiperazin-1-yl)-[1,2,4]triazolo[4,3-a]quinoxaline

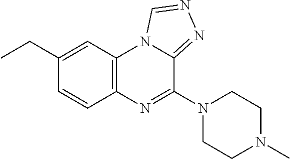

A 100 mL round bottom flask was charged with 4-(4-methylpiperazinyl)-8-vinyl-10-hydro-1,2,4-triazolo[4,3-a]quinoxaline (Example 88, 0.26 g, 0.88 mol), Pd/C (0.10 g) and THF (30 mL). The mixture was stirred under H₂ atmosphere for 1 h. Work-up: The reaction mixture was filtered. The filtrate was concentrated in vacuo, giving 0.18 g (69%) of the product as white solids. ¹H NMR (300 MHz, CDCl₃) δ: 9.17 (s, 1H), 7.61 (d, J=8.4 Hz, 1H), 7.62 (d, J=1.8 Hz, 1H), 7.31 (dd, J=8.4, 1.8 Hz, 1H), 4.43 (m, 4H), 2.79 (q, J=7.5 Hz, 2H), 2.60 (t, J=6.0 Hz, 4H), 2.37 (s, 3H), 1.32 (t, J=7.5 Hz, 3H). MS m/z: 297 (M+H⁺).

### EXAMPLE 91

### 8-ethyl-4-(piperazin-1-yl)-[1,2,4]triazolo[4,3-a]quinoxaline

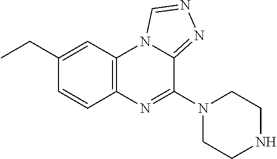

The HCl salt of the title compound was prepared as described in Example 52, except that tert-butyl 4-(8-ethyl-10-hydro-1,2,4-triazolo[4,3-a]quinoxalin-4-yl)piperazinecarboxylate (prepared as described in Example 90 and 88 from tert-butyl 4-(8-bromo-10-hydro-1,2,4-triazolo[4,3-a]quinoxalin-4-yl)piperazinecarboxylate) was substituted for tert-butyl 4-(8-bromo-10-hydro-1,2,4-triazolo[4,3-a]quinoxalin-4-yl)piperazinecarboxylate in step 6 of that route. ¹H NMR (300 MHz, D₂O) δ: 9.17 (s, 1H), 7.10-6.98 (m, 3H), 4.19 (t, J=4.8 Hz, 1H), 3.37 (t, J=5.4 Hz, 4H), 2.45 (q, J=7.5 Hz, 2H), 1.05 (t, J=7.5 Hz, 3H). MS m/z: 283 (M+H⁺).

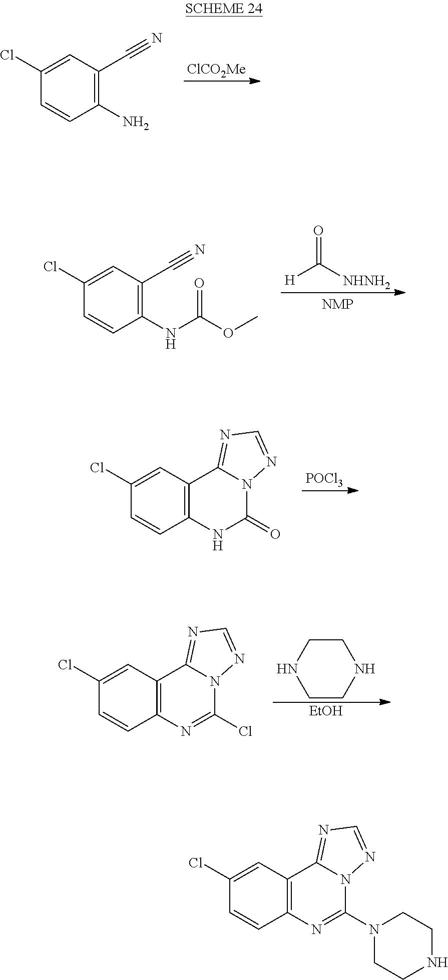

### EXAMPLE 92

### 9-Chloro-5-(piperazin-1-yl)-[1,2,4]triazolo[1,5-c]quinazoline

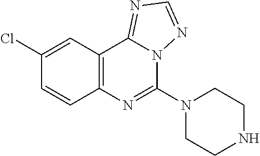

### Step 1

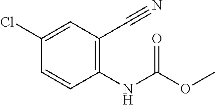

### Methyl 4-chloro-2-cyanophenylcarbamate

A 100 mL round bottom flask was charged with 2-amino-5-chlorobenzonitrile (0.76 g, 5.0 mmol), methyl chloroformate (0.43 mL, 5.40 mmol), NaHCO₃ (0.5 g, 6.0 mmol) and 2-butanone (25 mL). The resulting mixture was stirred overnight at reflux. Reaction progress was monitored by TLC (EtOAc/Petroleum ether=1:10). Work-up: the reaction mixture was filtered and the solid was washed more 2-butanone (20 mL×2). The filtrate was concentrated in vacuo, to give 0.95 g (97%) of the product as white solid.

### Step 2

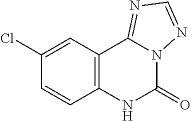

### 9-Chloro-[1,2,4]triazolo[1,5-c]quinazolin-5(6H)-one

A 100 mL round bottom flask was charged with methyl 4-chloro-2-cyanophenylcarbamate (0.9 g, 4.26 mmol), formic hydrazide (0.3 g, 5.12 mmol) and 1-methyl-2-pyrrolidone (25 mL). The resulting mixture was heated at 180° C. for 1.5 h. Reaction progress was monitored by TLC (EtOAc/Petroleum ether=1:2). Work-up: the solvent was evaporated under reduced pressure and the residue was poured into EtOAc (20 mL) and well-mixed by stirring. The solid was collected by filtration, washed with EtOAc (20 mL) and dried, to give 0.88 g (85%) of the product as light yellow crystalline solid. ¹H NMR (300 MHz, DMSO-d₆) δ: 12.45 (s, 1H), 8.55 (s, 1H), 8.12 (d, J=2.4 Hz, 1H), 7.75 (dd, J=9.0, 2.4 Hz, 1H), 7.45 (d, J=9.0 Hz, 1H). MS m/z: 219 (M−H⁺).

### Step 3

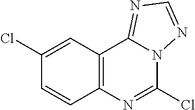

### 5,9-Dichloro-[1,2,4]triazolo[1,5-c]quinazoline

A 50 mL round bottom flask was charged with 9-chloro-[1,2,4]triazolo[1,5-c]quinazolin-5(6H)-one (0.88 g, 4.0 mmol) and phosphorus oxychloride (15 mL). To the above was added dropwise N,N-diisopropylethylamine (1.38 g, 8.0 mmol). The resulting mixture was heated at reflux for 8 h. Reaction progress was monitored by TLC (EtOAc/Petroleum ether=1:8). Work-up: the solvent was evaporated under reduced pressure and the residue was poured into EtOAc (20 mL) and well-mixed by stirring. The solid was collected by filtration, washed with CH₂Cl₂ (20 mL), and dried, to give 0.77 g (81%) of the product as light yellow crystalline solid. ¹H NMR (300 MHz, CDCl₃) δ: 8.51 (dd, J=2.4, 0.3 Hz, 1H), 8.48 (s, 1H), 7.97 (d, J=9.0 Hz, 1H), 7.81 (dd, J=9.0, 2.4 Hz, 1H). MS m/z: 239 (M+H⁺).

### Step 4

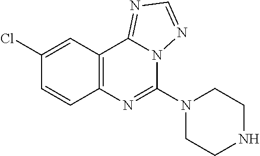

### 9-Chloro-5-(piperazin-1-yl)-[1,2,4]triazolo[1,5-c]quinazoline

A 5 mL microwave reaction tube was charged with 5,9-dichloro-[1,2,4]triazolo[1,5-c]quinazoline (0.12 g, 0.50 mmol), piperazine (0.103 g, 0.55 mmol) and EtOH (4 mL). The resulting mixture was heated at 130° C. for 1.5 h in a Biotage microwave reactor. Work-up: the solvent was evaporated under reduced pressure. The solid was collected by filtration, washed with H₂O (10 mL) and dried, to give 0.18 g (92%) of the product as light yellow crystalline solid. ¹H NMR (300 MHz, CD₃OD) δ: 8.52 (s, 1H), 8.32 (m, 1H), 7.75 (m, 2H), 4.33 (t, J=5.1 Hz, 4H), 3.48 (t, J=5.4 Hz, 4H). MS m/z: 289 (M+H⁺).

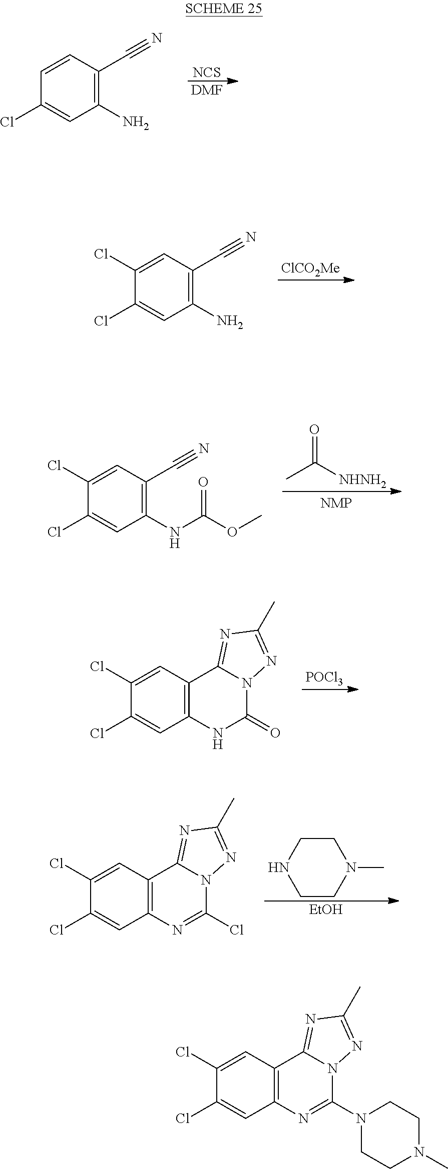

### EXAMPLE 93

### 8,9-Dichloro-2-methyl-5-(4-methylpiperazin-1-yl)-[1,2,4]triazolo[1,5-c]quinazoline

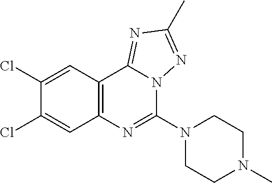

### Step 1

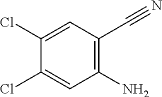

### 2-Amino-4,5-dichlorobenzonitrile

A 10 mL round bottom flask was charged with 2-amino-4-chlorobenzonitrile (0.2 g, 1.31 mmol), N-chlorosuccinimide (0.19 g, 1.44 mmol) and DMF (5 mL). The resulting mixture was stirred at 25° C. overnight. Reaction progress was monitored by TLC (EtOAc/Petroleum ether=1:10). Work-up: the reaction mixture was diluted with EtOAc (40 mL) and washed with brine (40 mL). The organic layer was dried over anhydrous Na₂SO₄ and concentrated in vacuo. The residue was purified by flash column chromatography on silica gel with a 1:10 EtOAc/Petroleum ether, to afford 170 mg (47%) of the product as white solid. ¹H NMR (300 MHz, CDCl₃) δ: 7.45 (s, 1H), 6.88 (s, 1H), 4.48 (br, 2H).

### Steps 2-5

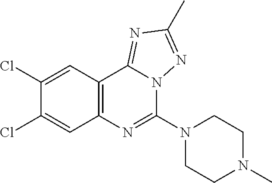

### 8,9-Dichloro-2-methyl-5-(4-methylpiperazin-1-yl)-[1,2,4]triazolo[1,5-c]quinazoline

The title compound was prepared as described in Example 92, except that N-methylpiperazine was substituted for piperazine in step 4,2-amino-4,5-dichlorobenzonitrile for 2-amino-5-chlorobenzonitrile in step 1, and acetic hydrazide for formic hydrazide in step 2. ¹H NMR (300 MHz, CD₃OD) δ: 8.26 (s, 1H), 7.77 (s, 1H), 4.12 (t, J=5.1 Hz, 4H), 2.67 (t, J=5.1 Hz, 4H), 2.58 (s, 3H), 2.38 (s, 3H). MS m/z: 351 (M+H⁺).

### EXAMPLE 94

### 8,9-Dichloro-2-methyl-5-(piperazin-1-yl)-[1,2,4]triazolo[1,5-c]quinazoline

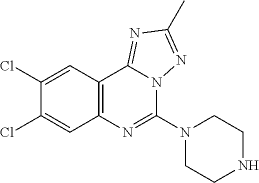

The title compound was prepared as described in Example 93, except that piperazine was substituted for N-methylpiperazine in step 5 of that route. ¹H NMR (300 MHz, CD₃OD) δ: 8.22 (s, 1H), 7.73 (s, 1H), 4.08 (m, 4H), 3.06 (m, 4H), 2.58 (s, 3H). MS m/z: 337 (M+H⁺).

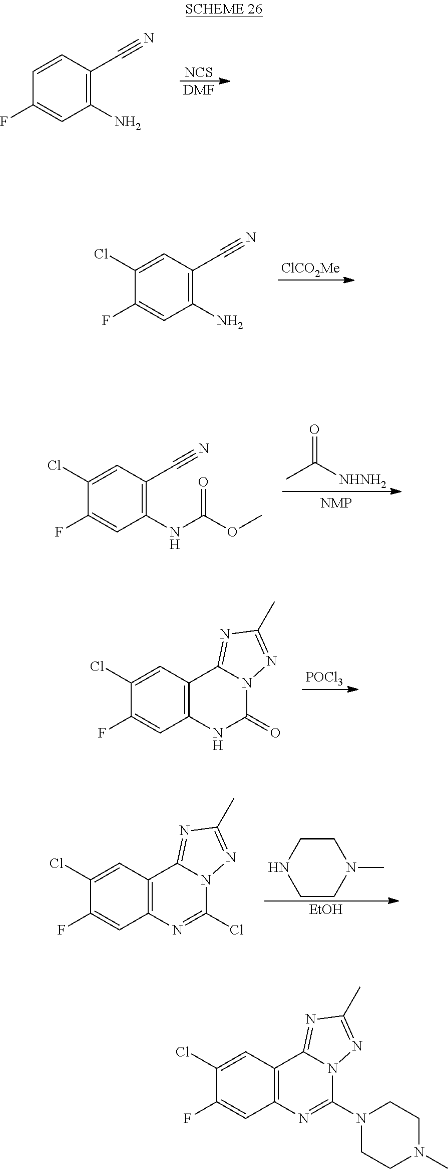

### EXAMPLE 95

### 9-Chloro-8-fluoro-2-methyl-5-(4-methylpiperazin-1-yl)-[1,2,4]triazolo[1,5-c]quinazoline

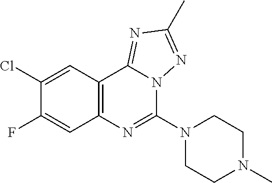

The title compound was prepared as described in Example 93, except that 2-amino-4-fluorobenzonitrile was substituted for 2-amino-4-chlorobenzonitrile in step 1 of that route. ¹H NMR (300 MHz, CD₃OD) δ: 8.22 (d, J=8.1 Hz, 1H), 7.42 (d, J=10.5 Hz, 1H), 4.12 (t, J=4.8 Hz, 4H), 2.66 (t, J=4.8 Hz, 4H), 2.57 (s, 3H), 2.37 (s, 3H). MS m/z: 335 (M+H⁺).

### EXAMPLE 96

### 9-Chloro-8-fluoro-2-methyl-5-(piperazin-1-yl)-[1,2,4]triazolo[1,5-c]quinazoline

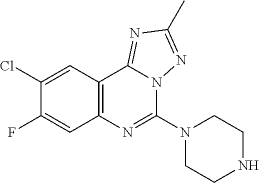

The title compound was prepared as described in Example 95, except that piperazine was substituted for N-methylpiperazine in step 5 of that route. ¹H NMR (300 MHz, CD₃OD) δ: 8.26 (d, J=7.8 Hz, 1H), 7.46 (d, J=10.8 Hz, 1H), 4.08 (m, 4H), 3.03 (m, 4H), 2.58 (s, 3H). MS m/z: 321 (M+H⁺).

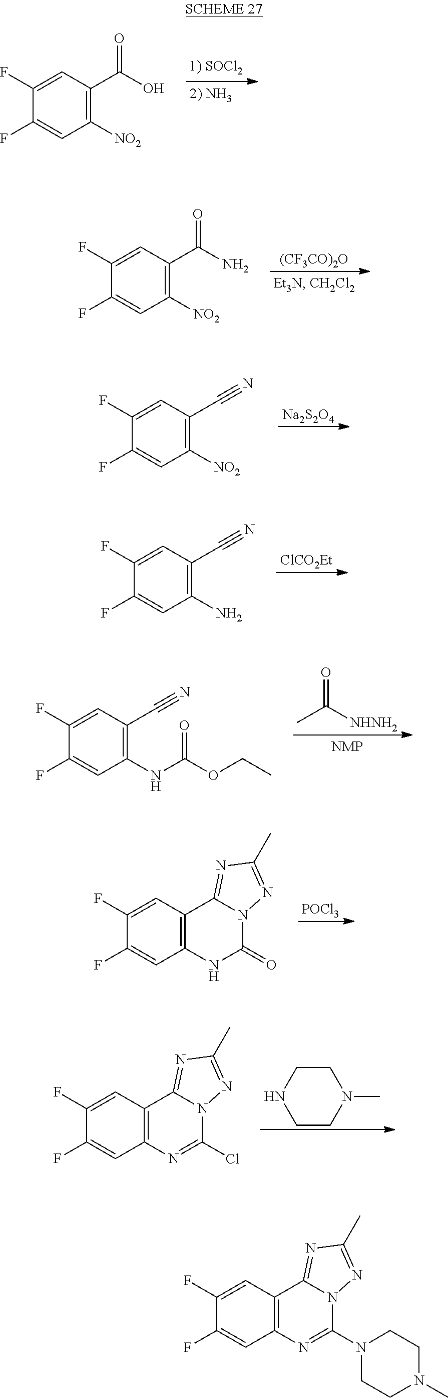

### EXAMPLE 97

### 8,9-Difluoro-2-methyl-5-(4-methylpiperazin-1-yl)-[1,2,4]triazolo[1,5-c]quinazoline

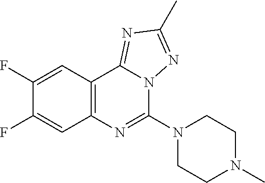

### Step 1

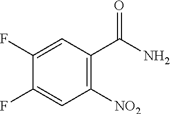

### 4,5-Difluoro-2-nitrobenzamide

A 100 mL round bottom flask was charged with 4,5-difluoro-2-nitrobenzoic acid (5.08 g, 25 mmol) and SOCl₂ (15 mL). The mixture was refluxed for 1 h then concentrated in vacuo. To the residue was added slowly 25% aqueous ammonia (30 mL) at 0° C. and the reaction mixture was stirred for further 2 h at 0° C. The reaction progress was monitored by TLC (EtOAc/Petroleum ether=1:1, Rf=0.4). Work-up: the solid was collected by filtration and dried to afford 4.06 g (80%) of the product as brown solid.

### Step 2

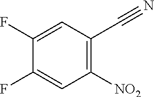

### 4,5-Difluoro-2-nitrobenzonitrile

A 250 mL round bottom flask was charged with 4,5-difluoro-2-nitrobenzamide (4.06 g, 20 mmol), (CF₃CO)₂O (5.6 mL, 40 mmol), Et₃N (5.6 mL, 40 mmol) and CH₂Cl₂ (120 mL). The resulting mixture was stirred for 1 h at room temperature. The reaction progress was monitored by TLC (EtOAc/Petroleum ether=1:4, Rf=0.7). Work-up: the reaction mixture was diluted with more CH₂Cl₂ (120 mL), washed with saturated aqueous NaHCO₃ (250 mL). The organic layer was dried over anhydrous Na₂SO₄ and concentrated in vacuo. The oil residue solidified after 1 h at room temperature, to afford 4.5 g (quantitative yield) of the product as orange solid. ¹H NMR (300 MHz, DMSO-d₆) δ: 8.70 (dd, J=10.3, 7.3 Hz, 1H), 8.58 (dd, J=10.1, 7.5 Hz, 1H).

### Step 3

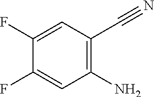

### 2-Amino-4,5-difluorobenzonitrile

A 250 mL round bottom flask was charged with 4,5-difluoro-2-nitrobenzonitrile (3.68 g, 20 mmol), Na₂S₂O₄ (85% purity, 8.19 g, 40 mmol), EtOH (150 mL) and H₂O (20 mL). The resulting mixture was stirred at reflux overnight and then concentrated to dryness under reduced pressure. The residue was suspended in saturated aqueous NaHCO₃ (200 mL) and extracted with ethyl ether (100 mL×3). The combined organic layers were dried over anhydrous Na₂SO₄ then concentrated in vacuo, to afford 1.2 g (39%) of the product as yellow solid. ¹H NMR (300 MHz, DMSO-d₆) δ: 7.64 (dd, J=10.8, 8.9 Hz, 1H), 6.72 (dd, J=13.1, 7.1 Hz, 1H), 6.24 (br, 2H).

### Step 4

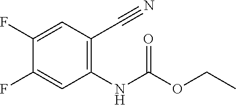

### Ethyl 2-cyano-4,5-difluorophenylcarbamate

A 100 mL round bottom flask was charged with 2-amino-4,5-difluorobenzonitrile (1.1 g, 7.1 mmol), ethyl chloroformate (25 mL, 260 mmol) and NaHCO₃ (0.72 g, 8.6 mmol). The resulting mixture was refluxed overnight (16 h) then cooled to room temperature. It was diluted with CH₂Cl₂ (200 mL) then filtered and concentrated under reduced pressure. The residue was purified by flash column chromatography on silica gel with 10% AcOEt in petroleum ether, to afford 1.36 g (84%) of the product as white solid. ¹H NMR (300 MHz, DMSO-d₆) δ: 9.91 (s, 1H), 8.11 (dd, J=10.4, 8.5 Hz, 1H), 7.65 (dd, J=12.1, 7.4 Hz, 1H), 4.16 (q, J=7.1 Hz, 2H), 1.25 (t, J=7.1 Hz, 3H).

### Step 5

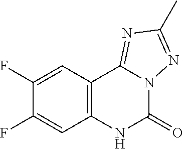

### 8,9-Difluoro-2-methyl-[1,2,4]triazolo[1,5-c]quinazolin-5(6H)-one

A 50 mL round bottom flask was charged with ethyl 2-cyano-4,5-difluorophenylcarbamate (1.36 g, 6.0 mmol), acetic hydrazide (0.535 g, 7.2 mmol) and 1-methyl-2-pyrrolidone (15 mL). The resulting solution was refluxed for 2 h. The 1-methyl-2-pyrrolidone was then removed under reduced pressure, to afford 1.42 g (quantitative) of the product as orange solid. It was used directly in the next step.

### Step 6

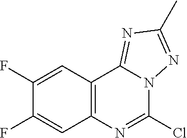

### 5-Chloro-8,9-difluoro-2-methyl-[1,2,4]triazolo[1,5-c]quinazoline

A 100 mL round bottom flask was charged with 8,9-difluoro-2-methyl-[1,2,4]triazolo[1,5-c]quinazolin-5(6H)-one (1.42 g, 6.0 mmol) and POCl₃ (20 mL). After N,N-diisopropylethylamine (2.1 mL, 12 mmol) was added dropwise at 0° C., the resulting mixture was refluxed overnight (16 h) and then concentrated under reduced pressure. The residue was carefully diluted with saturated aqueous NaHCO₃ (150 mL), then extracted with CH₂Cl₂ (150 mL×2). The combined organic layers were washed with brine (150 mL), dried over anhydrous Na₂SO₄ then concentrated in vacuo. The residue was purified by flash column chromatography on silica gel with 20-50% AcOEt in CH₂Cl₂ (containing 1% Et₃N), to afford 0.96 g (63%) of the product as light-orange solid. ¹H NMR (300 MHz, CDCl₃) δ: 8.19 (dd, J=9.4, 8.1 Hz, 1H), 7.75 (dd, J=10.3, 7.1 Hz, 1H), 2.66 (s, 3H).

### Step 7

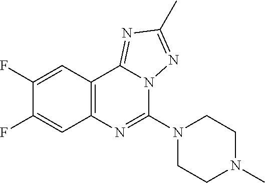

### 8,9-Difluoro-2-methyl-5-(4-methylpiperazin-1-yl)-[1,2,4]triazolo[1,5-c]quinazoline

A 100 mL round bottom flask was charged with 5-chloro-8,9-difluoro-2-methyl-[1,2,4]triazolo[1,5-c]quinazoline (0.2 g, 0.8 mmol), N-methylpiperazine (0.1 mL, 0.9 mmol), Et₃N (0.5 mL, 3.6 mmol), DMF (10 mL) and THF (10 mL). The resulting solution was stirred at room temperature for 1 h, and then was concentrated under reduced pressure. The residue was mixed with saturated aqueous NaHCO₃ (100 mL), then extracted with CHCl₃ (50 mL×3). The combined organic layers were dried over anhydrous Na₂SO₄ and concentrated in vacuo. The residue was purified by flash column chromatography on silica gel with 2-4% MeOH in CH₂Cl₂ (saturated with NH₃), to afford 0.085 g (34%) of the product as off-white solid. ¹H NMR (300 MHz, CDCl₃) δ: 8.05 (dd, J=9.8, 8.5 Hz, 1H), 7.46 (dd, J=11.4, 7.2 Hz, 1H), 4.09 (t, J=4.8 Hz, 4H), 2.66 (t, J=4.8 Hz, 4H), 2.62 (s, 3H), 2.40 (s, 3H). MS m/z: 319 (M+H⁺).

### EXAMPLE 98

### 8,9-Difluoro-2-methyl-5-(piperazin-1-yl)-[1,2,4]triazolo[1,5-c]quinazoline

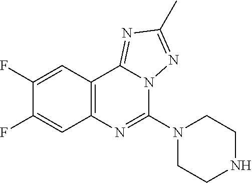

The title compound was prepared as described in Example 97, except that piperazine was substituted for N-methylpiperazine in step 7 of that route. ¹H NMR (300 MHz, CDCl₃) δ: 8.05 (dd, J=9.9, 8.4 Hz, 1H), 7.46 (dd, J=11.4, 7.1 Hz, 1H), 4.01 (t, J=5.1 Hz, 4H), 3.10 (t, J=5.1 Hz, 4H), 2.62 (s, 3H). MS m/z: 305 (M+H⁺).

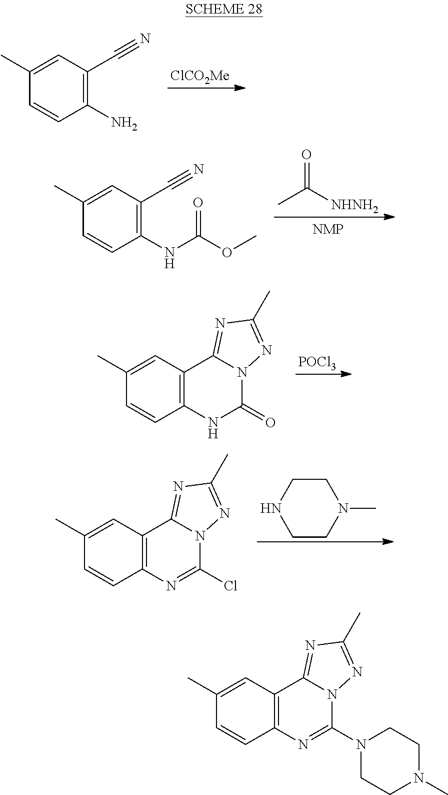

### EXAMPLE 99

### 2,9-Dimethyl-5-(4-methylpiperazin-1-yl)-[1,2,4]triazolo[1,5-c]quinazoline

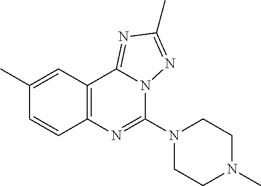

### Step 1

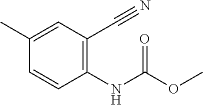

### Methyl 2-cyano-4-methylphenylcarbamate

A 100 mL round bottom flask was charged with 2-amino-5-methylbenzonitrile (3.5 g, 26.5 mmol), Na₂CO₃ (5.8 g, 54.7 mmol) and methyl chloroformate (50 mL). The resulting solution was heated at reflux overnight. The reaction mixture was concentrated. The resulting precipitate was collected by filtration, to afford 2.6 g (52%) of the product as yellow solid.

### Step 2

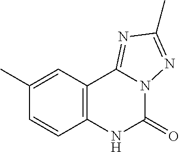

### 2,9-Dimethyl-[1,2,4]triazolo[1,5-c]quinazolin-5(6H)-one

A 100 mL round bottom flask was charged with methyl 2-cyano-4-methylphenylcarbamate (2.6 g, 13.7 mmol), acetic hydrazide (1.2 g, 16.2 mmol) and 1-methyl-2-pyrrolidone (50 mL). The resulting solution was heated at 180° C. for 1 h then concentrated in vacuo. The resulting precipitate was collected by filtration, washed with EtOAc and dried, to afford 2 g (68%) of the product.

### Step 3

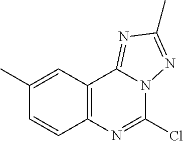

### 5-Chloro-2,9-dimethyl-[1,2,4]triazolo[1,5-c]quinazoline

A 100 mL round bottom flask was charged with 2,9-dimethyl-[1,2,4]triazolo[1,5-c]quinazolin-5(6H)-one (1 g, 1.07 mmol), N,N-dimethylanaline (0.26 mL, 2.14 mmol) and POCl₃ (10 mL). The resulting solution was heated at reflux for 3 h then concentrated in vacuo. The residue was poured into saturated aqueous Na₂CO₃ and extracted with CH₂Cl₂. The combined organic layers were dried over anhydrous Na₂SO₄ and concentrated in vacuo. The residue was purified by flash column chromatography on silica gel with 10% EtOAc in petroleum ether, to afford 300 mg (27%) of the product as white solid. MS m/z: 233 (M+H⁺).

### Step 4

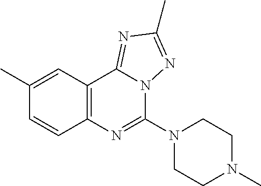

### 2,9-Dimethyl-5-(4-methylpiperazin-1-yl)-[1,2,4]triazolo[1,5-c]quinazoline

A 20 mL microwave reaction tube was charged with 5-chloro-2,9-dimethyl-[1,2,4]triazolo[1,5-c]quinazoline (150 mg, 0.64 mmol), N-methylpiperazine (0.22 mL, 1.98 mmol) and anhydrous EtOH (10 mL). The resulting solution was heated at 130° C. for 1 h in a Biotage microwave reactor. The solvent was evaporated and the residue was purified by flash column chromatography on silica gel with 10% MeOH in CH₂Cl₂ to afford 110 mg (57%) of the product as white solid. ¹H NMR (300 MHz, CD₃OD) δ: 8.00 (s, 1H), 7.53 (m, 2H), 3.99 (br, 4H), 2.66 (t, J=5.1 Hz, 4H), 2.57 (s, 3H), 2.48 (s, 3H), 2.37 (s, 3H). MS m/z: 297 (M+H⁺).

### EXAMPLE 100

### 2,9-Dimethyl-5-(piperazin-1-yl)-[1,2,4]triazolo[1,5-c]quinazoline

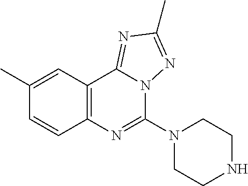

The title compound was prepared as described in Example 99, except that piperazine was substituted for N-methylpiperazine in step 4 of that route. ¹H NMR (300 MHz, CD₃OD) δ: 8.06 (d, J=1.2 Hz, 1H), 7.63 (d, J=8.7 Hz, 1H), 7.56 (dd, J=8.4, 1.5 Hz, 1H), 3.93 (m, 4H), 3.04 (m, 4H), 2.59 (s, 3H), 2.50 (s, 3H). MS m/z: 283 (M+H⁺).

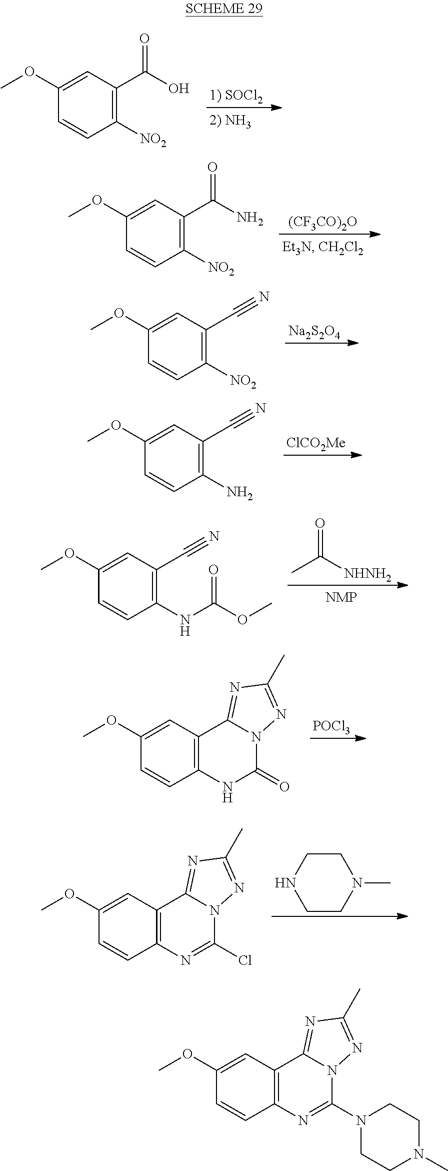

### EXAMPLE 101

### 9-Methoxy-2-methyl-5-(4-methylpiperazin-1-yl)-[1,2,4]triazolo[1,5-c]quinazoline

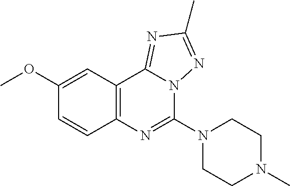

### Step 1

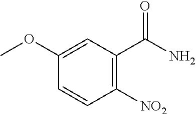

### 5-Methoxy-2-nitrobenzamide

A 100 mL round bottom flask was charged with 5-methoxy-2-nitrobenzoic acid (1.5 g, 7.61 mmol), DMF (1 mL) and SOCl₂ (15 mL). The resulting mixture was heated at reflux for 1 h then concentrated in vacuo. The residue was re-dissolved in DMF (3 mL) and the solution was added dropwise to aqueous ammonia (25%, 15 mL) at 0° C. with vigorous stirring. Work-up: the resulting solid was collected by filtration, washed with H₂O (20 mL) and dried, to give 1.2 g (80%) of the product as white solid.

### Step 2

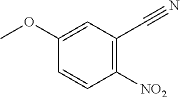

### 5-Methoxy-2-nitrobenzonitrile

A 100 mL round bottom flask was charged with 5-methoxy-2-nitrobenzamide (2.1 g, 0.01 mol), trifluoroacetic anhydride (2.2 mL), triethylamine (2.9 mL) and CH₂Cl₂ (30 mL). The resulting solution was stirred at room temperature for 1 h. Work-up: the reaction solution was washed with H₂O (30 mL×2). The organic layer was dried over anhydrous Na₂SO₄ and concentrated in vacuo, to give 1.75 g (92%) of the product as white solid. MS m/z: 179 (M+H⁺).

### Step 3

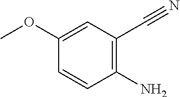

### 2-Amino-5-methoxybenzonitrile

A 100 mL round bottom flask was charged with 5-methoxy-2-nitrobenzonitrile (1.7 g, 9.55 mmol), sodium dithionite (4.99 g, 29 mmol), water (15 mL) and EtOH (50 mL). The resulting mixture was heated at reflux for 1 h. Work-up: the reaction mixture was concentrated in vacuo to remove ethanol then extracted with EtOAc (50 mL). The organic layer was dried over anhydrous Na₂SO₄ and concentrated in vacuo, to afford 1.4 g (quantitative) of the product as yellow oil. It was used in the next step without further purification.

### Steps 4-7

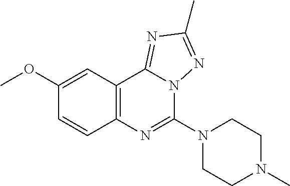

### 9-Methoxy-2-methyl-5-(4-methylpiperazin-1-yl)-[1,2,4]triazolo[1,5-c]quinazoline

The title compound was prepared as described in Examples 92, except that N-methylpiperazine was substituted for piperazine in step 4,2-amino-5-methoxybenzonitrile for 2-amino-5-chlorobenzonitrile in step 1, and acetic hydrazide for formic hydrazide in step 2. ¹H NMR (300 MHz, CDCl₃) δ: 7.67 (m, 2H), 7.29 (dd, J=9.0, 2.7 Hz, 1H), 3.99 (m, 4H), 3.93 (s, 3H), 2.68 (m, 4H), 2.65 (s, 3H), 2.40 (s, 3H). MS m/z: 313 (M+H⁺).

### EXAMPLE 102

### 9-Methoxy-2-methyl-5-(piperazin-1-yl)-[1,2,4]triazolo[1,5-c]quinazoline

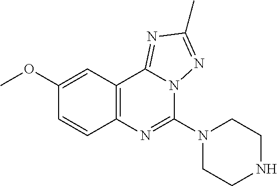

The title compound was prepared as described in Example 101, except that piperazine was substituted for N-methylpiperazine in step 7 of that route. ¹H NMR (300 MHz, CD₃OD) δ: 7.66 (m, 2H), 7.32 (dd, J=9.0, 3.0 Hz, 1H), 3.96-3.92 (m, 7H), 3.12 (t, J=5.1 Hz, 4H), 2.59 (s, 3H). MS m/z: 299 (M+H⁺).

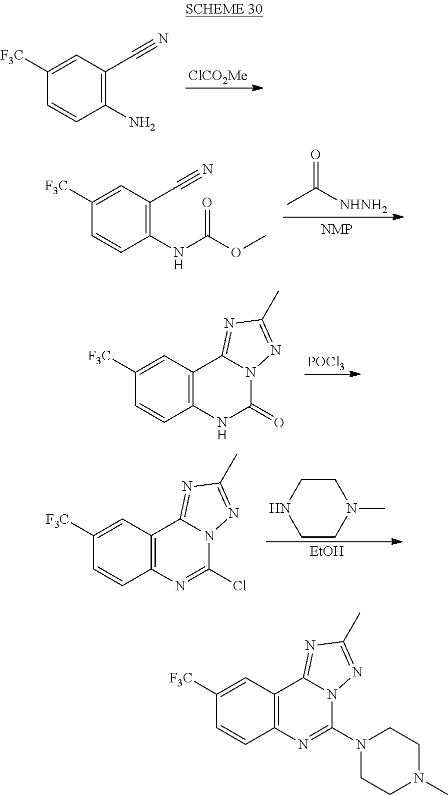

### EXAMPLE 103

### 2-Methyl-5-(4-methylpiperazin-1-yl)-9-(trifluoromethyl)-[1,2,4]triazolo[1,5-c]quinazoline

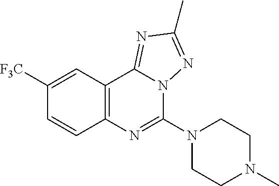

The title compound was prepared as described in Examples 92, except that N-methylpiperazine was substituted for piperazine in step 4,2-amino-5-(trifluoromethyl)benzonitrile for 2-amino-5-chlorobenzonitrile in step 1, and acetic hydrazide for formic hydrazide in step 2. ¹H NMR (300 MHz, CD₃OD) δ: 8.52 (s, 1H), 7.89 (dd, J=9.0, 2.4 Hz, 1H), 7.78 (dd, J=9.0, 0.6 Hz, 1H), 4.19 (t, J=5.1 Hz, 4H), 2.67 (t, J=5.1 Hz, 4H), 2.60 (s, 3H), 2.37 (s, 3H). MS m/z: 351 (M+H⁺).

### EXAMPLE 104

### 2-Methyl-5-(piperazin-1-yl)-9-(trifluoromethyl)-[1,2,4]triazolo[1,5-c]quinazoline

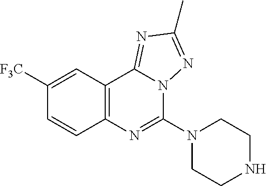

The title compound was prepared as described in Example 103, except that piperazine was substituted for N-methylpiperazine in step 4 of that route. ¹H NMR (300 MHz, CD₃OD) δ: 8.45 (d, J=0.3 Hz, 1H), 7.85 (dd, J=8.7, 2.4 Hz, 1H), 7.72 (d, J=8.7 Hz, 1H), 4.11 (m, 4H), 3.02 (t, J=4.8 Hz, 4H), 2.57 (s, 3H). MS m/z: 337 (M+H⁺).

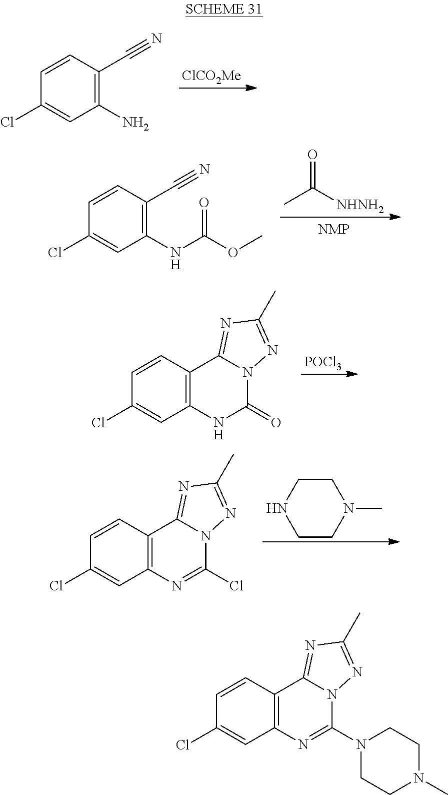

### EXAMPLE 105

### 8-Chloro-2-methyl-5-(4-methylpiperazin-1-yl)-[1,2,4]triazolo[1,5-c]quinazoline

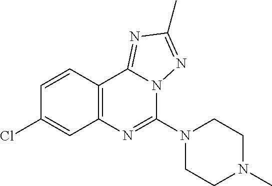

The title compound was prepared as described in Examples 92, except that N-methylpiperazine was substituted for piperazine in step 4,2-amino-4-chlorobenzonitrile for 2-amino-5-chlorobenzonitrile in step 1, and acetic hydrazide for formic hydrazide in step 2. ¹H NMR (300 MHz, CDCl₃) δ: 8.23 (d, J=8.7 Hz, 1H), 7.71 (d, J=2.1 Hz, 1H), 7.37 (dd, J=8.7, 2.1 Hz, 1H), 4.12 (m, 4H), 2.63 (m, 7H), 2.38 (s, 3H). MS m/z: 317 (M+H⁺).

### EXAMPLE 106

### 8-Chloro-2-methyl-5-(piperazin-1-yl)-[1,2,4]triazolo[1,5-c]quinazoline

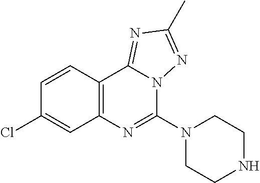

The title compound was prepared as described in Example 105, except that piperazine was substituted for N-methylpiperazine in step 4 of that route. ¹H NMR (300 MHz, CDCl₃) δ: 8.23 (d, J=8.4 Hz, 1H), 7.72 (d, J=1.8 Hz, 1H), 7.38 (dd, J=8.7, 2.1 Hz, 1H), 4.17 (t, J=4.8 Hz, 4H), 3.20 (t, J=4.8 Hz, 4H), 2.63 (s, 3H). MS m/z: 303 (M+H⁺).

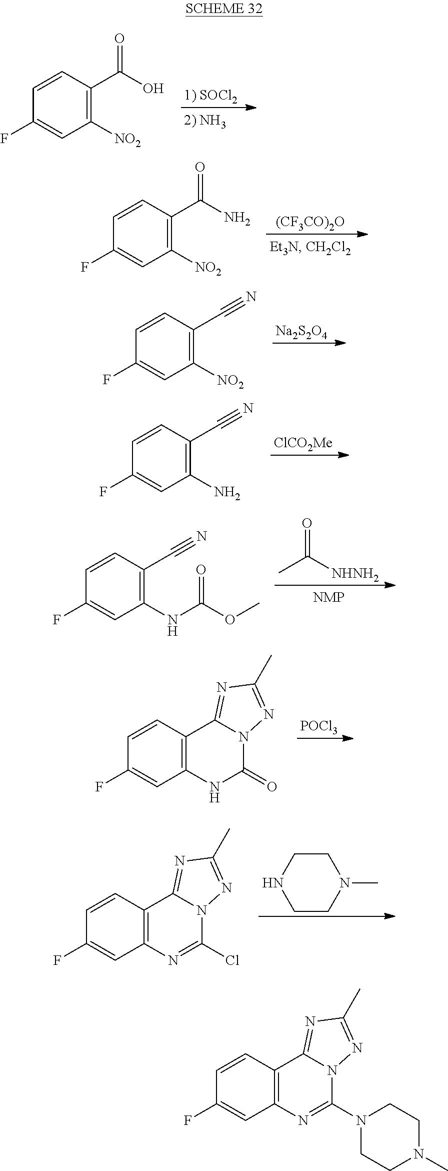

### EXAMPLE 107

### 8-Fluoro-2-methyl-5-(4-methylpiperazin-1-yl)-[1,2,4]triazolo[1,5-c]quinazoline

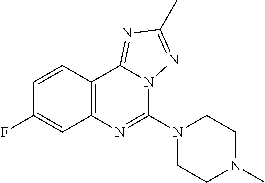

The title compound was prepared as described in Example 101, except that 4-fluoro-2-nitrobenzoic acid was substituted for 5-methoxy-2-nitrobenzoic acid in step 1 of that route. ¹H NMR (300 MHz, CD₃OD) δ: 8.26 (dd, J=8.7, 6.0 Hz, 1H), 7.35 (dd, J=10.5, 2.4 Hz, 1H), 7.24 (m, 1H), 4.12 (m, 4H), 2.68 (m, 4H), 2.58 (s, 3H), 2.37 (s, 3H). MS m/z: 301 (M+H⁺).

### EXAMPLE 108

### 8-Fluoro-2-methyl-5-(piperazin-1-yl)-[1,2,4]triazolo[1,5-c]quinazoline

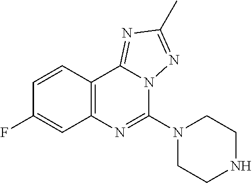

The title compound was prepared as described in Example 107, except that piperazine was substituted for N-methylpiperazine in step 7 of that route. ¹H NMR (300 MHz, CD₃OD) δ: 8.25 (dd, J=9.0, 6.0 Hz, 1H), 7.33 (dd, J=10.5, 2.7 Hz, 1H), 7.22 (m, 1H), 4.05 (m, 4H), 3.30 (m, 4H), 2.58 (s, 3H). MS m/z: 287 (M+H⁺).

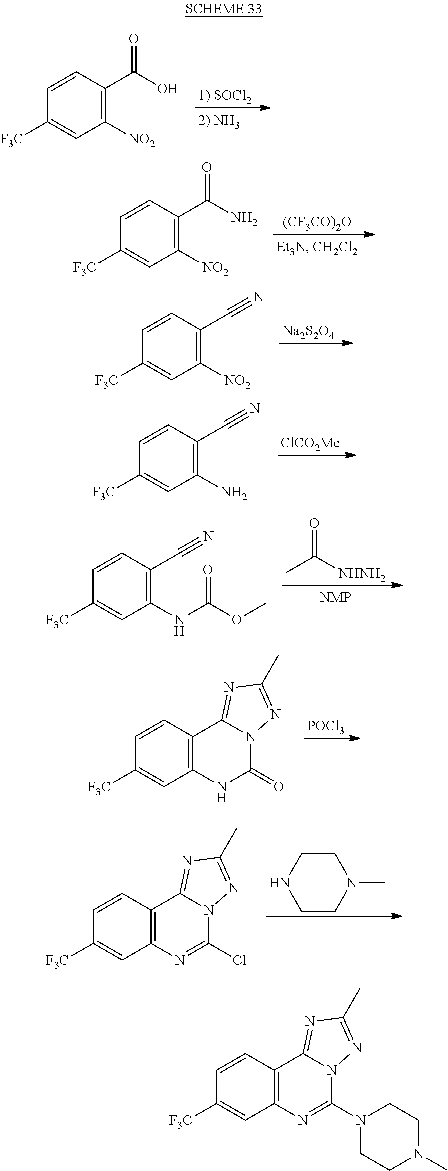

### EXAMPLE 109

### 2-Methyl-5-(4-methylpiperazin-1-yl)-8-(trifluoromethyl)-[1,2,4]triazolo[1,5-c]quinazoline

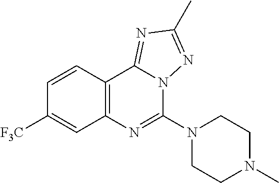

The title compound was prepared as described in Example 101, except that 2-nitro-4-(trifluoromethyl)benzoic acid was substituted for 5-methoxy-2-nitrobenzoic acid in step 1 of that route. ¹H NMR (300 MHz, CDCl₃) δ: 8.40 (d, J=8.4 Hz, 1H), 7.92 (s, 1H), 7.59 (d, J=8.4 Hz, 1H), 4.14 (br, 4H), 2.65 (m, 7H), 2.38 (s, 3H). MS m/z: 351 (M+H⁺).

### EXAMPLE 110

### 2-Methyl-5-(piperazin-1-yl)-8-(trifluoromethyl)-[1,2,4]triazolo[1,5-c]quinazoline

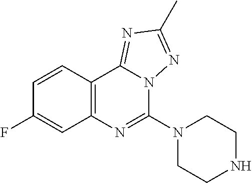

The title compound was prepared as described in Example 109, except that piperazine was substituted for N-methylpiperazine in step 7 of that route. ¹H NMR (300 MHz, CD₃OD) δ: 8.35 (d, J=8.4 Hz, 1H), 7.92 (s, 1H), 7.63 (dd, J=8.4, 1.5 Hz, 1H), 4.08 (m, 4H), 3.04 (m, 4H), 2.60 (s, 3H). MS m/z: 337 (M+H⁺).

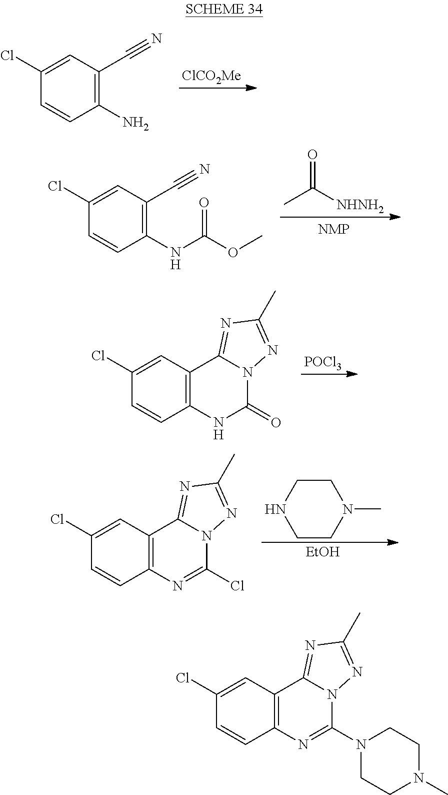

### EXAMPLE 111

### 9-chloro-5-(4-methylpiperazin-1-yl)-[1,2,4]triazolo[1,5-c]quinazoline

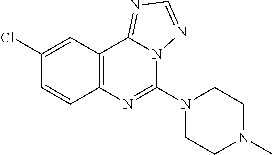

The title compound was prepared as described in Examples 92, except that N-methylpiperazine was substituted for piperazine in step 4. ¹H NMR (300 MHz, CDCl₃) δ: 8.29 (d, J=2.4 Hz, 1H), 7.61 (m, 2H), 4.08 (br, 4H), 2.64 (m, 7H), 2.38 (s, 3H). MS m/z: 317 (M+H⁺).

### EXAMPLE 112

### 9-Chloro-2-methyl-5-(piperazin-1-yl)-[1,2,4]triazolo[1,5-c]quinazoline

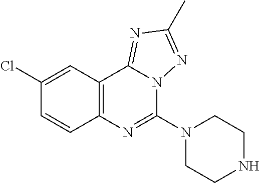

The title compound was prepared as described in Example 111, except that piperazine was substituted for N-methylpiperazine in step 4 of that route. MS m/z: 317 (M+H⁺).

### EXAMPLE 113

### 9-chloro-2-methyl-5-(4-methylpiperazin-1-yl)-[1,2,4]triazolo[1,5-c]quinazoline

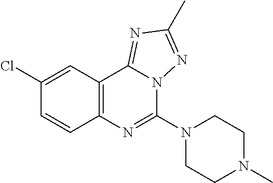

The title compound was prepared as described in Example 111, except that 5,9-dichloro-2-methyl-[1,2,4]triazolo[1,5-c]quinazoline was substituted for 5,9-dichloro-[1,2,4]triazolo[1,5-c]quinazoline in the final step of that route. ¹H NMR (300 MHz, CD₃OD) δ: 8.19 (s, 1H), 7.67 (m, 2H), 4.01 (m, 4H), 3.03 (t, J=5.1 Hz, 4H), 2.59 (s, 3H). MS m/z: 303 (M+H⁺).

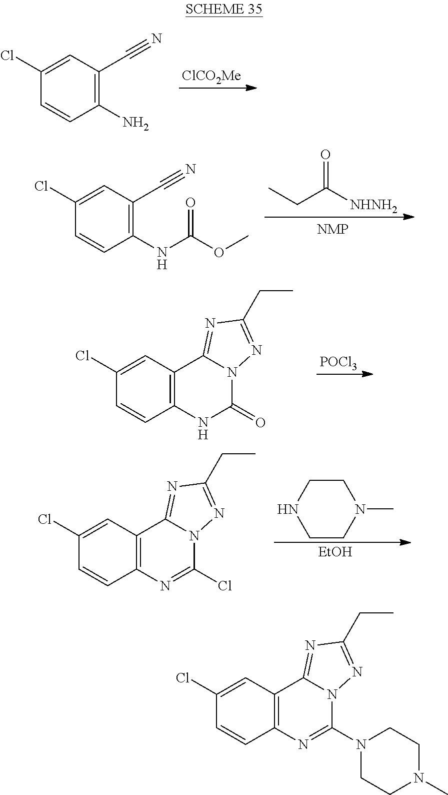

### EXAMPLE 114

### 9-Chloro-2-ethyl-5-(4-methylpiperazin-1-yl)-[1,2,4]triazolo[1,5-c]quinazoline

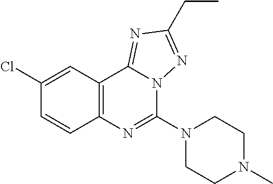

The title compound was prepared as described in Examples 92, except that N-methylpiperazine was substituted for piperazine in step 4, and propionic hydrazide for formic hydrazide in step 2. ¹H NMR (300 MHz, CDCl₃) δ: 8.30 (d, J=2.1 Hz, 1H), 7.60 (m, 2H), 4.09 (br, 4H), 2.97 (q, J=7.5 Hz, 2H), 2.65 (t, J=4.8 Hz, 4H), 2.38 (s, 3H), 1.44 (t, J=7.8 Hz, 3H). MS m/z: 331 (M+H⁺).

### EXAMPLE 115

### 9-Chloro-2-ethyl-5-(piperazin-1-yl)-[1,2,4]triazolo[1,5-c]quinazoline

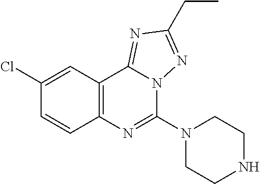

The title compound was prepared as described in Example 114, except that piperazine was substituted for N-methylpiperazine in step 4 of that route. ¹H NMR (300 MHz, CD₃OD) δ: 8.15 (s, 1H), 7.59 (s, 2H), 4.01 (t, J=5.1 Hz, 4H), 3.03 (t, J=4.8 Hz, 4H), 2.94 (q, J=7.5 Hz, 2H), 1.42 (t, J=7.2 Hz, 3H). MS m/z: 317 (M+H⁺).

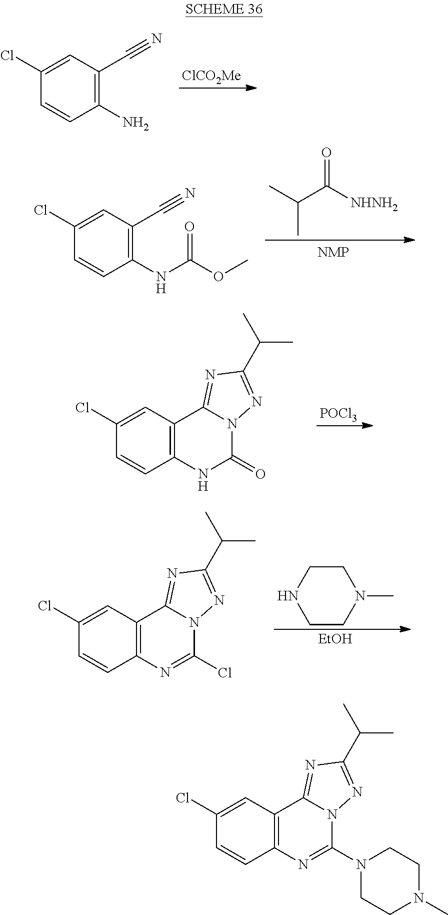

### EXAMPLE 116

### 9-Chloro-2-isopropyl-5-(4-methylpiperazin-1-yl)-[1,2,4]triazolo[1,5-c]quinazoline

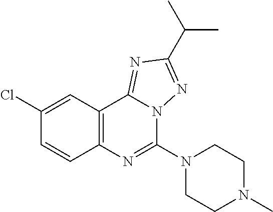

The title compound was prepared as described in Example 92, except that N-methylpiperazine was substituted for piperazine in step 4, and isobutyric hydrazide for formic hydrazide in step 2. ¹H NMR (300 MHz, CD₃OD) δ: 8.25 (t, J=1.5 Hz, 1H), 7.63 (d, J=1.5 Hz, 2H), 4.09 (t, J=4.5 Hz, 4H), 3.29 (m, 1H), 2.67 (t, J=4.5 Hz, 4H), 2.37 (s, 3H), 1.45 (d, J=6.9 Hz, 6H). MS m/z: 345 (M+H⁺).

### EXAMPLE 117

### 9-Chloro-2-isopropyl-5-(piperazin-1-yl)-[1,2,4]triazolo[1,5-c]quinazoline

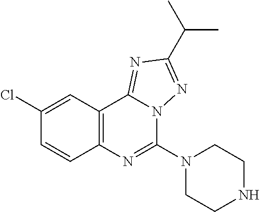

The title compound was prepared as described in Example 116, except that piperazine was substituted for N-methylpiperazine in step 4 of that route. ¹H NMR (300 MHz, CD₃OD) δ: 8.27 (m, 1H), 7.65 (m, 2H), 4.04 (m, 4H), 3.29 (m, 1H), 3.03 (m, 4H), 1.45 (d, J=6.9 Hz, 6H). MS m/z: 331 (M+H⁺).

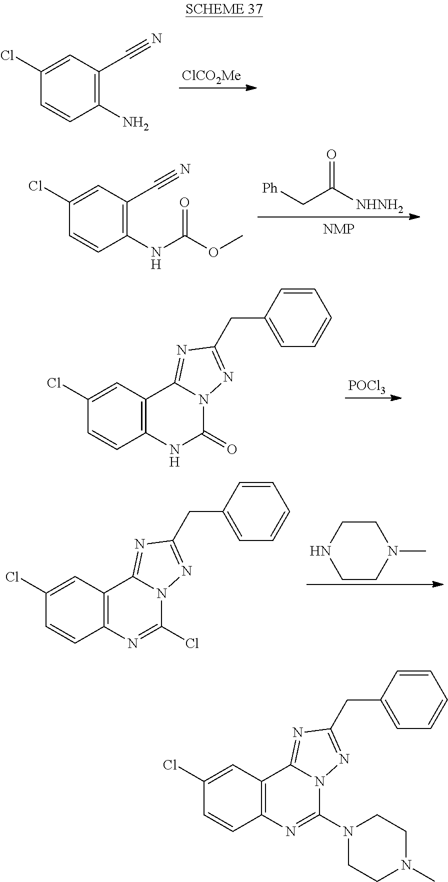

### EXAMPLE 118

### 2-Benzyl-9-chloro-5-(4-methylpiperazin-1-yl)-[1,2,4]triazolo[1,5-c]quinazoline

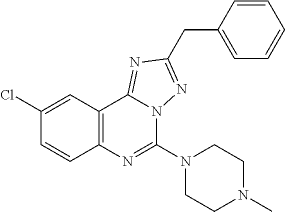

### Step 1

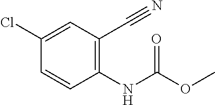

### Methyl 4-chloro-2-cyanophenylcarbamate

A 100 mL round bottom flask was charged with 2-amino-5-chlorobenzonitrile (0.76 g, 5.0 mmol), methyl chloroformate (0.43 mL, 5.40 mmol), NaHCO₃ (0.5 g, 6.0 mmol) and 2-butanone (25 mL). The resulting mixture was stirred overnight at reflux. Reaction progress was monitored by TLC (EtOAc/Petroleum ether=1:10). Work-up: the reaction mixture was filtered and the solid was washed more 2-butanone (20 mL×2). The filtrate was concentrated in vacuo, to give 0.95 g (97%) of the product as white solid.

### Step 2

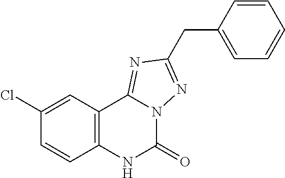

### 2-Benzyl-9-chloro-[1,2,4]triazolo[1,5-c]quinazolin-5(6H)-one

A 50 mL round bottom flask was charged with methyl 4-chloro-2-cyanophenylcarbamate (500 mg, 2.38 mmol), 2-phenylacetic hydrazide (430 mg, 2.86 mmol) and 1-methyl-2-pyrrolidone (20 mL). The resulting solution was heated at 180° C. for 1.5 h then concentrated in vacuo. The resulting precipitate was collected by filtration, washed with EtOAc and dried, to afford 610 mg (82%) of the product.

### Step 3

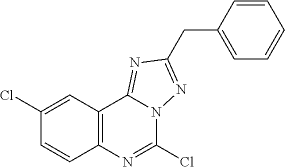

### 2-Benzyl-5,9-dichloro-[1,2,4]triazolo[1,5-c]quinazoline

A 50 mL round bottom flask was charged with 2-benzyl-9-chloro-[1,2,4]triazolo[1,5-c]quinazolin-5(6H)-one (610 mg, 1.97 mmol) and POCl₃ (15 mL). The resulting solution was heated at reflux for 1 h then concentrated in vacuo. The residue was poured into saturated aqueous Na₂CO₃ and extracted with CH₂Cl₂. The combined organic layers were dried over anhydrous Na₂SO₄ and concentrated in vacuo. The residue was purified by flash column chromatography on silica gel with 10% EtOAc in petroleum ether, to afford 330 mg (51%) of the product as white solid.

### Step 4

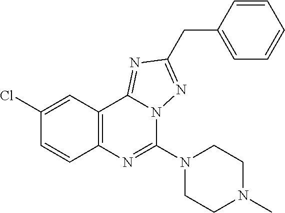

### 2-Benzyl-9-chloro-5-(4-methylpiperazin-1-yl)-[1,2,4]triazolo[1,5-c]quinazoline

A 50 mL round bottom flask was charged with 2-benzyl-5,9-dichloro-[1,2,4]triazolo[1,5-c]quinazoline (160 mg, 0.488 mmol), Et₃N (0.14 mL, 1.0 mmol), N-methylpiperazine (0.07 ml, 0.65 mmol) and anhydrous EtOH (15 mL). The resulting solution stirred at room temperature for 1.5 h then concentrated in vacuo. The resulting solid was washed with H₂O to give 115 mg (60%) of the product as white solid. ¹H NMR (300 MHz, CDCl₃) δ: 8.31 (d, J=2.1 Hz, 1H), 7.62 (d, J=8.7 Hz, 1H), 7.56 (dd, J=9.0, 2.4 Hz, 1H), 7.43-7.24 (m, 5H), 4.29 (s, 2H), 4.08 (m, 4H), 2.64 (t, J=4.8 Hz, 4H), 2.38 (s, 3H). MS m/z: 393 (M+H⁺).

### EXAMPLE 119

### 2-Benzyl-9-chloro-5-(piperazin-1-yl)-[1,2,4]triazolo[1,5-c]quinazoline

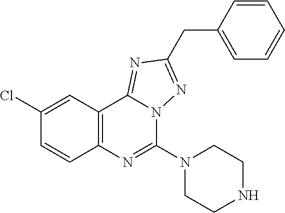

The title compound was prepared as described in Example 118, except that piperazine was substituted for N-methylpiperazine in step 4 of that route. ¹H NMR (300 MHz, CD₃OD) δ: 8.23 (s, 1H), 7.64 (m, 2H), 7.38-7.21 (m, 5H), 4.27 (s, 2H), 4.02 (t, J=4.8 Hz, 4H), 3.02 (t, J=4.8 Hz, 4H). MS m/z: 379 (M+H⁺).

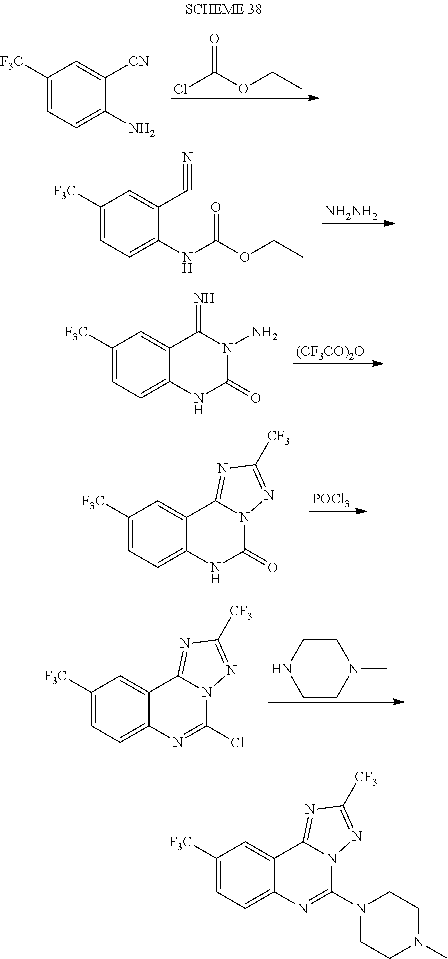

### EXAMPLE 120

### 5-(4-methylpiperazin-1-yl)-2,9-bis(trifluoromethyl)-[1,2,4]triazolo[1,5-c]quinazoline

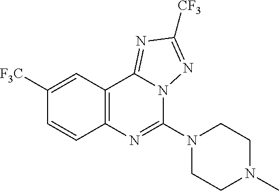

### Step 1

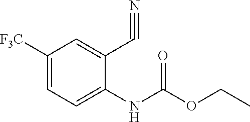

### Ethyl 2-cyano-4-(trifluoromethyl)phenylcarbamate

A 25 mL round bottom flask was charged with 2-amino-5-(trifluoromethyl)benzenecarbonitrile (1.0 g, 5.4 mmol), Na₂CO₃ (1.14 g, 10.8 mmol) and ethyl chloroformate (15 mL). The resulting mixture was stirred overnight at reflux. Reaction progress was monitored by TLC (EtOAc/Petroleum ether=1:6). Work-up: the mixture was filtered and the filter cake was washed 2-butanone (20 mL×2). The filtrate was concentrated to dryness, giving 1.35 g (98%) of the product as light yellow solids.

### Step 2

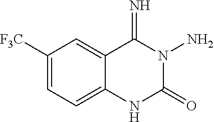

### 3-amino-4-imino-6-(trifluoromethyl)-3,4-dihydroquinazolin-2(1H)-one

A 25 mL round bottom flask was charged with N-[2-cyano-4-(trifluoromethyl)phenyl]ethoxycarboxamide (0.3 g, 1.2 mmol), hydrazine hydrate (0.07 g, 1.4 mmol) and THF (7 mL). The resulting mixture was heated at 60° C. overnight. Work-up: the precipitate was collected by filtration and washed with THF (20 mL×2), to afford 0.15 g (52%) of the product as light yellow solids. The filtrate was recovered and heated again at 60° C., to get another batch of 50 mg of the product in the same manner. MS m/z: 245 (M+H⁺).

### Step 3

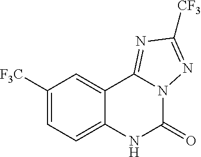

### 2,9-bis(trifluoromethyl)-[1,2,4]triazolo[1,5-c]quinazolin-5(6H)-one

A 15 mL tube was charged with 3-amino-4-imino-6-(trifluoromethyl)-3,4-dihydroquinazolin-2(1H)-one (0.24 g, 1.0 mmol) and trifluoroacetic anhydride (3 mL). The tube was sealed and the reaction mixture was heated at 85° C. overnight. Reaction progress was monitored by TLC (EtOAc/Petroleum ether=2:1). Work-up: the solvent was evaporated under reduced pressure. The crude material was purified by flash column chromatography on silica gel with a 1:40 MeOH/CH₂Cl₂, to afford 0.29 g (91%) of the product as light yellow crystals. ¹H NMR (300 MHz, DMSO-d₆) δ: 11.14 (s, 1H), 8.71 (d, J=1.8 Hz, 1H), 8.00 (dd, J=8.7, 1.8 Hz, 1H), 7.76 (d, J=8.7 Hz, 1H). MS m/z: 321 (M−H⁺).

### Step 4

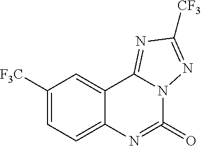

### 5-chloro-2,9-bis(trifluoromethyl)-[1,2,4]triazolo[1,5-c]quinazoline

A 25 mL round bottom flask was charged with 2,9-bis(trifluoromethyl)-5,7-dihydro-1,2,4-triazolo[1,5-c]quinazolin-6-one (0.16 g, 0.50 mmol) and phosphorus oxychloride (4 mL). To the resulting solution was added N,N-diisopropylethylamine (0.17 mL, 1.0 mmol). The mixture was heated at reflux for 1.5 h. Reaction progress was monitored by TLC (EtOAc/Petroleum ether=2:1). Work-up: the solvent was evaporated under reduced pressure. The crude material was purified by flash column chromatography on silica gel with a 1:15 EtOAc/Petroleum ether, to afford 0.16 g (95%) of the product as light yellow solids.

### Step 5

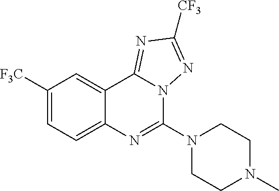

### 5-(4-methylpiperazin-1-yl)-2,9-bis(trifluoromethyl)-[1,2,4]triazolo[1,5-c]quinazoline

A 25 mL round bottom flask was charged with N-methylpiperazine (0.11 mL, 0.94 mmol) and acetonitrile (2 mL). To the resulting solution was added dropwise a solution of 5-chloro-2,9-bis(trifluoromethyl)-[1,2,4]triazolo[1,5-c]quinazoline (0.16 g, 0.47 mmol) in acetonitrile (2 mL). The mixture was stirred at room temperature for 30 minutes. Reaction progress was monitored by TLC (EtOAc/Petroleum ether=1:8). Work-up: the solvent was evaporated under reduced pressure. The residue was mixed with water (10 mL) and stirred for 20 minutes at room temperature. The solid was collected by filtration, washed with water (5 mL), and dried, to give 0.17 g (90%) of the product as light yellow crystals. ¹H NMR (300 MHz, CD₃OD) δ: 8.63 (d, J=1.8 Hz, 1H), 7.99 (dd, J=9.0, 1.8 Hz, 1H), 7.87 (d, J=9.0 Hz, 1H), 4.19 (t, J=4.5 Hz, 4H), 2.70 (t, J=4.5 Hz, 4H), 2.38 (s, 3H). MS m/z: 405 (M+H⁺).

### EXAMPLE 121

### 5-(piperazin-1-yl)-2,9-bis(trifluoromethyl)-[1,2,4]triazolo[1,5-c]quinazoline

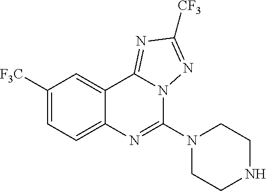

The title compound was prepared as described in Example 120, except that piperazine was substituted for N-methylpiperazine in step 5 of that route. ¹H NMR (300 MHz, CD₃OD) δ: 8.61 (d, J=0.6 Hz, 1H), 7.95 (dd, J=8.7, 0.6 Hz, 1H), 7.85 (d, J=8.7 Hz, 1H), 4.14 (t, J=4.8 Hz, 4H), 3.07 (t, J=4.8 Hz, 4H). MS m/z: 391 (M+H⁺).

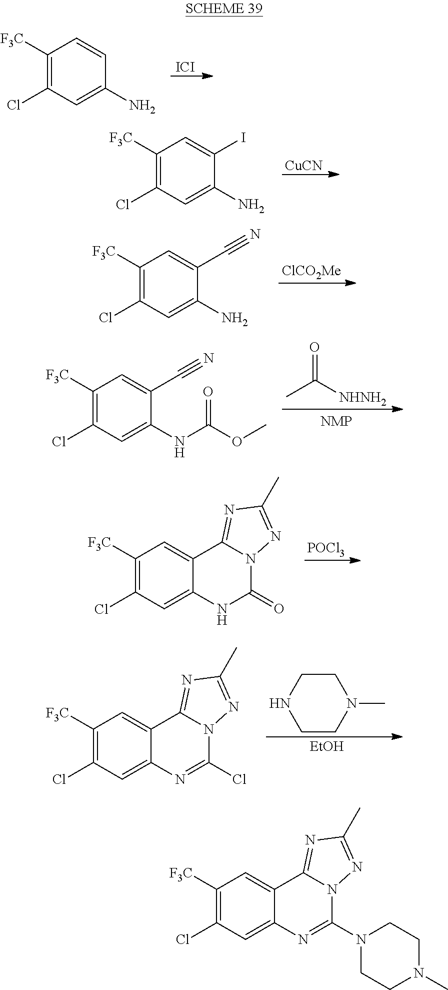

### EXAMPLE 122

### 8-chloro-2-methyl-5-(4-methylpiperazin-1-yl)-9-(trifluoromethyl)-[1,2,4]triazolo[1,5-c]quinazoline

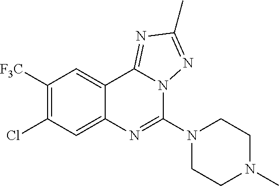

### Step 1

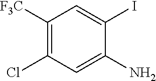

### 5-Chloro-2-iodo-4-(trifluoromethyl)phenylamine

A 250 mL 3-necked round bottom flask was charged with 3-chloro-4-(trifluoromethyl)aniline (4.5 g, 0.02 mol) and MeOH (50 mL). To the above was added dropwise a solution of ICl (4.8 g, 0.03 mol) in CH₂Cl₂ (100 mL) at 0° C. The resulting mixture was stirred at room temperature for 1 h. Reaction progress was monitored by TLC (EtOAc/Petroleum ether=1:20, Rf=0.6). Work-up: the mixture was concentrated in vacuo. The residue was re-dissolved in CH₂Cl₂, washed with water, dried over anhydrous Na₂SO₄ and concentrated in vacuo, to give 6.9 g (93%) of the product. MS m/z: 320 (M−H⁺).

### Step 2

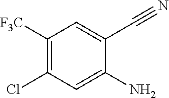

### 2-Amino-4-chloro-5-(trifluoromethyl)benzenecarbonitrile

A 250 mL round bottom flask was charged with 5-chloro-2-iodo-4-(trifluoromethyl)aniline (6.9 g, 0.02 mol), CuCN (3.85 g, 0.04 mol) and DMF (100 mL). The resulting mixture was stirred at 130° C. overnight. Reaction progress was monitored by TLC (EtOAc/Petroleum ether=1:4, Rf=0.5). Work-up: the mixture was concentrated in vacuo. The residue was purified by flash column chromatography on silica gel with 20% EtOAc in petroleum ether, to afford 3 g (63%) of the product. MS m/z: 221 (M+H⁺).

### Steps 3-6

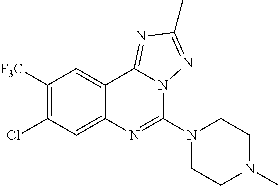

### 8-chloro-2-methyl-5-(4-methylpiperazin-1-yl)-9-(trifluoromethyl)-[1,2,4]triazolo[1,5-c]quinazoline

The HCl salt of the title compound was prepared as described in Example 93, except that 2-amino-4-chloro-5-(trifluoromethyl)benzenecarbonitrile was substituted for 2-amino-4,5-dichlorobenzonitrile in steps 2-5 of that route. ¹H NMR (300 MHz, D₂O) δ: 7.87 (s, 1H), 7.36 (s, 1H), 4.94-4.91 (m, 2H), 3.53-3.51 (m, 4H), 3.25-3.22 (m, 2H), 2.90 (s, 3H), 2.45 (s, 3H). MS m/z: 385 (M+H⁺).

### EXAMPLE 123

### 8-chloro-2-methyl-5-(piperazin-1-yl)-9-(trifluoromethyl)-[1,2,4]triazolo[1,5-c]quinazoline

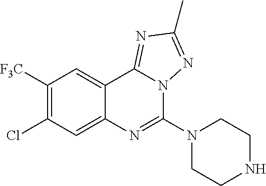

The HCl salt of the title compound was prepared as described in Example 122, except that piperazine was substituted for N-methylpiperazine in step 6 of that route. ¹H NMR (300 MHz, D₂O) δ: 7.83 (s, 1H), 7.32 (s, 1H), 4.18 (t, J=5.4 Hz, 4H), 3.04 (t, J=4.8 Hz, 4H), 2.45 (s, 3H). MS m/z: 371 (M+H⁺).

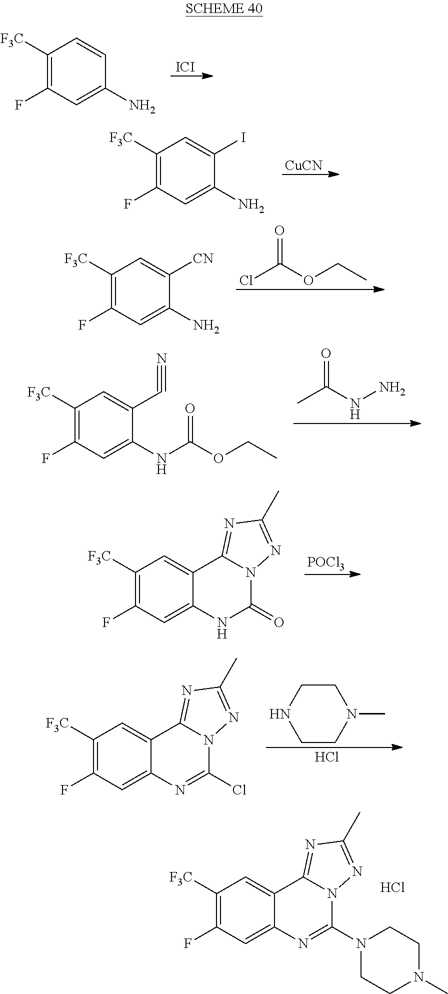

### EXAMPLE 124

### 8-fluoro-2-methyl-5-(4-methylpiperazin-1-yl)-9-(trifluoromethyl)-[1,2,4]triazolo[1,5-c]quinazoline hydrochloride

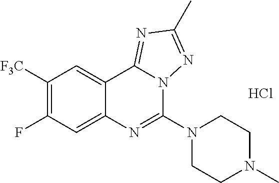

The title compound was prepared as described in Example 122, except that 3-fluoro-4-(trifluoromethyl)aniline was substituted for 3-chloro-4-(trifluoromethyl)aniline in step 1 of that route. ¹H NMR (300 MHz, DMSO-d₆) δ: 11.65 (s, 1H), 8.43 (d, J=8.1 Hz, 1H), 7.67 (d, J=12.6 Hz, 1H), 5.08 (d, J=12.9 Hz, 2H), 3.75-3.24 (m, 6H), 2.78 (s, 3H), 2.55 (s, 3H). MS m/z: 369 (M+H⁺).

### EXAMPLE 125

### 8-fluoro-2-methyl-5-(piperazin-1-yl)-9-(trifluoromethyl)-[1,2,4]triazolo[1,5-c]quinazoline hydrochloride

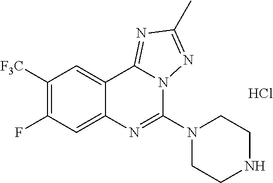

The title compound was prepared as described in Example 124, except that piperazine was substituted for N-methylpiperazine in step 6 of that route. ¹H NMR (300 MHz, DMSO-d₆) δ: 9.45 (s, 2H), 8.47 (d, J=7.8 Hz, 1H), 7.69 (d, J=12.3 Hz, 1H), 4.35 (t, J=4.5 Hz, 4H), 3.30 (t, J=4.5 Hz, 4H), 2.55 (s, 3H). MS m/z: 355 (M+H⁺).

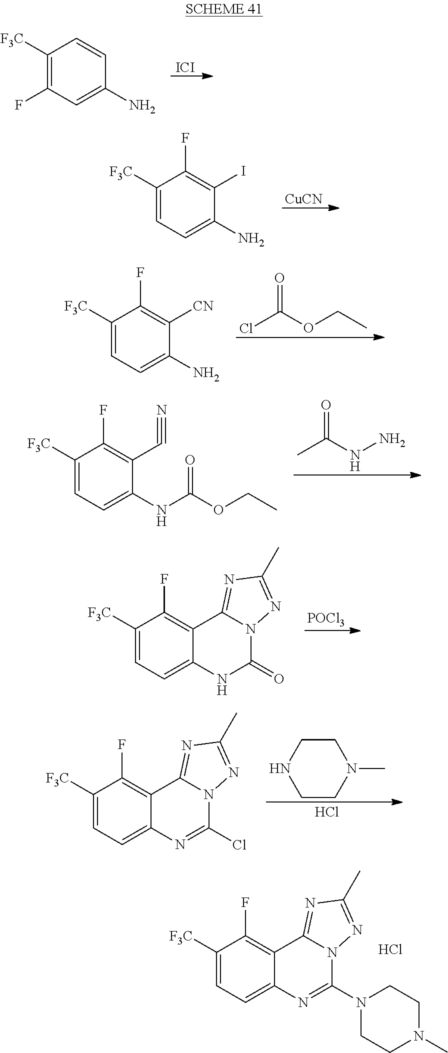

### EXAMPLE 126

### 10-fluoro-2-methyl-5-(4-methylpiperazin-1-yl)-9-(trifluoromethyl)-[1,2,4]triazolo[1,5-c]quinazoline hydrochloride

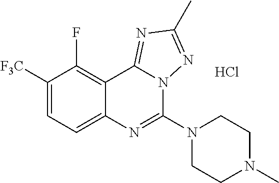

The title compound was prepared as described in Example 124, except that 3-fluoro-2-iodo-4-(trifluoromethyl)aniline, which was also obtained as the other isomer in step 1, was substituted for 5-fluoro-2-iodo-4-(trifluoromethyl)aniline in step 2 of that route. ¹H NMR (300 MHz, CD₃OD) δ: 7.94 (t, J=8.4 Hz, 1H), 7.66 (d, J=8.7 Hz, 1H), 5.35 (d, J=14.4 Hz, 2H), 3.73-3.62 (m, 4H), 3.43-3.35 (m, 2H), 2.99 (s, 3H), 2.64 (s, 3H). MS m/z: 369 (M+H⁺).

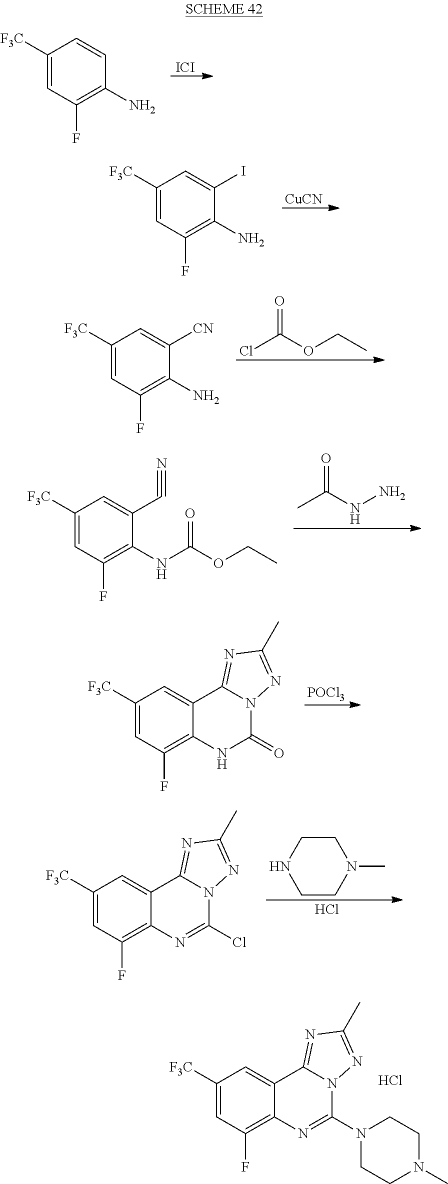

### EXAMPLE 127

### 7-fluoro-2-methyl-5-(4-methylpiperazin-1-yl)-9-(trifluoromethyl)-[1,2,4]triazolo[1,5-c]quinazoline hydrochloride

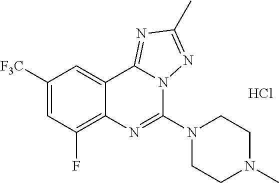

The title compound was prepared as described in Example 122, except that 2-fluoro-4-(trifluoromethyl)aniline was substituted for 3-chloro-4-(trifluoromethyl)aniline in step 1 of that route. ¹H NMR (300 MHz, D₂O) δ: 7.68 (s, 1H), 7.49 (d, J=10.5 Hz, 1H), 4.97 (d, J=14.4 Hz, 2H), 3.65 (d, J=12.8 Hz, 2H), 3.55-3.46 (m, 2H), 3.30-3.22 (m, 2H), 2.92 (s, 3H), 2.46 (s, 3H). MS m/z: 369 (M+H⁺).

### EXAMPLE 128

### 7-fluoro-2-methyl-5-(piperazin-1-yl)-9-(trifluoromethyl)-[1,2,4]triazolo[1,5-c]quinazoline hydrochloride

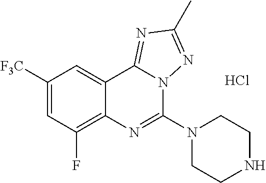

The title compound was prepared as described in Example 127, except that piperazine was substituted for N-methylpiperazine in step 6 of that route. ¹H NMR (300 MHz, D₂O) δ: 7.70 (s, 1H), 7.49 (d, J=10.5 Hz, 1H), 4.20 (br, 4H), 3.41 (br, 4H), 2.46 (s, 3H). MS m/z: 355 (M+H⁺).

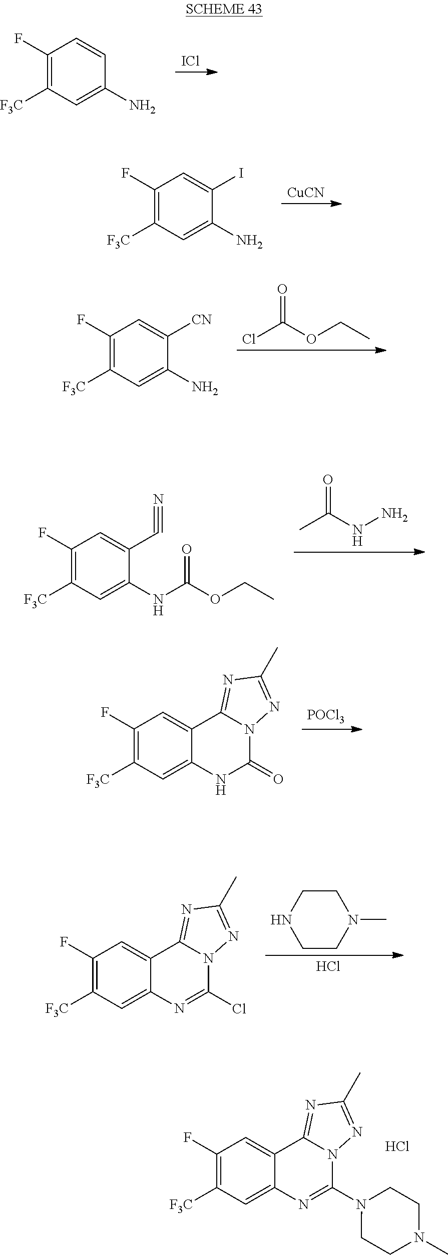

### EXAMPLE 129

### 9-fluoro-2-methyl-5-(4-methylpiperazin-1-yl)-8-(trifluoromethyl)-[1,2,4]triazolo[1,5-c]quinazoline hydrochloride

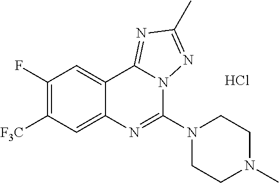

The title compound was prepared as described in Example 122, except that 4-fluoro-3-(trifluoromethyl)aniline was substituted for 3-chloro-4-(trifluoromethyl)aniline in step 1 of that route. ¹H NMR (300 MHz, D₂O) δ: 7.61 (d, J=6.0 Hz, 1H), 7.37 (d, J=12.0 Hz, 1H), 4.76-4.68 (m, 2H), 3.65-3.23 (m, 6H), 2.91 (s, 3H), 2.45 (s, 3H). MS m/z: 369 (M+H⁺).

### EXAMPLE 130

### 9-fluoro-2-methyl-5-(piperazin-1-yl)-8-(trifluoromethyl)-[1,2,4]triazolo[1,5-c]quinazoline hydrochloride

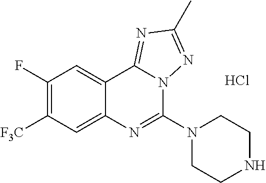

The title compound was prepared as described in Example 129, except that piperazine was substituted for N-methylpiperazine in step 6 of that route. ¹H NMR (300 MHz, D₂O) δ: 7.61 (d, J=6.0 Hz, 1H), 7.39 (d, J=12.0 Hz, 1H), 4.07-4.04 (m, 4H), 3.40-3.36 (m, 4H), 2.46 (s, 3H). MS m/z: 355 (M+H⁺).

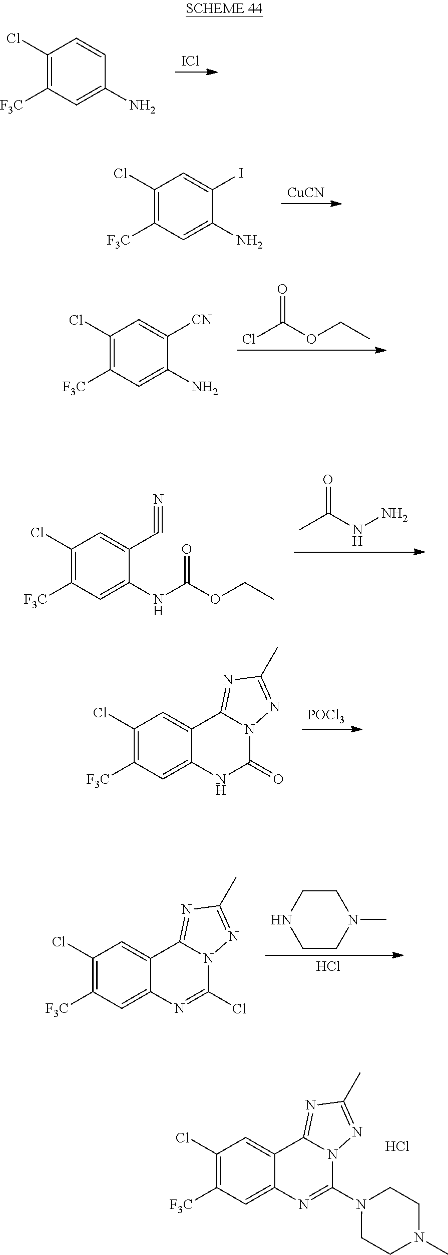

### EXAMPLE 131

### 9-chloro-2-methyl-5-(4-methylpiperazin-1-yl)-8-(trifluoromethyl)-[1,2,4]triazolo[1,5-c]quinazoline hydrochloride

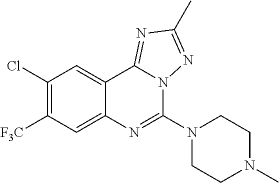

The title compound was prepared as described in Example 122, except that 4-chloro-3-(trifluoromethyl)aniline was substituted for 3-chloro-4-(trifluoromethyl)aniline in step 1 of that route. ¹H NMR (300 MHz, D₂O) δ: 7.57 (s, 1H), 7.56 (s, 1H), 4.83 (d, J=14.4 Hz, 2H), 3.62 (d, J=12.8 Hz, 2H), 3.51-3.41 (m, 2H), 3.29-3.24 (m, 2H), 2.90 (s, 3H), 2.46 (s, 3H). MS m/z: 385 (M+H⁺).

### EXAMPLE 132

### 9-chloro-2-methyl-5-(piperazin-1-yl)-8-(trifluoromethyl)-[1,2,4]triazolo[1,5-c]quinazoline hydrochloride

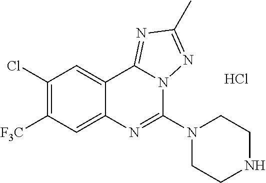

The title compound was prepared as described in Example 131, except that piperazine was substituted for N-methylpiperazine in step 6 of that route. ¹H NMR (300 MHz, D₂O) δ: 7.60 (br, 2H), 4.11 (br, 4H), 3.40 (br, 4H), 2.47 (s, 3H). MS m/z: 371 (M+H⁺).

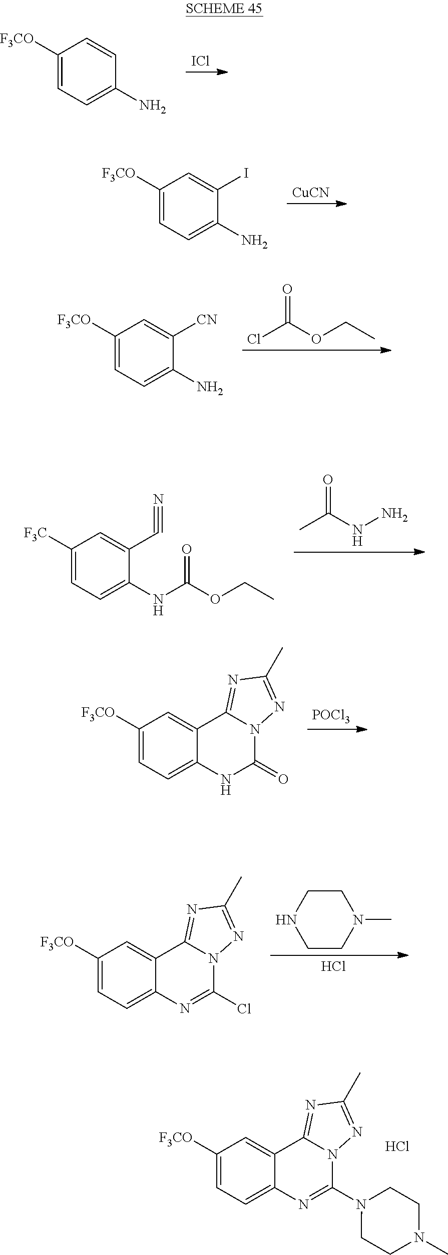

### EXAMPLE 133

### 2-methyl-5-(4-methylpiperazin-1-yl)-9-(trifluoromethoxy)-[1,2,4]triazolo[1,5-c]quinazoline hydrochloride

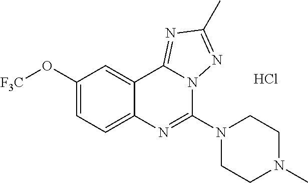

The title compound was prepared as described in Example 122, except that 4-(trifluoromethoxy)aniline was substituted for 3-chloro-4-(trifluoromethyl)aniline in step 1 of that route. ¹H NMR (300 MHz, CD₃OD) δ: 8.19 (dd, J=2.4, 1.2 Hz, 1H), 7.88 (d, J=9.0 Hz, 1H), 7.72-7.68 (m, 1H), 5.17 (dd, J=14.1, 2.1 Hz, 2H), 3.72-3.58 (m, 4H), 3.46-3.42 (m, 2H), 2.99 (s, 3H), 2.65 (s, 3H). MS m/z: 367 (M+H⁺).

### EXAMPLE 134

### 2-methyl-5-(piperazin-1-yl)-9-(trifluoromethoxy)-[1,2,4]triazolo[1,5-c]quinazoline hydrochloride

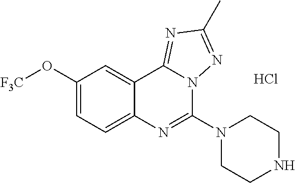

The title compound was prepared as described in Example 133, except that piperazine was substituted for N-methylpiperazine in step 6 of that route. ¹H NMR (300 MHz, DMSO-d₆) δ: 9.50 (br, 2H), 8.10 (d, J=0.6 Hz, 1H), 7.81 (d, J=9.3 Hz, 1H), 7.73 (dd, J=9.3, 0.6 Hz, 1H), 4.23 (t, J=5.1 Hz, 4H), 3.29 (br, 4H), 2.55 (s, 3H). MS m/z: 353 (M+H⁺).

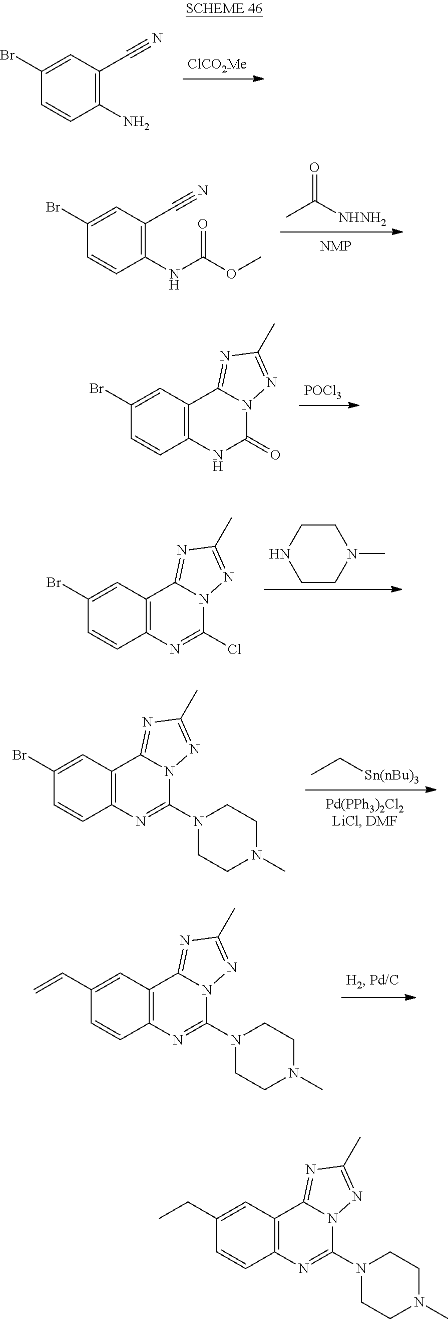

### EXAMPLE 135

### 9-bromo-2-methyl-5-(4-methylpiperazin-1-yl)-[1,2,4]triazolo[1,5-c]quinazoline

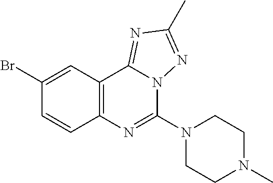

The HCl salt of the title compound was prepared as described in Example 93, except that 2-amino-5-bromobenzonitrile was substituted for 2-amino-4,5-dichlorobenzonitrile in steps 2-5 of that route. ¹H NMR (300 MHz, CD₃OD) δ: 8.31 (d, J=2.1 Hz, 1H), 7.88 (dd, J=8.7, 2.1 Hz, 1H), 7.65 (d, J=8.7 Hz, 1H), 5.18-5.13 (m, 2H), 3.70-3.58 (m, 4H), 3.43-3.39 (m, 2H), 2.98 (s, 3H), 2.63 (s, 3H). MS m/z: 361 (M+H⁺).

### EXAMPLE 136

### 9-bromo-2-methyl-5-(piperazin-1-yl)-[1,2,4]triazolo[1,5-c]quinazoline

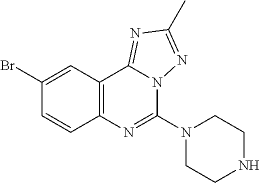

The HCl salt of the title compound was prepared as described in Example 135, except that piperazine was substituted for N-methylpiperazine in step 4 of that route. ¹H NMR (300 MHz, CD₃OD) δ: 8.42-8.40 (m, 1H), 7.88 (dd, J=8.7, 2.4 Hz, 1H), 7.67-7.64 (m, 1H), 4.32 (t, J=5.1 Hz, 4H), 3.49-3.31 (m, 4H), 2.64 (s, 3H). MS m/z: 347 (M+H⁺).

### EXAMPLE 137

### 2-methyl-5-(4-methylpiperazin-1-yl)-9-vinyl-[1,2,4]triazolo[1,5-c]quinazoline

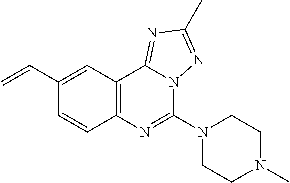

The HCl salt of the title compound was prepared as described in Example 88, except that 9-bromo-2-methyl-5-(4-methylpiperazin-1-yl)-[1,2,4]triazolo[1,5-c]quinazoline was substituted for 8-bromo-4-(4-methylpiperazin-1-yl)-[1,2,4]triazolo[4,3-a]quinoxaline, in step 1 of that route. ¹H NMR (300 MHz, CD₃OD) δ: 8.27 (d, J=1.8 Hz, 1H), 8.02 (dd, J=8.7, 2.1 Hz, 1H), 7.80 (d, J=8.4 Hz, 1H), 6.92 (dd, J=17.7, 11.1 Hz, 1H), 5.99 (d, J=17.4 Hz, 1H), 5.44 (d, J=11.4 Hz, 1H), 5.10-5.05 (m, 2H), 3.73-3.66 (m, 4H), 3.45-3.37 (m, 2H), 2.99 (s, 3H), 2.71 (s, 3H). MS m/z: 309 (M+H⁺).

### EXAMPLE 138

### 2-methyl-5-(piperazin-1-yl)-9-vinyl-[1,2,4]triazolo[1,5-c]quinazoline

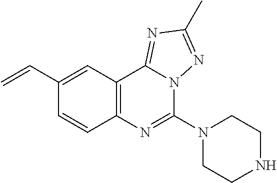

The HCl salt of the title compound was prepared as described in Example 89, except that piperazine was substituted for N-methylpiperazine in step 4 of that route. ¹H NMR (300 MHz, CD₃OD) δ: 8.28 (d, J=1.8 Hz, 1H), 8.01 (dd, J=8.4, 2.1 Hz, 1H), 7.79 (d, J=8.4 Hz, 1H), 6.92 (dd, J=17.7, 11.1 Hz, 1H), 5.98 (d, J=17.4 Hz, 1H), 5.44 (d, J=10.8 Hz, 1H), 4.31 (t, J=5.1 Hz, 4H), 3.49 (t, J=5.1 Hz, 4H), 2.70 (s, 3H). MS m/z: 295 (M+H⁺).

### EXAMPLE 139

### 9-ethyl-2-methyl-5-(4-methylpiperazin-1-yl)-[1,2,4]triazolo[1,5-c]quinazoline

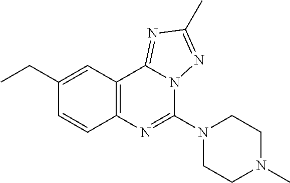

The HCl salt of the title compound was prepared as described in Example 90, except that 2-methyl-5-(4-methylpiperazin-1-yl)-9-vinyl-[1,2,4]triazolo[1,5-c]quinazoline was substituted for 4-(4-methylpiperazin-1-yl)-8-vinyl-[1,2,4]triazolo[4,3-a]quinoxaline, in step 1 of that route. ¹H NMR (300 MHz, CD₃OD) δ: 8.14 (s, 1H), 7.80 (m, 2H), 5.05-5.00 (m, 2H), 3.72-3.58 (m, 4H), 3.46-3.37 (m, 2H), 2.99 (s, 3H), 2.88 (q, J=7.8 Hz, 2H), 2.71 (s, 3H), 1.35 (t, J=7.8 Hz, 3H). MS m/z: 311 (M+H⁺).

### EXAMPLE 140

### 9-ethyl-2-methyl-5-(piperazin-1-yl)-[1,2,4]triazolo[1,5-c]quinazoline

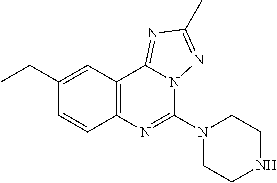

The HCl salt of the title compound was prepared as described in Example 91, except that piperazine was substituted for N-methylpiperazine in step 4 of that route. ¹H NMR (300 MHz, CD₃OD) δ: 8.15 (s, 1H), 7.83 (m, 2H), 4.29 (t, J=4.8 Hz, 4H), 3.49 (t, J=5.1 Hz, 4H), 2.89 (q, J=7.5 Hz, 2H), 2.85 (s, 3H), 1.35 (t, J=7.8 Hz, 3H). MS m/z: 297 (M+H⁺).

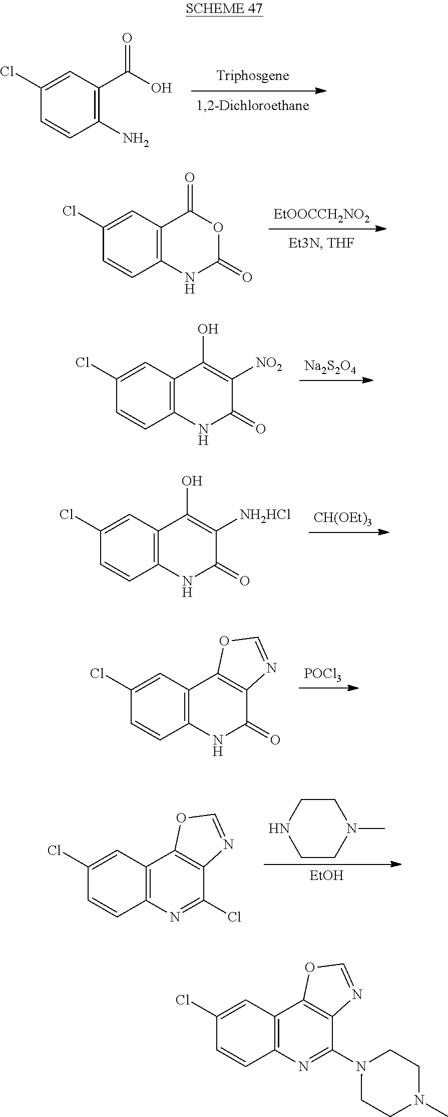

### EXAMPLE 141

### 8-chloro-4-(4-methylpiperazin-1-yl)oxazolo[4,5-c]quinoline

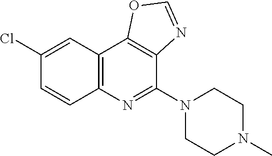

### Step 1

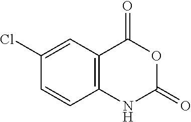

### 6-Chloro-1H-benzo[d]1,3-oxazine-2,4-dione

A 500 mL 3-necked round bottom flask was charged with 2-amino-5-chlorobenzoic acid (17 g, 0.1 mol) and 1,2-dichloroethane (200 mL). To the above was added dropwise a solution of triphosgene (21 g, 0.21 mol) in 1,2-dichloroethane (100 mL) at 80° C. The resulting mixture was heated at 80° C. for further 3 h then cooled in ice-water. The precipitate was collected by filtration and dried to afford 19 g (97%) of the product as white solid. ¹H NMR (300 MHz, DMSO-d₆) δ: 11.85 (br, 1H), 7.88 (d, J=2.4 Hz, 1H), 7.78 (dd, J=8.7, 2.4 Hz, 1H), 7.15 (d, J=8.7 Hz, 1H).

### Step 2

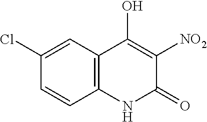

### 6-Chloro-4-hydroxy-3-nitrohydroquinolin-2-one

A 500 mL 3-necked round bottom flask was charged with ethyl nitroacetate (16 mL, 144 mmol), Et₃N (20 mL, 144 mmol) and anhydrous THF (400 mL). To the above was added dropwise a solution of 6-chloro-1H-benzo[d]1,3-oxazine-2,4-dione (19 g, 96 mmol) in THF (100 mL). The resulting solution was heated at 55° C. overnight then concentrated under reduced pressure. The residue was washed with Et₂O then dissolved in water and acidified with 6 M HCl. The precipitate was collected by filtration and dried to afford 8 g (34%) of the product as yellow solid. ¹H NMR (300 MHz, DMSO-d₆) δ: 11.85 (br, 1H), 8.00 (d, J=2.7 Hz, 1H), 7.64 (dd, J=8.4, 2.1 Hz, 1H), 7.31 (d, J=9.0 Hz, 1H).

### Step 3

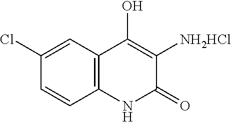

### 3-Amino-6-chloro-4-hydroxyhydroquinolin-2-one hydrochloride salt

A 250 mL round bottom flask was charged with 6-chloro-4-hydroxy-3-nitrohydroquinolin-2-one (2.4 g, 10 mmol) and 1 M NaOH aqueous solution (100 mL). To the above was added Na₂S₂O₄ (12 g, 59 mmol) portion-wise. The resulting solution was stirred in the dark for 30 min. It was then cooled to 0° C. and acidified with 6 M HCl. The precipitate was collected by filtration, washed with small amount of acetone, and dried, to afford 2 g (83%) of the product as white solid. ¹H NMR (300 MHz, DMSO-d₆) δ: 12.06 (br, 1H), 8.04 (d, J=2.4 Hz, 1H), 7.54 (dd, J=9.3, 2.4 Hz, 1H), 7.35 (d, J=8.7 Hz, 1H), 5.0 (br, 3H). MS m/z: 211 (M+H⁺).

### Step 4

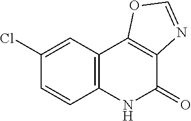

### 8-chlorooxazolo[4,5-c]quinolin-4(5H)-one

A 100 mL round bottom flask was charged with 3-amino-6-chloro-4-hydroxyhydroquinolin-2-one hydrochloride salt (2 g, 8.1 mmol) and triethylorthoformate (30 mL). The resulting solution was heated at reflux for 30 min then cooled in ice-water. The precipitate was collected by filtration, washed with CH₂Cl₂, and dried, to afford 1.5 g (84%) of the product as yellow solid. ¹H NMR (300 MHz, DMSO-d₆) δ: 12.15 (br, 1H), 8.87 (s, 1H), 7.96 (d, J=2.1 Hz, 1H), 7.62 (dd, J=8.7, 2.1 Hz, 1H), 7.50 (d, J=8.4 Hz, 1H). MS m/z: 221 (M+H⁺).

### Step 5

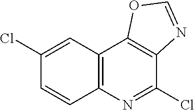

### 4,8-dichlorooxazolo[4,5-c]quinoline

A 100 mL round bottom flask was charged with 8-chloro-5-hydro-1,3-oxazolo[4,5-c]quinolin-4-one (1.7 g, 7.7 mmol) and POCl₃ (20 mL). The resulting solution was heated at reflux for 20 min then concentrated in vacuo. The residue was mixed with saturated aqueous Na₂CO₃ and extracted with CH₂Cl₂. The combined organic layers were dried over anhydrous Na₂SO₄ and concentrated in vacuo. It was further purified by flash column chromatography on silica gel with 10% EtOAc in petroleum ether, to afford 480 mg (26%) of the product as white solid. ¹H NMR (300 MHz, CDCl₃) δ: 9.19 (s, 1H), 8.38 (dd, J=2.4, 0.3 Hz, 1H), 8.15 (dd, J=9.0, 0.3 Hz, 1H), 7.91 (dd, J=8.7, 2.4 Hz, 1H). MS m/z: 239 (M+H⁺).

### Step 6

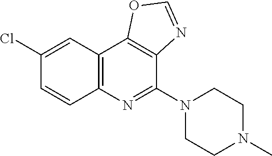

### 8-chloro-4-(4-methylpiperazin-1-yl)oxazolo[4,5-c]quinoline

A 20 mL microwave reaction tube was charged with 4,8-dichlorooxazolo[4,5-c]quinoline (320 mg, 1.3 mmol), N-methylpiperazine (0.16 mL, 1.4 mmol), Et₃N (0.6 mL, 4.3 mmol) and anhydrous EtOH (15 mL). The resulting solution was heated at 130° C. for 1 h in a Biotage microwave reactor. The solvent was evaporated and the residue was purified by flash column chromatography on silica gel with 10% MeOH in CH₂Cl₂, to afford 100 mg (25%) of the product as white solid. ¹H NMR (300 MHz, CD₃OD) δ: 8.51 (s, 1H), 7.94 (d, J=2.7 Hz, 1H), 7.69 (d, J=9.0 Hz, 1H), 7.49 (dd, J=9.0, 2.7 Hz, 1H), 4.26 (t, J=5.1 Hz, 4H), 2.65 (t, J=5.1 Hz, 4H), 2.38 (s, 3H). MS m/z: 303 (M+H⁺).

### EXAMPLE 142

### 8-Chloro-4-piperazinyl-1,3-oxazolo[4,5-c]quinoline

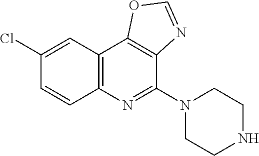

The title compound was prepared as described in Example 141, except that piperazine was substituted for N-methylpiperazine in step 6 of that route. ¹H NMR (300 MHz, CD₃OD) δ: 8.60 (s, 1H), 8.05 (d, J=2.4 Hz, 1H), 7.78 (d, J=9.0 Hz, 1H), 7.58 (dd, J=9.0, 2.4 Hz, 1H), 4.47 (t, J=5.4 Hz, 4H), 3.36 (t, J=5.4 Hz, 4H). MS m/z: 289 (M+H⁺).

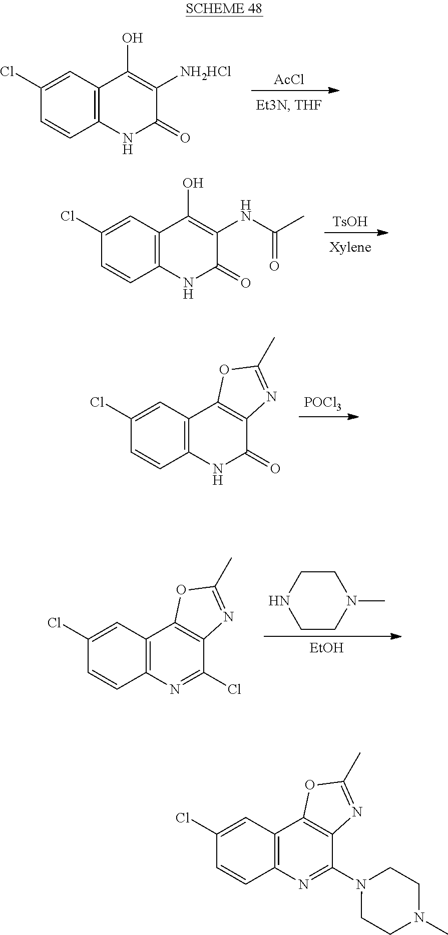

### EXAMPLE 143

### 8-chloro-2-methyl-4-(4-methylpiperazin-1-yl)oxazolo[4,5-c]quinoline

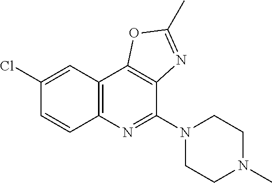

### Step 1

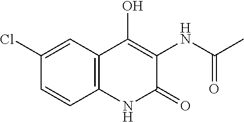

### N-(6-Chloro-4-hydroxy-2-oxo-3-hydroquinolyl)acetamide

A 500 mL round bottom flask was charged with 3-amino-6-chloro-4-hydroxyhydroquinolin-2-one hydrochloride salt (prepared in Example 141 step 1-3, 6.8 g, 28 mmol) and anhydrous THF (150 mL). To the above were added dropwise anhydrous Et₃N (9.6 mL, 69 mmol) and acetyl chloride (3 mL, 42 mmol). The resulting solution was heated at reflux for 6 h, cooled to room temperature, diluted with H₂O and acidified with 6N HCl. The precipitate was collected by filtration and washed with H₂O, to afford 6 g (86%) of the product as yellow solid. ¹H NMR (300 MHz, DMSO-d₆) δ: 12.07 (br, 1H), 11.94 (br, 1H), 9.76 (br, 1H), 7.79 (d, J=2.7 Hz, 1H), 7.54 (dd, J=8.7, 2.4 Hz, 1H), 7.29 (d, J=8.7 Hz, 1H), 2.23 (s, 3H).

### Step 2

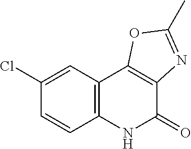

### 8-chloro-2-methyloxazolo[4,5-c]quinolin-4(5H)-one

A 500 mL round bottom flask was charged with N-(6-chloro-4-hydroxy-2-oxo-3-hydroquinolyl)acetamide (3 g, 12 mmol) and xylene (250 mL). The resulting solution was heated at 190° C. for 4 h. The solvent was evaporated under reduced pressure and the residue was re-dissolved in EtOAc and washed with H₂O. The organic layer was dried over anhydrous Na₂SO₄ and concentrated in vacuo, to afford 1 g (36%) of the product which was used as such in the next step. ¹H NMR (300 MHz, DMSO-d₆) δ: 12.06 (br, 1H), 7.89 (d, J=2.1 Hz, 1H), 7.59 (dd, J=9.0, 2.4 Hz, 1H), 7.48 (d, J=9.0 Hz, 1H), 2.65 (s, 3H).

### Step 3

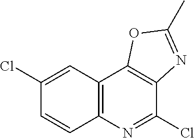

### 4,8-dichloro-2-methyloxazolo[4,5-c]quinoline

A 50 mL round bottom flask was charged with 8-chloro-2-methyloxazolo[4,5-c]quinolin-4(5H)-one (1.0 g, 4.3 mmol) and POCl₃ (20 mL). The resulting solution was heated at reflux for 20 min. After evaporation of the solvent, the residue was poured into saturated aqueous Na₂CO₃ and extracted with CH₂Cl₂. The combined organic layers were dried over Na₂SO₄ and concentrated in vacuo. The residue was purified by flash column chromatography on silica gel with 10% EtOAc in petroleum ether, to afford 730 mg (68%) of the product as white solid. MS m/z: 328 (M+H⁺).

### Step 4

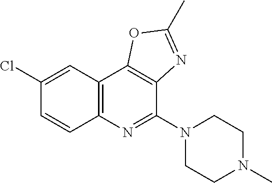

### 8-chloro-2-methyl-4-(4-methylpiperazin-1-yl)oxazolo[4,5-c]quinoline

A 20 mL microwave reaction tube was charged with 4,8-dichloro-2-methyloxazolo[4,5-c]quinoline (300 mg, 1.2 mmol), N-methylpiperazine (0.16 mL, 1.4 mmol), Et₃N (0.31 ml, 2.2 mmol) and anhydrous EtOH (15 mL). The resulting solution was heated at 100° C. for 1 h in a Biotage microwave reactor. The solvent was evaporated and the residue was purified by flash column chromatography on silica gel with 10% MeOH in CH₂Cl₂, to afford 110 mg (29%) of the product as white solid. ¹H NMR (300 MHz, CD₃OD) δ: 7.81 (d, J=2.4 Hz, 1H), 7.63 (d, J=8.7 Hz, 1H), 7.44 (dd, J=9.0, 2.4 Hz, 1H), 4.19 (t, J=4.5 Hz, 4H), 2.67 (s, 3H), 2.60 (t, J=4.8 Hz, 4H), 2.35 (s, 3H). MS m/z: 316 (M+H⁺).

### EXAMPLE 144

### 8-chloro-2-methyl-4-(piperazin-1-yl)oxazolo[4,5-c]quinoline

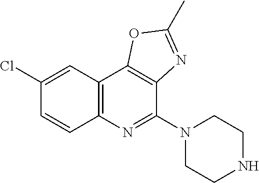

The title compound was prepared as described in Example 143, except that piperazine was substituted for N-methylpiperazine in step 4 of that route. ¹H NMR (300 MHz, CD₃OD) δ: 7.87 (d, J=2.7 Hz, 1H), 7.65 (d, J=9.0 Hz, 1H), 7.46 (dd, J=9.0, 2.4 Hz, 1H), 4.16 (t, J=5.4 Hz, 4H), 2.97 (t, J=5.1 Hz, 4H), 2.69 (s, 3H). MS m/z: 302 (M+H⁺).

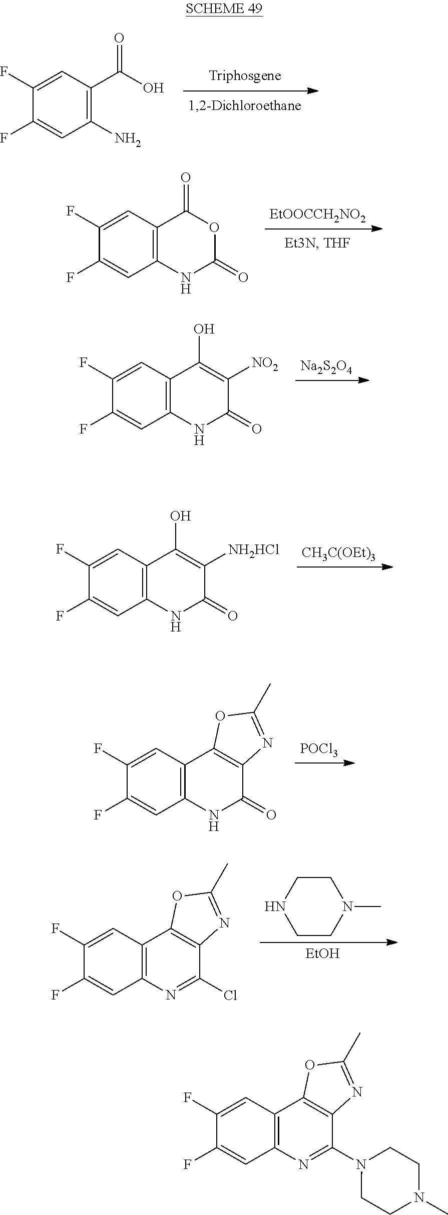

### EXAMPLE 145

### 7,8-difluoro-2-methyl-4-(4-methylpiperazin-1-yl)oxazolo[4,5-c]quinoline

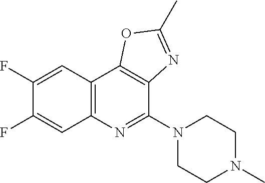

The title compound was prepared as described in Example 141, except that 2-amino-4,5-difluorobenzoic acid was substituted for 2-amino-5-chlorobenzoic acid in step 1, and ethyl orthoacetate was substituted for ethyl orthoformate in step 4 of that route. ¹H NMR (300 MHz, CD₃OD) δ: 7.65-7.63 (m, 1H), 7.45-7.43 (m, 1H), 4.18 (t, J=4.8 Hz, 4H), 2.67 (s, 3H), 2.59 (t, J=5.1 Hz, 4H), 2.35 (s, 3H). MS m/z: 319 (M+H⁺).

### EXAMPLE 146

### 7,8-difluoro-2-methyl-4-(piperazin-1-yl)oxazolo[4,5-c]quinoline

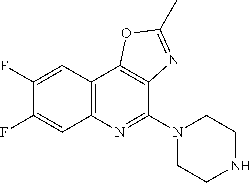

The title compound was prepared as described in Example 145, except that piperazine was substituted for N-methylpiperazine in step 6 of that route. ¹H NMR (300 MHz, DMSO-d₆) δ: 7.87-7.85 (m, 1H), 7.56-7.54 (m, 1H), 4.04 (t, J=4.8 Hz, 4H), 2.82 (t, J=5.1 Hz, 4H), 2.69 (s, 3H). MS m/z: 305 (M+H⁺).

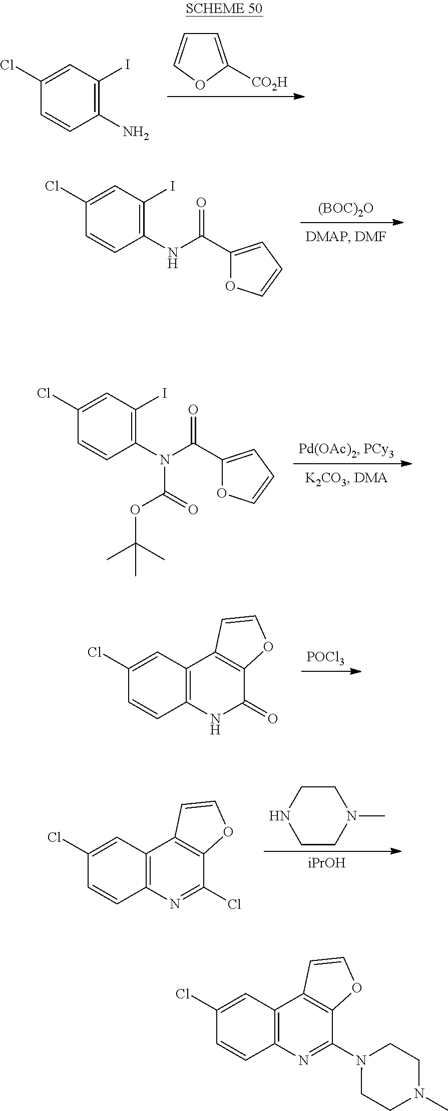

### EXAMPLE 147

### 8-Chloro-4-(4-methylpiperazin-1-yl)furo[2,3-c]quinoline

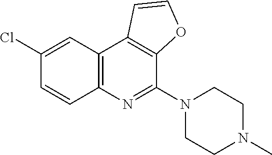

### Step 1

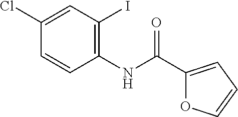

### N-(4-Chloro-2-iodophenyl)furan-2-carboxamide

A 100 mL round bottom flask was charged with furan-2-carboxylic acid (1.0 g, 7.8 mmol) and SOCl₂ (15 mL). The resulting mixture was stirred at reflux for 2.5 h then concentrated in vacuo. The residue was dissolved in CH₂Cl₂ (10 mL) and to the solution was added dropwise a solution of 4-chloro-2-iodophenylamine (1.8 g, 7.1 mmol) and Et₃N (1.3 mL, 9.2 mmol) in CH₂Cl₂ (20 mL) at 0° C. The resulting solution was stirred at room temperature for 18 h, then diluted with CH₂Cl₂ (200 mL) and washed with H₂O (100 mL). The organic layer was dried over anhydrous Na₂SO₄ and concentrated in vacuo. The residue was purified by flash column chromatography on silica gel with 4% EtOAc in petroleum ether, to afford 2.0 g (71%) of the product. MS m/z: 347 (M+H⁺).

### Step 2

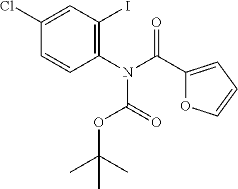

### tert-Butyl 4-chloro-2-iodophenyl(furan-2-carbonyl)carbamate

A 100 mL round bottom flask was charged with N-(4-chloro-2-iodophenyl)furan-2-carboxamide (3.70 g, 10.6 mmol), 4-dimethylaminopyridine (1.30 g, 10.6 mmol) and DMF (30 mL). To the above was added dropwise a solution of di-tert-butyl dicarbonate (7.0 g, 31.8 mmol) in DMF (10 mL) at 0° C. The resulting solution was stirred at 60° C. for 18 h then cooled to room temperature. It was diluted with H₂O (100 mL) and extracted with EtOAc (100 mL×3). The combined organic layers were washed with brine (100 mL), dried over anhydrous Na₂SO₄ and concentrated in vacuo. The residue was purified by flash column chromatography on silica gel with a 1:16 EtOAc/Petroleum ether, to give 2.50 g (53%) of the product as white solid. ¹H NMR (300 MHz, CDCl₃) δ: 7.90 (d, J=2.3 Hz, 1H), 7.56 (dd, J=1.8, 0.8 Hz, 1H), 7.38 (dd, J=8.3, 2.3 Hz, 1H), 7.20 (d, J=8.3 Hz, 1H), 7.14 (dd, J=3.5, 0.8 Hz, 1H), 6.54 (dd, J=3.5, 1.8 Hz, 1H), 1.40 (s, 9H).

### Step 3

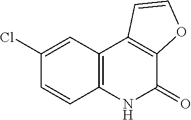

### 8-Chlorofuro[2,3-c]quinolin-4(5H)-one

A 20 mL microwave reaction tube was charged with tert-butyl 4-chloro-2-iodophenyl(furan-2-carbonyl)carbamate (0.45 g, 1.0 mmol), palladium(II) acetate (0.023 g, 0.1 mmol), tricyclohexylphosphine (0.028 g, 0.1 mmol), K₂CO₃ (0.28 g, 2.0 mmol) and N,N-dimethylacetamide (10 mL). After the air was purged by bubbling argon into the reaction solution, the tube was sealed and heated at 140° C. for 1 h in a Biotage microwave reactor. It was diluted with H₂O (100 mL) and extracted with EtOAc (100 mL×2). The combined organic layers were washed with brine (100 mL), dried over anhydrous Na₂SO₄ and concentrated in vacuo. The residue was purified by flash column chromatography on silica gel with 20-100% EtOAc in petroleum ether, to afford 0.10 g (53%) of the product as white solid.

### Step 4

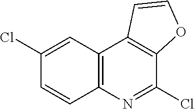

### 4,8-Dichlorofuro[2,3-c]quinoline

A 100 mL round bottom flask was charged with 8-chlorofuro[2,3-c]quinolin-4(5H)-one (100 mg, 0.46 mmol) and POCl₃ (20 mL). The resulting solution was heated at reflux for 2 h then concentrated under reduced pressure. The residue was mixed with saturated aqueous Na₂CO₃ and extracted with EtOAc (50 mL×4). The combined organic layers were dried over anhydrous Na₂SO₄ and concentrated in vacuo. The resulting solid was washed with EtOH to afford 100 mg (93%) of the product as white solid. ¹H NMR (300 MHz, CDCl₃) δ: 8.10-8.05 (m, 2H), 7.95 (d, J=2.0 Hz, 1H), 7.65 (dd, J=9.1, 2.1 Hz, 1H), 7.30 (d, J=2.1 Hz, 1H).

### Step 5

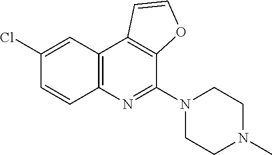

### 8-Chloro-4-(4-methylpiperazin-1-yl)furo[2,3-c]quinoline

A 20 mL microwave reaction tube was charged with 4,8-dichlorofurano[2,3-c]quinoline (110 mg, 0.46 mmol), N-methylpiperazine (0.15 mL, 1.4 mmol) and anhydrous iPrOH (10 mL). The resulting solution was heated at 130° C. for 1 h in a Biotage microwave reactor. The solvent was evaporated and the residue was purified by flash column chromatography on silica gel with 10% MeOH in CH₂Cl₂ to afford 100 mg (72%) of the product as white solid. ¹H NMR (300 MHz, CDCl₃) δ: 7.85 (d, J=2.4 Hz, 1H), 7.75 (m, 2H), 7.53 (dd, J=6.3, 2.4 Hz, 1H), 7.13 (d, J=1.8 Hz, 1H), 4.06 (t, J=5.1 Hz, 4H), 2.61 (t, J=5.1 Hz, 4H), 2.38 (s, 3H). MS m/z: 302 (M+H⁺).

### EXAMPLE 148

### 8-Chloro-4-(piperazin-1-yl)furo[2,3-c]quinoline

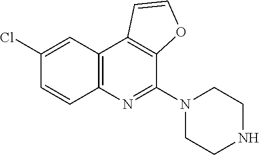

The title compound was prepared as described in Example 147, except that piperazine was substituted for N-methylpiperazine in step 5 of that route. ¹H NMR (300 MHz, D₂O) δ: 8.14 (d, J=1.5 Hz, 1H), 8.00 (s, 1H), 7.73 (d, J=9.0 Hz, 1H), 7.59 (m, 1H), 7.35 (s, 1H), 4.31 (t, J=5.1 Hz, 4H), 3.48 (t, J=5.1 Hz, 4H). MS m/z: 288 (M+H⁺).

### EXAMPLE 149

### 8-Chloro-4-(4-methylpiperazin-1-yl)thieno[2,3-c]quinoline

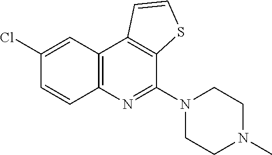

The title compound was prepared as described in Example 147, except that thiophene-2-carboxylic acid was substituted for furan-2-carboxylic acid in step 1 of that route. ¹H NMR (300 MHz, CDCl₃) δ: 8.07 (d, J=2.1 Hz, 1H), 7.83 (m, 2H), 7.73 (d, J=5.4 Hz, 1H), 7.50 (m, 1H), 3.85 (t, J=5.1 Hz, 4H), 2.66 (t, J=4.8 Hz, 4H), 2.40 (s, 3H). MS m/z: 318 (M+H⁺).

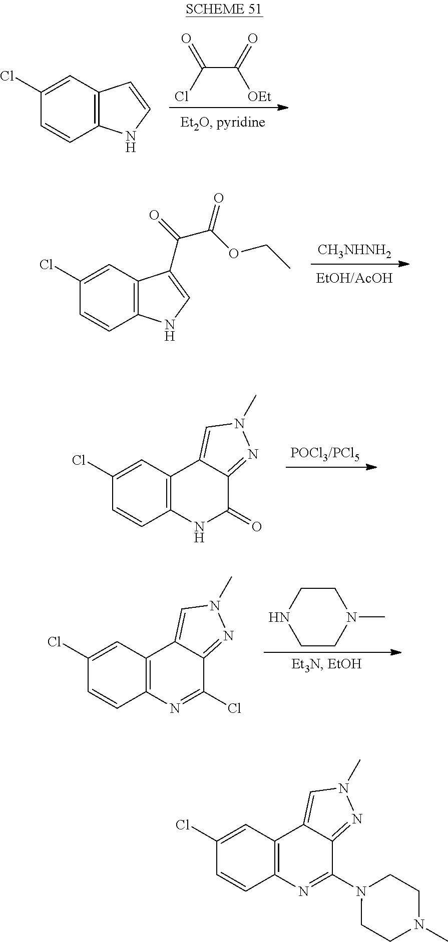

### EXAMPLE 150

### 8-Chloro-2-methyl-4-(4-methylpiperazin-1-yl)-2H-pyrazolo[3,4-c]quinoline

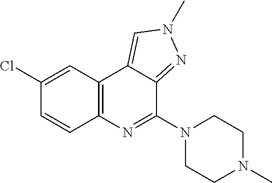

### Step 1

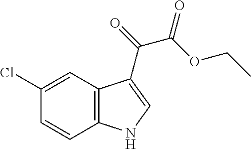

### Ethyl 2-(5-chloro-1H-indol-3-yl)-2-oxoacetate

A 500 mL 3-necked round bottom flask was charged with 5-chloroindole (15.2 g, 0.10 mol), pyridine (10.5 mL) and anhydrous ethyl ether (200 mL). To the above was added dropwise a solution of ethyl oxalylchloride (16.4 g, 0.12 mol) in anhydrous ethyl ether (50 mL) at 0-5° C. The resulting mixture was stirred at 0° C. for 1 h. Reaction progress was monitored by TLC (EtOAc/Petroleum ether=1:1, Rf=0.3). Work-up: the mixture was concentrated in vacuo. The resulting solid was washed with a small amount of ethyl ether, then with water, and dried, to give 19.3 g (77%) of the product. MS m/z: 252 (M+H⁺).

### Step 2

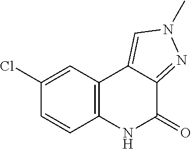

### 8-Chloro-2-methyl-2H-pyrazolo[3,4-c]quinolin-4(5H)-one

A 250 mL round bottom flask was charged with ethyl 2-(5-chloro-1H-indol-3-yl)-2-oxoacetate (3 g, 12 mmol), methylhydrazine hydrochloride salt (3 g, 16 mmol), absolute ethanol (150 mL) and acetic acid (3 mL). The resulting mixture was heated at reflux for 24 h. Work-up: the solvent was evaporated under reduced pressure. The residue was purified by flash column chromatography on silica gel with a 1:40 MeOH/CH₂Cl₂, to give 2.2 g (79%) of the product. ¹H NMR (300 MHz, DMSO-d₆) δ: 11.43 (s, 1H), 8.68 (s, 1H), 7.99 (s, 1H), 7.39-7.30 (m, 2H), 4.12 (s, 3H). MS m/z: 234 (M+H⁺).

### Step 3

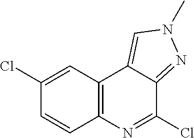

### 4,8-Dichloro-2-methyl-2H-pyrazolo[3,4-c]quinoline

A 100 mL round bottom flask was charged with 8-chloro-2-methyl-2H-pyrazolo[3,4-c]quinolin-4(5H)-one (2.2 g, 9.4 mmol), PCl₅ (0.28 g, 1.9 mmol) and POCl₃ (40 mL). The resulting mixture was heated at reflux for 2 h. Reaction progress was monitored by TLC (EtOAc/Petroleum ether=1:10, Rf=0.3). Work-up: POCl₃ was evaporated under reduced pressure. The residue was carefully poured into ice-cooled saturated aqueous NaHCO₃ (100 mL) and extracted with CH₂Cl₂ (50 mL×4). The combined organic layers were dried over anhydrous Na₂SO₄ and concentrated in vacuo. The residue was purified by flash column chromatography on silica gel with a 1:4 EtOAC/Petroleum ether, to give 1.67 g (70%) of the product. MS m/z: 253 (M+H⁺).

### Step 4

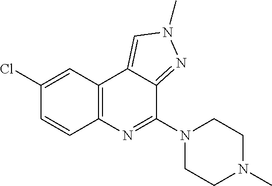

### 8-Chloro-2-methyl-4-(4-methylpiperazin-1-yl)-2H-pyrazolo[3,4-c]quinoline

A 100 mL round bottom flask was charged with 4,8-dichloro-2-methyl-2H-pyrazolo[3,4-c]quinoline (0.504 g, 2 mmol), N-methylpiperazine (0.6 g, 6 mmol), Et₃N (0.84 mL, 6.1 mmol) and absolute ethanol (35 mL). The resulting mixture was heated at reflux for 24 h. Work-up: the reaction mixture was concentrated under reduced pressure. The residue was purified by flash column chromatography on silica gel with a 1:20 MeOH/CH₂Cl₂, to give 300 mg (47%) of the product. ¹H NMR (300 MHz, CD₃OD) δ: 8.44 (s, 1H), 7.84 (d, J=2.7 Hz, 1H), 7.54 (d, J=8.7 Hz, 1H), 7.30 (dd, J=8.7, 2.4 Hz, 1H), 4.36 (br, 4H), 4.18 (s, 3H), 2.91 (t, J=5.1 Hz, 4H), 2.57 (s, 3H). MS m/z: 316 (M+H⁺).

### EXAMPLE 151

### 8-Chloro-2-methyl-4-(piperazin-1-yl)-2H-pyrazolo[3,4-c]quinoline

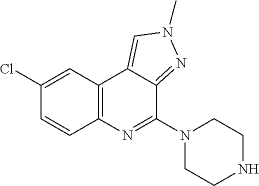

The HCl salt of the title compound was prepared as described in Example 150, except that tert-butyl piperazine-1-carboxylate was substituted for N-methylpiperazine in step 4 of that route. The resulting tert-butyl 4-(8-chloro-2-methyl-2H-pyrazolo[3,4-c]quinolin-4-yl)piperazine-1-carboxylate was treated with 3 M HCl in methanol solution overnight at room temperature. The solid was collected by filtration, washed with methanol, and dried, to afford the HCl salt of the title compound as white solid. ¹H NMR (300 MHz, DMSO-d₆) δ: 9.83 (br, 2H), 9.04 (s, 1H), 8.35 (m, 1H), 8.22 (d, J=2.1 Hz, 1H), 7.57 (dd, J=8.7, 2.1 Hz, 1H), 4.72 (br, 4H), 4.22 (s, 3H), 3.78 (m, 4H). MS m/z: 288 (M+H⁺).

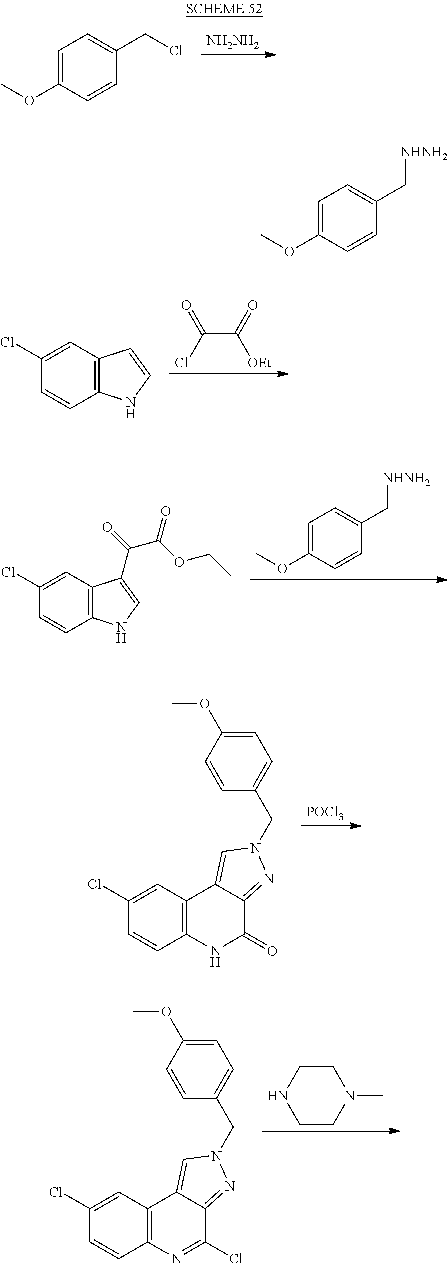

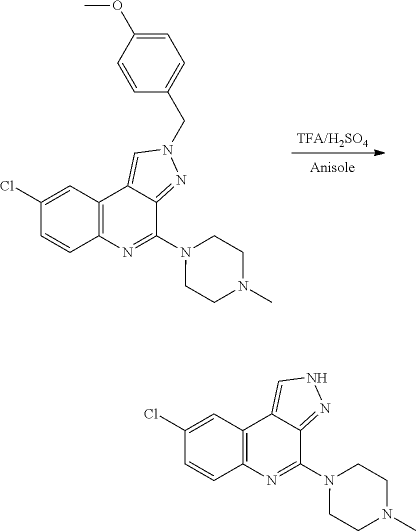

### EXAMPLE 152

### 8-Chloro-4-(4-methylpiperazin-1-yl)-2H-pyrazolo[3,4-c]quinoline

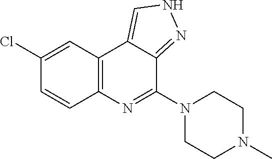

### Step 1

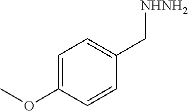

### (4-Methoxybenzyl)hydrazine HCl salt

A 500 mL 3-necked round bottom flask was charged with hydrazine hydrate (40 g, 0.80 mol) and EtOH (280 mL). To the above solution was added dropwise a solution of 4-methoxybenzylchloride (12.5 g, 0.080 mol) in EtOH (30 mL) at room temperature. The resulting mixture was stirred at 90° C. for 2 h. Work-up: the reaction mixture was concentrated in vacuo then re-dissolved in EtOH (150 mL). The solution was acidified with 5 M HCl (120 mL) at 0° C. The resulting precipitate was collected by filtration and dried to afford 8.72 g (72%) of the product as white solid. MS m/z: 153 (M+H⁺).

### Steps 2-3

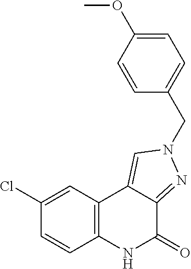

### 8-Chloro-2-(4-methoxybenzyl)-2H-pyrazolo[3,4-c]quinolin-4(5H)-one

The title compound was prepared as described in Example 150, except that (4-methoxybenzyl)hydrazine HCl salt was substituted for methylhydrazine HCl salt in step 2 of that route. ¹H NMR (300 MHz, DMSO-d₆) δ: 11.43 (s, 1H), 8.77 (s, 1H), 8.01 (d, J=2.1 Hz, 1H), 7.38-7.29 (m, 4H), 6.96-6.93 (m, 2H), 5.52 (s, 2H), 3.74 (s, 3H). MS m/z: 340 (M+H⁺).

### Step 4

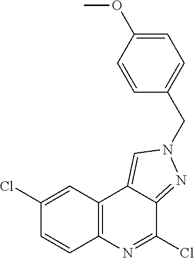

### 4,8-Dichloro-2-(4-methoxybenzyl)-2H-pyrazolo[3,4-c]quinoline

The title compound was prepared as described in Example 150, except that 8-chloro-2-(4-methoxybenzyl)-2H-pyrazolo[3,4-c]quinolin-4(5H)-one was substituted for 8-chloro-2-methyl-2H-pyrazolo[3,4-c]quinolin-4(5H)-one in step 3 of that route. MS m/z: 359 (M+H⁺).

### Step 5

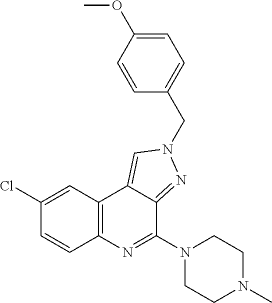

### 8-Chloro-2-(4-methoxybenzyl)-4-(4-methylpiperazin-1-yl)-2H-pyrazolo[3,4-c]quinoline

The title compound was prepared as described in Example 150, except that 4,8-dichloro-2-(4-methoxybenzyl)-2H-pyrazolo[3,4-c]quinoline was substituted for 4,8-dichloro-2-methyl-2H-pyrazolo[3,4-c]quinoline in step 4 of that route. ¹H NMR (300 MHz, DMSO-d₆) δ: 8.20 (s, 1H), 7.83 (s, 1H), 7.52 (m, 1H), 7.38-7.22 (m, 3H), 6.88 (m, 2H), 5.52 (s, 2H), 4.29 (m, 4H), 3.74 (s, 3H), 2.66 (m, 4H), 2.38 (s, 3H). MS m/z: 422 (M+H⁺).

### Step 6

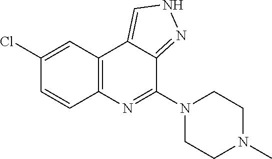

### 8-Chloro-4-(4-methylpiperazin-1-yl)-2H-pyrazolo[3,4-c]quinoline

A 50 mL 3-necked round bottom flask was charged with 8-chloro-2-(4-methoxybenzyl)-4-(4-methylpiperazin-1-yl)-2H-pyrazolo[3,4-c]quinoline (1.28 g, 3.0 mmol), trifluoroacetic acid (30 mL), anisole (881 mg, 8.2 mmol) and concentrated H₂SO₄ (0.45 mL). The resulting mixture was stirred at 0° C. for 2 h and then at 50° C. overnight. Work-up: the reaction solution was added dropwise to an ice-cooled saturated aqueous Na₂CO₃ (100 mL) and extracted with EtOAc (50 mL×3). The combined organic layers were washed with brine, dried over anhydrous Na₂SO₄ and concentrated in vacuo. The residue was purified by flash column chromatography on silica gel with a 1:20 MeOH/CH₂Cl₂, to afford 400 mg (44%) of the product. ¹H NMR (300 MHz, CD₃OD) δ: 8.57 (s, 1H), 7.97 (d, J=2.1 Hz, 1H), 7.60 (d, J=8.7 Hz, 1H), 7.33 (dd, J=8.7, 2.4 Hz, 1H), 4.22 (m, 4H), 2.64 (t, J=5.1 Hz, 4H), 2.36 (s, 3H). MS m/z: 302 (M+H⁺).

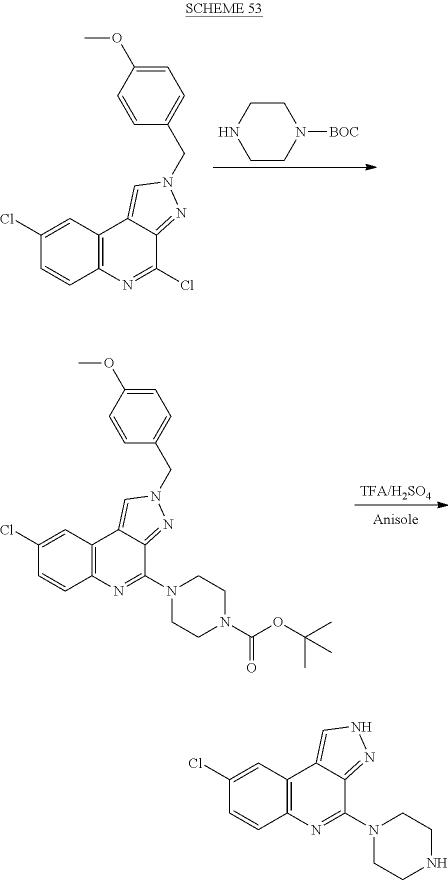

### EXAMPLE 153

### 8-Chloro-4-(piperazin-1-yl)-2H-pyrazolo[3,4-c]quinoline

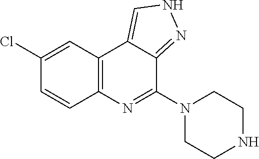

The title compound was prepared as described in Example 152, except that tert-butyl piperazine-1-carboxylate was substituted for N-methylpiperazine in step 5 of that route. ¹H NMR (300 MHz, CD₃OD) δ: 8.93 (s, 1H). 8.24 (d, J=2.1 Hz, 1H), 8.00 (d, J=9.0 Hz, 1H), 7.60 (dd, J=8.7, 2.1 Hz, 1H), 3.62 (m, 4H), 3.30 (m, 4H). MS m/z: 288 (M+H⁺).

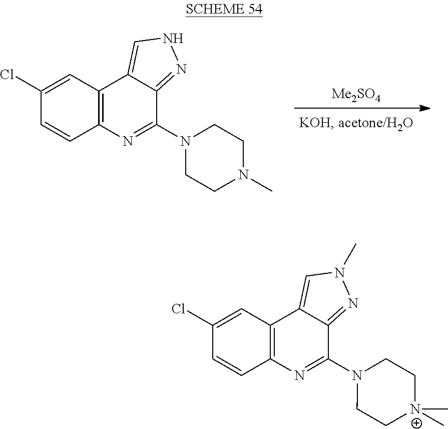

### EXAMPLE 154

### 4-(8-Chloro-2-methyl-2H-pyrazolo[3,4-c]quinolin-4-yl)-1,1-dimethylpiperazin-1-ium

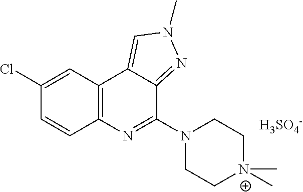

### Step 1

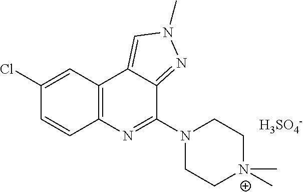

### 4-(8-Chloro-2-methyl-2H-pyrazolo[3,4-c]quinolin-4-yl)-1,1-dimethylpiperazin-1-ium

A 25 mL 3-necked round bottom flask was charged with 8-chloro-4-(4-methylpiperazin-1-yl)-2H-pyrazolo[3,4-c]quinoline (152, 200 mg, 0.664 mmol) and KOH (372 mg, 6.64 mmol) and H₂O (10 mL). To the above was added dropwise a solution of dimethyl sulfate (418 mg, 3.32 mmol) in acetone (2 mL). The resulting mixture was stirred at room temperature for 0.5 h. Reaction progress was monitored by TLC (MeOH/CH₂Cl₂=10:1, Rf=0.3). Work-up: the reaction mixture was concentrated under reduced pressure and the residue was purified by preparative HPLC to give 100 mg (46%) of the product. ¹H NMR (300 MHz, DMSO-d₆) δ: 8.91 (s, 1H), 8.12 (d, J=2.1 Hz, 1H), 7.60 (d, J=8.7 Hz, 1H), 7.42 (dd, J=8.7, 2.4 Hz, 1H), 4.55 (br, 4H), 4.22 (s, 3H), 3.60 (m, 4H), 3.24 (s, 6H). MS m/z: 330 (M+H⁺).

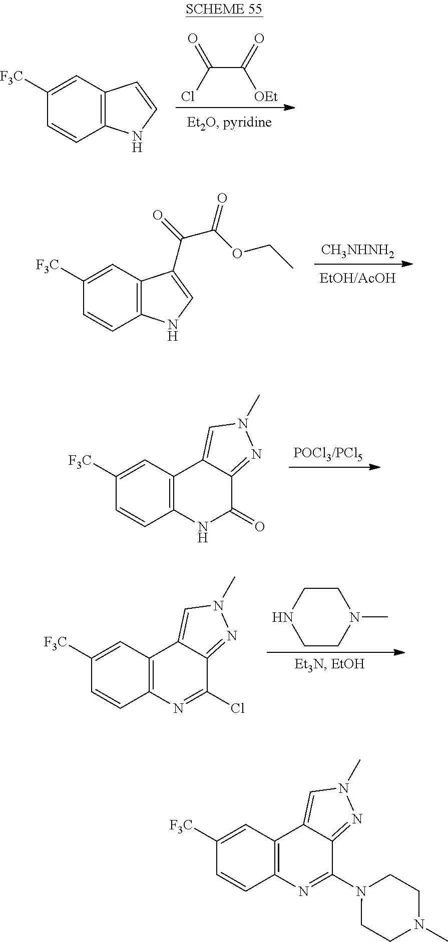

### EXAMPLE 155

### 2-Methyl-4-(4-methylpiperazinyl)-8-(trifluoromethyl)pyrazolo[3,4-c]quinoline

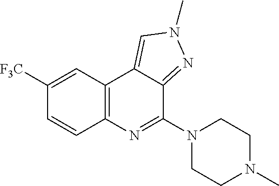

The title compound was prepared as described in Example 150, except that 5-trifluoromethylindole was substituted for 5-chloroindole in step 1 of that route. ¹H NMR (300 MHz, CD₃OD) δ: 8.57 (s, 1H), 8.18 (s, 1H), 7.67 (d, J=9.0 Hz, 1H), 7.55 (d, J=9.0 Hz, 1H), 4.36-4.32 (m, 4H), 4.21 (s, 3H), 2.64-2.61 (m, 4H), 2.36 (s, 3H). MS m/z: 350 (M+H⁺).

### EXAMPLE 156

### 2-Methyl-4-piperazinyl-8-(trifluoromethyl)pyrazolo[3,4-c]quinoline HCl salt

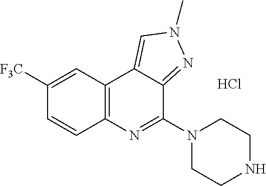

The title compound was prepared as described in Example 155, except that piperazine was substituted for N-methylpiperazine in step 4 of that route. ¹H NMR (300 MHz, CD₃OD) δ: 8.98 (s, 1H), 8.51 (s, 1H), 8.18 (d, J=9.0 Hz, 1H), 7.88 (d, J=9.0 Hz, 1H), 4.34 (s, 3H), 3.66-3.63 (m, 4H), 3.31-3.29 (m, 4H). MS m/z: 336 (M+H⁺).

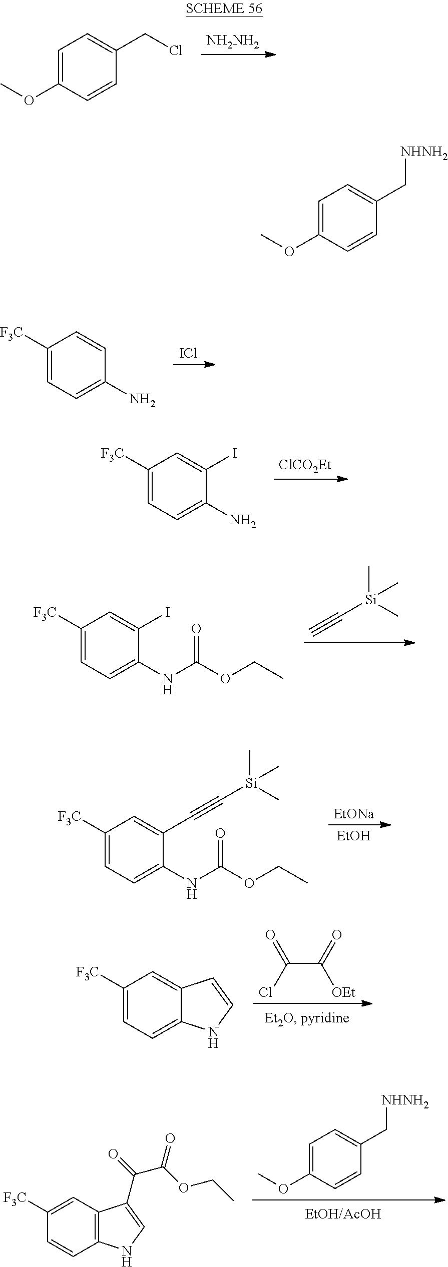

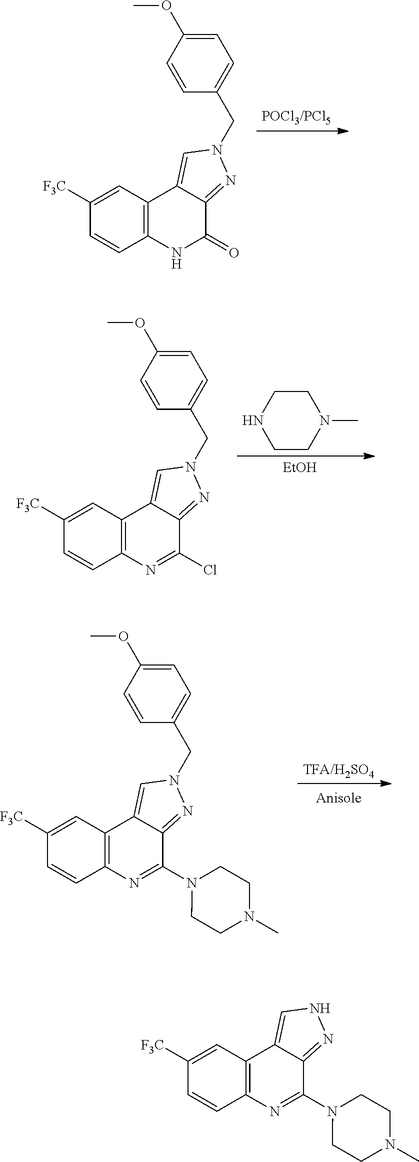

### EXAMPLE 157

### 4-(4-Methylpiperazinyl)-8-(trifluoromethyl)pyrazolo[3,4-c]quinoline

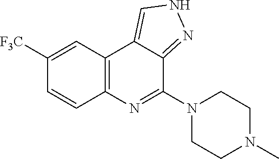

### Step 1

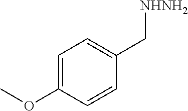

### (4-Methoxybenzyl)hydrazine

The HCl salt of the title compound was prepared as described in Example 152.

### Step 2

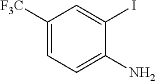

### 2-Iodo-4-(trifluoromethyl)aniline

A 500 mL 3-necked round bottom flask was charged with 4-(trifluoromethyl)aniline (22.5 g, 0.14 mol) and MeOH (100 mL). To the above was added dropwise a solution of ICl (25 g, 0.15 mol) in CH₂Cl₂ (100 mL) at 0° C. The resulting mixture was stirred at room temperature for 1 h. Reaction progress was monitored by TLC (EtOAc/Petroleum ether=1:10, Rf=0.5). Work-up: the mixture was concentrated in vacuo. The residue was re-dissolved in CH₂Cl₂, washed with water, dried over anhydrous Na₂SO₄ and concentrated in vacuo, to give 37.8 g (97%) of the product. ¹H NMR (300 MHz, CDCl₃) δ: 7.86 (d, J=1.2 Hz, 1H), 7.36 (dd, J=8.4, 1.8 Hz, 1H), 6.73 (d, J=8.7 Hz, 1H), 4.41 (br, 2H).

### Step 3

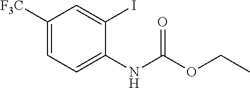

### Ethoxy-N-[2-iodo-4-(trifluoromethyl)phenyl]carboxamide

A 500 mL 3-necked round bottom flask was charged with 2-iodo-4-(trifluoromethyl)aniline (63 g, 0.22 mol) and pyridine (300 mL). To the above was added dropwise ethyl chloroformate (36 g, 0.33 mol) at 0° C. The resulting mixture was stirred at room temperature for 1 h. Reaction progress was monitored by TLC (EtOAc/Petroleum ether=1:20, Rf=0.5). Work-up: the mixture was concentrated in vacuo. The residue was re-dissolved in CH₂Cl₂, washed with saturated NH₄Cl, dried over anhydrous Na₂SO₄ and concentrated in vacuo, to give 43.5 g (55%) of the product. MS m/z: 358 (M−H⁺).

### Step 4

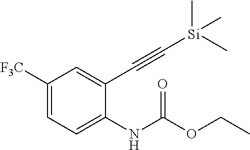

### N-[2-(3,3-Dimethyl-3-silabut-1-ynyl)-4-(trifluoromethyl)phenyl]ethoxycarboxamide

A 250 mL 3-necked round bottom flask was charged with ethoxy-N-[2-iodo-4-(trifluoromethyl)phenyl]carboxamide (50 g, 0.14 mol), CuI (1.5 g, 7.87 mmol), (1,1′-bis(diphenylphosphino)ferrocene)dichloropalladium(II) (5.0 g, 7.2 mmol), Et₃N (200 mL) and THF (400 mL). To the above was added dropwise 2,2-dimethyl-2-silabut-3-yne (21.7 mL, 0.15 mol). The resulting mixture was stirred at room temperature for 0.5 h under N₂ atmosphere. Reaction progress was monitored by TLC (EtOAc/Petroleum ether=1:20). Work-up: the mixture was concentrated in vacuo. The residue was purified by flash column chromatography on silica gel with 5% EtOAc in petroleum ether, to afford 31.5 g (74%) of the product. ¹H NMR (300 MHz, DMSO-d₆) δ: 8.28 (d, J=8.7 Hz, 1H), 7.64 (m, 1H), 7.55 (m, 2H), 4.26 (q, J=6.9 Hz, 2H), 1.34 (t, J=7.2 Hz, 3H), 0.31 (s, 9H).

### Step 5

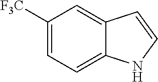

### 5-(Trifluoromethyl)indole

A 250 mL 3-necked round bottom flask was charged with N-[2-(3,3-dimethyl-3-silabut-1-ynyl)-4-(trifluoromethyl)phenyl]ethoxycarboxamide (31.5 g, 0.1 mol), EtONa (32.5 g, 0.48 mol) and ethanol (200 mL). The resulting mixture was heated at reflux for 2 h. Work-up: the mixture was concentrated in vacuo. The residue was purified by flash column chromatography on silica gel with 25% EtOAc in petroleum ether, to afford 14 g (77%) of the product. ¹H NMR (300 MHz, DMSO-d₆) δ: 8.36 (s, 1H), 7.96-7.94 (m, 1H), 7.46-7.44 (m, 2H), 7.32-7.30 (m, 1H), 6.66-6.64 (m, 1H).

### Steps 6-9

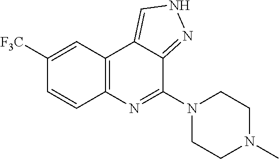

### 4-(4-Methylpiperazinyl)-8-(trifluoromethyl)pyrazolo[3,4-c]quinoline

The HCl salt of the title compound was prepared as described in Example 152, except that 5-(trifluoromethyl)indole was substituted for 5-chloroindole in step 2 of that route. ¹H NMR (300 MHz, D₂O) δ: 8.52-8.50 (m, 1H), 8.12-8.10 (m, 1H), 7.72-7.69 (m, 1H), 7.62-7.59 (m, 1H), 5.38-5.35 (m, 2H), 3.74-3.71 (m, 4H), 3.32-3.28 (m, 2H), 2.87 (s, 3H). MS m/z: 336 (M+H⁺).

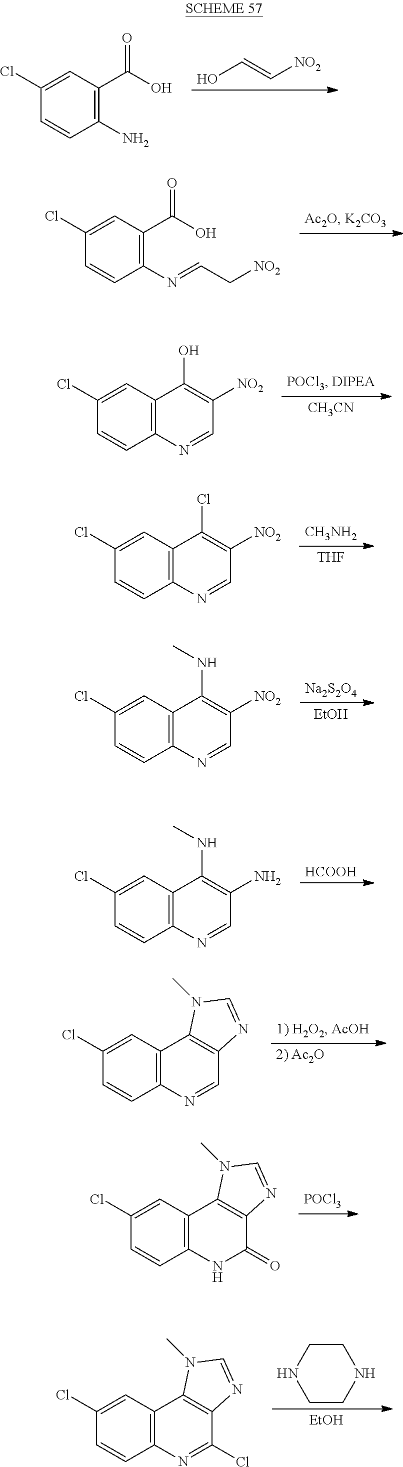

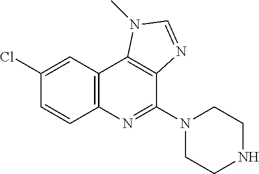

### EXAMPLE 158

### 8-Chloro-1-methyl-4-(piperazin-1-yl)-1H-imidazo[4,5-c]quinoline

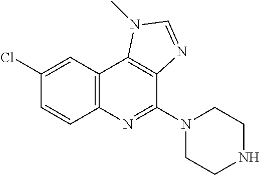

### Step 1

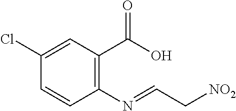

### 5-Chloro-2-(2-nitroethylideneamino)benzoic acid

A 100 mL round bottom flask was charged with NaOH (2.33 g, 0.058 mol) and H₂O (10 mL). To the above was added dropwise nitromethane (3.1 mL, 3.56 g, 0.058 mol) at room temperature. The resulting solution was slowly warmed to 45° C. for 5 min then cooled to 0° C. and acidified with concentrated HCl. It was added to a suspension of 2-amino-5-chlorobenzoic acid (5.0 g, 0.029 mol) in concentrated HCl (50 mL) and H₂O (20 mL). The reaction solution was allowed to stand overnight at room temperature. The solid was collected by filtration, washed with H₂O, and dried, to afford 4.7 g (66%) of the product.

### Step 2

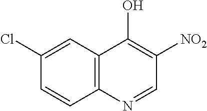

### 6-Chloro-3-nitroquinolin-4-ol

A 500 mL round bottom flask was charged with 5-chloro-2-(2-nitroethylideneamino)benzoic acid (25 g, 0.10 mol), K₂CO₃ (42.6 g, 0.30 mol) and acetic anhydride (250 mL). The resulting mixture was heated to 90° C. for 1 h. Work-up: the resulting solid was collected by filtration, washed with water and dried to give 17.5 g (76%) of the product as grey solid. ¹H NMR (300 MHz, DMSO-d₆) δ: 9.12 (s, 1H), 8.15 (s, 1H), 7.72 (s, 2H). MS m/z: 224 (M+H⁺).

### Step 3

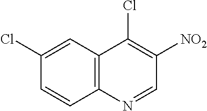

### 4,6-Dichloro-3-nitroquinoline

A 500 mL round bottom flask was charged with 6-chloro-3-nitroquinolin-4-ol (2.41 g, 10.8 mmol), acetonitrile (50 mL), N,N-diisopropylethylamine (2.49 g, 21.6 mmol) and POCl₃ (1.5 mL, 16.2 mmol). The resulting solution was heated at reflux for 1 h. Work-up: the solvent was removed, and the residue was purified by flash column chromatography on silica gel with a 1:15 EtOAc/Petroleum ether, to give 2.0 g (77%) of the product as white solid. ¹H NMR (300 MHz, CDCl₃) δ: 9.23 (s, 1H), 8.40 (d, J=2.1 Hz, 1H), 8.16 (d, J=9.0 Hz, 1H), 7.89 (dd, J=9.0, 2.4 Hz, 1H).

### Step 4

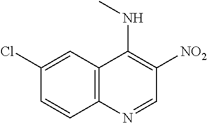

### 6-Chloro-N-methyl-3-nitroquinolin-4-amine

A 100 mL round bottom flask was charged with 4,6-dichloro-3-nitroquinoline (2.0 g, 8.3 mmol) and THF (50 mL). To the above was added methylamine (2 M in THF, 6.2 mL) at 0° C. The resulting solution was stirred at room temperature for 1 h. Work-up: the solvent was removed. The residue was dissolved in CH₂Cl₂ (300 mL) and washed with water (50 mL). The organic layer was dried over anhydrous Na₂SO₄ and concentrated in vacuo. It was further purified by flash column chromatography on silica gel with a 1:2:2 EtOAc/Petroleum ether/CH₂Cl₂, to give 1.8 g (91%) of the product as yellow solid. MS m/z: 238 (M+H⁺).

### Step 5

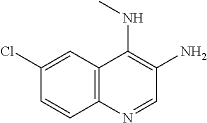

### 6-Chloro-N⁴-methylquinoline-3,4-diamine

A 100 mL round bottom flask was charged with 6-chloro-N-methyl-3-nitroquinolin-4-amine (1.1 g, 4.6 mmol), sodium dithionite (1.62 g, 9.2 mmol), water (10 mL) and EtOH (50 mL). The resulting mixture was heated at reflux for 1 h. Work-up: the solvent was removed and the residue was washed with water (5 mL) and dried to afford 0.96 g (quantitative) of the product, which was used as such for the next step. MS m/z: 208 (M+H⁺).

### Step 6

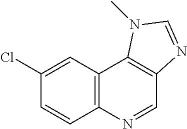

### 8-Chloro-1-methyl-1H-imidazo[4,5-c]quinoline

A 100 mL round bottom flask was charged with 6-chloro-N⁴-methylquinoline-3,4-diamine (0.96 g, 4.6 mmol), HCOOH (30 mL) and concentrated HCl (5 mL). The resulting mixture was heated at reflux for 30 min. Work-up: the solvent was removed. The residue was poured into 50% aqueous NaOH at 0° C. and extracted with CH₂Cl₂ (100 mL×4). The combined organic layers were dried over anhydrous Na₂SO₄ and concentrated in vacuo. It was further purified by flash column chromatography on silica gel with 33% EtOAc in petroleum ether then 3% MeOH in CH₂Cl₂, to give 0.47 g (46%) of the product as white solid. ¹H NMR (300 MHz, CDCl₃) δ: 9.30 (s, 1H), 8.23 (d, J=4.2 Hz, 1H), 8.19 (s, 1H), 7.94 (s, 1H), 7.63 (d, J=6.6 Hz, 1H), 4.28 (s, 3H). MS m/z: 218 (M+H⁺).

### Step 7

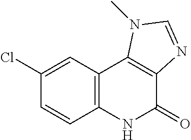

### 8-Chloro-1-methyl-1H-imidazo[4,5-c]quinolin-4(5H)-one

A 50 mL round bottom flask was charged with 8-chloro-1-methyl-1H-imidazo[4,5-c]quinoline (1.4 g, 6.46 mmol), 30% H₂O₂ (1.5 mL) and acetic acid (20 mL). The reaction mixture was stirred at 80° C. overnight then concentrated under reduced pressure. The residue was neutralized with saturated aqueous NaHCO₃ and the resulting precipitate was collected by filtration and dried. It was re-suspended in acetic anhydride (15 mL) and heated at reflux for 1 h. The solvent was removed and methanol (10 mL) was added to the residue, followed by dropwise addition of a solution of 28% sodium methoxide in methanol until PH 10. The solid was collected by filtration and dried to give 0.40 g (27%) of the product as yellow solid. ¹H NMR (300 MHz, DMSO-d₆) δ: 11.70 (s, 1H), 8.12 (s, 1H), 8.05 (s, 1H), 7.53-7.44 (m, 2H), 4.17 (s, 3H). MS m/z: 234 (M+H⁺).

### Step 8

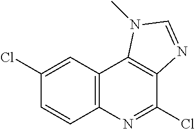

### 4,8-Dichloro-1-methyl-1H-imidazo[4,5-c]quinoline

A 50 mL round bottom flask was charged with 8-chloro-1-methyl-1H-imidazo[4,5-c]quinolin-4(5H)-one (0.20 g, 0.86 mmol) and POCl₃ (5 mL). The mixture was heated at reflux for 1 h. Work-up: the solvent was removed under reduced pressure. The residue was treated with saturated aqueous Na₂CO₃ at 0° C., extracted with CH₂Cl₂ (50 mL×2), concentrated in vacuo and further purified by flash column chromatography on silica gel with 5% MeOH in CH₂Cl₂, to give 0.12 g (56%) of the product as yellow solid. MS m/z: 252 (M+H⁺).

### Step 9

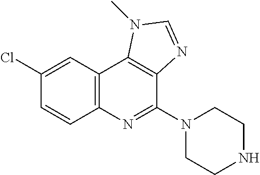

### 8-Chloro-1-methyl-4-(piperazin-1-yl)-1H-imidazo[4,5-c]quinoline

A 20 mL microwave reaction tube was charged with 4,8-dichloro-1-methyl-1H-imidazo[4,5-c]quinoline (0.21 g, 0.84 mmol), piperazine (0.14 g, 1.68 mmol) and EtOH (10 mL). The resulting mixture was heated at 140° C. for 2 h in a Biotage microwave reactor. Work-up: the solvent was removed. The residue was diluted with CH₂Cl₂ (50 mL) and washed with water (30 mL×2). The organic layer was dried over anhydrous Na₂SO₄ and concentrated in vacuo. The residue was then treated with 3 M HCl (2.0 mL) and THF (20 mL). The resulting white solid was collected by filtration and dried, to give 160 mg (57%) of the HCl salt of the product as white solid. ¹H NMR (300 MHz, D₂O) δ: 8.17 (s, 1H), 7.93 (d, J=2.1 Hz, 1H), 7.70 (d, J=9.3 Hz, 1H), 7.44 (dd, J=9.0, 2.1 Hz, 1H), 4.53 (t, J=5.4 Hz, 4H), 4.03 (s, 3H), 3.50 (t, J=5.4 Hz, 4H). MS m/z: 302 (M+H⁺).

### EXAMPLE 159

### 8-Chloro-1-methyl-4-(4-methylpiperazin-1-yl)-1H-imidazo[4,5-c]quinoline

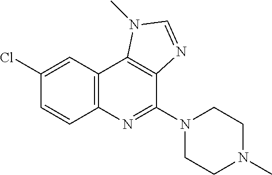

The title compound was prepared as described in Example 158, except that N-methylpiperazine was substituted for piperazine in step 9 of that route. ¹H NMR (300 MHz, DMSO-d₆) δ: 8.32 (s, 1H), 8.22 (d, J=2.1 Hz, 1H), 7.73 (d, J=8.7 Hz, 1H), 7.54 (dd, J=9.0, 2.1 Hz, 1H), 4.26 (s, 3H), 3.26 (br, 8H), 2.75 (s, 3H). MS m/z: 316 (M+H⁺).

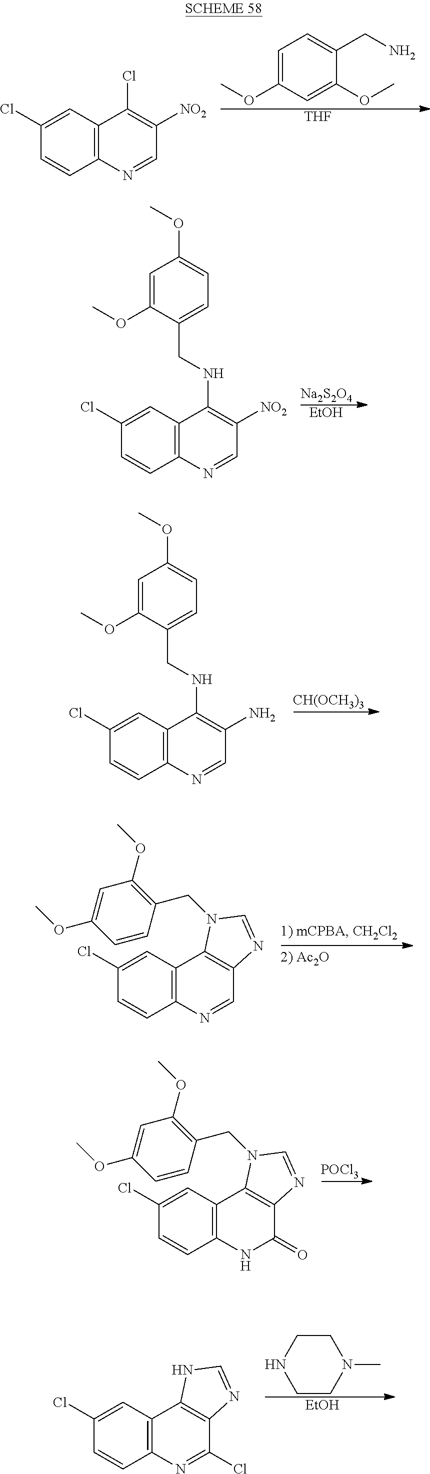

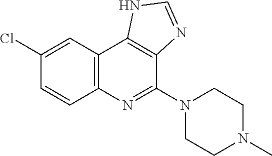

### EXAMPLE 160

### 8-Chloro-4-(4-methylpiperazin-1-yl)-1H-imidazo[4,5-c]quinoline

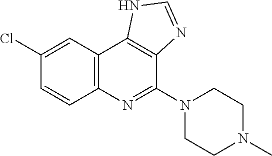

### Step 1

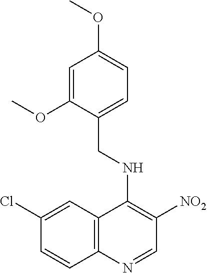

### 6-Chloro-N-(2,4-dimethoxybenzyl)-3-nitroquinolin-4-amine

The title compound was prepared as described in Example 158, except that 2,4-dimethoxybenzylamine was substituted for methylamine in step 4 of that route. MS m/z: 373 (M+H⁺).

### Step 2

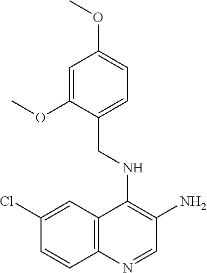

### 6-Chloro-N⁴-(2,4-dimethoxybenzyl)quinoline-3,4-diamine

The title compound was prepared as described in Example 158, except that 6-chloro-N-(2,4-dimethoxybenzyl)-3-nitroquinolin-4-amine was substituted for 6-chloro-N-methyl-3-nitroquinolin-4-amine in step 5 of that route.

### Step 3

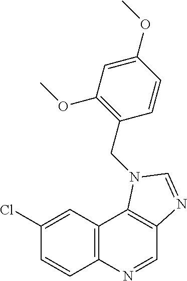

### 8-Chloro-1-(2,4-dimethoxybenzyl)-1H-imidazo[4,5-c]quinoline

The title compound was prepared as described in Example 158, except that 6-chloro-N⁴-(2,4-dimethoxybenzyl)quinoline-3,4-diamine was substituted for 6-chloro-N⁴-methylquinoline-3,4-diamine, and methyl orthoformate for HCOOH and concentrated HCl in step 6 of that route.

### Step 4

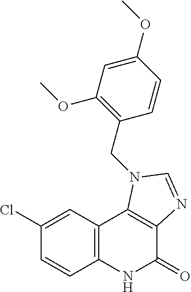

### 8-Chloro-1-(2,4-dimethoxybenzyl)-1H-imidazo[4,5-c]quinolin-4(5H)-one

A 100 mL round bottom flask was charged with 8-chloro-1-(2,4-dimethoxybenzyl)-1H-imidazo[4,5-c]quinoline (2.10 g, 5.94 mmol), 3-chloroperbenzoic acid (1.23 g, 7.13 mmol) and CH₂Cl₂ (50 mL). The resulting solution was stirred at room temperature for 3 h. Reaction progress was monitored by TLC (MeOH/CH₂Cl₂=1:20, Rf=0.4). Work-up: the mixture was concentrated and the residue was purified by flash column chromatography on silica gel with a 1:20 MeOH/CH₂Cl₂, to give 1.7 g (77%) of white solid, which was suspended in acetic anhydride (20 mL) and stirred at reflux for 1 h. The mixture was concentrated and the residue was diluted with methanol (5 mL), followed by dropwise addition of a solution of 28% sodium methoxide in methanol until PH 10. The solid was collected by filtration and dried to give 1.5 (68%) of the product as white solid. ¹H NMR (300 MHz, DMSO-d₆) δ: 8.17 (s, 1H), 7.60 (s, 1H), 7.34 (d, J=8.1 Hz, 1H), 7.25 (d, J=7.8 Hz, 1H), 6.68 (s, 1H), 6.53 (d, J=8.4 Hz, 1H), 6.40 (d, J=9.0 Hz, 1H), 5.61 (s, 2H), 3.94 (s, 3H), 3.71 (s, 3H).

### Step 5

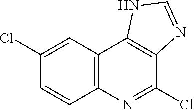

### 4,8-Dichloro-1H-imidazo[4,5-c]quinoline

A 50 mL round bottom flask was charged with 8-chloro-1-(2,4-dimethoxybenzyl)-1H-imidazo[4,5-c]quinolin-4(5H)-one (0.80 g, 2.17 mmol), POCl₃ (15 mL) and N,N-diisopropylethylamine (0.50 g, 4.34 mmol). The resulting mixture was stirred overnight at reflux. Work-up: the mixture was concentrated and the residue was purified by flash column chromatography on silica gel with a 1:20 MeOH/CH₂Cl₂, to give 0.20 g (40%) of the product as white solid. MS m/z: 238 (M+H⁺).

### Step 6

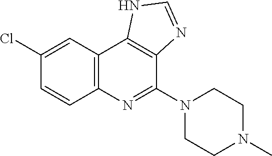

### 8-Chloro-4-(4-methylpiperazin-1-yl)-1H-imidazo[4,5-c]quinoline

The title compound was prepared as described in Example 158, except that 4,8-dichloro-1H-imidazo[4,5-c]quinoline was substituted for 4,8-dichloro-1-methyl-1H-imidazo[4,5-c]quinoline, and N-methylpiperazine for piperazine in step 9 of that route. ¹H NMR (300 MHz, DMSO-d₆) δ: 8.33 (s, 1H), 8.17 (d, J=1.8 Hz, 1H), 7.61 (d, J=8.7 Hz, 1H), 7.42 (dd, J=9.0, 2.7 Hz, 1H), 4.24 (br, 4H), 2.49 (m, 4H), 2.22 (s, 3H). MS m/z: 302 (M+H⁺).

### EXAMPLE 161

### 8-Chloro-4-(piperazin-1-yl)-1H-imidazo[4,5-c]quinoline

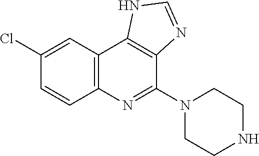

The title compound was prepared as described in Example 160, except that piperazine was substituted for N-methylpiperazine in step 6 of that route. ¹H NMR (300 MHz, CD₃OD) δ: 8.18 (s, 1H), 8.03 (d, J=2.1 Hz, 1H), 7.70 (d, J=9.0 Hz, 1H), 7.42 (dd, J=9.0, 2.4 Hz, 1H), 4.15 (t, J=5.0 Hz, 4H), 3.03 (t, J=5.1 Hz, 4H). MS m/z: 288 (M+H⁺).

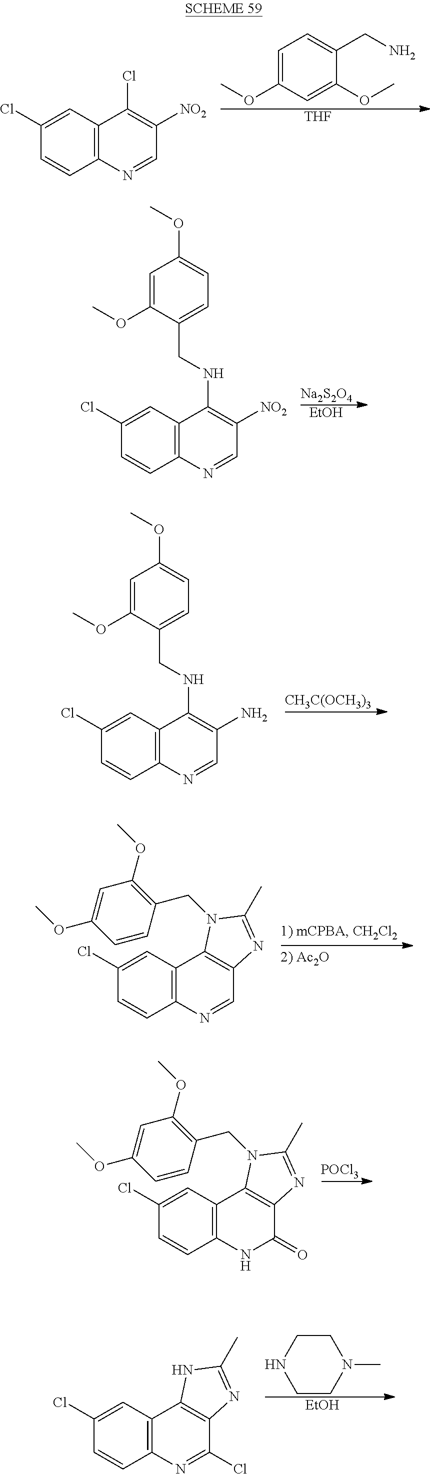

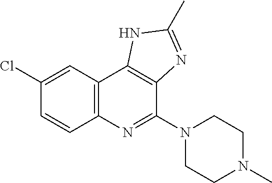

### EXAMPLE 162

### 8-Chloro-2-methyl-4-(4-methylpiperazinyl)imidazo[4,5-c]quinoline

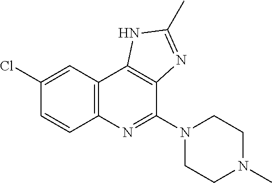

The title compound was prepared as described in Example 160, except that triethyl orthoacetate was substituted for triethyl orthoformate in step 3 of that route. ¹H NMR (300 MHz, CD₃OD) δ: 7.97 (d, J=2.4 Hz, 1H), 7.74 (d, J=9.0 Hz, 1H), 7.45 (dd, J=9.0, 2.4 Hz, 1H), 4.88 (m, 4H), 3.39 (m, 4H), 2.92 (s, 3H), 2.64 (s, 3H). MS m/z: 316 (M+H⁺).

### EXAMPLE 163

### 8-Chloro-2-methyl-4-piperazinylimidazo[4,5-c]quinoline

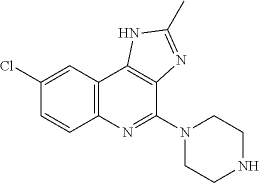

The title compound was prepared as described in Example 162, except that piperazine was substituted for N-methylpiperazine in step 6 of that route. ¹H NMR (300 MHz, D₂O) δ: 7.49 (d, J=9.0 Hz, 1H), 7.24 (dd, J=9.0, 2.1 Hz, 1H), 7.19 (d, J=2.1 Hz, 1H), 4.42 (t, J=5.1 Hz, 4H), 3.45 (t, J=5.1 Hz, 4H), 2.45 (s, 3H). MS m/z: 302 (M+H⁺).

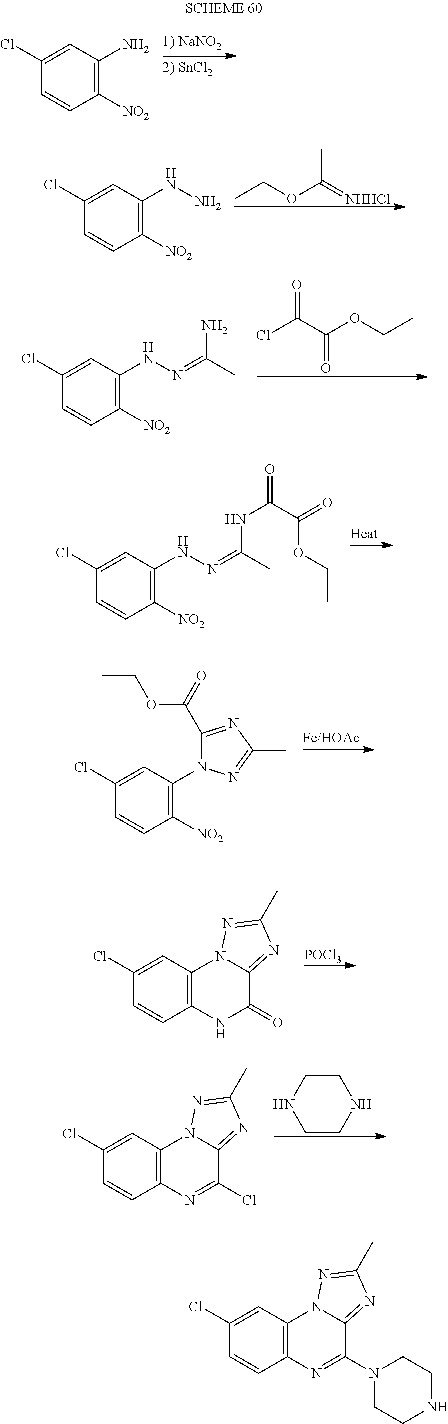

### EXAMPLE 164

### 8-chloro-2-methyl-4-(piperazin-1-yl)-[1,2,4]triazolo[1,5-a]quinoxaline

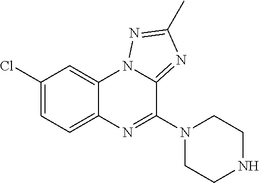

### Step 1

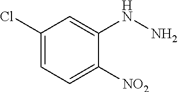

### 5-Chloro-2-nitrophenylhydrazine

A 500 mL round bottom flask was charged with 5-chloro-2-nitroaniline (17.25 g, 0.1 mol) and 6 N HCl (100 mL). To the above was added dropwise a solution of NaNO₂ (7.7 g, 0.105 mol) in water (30 mL) at 0-5° C. and the resulting mixture was stirred for 1 h. The diazotized solution was filtered and added slowly with stirring to an ice cold solution of SnCl₂ (56.4 g, 0.25 mol) in concentrated HCl (70 mL). The reaction progress was monitored by TLC (EtOAc/Petroleum ether=1:4, Rf=0.3). Work up: the yellow precipitate was collected by filtration, then partitioned between EtOAc (300 mL) and saturated aqueous NaOAc solution (200 mL). The organic layer was separated, dried over anhydrous MgSO₄, and concentrated in vacuo, to give 8.4 g (45%) of the product. ¹H NMR (300 MHz, CDCl₃) δ: 8.94 (s, 1H), 8.06 (d, J=10.8 Hz, 1H), 7.70 (d, J=2.4 Hz, 1H), 6.66-6.62 (m, 1H), 3.81 (s, 2H).

### Step 2

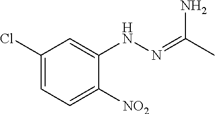

### ((1Z)-2-Amino-1-azaprop-1-enyl)(5-chloro-2-nitrophenyl)amine

A 250 mL round bottom flask was charged with 5-chloro-2-nitrophenylhydrazine (8.06 g, 0.043 mol), ethyl acetimidate hydrochloride (5.3 g, 0.043 mol) and pyridine (120 mL). The resulting mixture was stirred at room temperature overnight. The reaction progress was monitored by TLC (EtOAc/Petroleum ether=1:1, Rf=0.4). Work up: the solvent was evaporated under reduced pressure. The residue was partitioned between EtOAc (200 mL) and saturated aqueous Na₂CO₃ solution (200 mL). The organic layer was separated, dried over anhydrous MgSO₄, and concentrated in vacuo, to give 6.4 g (65%) of the product. ¹H NMR (300 MHz, CDCl₃) δ: 9.56 (s, 1H), 8.07 (d, J=9.0 Hz, 1H), 7.54 (d, J=2.4 Hz, 1H), 6.66 (dd, J=9.0, 2.1 Hz, 1H), 4.73 (s, 2H), 2.10 (s, 3H). MS m/z: 229 (M+H⁺).

### Step 3

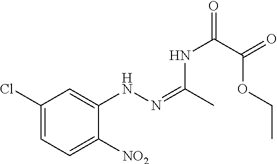

### Ethyl (N-{(1Z)-2-[(5-chloro-2-nitrophenyl)amino]-1-methyl-2-azavinyl}carbamoyl)formate

A 500 mL round bottom flask was charged with (1Z)-2-amino-1-azaprop-1-enyl)(5-chloro-2-nitrophenyl)amine (6.4 g, 28 mmol) and ethyl ether (25 mL). To the above was added dropwise a solution of ethyl 2-(chlorocarbonyl)acetate (7.65 g, 56 mmol) in ethyl ether (20 mL) at room temperature. The resulting mixture turned yellow from red. The reaction mixture was stirred at room temperature for 1 h, then mixed with anhydrous toluene (200 mL) and heated at reflux for 1 h. Work up: the reaction mixture was filtered. The filtrate was concentrated in vacuo, to give 4.2 g of the product, which was used directly in the next step without further purification.

### Step 4

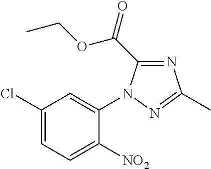

### Ethyl 1-(5-chloro-2-nitrophenyl)-3-methyl-1,2,4-triazole-5-carboxylate

A 50 mL round bottom flask was charged with ethyl (N-{(1Z)-2-[(5-chloro-2-nitrophenyl)amino]-1-methyl-2-azavinyl}carbamoyl)formate (4.2 g). It was heated at 180° C. for 1 h under N₂. The reaction progress was monitored by TLC (EtOAc/Petroleum ether=1:1, Rf=0.3). Work up: the cooled mass was dissolved in CH₂Cl₂ (100 mL), washed with 0.5 N KOH solution (20 mL) and brine (30 mL) subsequently. The organic layer was dried over anhydrous MgSO₄ and concentrated in vacuo. The residue was further purified by flash column chromatography on silica gel with a 1:6 EtOAc/Petroleum ether, to give 1.8 g of the product. ¹H NMR (300 MHz, CDCl₃) δ: 8.20 (d, J=8.7 Hz, 1H), 7.66 (dd, J=9.3, 2.4 Hz, 1H), 7.60 (d, J=2.1 Hz, 1H), 4.38-4.31 (m, 2H), 2.53 (s, 3H), 1.37-1.25 (m, 3H).

### Step 5

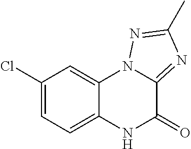

### 8-chloro-2-methyl-[1,2,4]triazolo[1,5-a]quinoxalin-4(5H)-one

A 100 mL round bottom flask was charged with ethyl 1-(5-chloro-2-nitrophenyl)-3-methyl-1,2,4-triazole-5-carboxylate (1.8 g, 5.8 mmol), iron powder (5.87 g, 87 mmol) and HOAc (40 mL). The resulting mixture was heated at 90° C. for 1 h. Work up: the reaction mixture was filtered. The filtrate was concentrated in vacuo and mixed with 6 N HCl (50 mL). The precipitate formed was collected by filtration and dried, to give 0.8 g of the product which was used directly in the next step without further purification. MS m/z: 233 (M−H⁺).

### Step 6

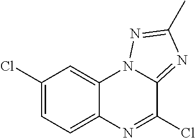

### 4,8-dichloro-2-methyl-[1,2,4]triazolo[1,5-a]quinoxaline

The title compound was prepared as described in Example 92, except that 8-chloro-2-methyl-[1,2,4]triazolo[1,5-a]quinoxalin-4(5H)-one was substituted for 9-chloro-[1,2,4]triazolo[1,5-c]quinazolin-5(6H)-one in step 3 of that route. ¹H NMR (300 MHz, CDCl₃) δ: 8.39 (d, J=2.1 Hz, 1H), 8.04 (d, J=8.7 Hz, 1H), 7.65 (dd, J=9.0, 2.4 Hz, 1H), 2.75 (s, 3H).

### Step 7

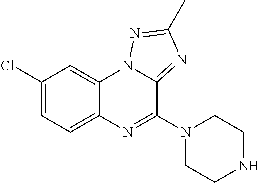

### 8-chloro-2-methyl-4-(piperazin-1-yl)-[1,2,4]triazolo[1,5-a]quinoxaline

The title compound was prepared as described in Example 92, except that 4,8-dichloro-2-methyl-[1,2,4]triazolo[1,5-a]quinoxaline was substituted for 5,9-dichloro-[1,2,4]triazolo[1,5-c]quinazoline in step 1 of that route. ¹H NMR (300 MHz, CD₃OD) δ: 8.16-8.15 (m, 1H), 7.62-7.59 (m, 1H), 7.41-7.37 (m, 1H), 4.33-4.30 (m, 4H), 3.07-3.04 (m, 4H), 2.64 (s, 3H). MS m/z: 303 (M+H⁺).

### EXAMPLE 165

### 8-chloro-2-methyl-4-(4-methylpiperazin-1-yl)-[1,2,4]triazolo[1,5-a]quinoxaline

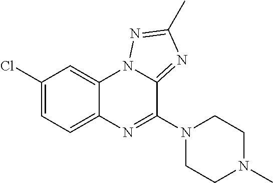

The title compound was prepared as described in Example 164, except that N-methylpiperazine was substituted for piperazine in step 7 of that route. ¹H NMR (300 MHz, CD₃OD) δ: 8.17 (d, J=2.4 Hz, 1H), 7.62 (d, J=9.0 Hz, 1H), 7.40 (dd, J=8.7, 2.4 Hz, 1H), 4.38-4.35 (m, 4H), 2.64 (s, 3H), 2.61-2.58 (m, 4H), 2.37 (s, 3H). MS m/z: 317 (M+H⁺).

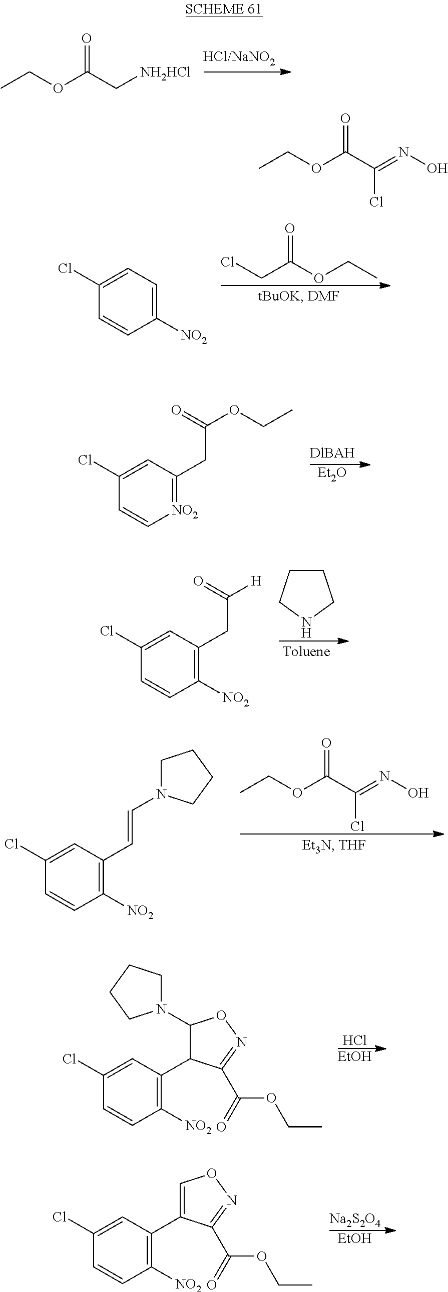

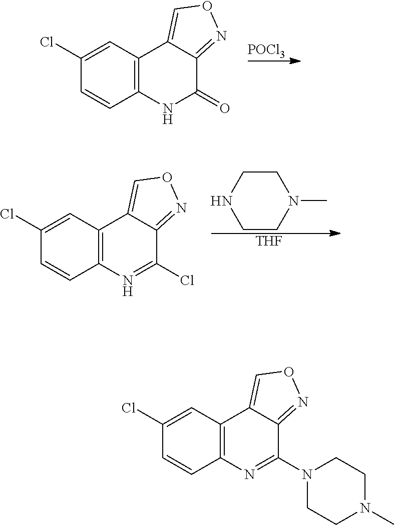

### EXAMPLE 166

### 8-Chloro-4-(4-methylpiperazin-1-yl)isoxazolo[3,4-c]quinoline

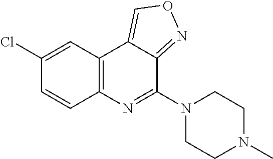

### Step 1

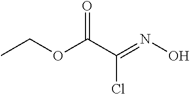

### Ethyl chlorooximidoacetate

A 250 mL round bottom flask was charged with glycine ethyl ester hydrochloride (40 g, 0.29 mol), concentrated HCl (24 mL, 0.29 mol) and water (55 mL). To the above was added dropwise a solution of sodium nitrite (20 g, 0.29 mol) in water (30 mL) at −5° C. A second equivalent of hydrochloric acid and sodium nitrite were then added in the same manner. The resulting mixture was stirred at −5° C. for 20 min then extracted with ethyl ether (250 mL). The extract was dried over anhydrous MgSO₄ and concentrated in vacuo. The yellowish oil residue was crystallized from hexane to afford 17 g (39%) of the product as white crystals. ¹H NMR (300 MHz, CDCl₃) δ: 9.92 (br, 1H), 4.39 (q, J=7.1 Hz, 2H), 1.38 (t, J=7.1 Hz, 3H). ¹³C NMR (75 MHz, CDCl₃) δ: 158.5, 132.9, 63.8, 13.9.

### Step 2

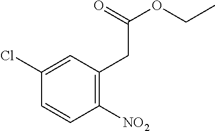

### Ethyl 2-(5-chloro-2-nitrophenyl)acetate

A 500 mL round bottom flask was charged with potassium t-butoxide (17.8 g, 0.16 mol) and dry DMF (200 mL). To the above was added dropwise a solution of 1-chloro-4-nitrobenzene (10 g, 0.063 mol) and ethyl chloroacetate (7.1 mL, 0.067 mol) in dry DMF (50 mL) at −5° C. The resulting dark-blue mixture was stirred at −5° C. for further 20 min then poured into 1 M HCl (500 mL) and extracted with ethyl ether (100 mL×5). The combined organic layers were washed with saturated aqueous NaHCO₃ (250 mL) and brine (250 mL), dried over anhydrous Na₂SO₄ and concentrated in vacuo. The residue was further purified by flash column chromatography on silica gel with 2-4% ethyl ether in petroleum ether, to afford 11.8 g (76%) of the product as orange oil. ¹H NMR (300 MHz, CDCl₃) δ: 8.06 (d, J=8.8 Hz, 1H), 7.42 (dd, J=8.8, 2.3 Hz, 1H), 7.34 (d, J=2.3 Hz, 1H), 4.16 (q, J=7.1 Hz, 2H), 3.98 (s, 2H), 1.24 (t, J=7.1 Hz, 3H). MS m/z: 242 (M−H⁺).

### Step 3

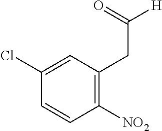

### 2-(5-Chloro-2-nitrophenyl)acetaldehyde

A 250 mL 3-necked round bottom flask was charged with ethyl 2-(5-chloro-2-nitrophenyl)acetate (2.0 g, 8.2 mmol) and dry ethyl ether (50 mL). To the above was added dropwise a solution of 1.5 M diisobutylaluminum hydride in toluene (11 ml, 16.5 mmol) at −78° C. The resulting mixture was stirred at −78° C. for further 1 h then quenched by slow addition of methanol (10 mL). The mixture was poured into 1 M HCl (200 mL) and extracted with ethyl ether (100 mL×2). The combined organic layers were washed with saturated aqueous NaHCO₃ (100 mL) and brine (100 mL), dried over anhydrous Na₂SO₄ and concentrated in vacuo. The residue was further purified by flash column chromatography on silica gel with 4-20% ethyl ether in petroleum ether, to afford 1.1 g (70%) of the product as orange oil. ¹H NMR (300 MHz, CDCl₃) δ: 9.83 (t, J=0.7 Hz, 1H), 8.12 (d, J=8.8 Hz, 1H), 7.46 (dd, J=8.8, 2.3 Hz, 1H), 7.31 (d, J=2.3 Hz, 1H), 4.13 (s, 2H). MS m/z: 198 (M−H⁺).

### Steps 4-6

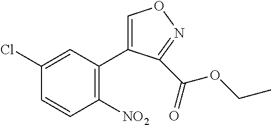

### Ethyl 4-(5-chloro-2-nitrophenyl)isoxazole-3-carboxylate

A 1 L round bottom flask was charged with 2-(5-chloro-2-nitrophenyl)acetaldehyde (8.5 g, 43 mmol), pyrrolidine (4.3 mL, 51 mmol), crushed 4 A molecular sieves (18 g) and dry toluene (50 mL). The reaction mixture was stirred for 2 h at room temperature under N₂ and developed a dark-red color.

To the above dark-red mixture were added Et₃N (12 mL, 86 mmol) and THF (150 mL), followed by a very slow addition in the dark of a solution of ethyl chlorooximidoacetate (13 g, 86 mmol) in THF (250 mL). The resulting mixture was stirred in the dark overnight at room temperature, and then filtered and concentrated in vacuo.

The residue was added to EtOH (150 mL) and concentrated HCl (36 mL, 0.43 mol). The resulting mixture was stirred at 50° C. overnight then concentrated in vacuo. It was poured into saturated aqueous NaHCO₃ (300 mL) and extracted with CHCl₃ (100 mL×5). The combined organic layers were dried over anhydrous Na₂SO₄ and concentrated in vacuo. The residue was further purified by flash column chromatography on silica gel with 40-100% CH₂Cl₂ in petroleum ether, to afford 8.8 g (70%) of the product as dark-red oil. ¹H NMR (300 MHz, CDCl₃) δ: 8.59 (s, 1H), 8.18 (d, J=8.8 Hz, 1H), 7.57 (dd, J=8.8, 2.2 Hz, 1H), 7.39 (d, J=2.2 Hz, 1H), 4.31 (q, J=7.1 Hz, 2H), 1.29 (t, J=7.1 Hz, 3H). ¹³C NMR (75 MHz, CDCl₃) δ: 159.3, 157.3, 152.9, 146.6, 139.7, 132.6, 129.9, 126.6, 125.2, 118.1, 62.5, 13.8.

### Step 7

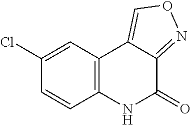

### 8-Chloroisoxazolo[3,4-c]quinolin-4(5H)-one

A 250 mL round bottom flask was charged with ethyl 4-(5-chloro-2-nitrophenyl)isoxazole-3-carboxylate (3.4 g, 11 mmol), Na₂S₂O₄ (85% purity, 4.7 g, 23 mmol), EtOH (120 mL) and H₂O (50 mL). The resulting mixture was stirred at reflux overnight and then concentrated in vacuo. The residue was mixed with saturated aqueous NaHCO₃ (200 mL) and extracted with CHCl₃ (100 mL×5). The combined organic layers were dried over anhydrous Na₂SO₄ then concentrated in vacuo. The residue was further purified by flash column chromatography on silica gel with 5-20% MeOH in CH₂Cl₂, to afford 1.2 g (47%) of the product as white solid. ¹H NMR (300 MHz, DMSO-d₆) δ: 11.83 (br, 1H), 10.05 (s, 1H), 8.14 (s, 1H), 7.48 (d, J=8.7 Hz, 1H), 7.31 (d, J=8.7 Hz, 1H).

### Step 8

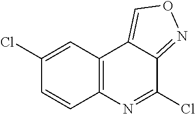

### 4,8-Dichloroisoxazolo[3,4-c]quinoline

A 100 mL round bottom flask was charged with 8-chloroisoxazolo[3,4-c]quinolin-4(5H)-one (1.2 g, 5.5 mmol) and POCl₃ (50 mL). After N,N-diisopropylethylamine (0.95 mL, 5.5 mmol) was added dropwise at 0° C., the resulting mixture was refluxed overnight (16 h) and then concentrated under reduced pressure. The residue was carefully diluted with saturated aqueous NaHCO₃ (150 mL), then extracted with CH₂Cl₂ (100 mL×3). The combined organic layers were dried over anhydrous Na₂SO₄ then concentrated in vacuo. The residue was purified by flash column chromatography on silica gel with CH₂Cl₂ (containing 1% Et₃N), to afford 0.50 g (38%) of the product as light-yellow solid. ¹H NMR (300 MHz, CDCl₃) δ: 9.47 (s, 1H), 7.99 (d, J=2.4 Hz, 1H), 7.96 (d, J=8.9 Hz, 1H), 7.62 (dd, J=8.9, 2.4 Hz, 1H).

### Step 9

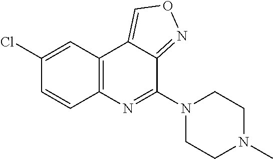

### 8-Chloro-4-(4-methylpiperazin-1-yl)isoxazolo[3,4-c]quinoline

A 20 mL microwave reaction tube was charged with 4,8-dichloroisoxazolo[3,4-c]quinoline (200 mg, 0.84 mmol), N-methylpiperazine (0.28 mL, 2.5 mmol) and THF (10 mL). The tube was sealed and heated at 90° C. for 1 h in a Biotage microwave reactor. Work-up: the reaction mixture was poured into saturated aqueous NaHCO₃ (100 mL) and extracted with CH₂Cl₂ (50 mL×3). The combined organic layers were dried over anhydrous Na₂SO₄ and concentrated in vacuo. The residue was purified by flash column chromatography on silica gel with CH₂Cl₂ (saturated with NH₃), to afford 150 mg (59%) of the product as tan solid. ¹H NMR (300 MHz, CD₃OD) δ: 9.73 (s, 1H), 7.91 (d, J=2.4 Hz, 1H), 7.47 (d, J=8.8 Hz, 1H), 7.35 (dd, J=8.8, 2.4 Hz, 1H), 4.23 (m, 4H), 2.62 (m, 4H), 2.36 (s, 3H). MS m/z: 303 (M+H⁺).

### EXAMPLE 167

### 8-Chloro-4-(piperazin-1-yl)isoxazolo[3,4-c]quinoline

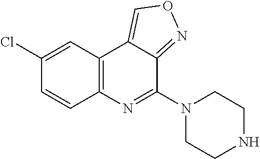

The title compound was prepared as described in Example 166, except that piperazine was substituted for N-methylpiperazine in step 9 of that route. ¹H NMR (300 MHz, CD₃OD) δ: 9.72 (s, 1H), 7.90 (d, J=2.4 Hz, 1H), 7.46 (d, J=8.8 Hz, 1H), 7.35 (dd, J=8.8, 2.4 Hz, 1H), 4.18 (m, 4H), 2.97 (m, 4H). MS m/z: 289 (M+H⁺).

### EXAMPLE 168

### 7,8-dichloro-4-(piperazin-1-yl)-[1,2,4]triazolo[4,3-a]quinoxaline

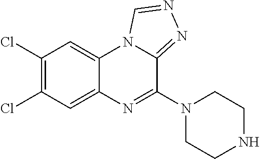

The title compound was prepared as described in Example 39, except that piperazine was substituted for N-methylpiperazine in step 3 of that route. ¹H NMR (300 MHz, CD₃OD) δ: 9.70 (s, 1H), 8.22 (s, 1H), 7.62 (s, 1H), 4.41-4.38 (m, 4H), 3.08 (t, J=5.4 Hz, 4H). MS m/z: 323 (M+H⁺).

### EXAMPLE 169

### 9-fluoro-4-(piperazin-1-yl)-8-(trifluoromethyl)-[1,2,4]triazolo[4,3-a]quinoxaline

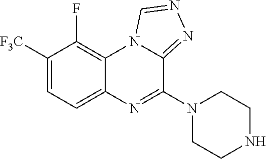

The title compound was prepared as described in Example 236, except that piperazine was substituted for N-methylpiperazine in step 8 of that route. ¹H NMR (300 MHz, CDCl₃) δ: 9.40 (d, J=2.4 Hz, 1H), 7.61 (t, J=8.4 Hz, 1H), 7.47 (d, J=8.4 Hz, 1H), 4.55 (br, 4H), 3.08 (t, J=5.4 Hz, 4H). MS m/z: 341 (M+H⁺).

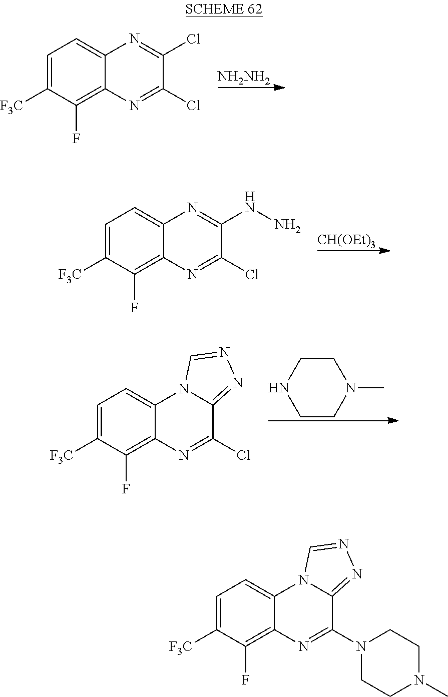

### EXAMPLE 170

### 6-fluoro-4-(4-methylpiperazin-1-yl)-7-(trifluoromethyl)-[1,2,4]triazolo[4,3-a]quinoxaline

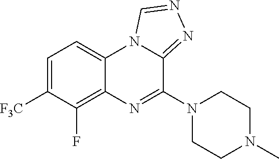

The title compound was prepared as described in Example 21, except that 2,3-dichloro-5-fluoro-6-(trifluoromethyl)quinoxaline (prepared as described in Example 132, step 7) was substituted for 2,3-dichloro-6-methylquinoxaline as the starting material. ¹H NMR (300 MHz, DMSO-d₆) δ: 10.08 (s, 1H), 8.17 (d, J=8.7 Hz, 1H), 7.64 (t, J=6.9 Hz, 1H), 4.34 (br, 4H), 2.52 (m, 4H), 2.23 (s, 3H). MS m/z: 355 (M+H⁺).

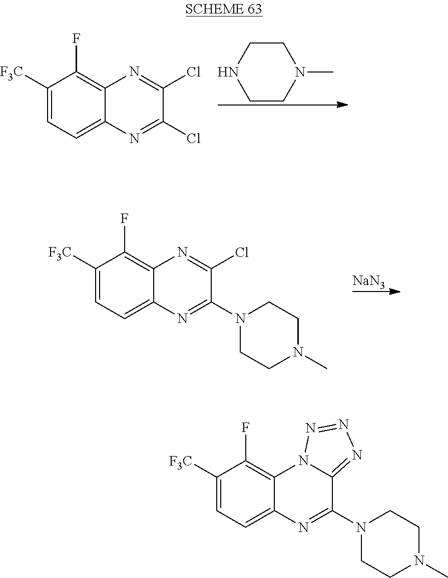

### EXAMPLE 171

### 9-Fluoro-4-(4-methylpiperazin-1-yl)-8-(trifluoromethyl)tetrazolo[1,5-a]quinoxaline

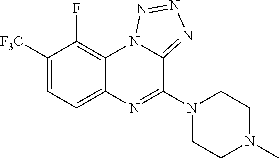

The title compound was prepared as described in Example 27, except that 2,3-dichloro-5-fluoro-6-(trifluoromethyl)quinoxaline (prepared as described in Example 132, step 7) was substituted for 2,3-dichloro-6-(trifluoromethyl)quinoxaline as the starting material. ¹H NMR (300 MHz, DMSO-d₆) δ: 7.89 (t, J=8.7 Hz, 1H), 7.62 (d, J=8.7 Hz, 1H), 4.46 (br, 4H), 2.53 (t, J=5.4 Hz, 4H), 2.24 (s, 3H). MS m/z: 356 (M+H⁺).

### EXAMPLE 172

### 9-fluoro-4-(piperazin-1-yl)-8-(trifluoromethyl)tetrazolo[1,5-a]quinoxaline

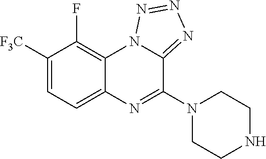

The title compound was prepared as described in Example 171, except that piperazine was substituted for N-methylpiperazine in step 1 of that route. ¹H NMR (300 MHz, DMSO-d₆) δ: 7.77 (t, J=8.4 Hz, 1H), 7.53 (d, J=9.0 Hz, 1H), 4.56 (m, 4H), 3.44 (m, 4H). MS m/z: 342 (M+H⁺).

### EXAMPLE 173

### 8-isopropyl-4-(4-methylpiperazin-1-yl)-[1,2,4]triazolo[4,3-a]quinoxaline

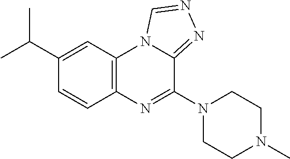

The title compound was prepared as described in Examples 88 and 90, except that 2-(tributylstannyl)propene was substituted for tri-n-butyl(vinyl)tin as the coupling reactant. ¹H NMR (300 MHz, CDCl₃) δ: 9.17 (s, 1H), 7.62 (d, J=8.4 Hz, 1H), 7.53 (s, 1H), 7.37 (d, J=8.7 Hz, 1H), 4.44 (br, 4H), 3.06 (m, 1H), 2.61 (t, J=5.1 Hz, 4H), 2.37 (s, 3H), 1.33 (d, J=6.9 Hz, 6H). MS m/z: 310 (M+H⁺).

### EXAMPLE 174

### (E)-4-(4-methylpiperazin-1-yl)-8-(prop-1-enyl)-[1,2,4]triazolo[4,3-a]quinoxaline

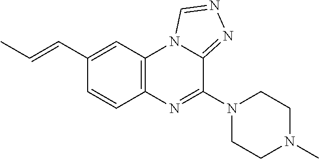

The title compound was prepared as described in Example 88, except that 1-propenyltributyltin was substituted for tri-n-butyl(vinyl)tin as the coupling reactant. ¹H NMR (300 MHz, CDCl₃) δ: 9.14 (s, 1H), 7.64-7.57 (m, 2H), 7.48 (m, 1H), 6.53-6.45 (m, 1H), 6.39-6.27 (m, 0.5H), 5.95-5.84 (m, 0.5H), 4.48 (br, 4H), 2.65 (t, J=4.8 Hz, 4H), 2.40 (s, 3H), 1.98-1.92 (m, 3H). MS m/z: 308 (M+H⁺).

### EXAMPLE 175

### 4-(4-methylpiperazin-1-yl)-8-propyl-[1,2,4]triazolo[4,3-a]quinoxaline

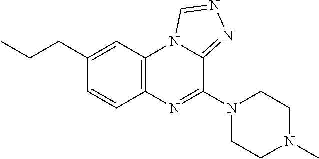

The title compound was prepared as described in Example 90, except that (E)-4-(4-methylpiperazin-1-yl)-8-(prop-1-enyl)-[1,2,4]triazolo[4,3-a]quinoxaline (Example 174) was substituted for 4-(4-methylpiperazin-1-yl)-8-vinyl-[1,2,4]triazolo[4,3-a]quinoxaline (Example 88) as the starting material. ¹H NMR (300 MHz, CDCl₃) δ: 9.15 (s, 1H), 7.59 (d, J=8.4 Hz, 1H), 7.50 (s, 1H), 7.28 (d, J=8.1 Hz, 1H), 4.43 (br, 4H), 2.71 (t, J=7.6 Hz, 2H), 2.60 (t, J=4.8 Hz, 4H), 2.36 (s, 3H), 1.71 (m, 2H), 0.97 (t, J=7.4 Hz, 3H). MS m/z: 310 (M+H⁺).

### EXAMPLE 176

### N-isopropyl-4-(4-methylpiperazin-1-yl)-[1,2,4]triazolo[4,3-a]quinoxalin-8-amine

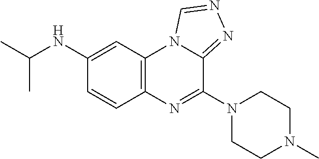

A 50 mL round bottom flask was charged with 8-bromo-4-(4-methylpiperazin-1-yl)-[1,2,4]triazolo[4,3-a]quinoxaline (Example 54, 0.20 g, 0.6 mmol), isopropylamine (1 mL), L-proline (0.13 g, 1.13 mmol), Cul (0.11 g, 0.6 mmol), K₃PO₄ (0.11 g, 1.2 mmol) and DMSO (20 mL). The resulting mixture was heated at 90° C. overnight. Work-up: the reaction mixture was diluted with water (100 mL) and extracted with EtOAc (100 mL×2). The combined organic layers were dried over anhydrous Na₂SO₄ and then concentrated in vacuo. The residue was purified by flash column chromatography on silica gel with 5% MeOH in CH₂Cl₂, to afford 80 mg (43%) of the product as yellow solid. ¹H NMR (300 MHz, CDCl₃) δ: 9.05 (s, 1H), 7.51 (d, J=9.0 Hz, 1H), 6.77 (s, 1H), 6.75 (d, J=8.4 Hz, 1H), 4.31 (t, J=4.8 Hz, 4H), 3.72 (m, 1H), 2.63 (t, J=5.1 Hz, 4H), 2.38 (s, 3H), 1.28 (d, J=6.0 Hz, 6H). MS m/z: 326 (M+H⁺).

### EXAMPLE 177

### 4-(4-methylpiperazin-1-yl)-8-(trifluoromethypimidazo[1,2-a]quinoxaline

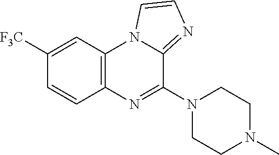

The title compound was prepared as described in Example 54, except that 4-(piperazin-1-yl)-8-(trifluoromethyl)imidazo[1,2-a]quinoxaline hydrochloride (EXAMPLE 178) was substituted for 8-bromo-4-piperazinyl-10-hydro-1,2,4-triazolo[4,3-a]quinoxaline HCl salt (Example 52). ¹H NMR (300 MHz, CDCl₃) δ: 8.00 (d, J=1.5 Hz, 1H), 7.90 (s, 1H), 7.72 (d, J=8.7 Hz, 1H), 7.65-7.59 (m, 2H), 4.52 (br, 4H), 2.63 (t, J=4.8 Hz, 4H), 2.39 (s, 3H). MS m/z: 336 (M+H⁺).

### EXAMPLE 178

### 4-(piperazin-1-yl)-8-(trifluoromethypimidazo[1,2-a]quinoxaline hydrochloride

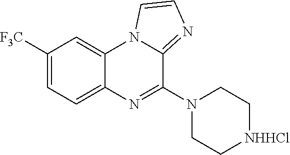

The title compound was prepared as described as in Example 180, except that 4-(trifluoromethyl)benzene-1,2-diamine was substituted for 4-chloro-5-fluorobenzene-1,2-diamine as the starting material. ¹H NMR (300 MHz, D₂O) δ: 8.10 (d, J=1.5 Hz, 1H), 7.88 (s, 1H), 7.59 (d, J=1.5 Hz, 1H), 7.54-7.47 (m, 2H), 4.34 (t, J=5.1 Hz, 4H), 3.42 (t, J=5.1 Hz, 4H). MS m/z: 322 (M+H⁺).

### EXAMPLE 179

### 8-chloro-7-fluoro-4-(piperazin-1-yl)imidazo[1,2-a]quinoxaline

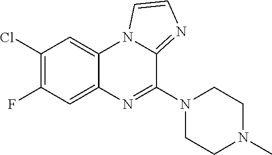

The title compound was prepared as described in Example 54, except that 8-chloro-7-fluoro-4-(piperazin-1-yl)imidazo[1,2-a]quinoxaline HCl salt (Example 180) was substituted for 8-bromo-4-piperazinyl-10-hydro-1,2,4-triazolo[4,3-a]quinoxaline HCl salt (Example 52). ¹H NMR (300 MHz, CDCl₃) δ: 7.85 (d, J=1.5 Hz, 1H), 7.67 (d, J=6.9 Hz, 1H), 7.60 (d, J=1.5 Hz, 1H), 7.39 (d, J=10.2 Hz, 1H), 4.44 (t, J=4.5 Hz, 4H), 2.58 (t, J=5.1 Hz, 4H), 2.35 (s, 3H). MS m/z: 320 (M+H⁺).

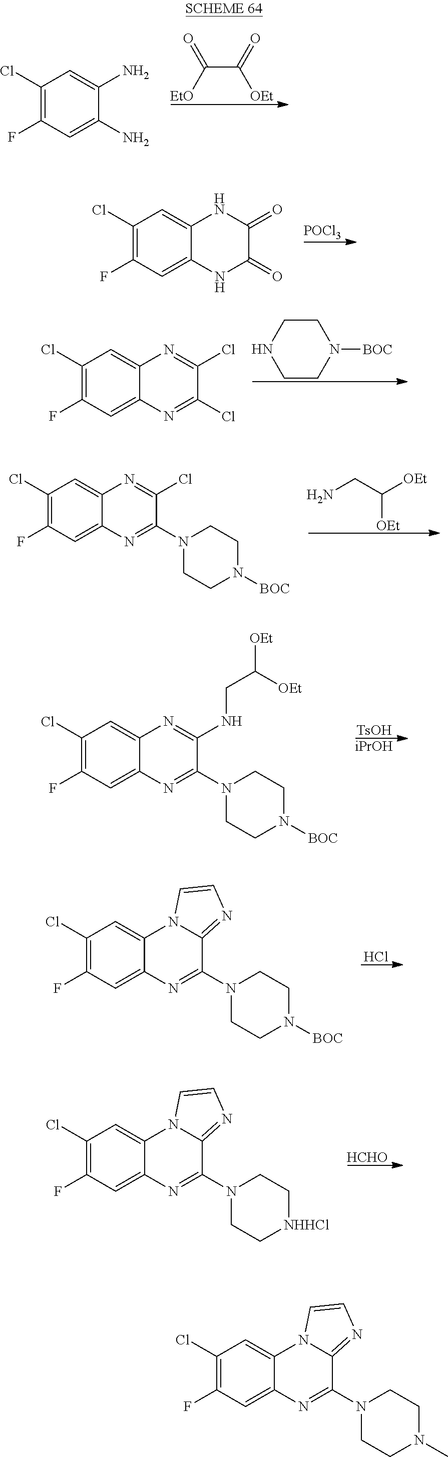

### EXAMPLE 180

### 8-chloro-7-fluoro-4-(piperazin-1-yl)imidazo[1,2-a]quinoxaline

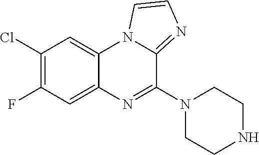

### Step 4

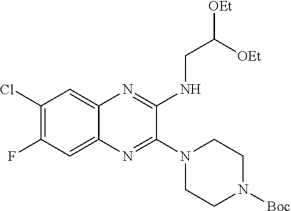

### tert-butyl 4-(6-chloro-3-(2,2-diethoxyethylamino)-7-fluoroquinoxalin-2-yl)piperazine-1-carboxylate

A 50 mL round bottom flask was charged with tert-butyl 4-(3,6-dichloro-7-fluoroquinoxalin-2-yl)piperazinecarboxylate (prepared as described in Example 31, 1.5 g, 3.6 mmol) and 2,2-diethoxyethylamine (10 mL). The resulting mixture was stirred at reflux for 1 h. Reaction progress was monitored by TLC (EtOAc/Petroleum ether=1:5). Work-up: the reaction mixture was concentrated in vacuo. The residue was re-dissolved in EtOAc (200 mL) and washed with brine (100 mL). The organic layer was dried over anhydrous Na₂SO₄ and then concentrated in vacuo to afford the title compound.

### Step 5

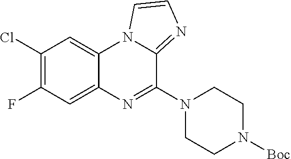

### tert-butyl 4-(8-chloro-7-fluoroimidazo[1,2-a]quinoxalin-4-yl)piperazine-1-carboxylate

A 50 mL round bottom flask was charged with tert-butyl 4-{3-[(2,2-diethoxyethyl)amino]-6-chloro-7-fluoroquinoxalin-2-yl}piperazinecarboxylate from step 4, p-toluenesulfonic acid (1.37 g, 7.3 mmol) and isopropanol (25 mL). The resulting mixture was stirred at reflux for 1 h. Reaction progress was monitored by TLC (EtOAc/Petroleum ether=1:3). Work-up: the reaction mixture was concentrated in vacuo. The residue was re-dissolved in EtOAc (200 mL) and washed with brine (100 mL). The organic layer was dried over anhydrous Na₂SO₄ and then concentrated in vacuo. The residue was further purified by flash column chromatography on silica gel with a 1:3 EtOAc/Petroleum ether to afford the title compound.

### Step 6

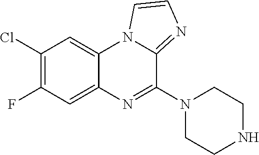

### 8-chloro-7-fluoro-4-(piperazin-1-yl)imidazo[1,2-a]quinoxaline

The HCl salt of the title compound was prepared as described in Example 52 step 6, except that tert-butyl 4-(8-chloro-7-fluoro-10-hydroimidazo[1,2-a]quinoxalin-4-yl)piperazinecarboxylate was substituted for tert-butyl 4-(8-bromo-10-hydro-1,2,4-triazolo[4,3-a]quinoxalin-4-yl)piperazinecarboxylate. ¹H NMR (300 MHz, CDCl₃) δ: 7.83 (d, J=5.4 Hz, 1H), 7.66 (d, J=6.9 Hz, 1H), 7.59 (s, 1H), 7.38 (d, J=9.9 Hz, 1H), 4.34 (br, 4H), 3.02 (br, 4H). MS m/z: 306 (M+H⁺).

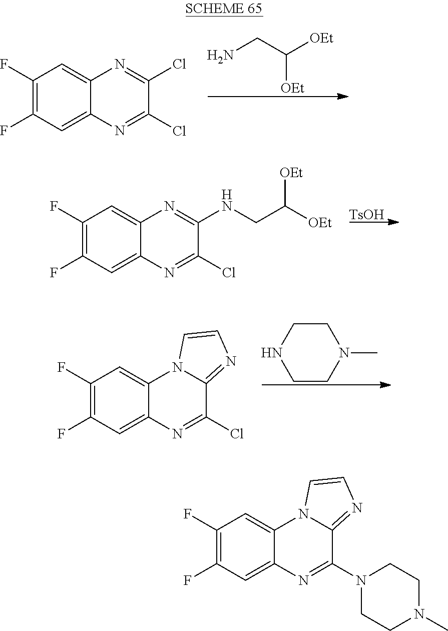

### EXAMPLE 181

### 7,8-difluoro-4-(4-methylpiperazin-1-yl)imidazo[1,2-a]quinoxaline

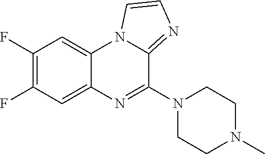

The title compound was prepared as described in Examples 37 and 179, except that 2,3-dichloro-6,7-difluoroquinoxaline was substituted for 2,3,7-trichloro-6-fluoroquinoxaline in step 3 of that route. ¹H NMR (300 MHz, DMSO-d₆) δ: 8.61 (d, J=1.5 Hz, 1H), 8.38 (dd, J=11.1, 7.8 Hz, 1H), 7.68 (d, J=1.2 Hz, 1H), 7.56 (dd, J=12.0, 8.1 Hz, 1H), 4.31 (br, 4H), 2.49 (m, 4H), 2.23 (s, 3H). MS m/z: 304 (M+H⁺).

### EXAMPLE 182

### 7,8-difluoro-4-(piperazin-1-yl)imidazo[1,2-a]quinoxaline

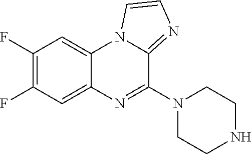

The title compound was prepared as described in Example 181, except that piperazine was substituted for N-methylpiperazine in the last step of that route. ¹H NMR (300 MHz, CD₃OD) δ: 8.29 (d, J=1.5 Hz, 1H), 7.98 (dd, J=11.1, 7.8 Hz, 1H), 7.60 (d, J=1.5 Hz, 1H), 7.46 (dd, J=11.7, 8.1 Hz, 1H), 4.29 (t, J=5.1 Hz, 4H), 3.00 (t, J=5.1 Hz, 4H). MS m/z: 290 (M+H⁺).

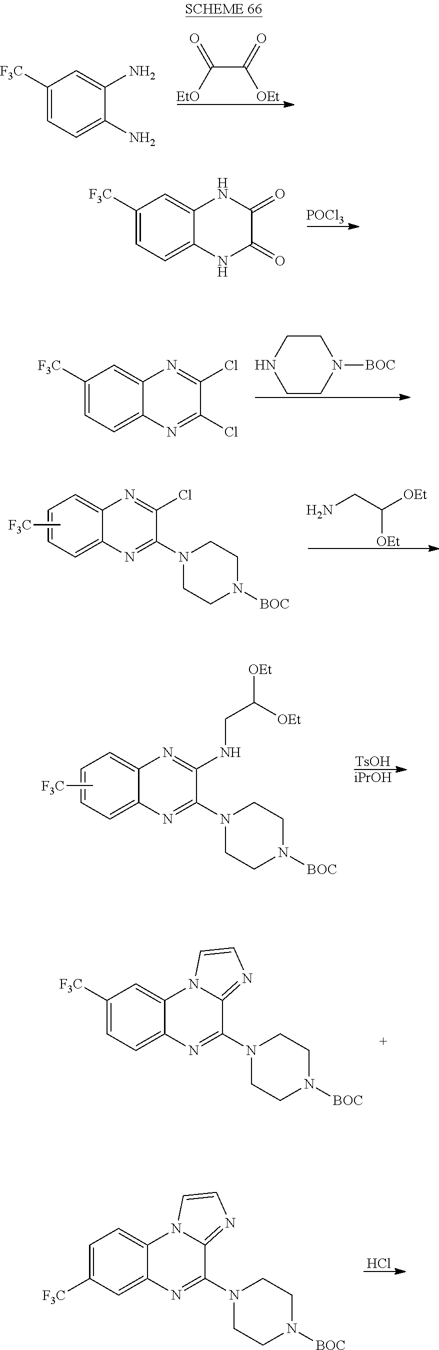

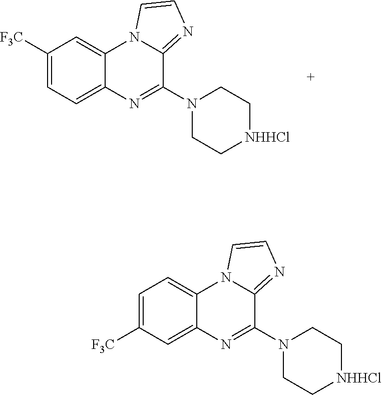

### EXAMPLE 183

### 4-(piperazin-1-yl)-7-(trifluoromethyl)imidazo[1,2-a]quinoxaline hydrochloride

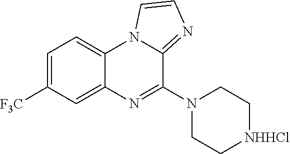

The title compound was prepared as described as in Example 178. It was separated from the other regio-isomer by flash column chromatography. ¹H NMR (300 MHz, DMSO-d₆) δ: 9.60 (br, 2H), 8.83 (d, J=1.8 Hz, 1H), 8.41 (d, J=8.4 Hz, 1H), 7.92 (d, J=1.5 Hz, 1H), 7.77 (d, J=1.5 Hz, 1H), 7.70 (dd, J=8.4, 1.8 Hz, 1H), 4.62 (br, 4H), 3.28 (br, 4H). MS m/z: 322 (M+H⁺).

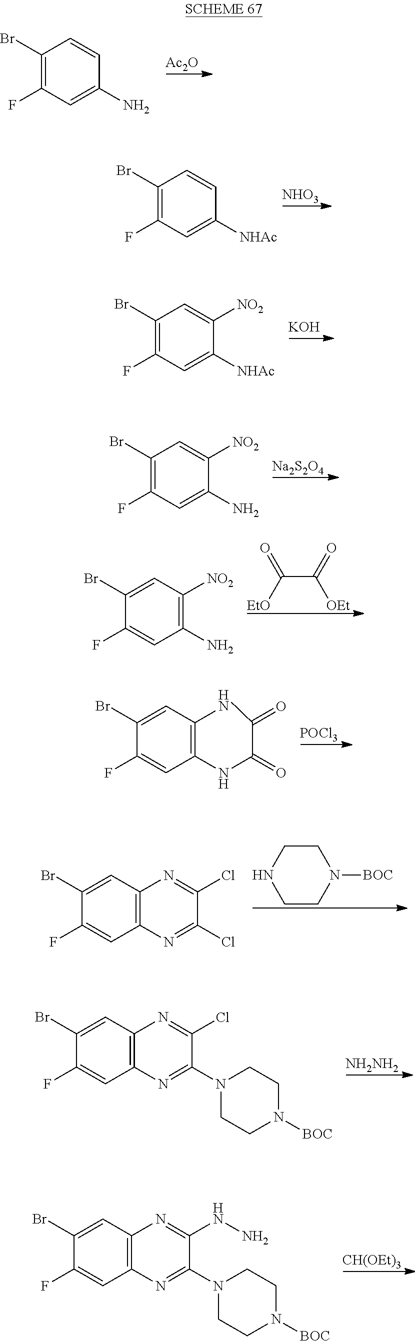

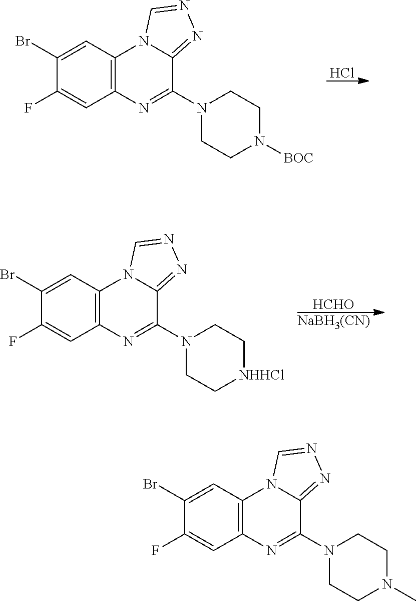

### EXAMPLE 184

### 8-bromo-7-fluoro-4-(piperazin-1-yl)-[1,2,4]triazolo[4,3-a]quinoxaline

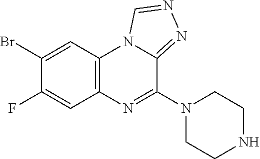

The HCl salt of the title compound was prepared as described in Example 34, except that 4-bromo-3-fluoroaniline was substituted for 4-fluoro-3-methylaniline as the starting material. ¹H NMR (300 MHz, DMSO-d₆) δ: 9.97 (s, 1H), 8.65 (d, J=6.6 Hz, 1H), 7.46 (d, J=10.5 Hz, 1H), 4.37 (br, 4H), 3.01 (t, J=5.1 Hz, 4H). MS m/z: 351 (M+H⁺).

### EXAMPLE 185

### 8-bromo-7-fluoro-4-(4-methylpiperazin-1-yl)-[1,2,4]triazolo[4,3-a]quinoxaline

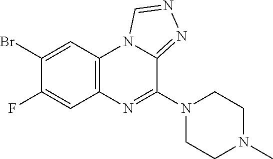

The title compound was prepared as described in Example 54, except that 8-bromo-7-fluoro-4-(piperazin-1-yl)-[1,2,4]triazolo[4,3-a]quinoxaline HCl salt (Example 184) was substituted for 8-bromo-4-piperazinyl-10-hydro-1,2,4-triazolo[4,3-a]quinoxaline HCl salt (Example 52). ¹H NMR (300 MHz, DMSO-d₆) δ: 9.95 (s, 1H), 8.62 (d, J=6.6 Hz, 1H), 7.45 (d, J=10.2 Hz, 1H), 4.32 (br, 4H), 3.29 (m, 4H), 2.22 (s, 3H). MS m/z: 365 (M+H⁺).

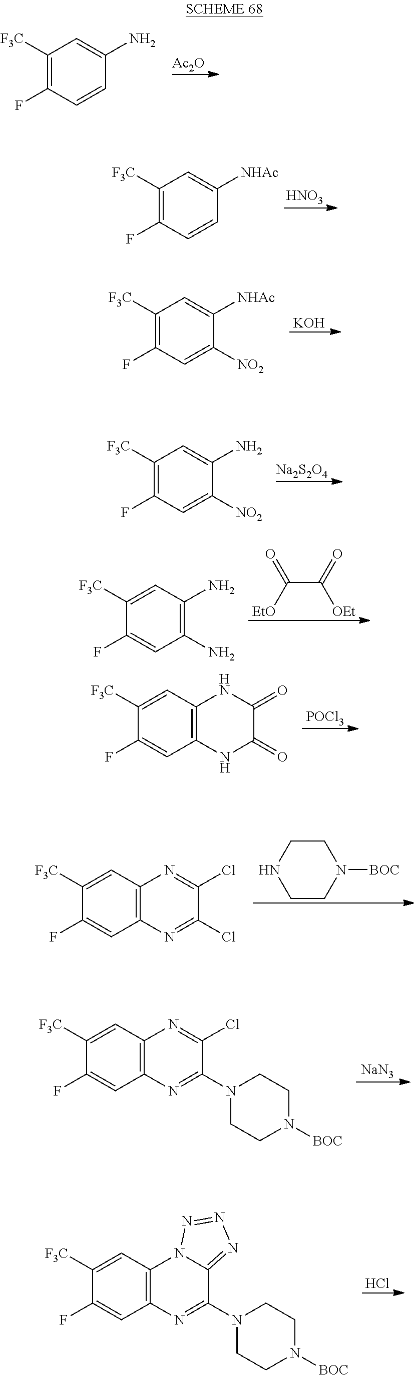

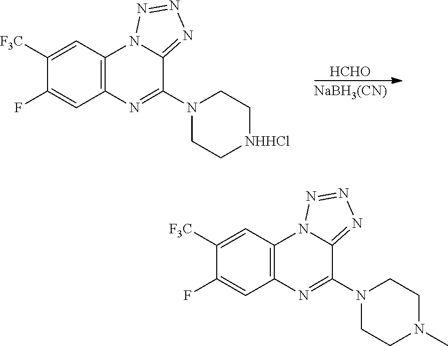

### EXAMPLE 186

### 7-fluoro-4-(piperazin-1-yl)-8-(trifluoromethyl)tetrazolo[1,5-a]quinoxaline

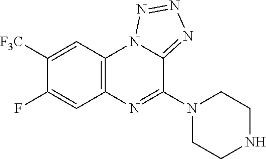

The HCl salt of the title compound was prepared as described in Example 29, except that 5-fluoro-4-(trifluoromethyl)benzene-1,2-diamine (prepared according to Example 34) was substituted for 4-(trifluoromethyl)benzene-1,2-diamine as the starting material. ¹H NMR (300 MHz, DMSO-d₆) δ: 9.59 (br, 1H), 8.64 (d, J=7.2 Hz, 1H), 7.84 (d, J=11.7 Hz, 1H), 4.65-4.33 (m, 8H). MS m/z: 342 (M+H⁺).

### EXAMPLE 187

### 7-fluoro-4-(4-methylpiperazin-1-yl)-8-(trifluoromethyl)tetrazolo[1,5-a]quinoxaline

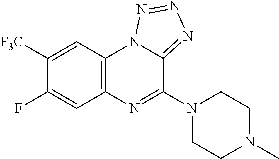

The title compound was prepared as described in Example 54, except that 7-fluoro-4-(piperazin-1-yl)-8-(trifluoromethyl)tetrazolo[1,5-a]quinoxaline HCl salt was substituted for 8-bromo-4-piperazinyl-10-hydro-1,2,4-triazolo[4,3-a]quinoxaline HCl salt as the starting material. ¹H NMR (300 MHz, CDCl₃) δ: 8.62 (d, J=6.9 Hz, 1H), 7.50 (d, J=11.4 Hz, 1H), 4.80-4.22 (m, 4H), 2.63 (m, 4H), 2.40 (s, 3H). MS m/z: 356 (M+H⁺).

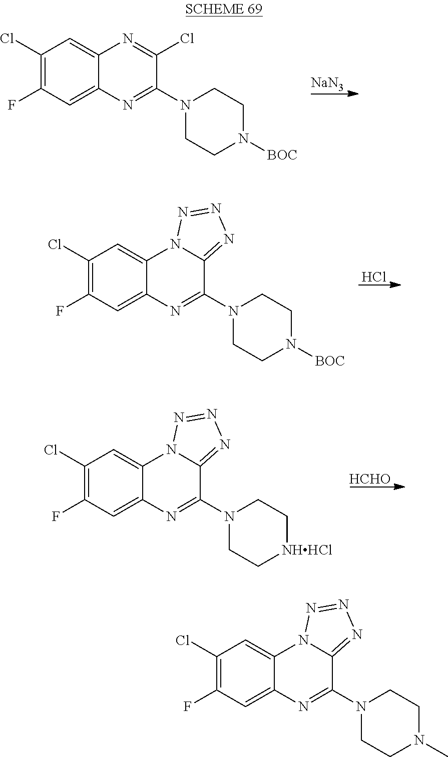

### EXAMPLE 188

### 8-chloro-7-fluoro-4-(piperazin-1-yl)tetrazolo[1,5-a]quinoxaline

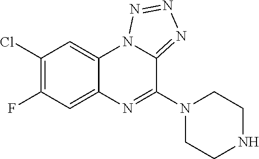

The HCl salt of the title compound was prepared as described in Examples 29 and 180, except that 4-chloro-5-fluorobenzene-1,2-diamine was substituted for 4-(trifluoromethyl)benzene-1,2-diamine as the starting material. ¹H NMR (300 MHz, DMSO-d₆) δ: 8.48 (d, J=7.2 Hz, 1H), 7.67 (d, J=10.8 Hz, 1H), 4.23 (br, 4H), 2.86 (m, 4H). MS m/z: 307 (M+H⁺).

### EXAMPLE 189

### 8-chloro-7-fluoro-4-(4-methylpiperazin-1-yl)tetrazolo[1,5-a]quinoxaline

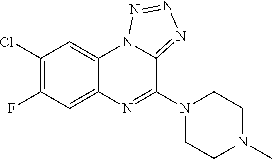

The title compound was prepared as described in Example 54, except that 8-chloro-7-fluoro-4-(piperazin-1-yl)tetrazolo[1,5-a]quinoxaline HCl salt (Example 188) was substituted for 8-bromo-4-piperazinyl-10-hydro-1,2,4-triazolo[4,3-a]quinoxaline HCl salt (Example 52). ¹H NMR (300 MHz, DMSO-d₆) δ: 8.52 (d, J=7.5 Hz, 1H), 7.72 (d, J=10.2 Hz, 1H), 4.30 (br, 4H), 2.57 (br, 4H), 2.28 (s, 3H). MS m/z: 321 (M+H⁺).

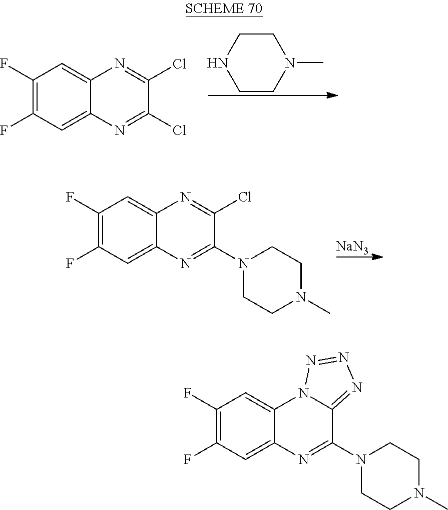

### EXAMPLE 190

### 7,8-difluoro-4-(4-methylpiperazin-1-yl)tetrazolo[1,5-a]quinoxaline

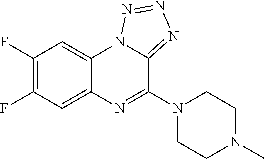

The title compound was prepared as described in Examples 37 and 27, except that 2,3-dichloro-6,7-difluoroquinoxaline was substituted for 2,3-dichloro-6-(trifluoromethyl)quinoxaline as the starting material of that route. ¹H NMR (300 MHz, DMSO-d₆) δ: 8.02 (dd, J=10.2, 7.8 Hz, 1H), 7.78 (dd, J=11.4, 7.8 Hz, 1H), 4.26 (br, 4H), 2.50 (m, 4H), 2.24 (s, 3H). MS m/z: 306 (M+H⁺).

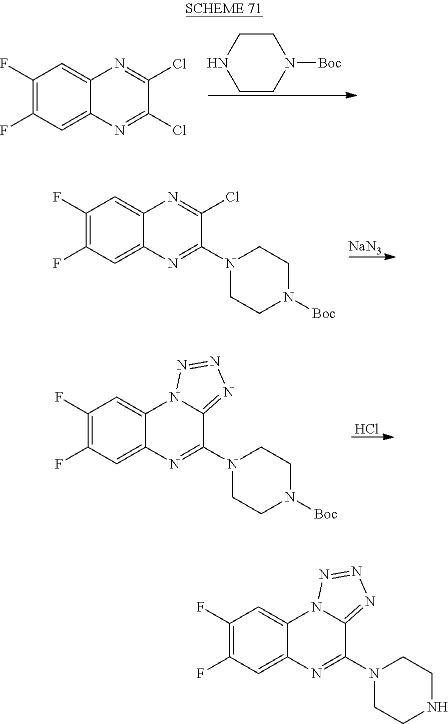

### EXAMPLE 191

### 7,8-difluoro-4-(piperazin-1-yl)tetrazolo[1,5-a]quinoxaline

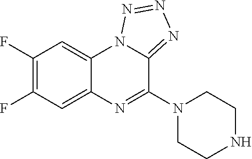

The HCl salt of the title compound was prepared as described in Examples 37 and 29, except that 2,3-dichloro-6,7-difluoroquinoxaline was substituted for 2,3-dichloro-6-(trifluoromethyl)quinoxaline as the starting material of that route. ¹H NMR (300 MHz, DMSO-d₆) δ: 9.65 (br, 3H), 8.55 (dd, J=10.2, 7.8 Hz, 1H), 7.87 (dd, J=11.7, 7.8 Hz, 1H), 4.50 (br, 4H), 3.30 (m, 4H). MS m/z: 292 (M+H⁺).

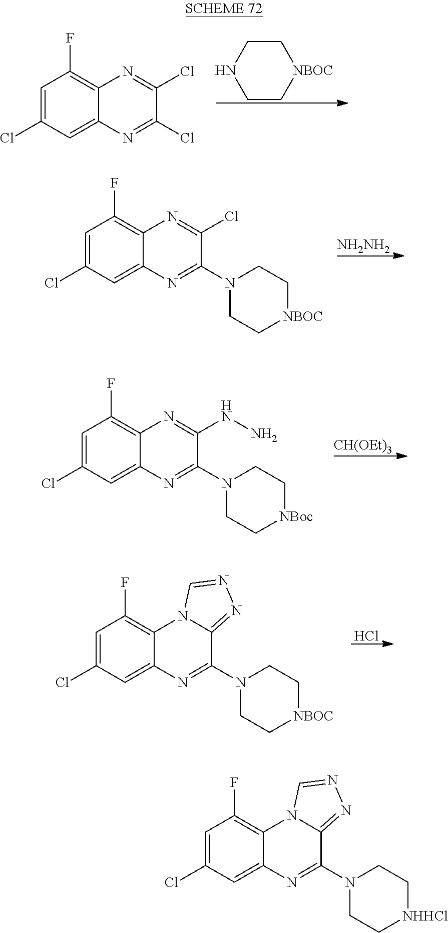

### EXAMPLE 192

### 7-chloro-9-fluoro-4-(piperazin-1-yl)-[1,2,4]triazolo[4,3-a]quinoxaline

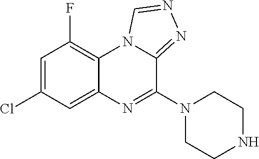

The HCl salt of the title compound was prepared as described in Examples 23 and 196, except that 5-chloro-3-fluorobenzene-1,2-diamine was substituted for 4-(trifluoromethyl)benzene-1,2-diamine. ¹H NMR (300 MHz, DMSO-d₆) δ: 9.59 (s, 1H), 7.46-7.39 (m, 2H), 4.29 (br, 4H), 2.87 (br, 4H). MS m/z: 307 (M+H⁺).

### EXAMPLE 193

### 7-bromo-4-(4-methylpiperazin-1-yl)-[1,2,4]triazolo[4,3-a]quinoxaline

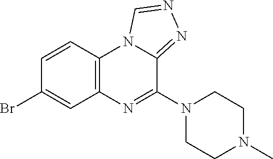

The title compound was prepared as described in Example 54, except that 7-bromo-4-(piperazin-1-yl)-[1,2,4]triazolo[4,3-a]quinoxaline HCl salt (Example 53) was substituted for 8-bromo-4-piperazinyl-10-hydro-1,2,4-triazolo[4,3-a]quinoxaline HCl salt (Example 52). ¹H NMR (300 MHz, CDCl₃) δ: 9.12 (s, 1H), 7.83 (d, J=2.1 Hz, 1H), 7.57 (d, J=8.7 Hz, 1H), 7.38 (dd, J=8.7, 2.1 Hz, 1H), 4.48 (br, 4H), 2.60 (t, J=5.1 Hz, 4H), 2.36 (s, 3H). MS m/z: 347 (M+H⁺).

### EXAMPLE 194

### 7-bromo-4-(piperazin-1-yl)tetrazolo[1,5-a]quinoxaline hydrochloride

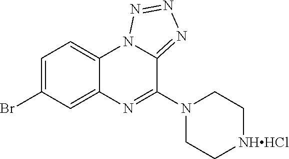

The title compound was prepared as described in Examples 29 and 52, except that 4-bromobenzene-1,2-diamine was substituted for 4-(trifluoromethyl)benzene-1,2-diamine as the starting material. ¹H NMR (300 MHz, DMSO-d₆) δ: 9.58 (s, 2H), 8.30 (d, J=9.0 Hz, 1H), 7.96 (s, 1H), 7.70 (d, J=9.0 Hz, 1H), 4.52 (br, 4H), 3.29 (br, 4H). MS m/z: 334 (M+H⁺).

### EXAMPLE 195

### 7-bromo-4-(4-methylpiperazin-1-yl)tetrazolo[1,5-a]quinoxaline

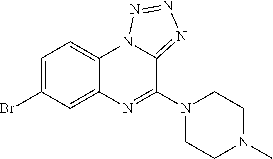

The title compound was prepared as described in Example 54, except that 7-bromo-4-(piperazin-1-yl)tetrazolo[1,5-a]quinoxaline hydrochloride (Example 194) was substituted for 8-bromo-4-piperazinyl-10-hydro-1,2,4-triazolo[4,3-a]quinoxaline HCl salt (Example 52). ¹H NMR (300 MHz, CDCl₃) δ: 8.21 (d, J=8.4 Hz, 1H), 7.92 (s, 1H), 7.51 (d, J=8.7 Hz, 1H), 4.44 (br, 4H), 2.61 (t, J=4.8 Hz, 4H), 2.37 (s, 3H). MS m/z: 348 (M+H⁺).

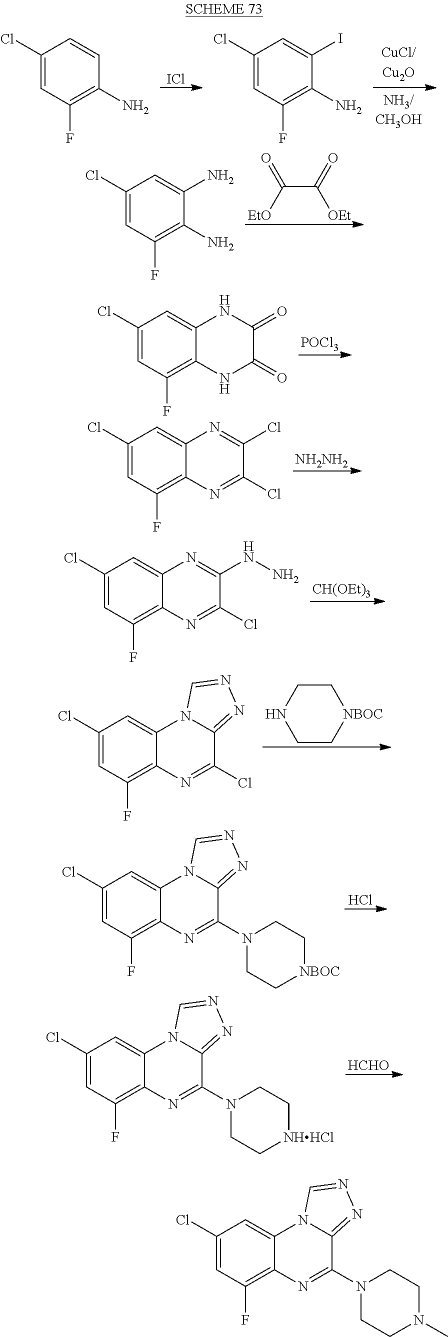

### EXAMPLE 196

### 8-chloro-6-fluoro-4-(piperazin-1-yl)-[1,2,4]triazolo[4,3-a]quinoxaline

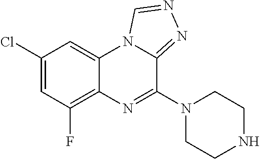

### Step 1

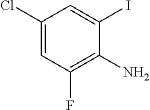

### 4-Chloro-2-fluoro-6-iodoaniline

The title compound was prepared as described in Example 122 step 1, except that 4-chloro-2-fluoroaniline was substituted for 3-chloro-4-(trifluoromethyl)aniline.

### Step 2

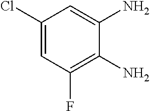

### 5-chloro-3-fluorobenzene-1,2-diamine

The title compound was prepared as described in Example 236 step 5, except that 4-chloro-2-fluoro-6-iodoaniline was substituted for 6-bromo-2-fluoro-3-(trifluoromethyl)aniline.

### Steps 3-8

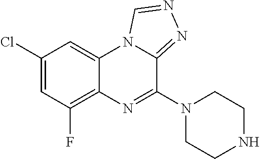

### 8-chloro-6-fluoro-4-(piperazin-1-yl)-[1,2,4]triazolo[4,3-a]quinoxaline

The HCl salt of the title compound was prepared as described in Example 21, except that 5-chloro-3-fluorobenzene-1,2-diamine was substituted for 4-methylbenzene-1,2-diamine as the starting material. ¹H NMR (300 MHz, DMSO-d₆) δ: 9.99 (s, 1H), 8.23 (s, 1H), 7.49 (d, J=10.8 Hz, 1H), 4.26 (br, 4H), 2.85 (br, 4H). MS m/z: 307 (M+H⁺).

### EXAMPLE 197

### 8-chloro-6-fluoro-4-(4-methylpiperazin-1-yl)-[1,2,4]triazolo[4,3-a]quinoxaline

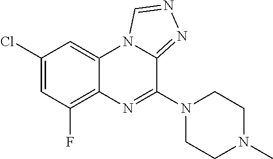

The title compound was prepared as described in Example 54, except that 8-chloro-6-fluoro-4-(piperazin-1-yl)-[1,2,4]triazolo[4,3-a]quinoxaline HCl salt (Example 196) was substituted for 8-bromo-4-piperazinyl-10-hydro-1,2,4-triazolo[4,3-a]quinoxaline HCl salt (Example 52). ¹H NMR (300 MHz, DMSO-d₆) δ: 9.99 (s, 1H), 8.24 (s, 1H), 7.53 (d, J=10.5 Hz, 1H), 4.33 (br, 4H), 3.30 (br, 4H), 2.23 (s, 3H). MS m/z: 321 (M+H⁺).

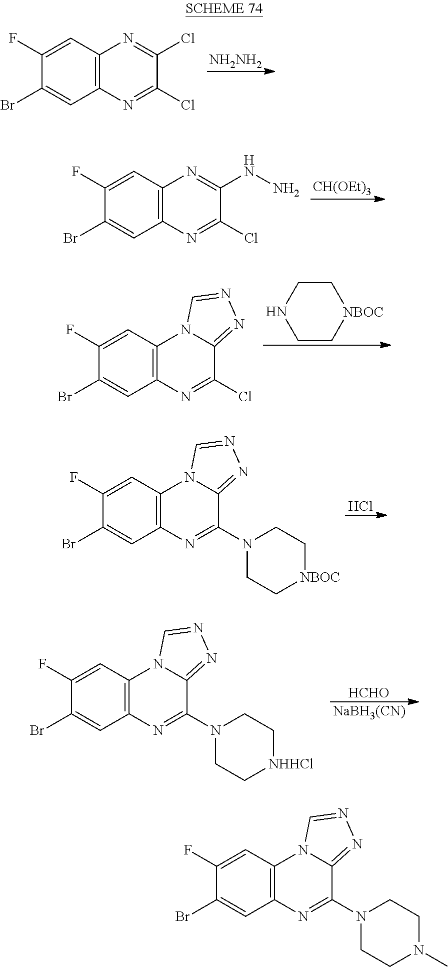

### EXAMPLE 198

### 7-bromo-8-fluoro-4-(piperazin-1-yl)-[1,2,4]triazolo[4,3-a]quinoxaline

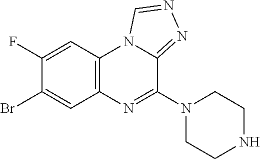

The HCl salt of the title compound was prepared as described in Examples 21 and 184, except that 4-bromo-5-fluorobenzene-1,2-diamine was substituted for 4-methylbenzene-1,2-diamine. ¹H NMR (300 MHz, DMSO-d₆) δ: 9.86 (s, 1H), 8.27 (d, J=9.3 Hz, 1H), 7.73 (d, J=6.6 Hz, 1H), 4.21 (br, 4H), 2.80 (br, 4H). MS m/z: 351 (M+H⁺).

### EXAMPLE 199

### 7-bromo-8-fluoro-4-(4-methylpiperazin-1-yl)-[1,2,4]triazolo[4,3-a]quinoxaline

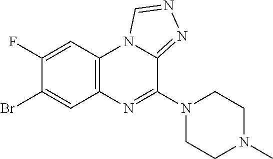

The title compound was prepared as described in Example 54, except that 7-bromo-8-fluoro-4-(piperazin-1-yl)-[1,2,4]triazolo[4,3-a]quinoxaline HCl salt (Example 198) was substituted for 8-bromo-4-piperazinyl-10-hydro-1,2,4-triazolo[4,3-a]quinoxaline HCl salt (Example 52). ¹H NMR (300 MHz, DMSO-d₆) δ: 9.90 (s, 1H), 8.31 (d, J=9.6 Hz, 1H), 7.79 (d, J=6.6 Hz, 1H), 4.27 (br, 4H), 2.46 (br, 4H), 2.22 (s, 3H). MS m/z: 365 (M+H⁺).

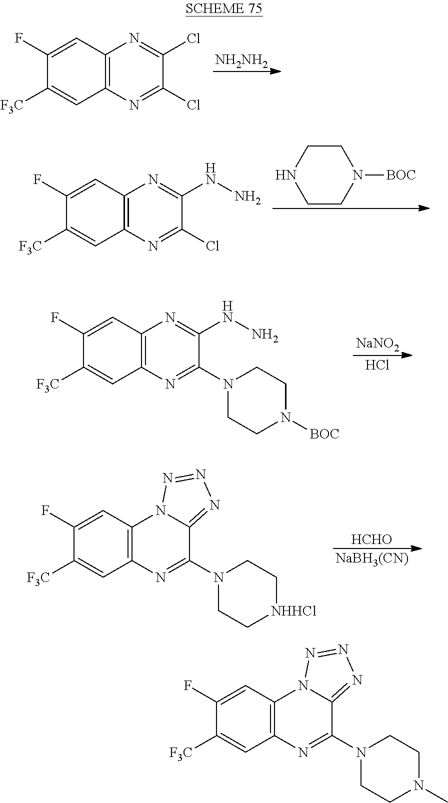

### EXAMPLE 200

### 8-fluoro-4-(piperazin-1-yl)-7-(trifluoromethyl)tetrazolo[1,5-a]quinoxaline

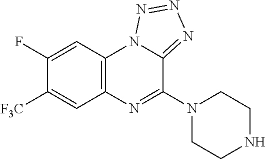

The HCl salt of the title compound was prepared as described in Examples 18 and 186, except that 5-fluoro-4-(trifluoromethyl)benzene-1,2-diamine (prepared according to Example 34) was substituted for 4-methylbenzene-1,2-diamine as the starting material. ¹H NMR (300 MHz, CDCl₃) δ: 8.19 (d, J=9.3 Hz, 1H), 8.04 (d, J=6.6 Hz, 1H), 4.41 (br, 4H), 3.09 (m, 4H). MS m/z: 342 (M+H⁺).

### EXAMPLE 201

### 8-fluoro-4-(4-methylpiperazin-1-yl)-7-(trifluoromethyl)tetrazolo[1,5-a]quinoxaline

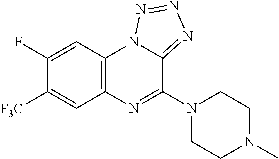

The title compound was prepared as described in Example 54, except that 8-fluoro-4-(piperazin-1-yl)-7-(trifluoromethyl)tetrazolo[1,5-a]quinoxaline HCl salt was substituted for 8-bromo-4-piperazinyl-10-hydro-1,2,4-triazolo[4,3-a]quinoxaline HCl salt as the starting material. ¹H NMR (300 MHz, CDCl₃) δ: 8.19 (d, J=9.3 Hz, 1H), 8.04 (d, J=6.3 Hz, 1H), 4.44 (br, 4H), 2.62 (m, 4H), 2.38 (s, 3H). MS m/z: 356 (M+H⁺).

### EXAMPLE 202

### 7-chloro-8-fluoro-4-(piperazin-1-yl)tetrazolo[1,5-a]quinoxaline

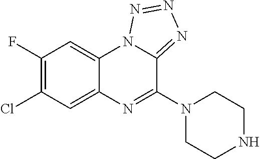

The title compound was prepared as described in Example 18, except that 4-chloro-5-fluorobenzene-1,2-diamine was substituted for 4-methylbenzene-1,2-diamine as the starting material of that route. ¹H NMR (300 MHz, CDCl₃) δ: 8.14 (d, J=8.1 Hz, 1H), 7.83 (d, J=7.2 Hz, 1H), 4.38 (br, 4H), 3.08 (t, J=5.1 Hz, 4H). MS m/z: 308 (M+H⁺).

### EXAMPLE 203

### 7-chloro-8-fluoro-4-(4-methylpiperazin-1-yl)tetrazolo[1,5-a]quinoxaline

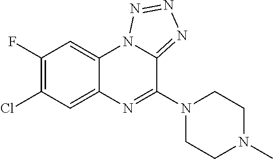

The title compound was prepared as described in Example 54, except that 7-chloro-8-fluoro-4-(piperazin-1-yl)tetrazolo[1,5-a]quinoxaline (Example 202) was substituted for 8-bromo-4-piperazinyl-10-hydro-1,2,4-triazolo[4,3-a]quinoxaline HCl salt (Example 52). ¹H NMR (300 MHz, CDCl₃) δ: 8.14 (d, J=7.8 Hz, 1H), 7.83 (d, J=6.9 Hz, 1H), 4.42 (br, 4H), 2.61 (t, J=5.1 Hz, 4H), 2.38 (s, 3H). MS m/z: 322 (M+H⁺).

### EXAMPLE 204

### 8-bromo-4-(4-methylpiperazin-1-yl)tetrazolo[1,5-a]quinoxaline

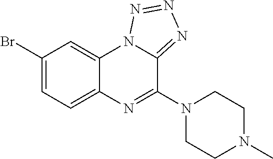

The title compound was prepared as described in Example 18, except that 4-bromobenzene-1,2-diamine was substituted for 4-methylbenzene-1,2-diamine as the starting material and N-methylpiperazine for piperazine in step 4 of that route. ¹H NMR (300 MHz, CDCl₃) δ: 8.52 (d, J=2.1 Hz, 1H), 7.68 (dd, J=8.7, 2.1 Hz, 1H), 7.60 (d, J=8.7 Hz, 1H), 4.43 (br, 4H), 2.62 (t, J=5.3 Hz, 4H), 2.38 (s, 3H). MS m/z: 348 (M+H⁺).

### EXAMPLE 205

### 8-bromo-4-(piperazin-1-yOtetrazolo[1,5-a]quinoxaline

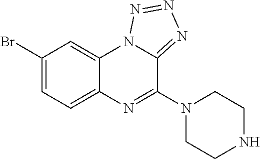

The title compound was prepared as described in Example 18, except that 4-bromobenzene-1,2-diamine was substituted for 4-methylbenzene-1,2-diamine as the starting material of that route. ¹H NMR (300 MHz, CDCl₃) δ: 8.50 (d, J=2.1 Hz, 1H), 7.67 (dd, J=8.7, 2.1 Hz, 1H), 7.59 (d, J=8.7 Hz, 1H), 4.38 (br, 4H), 3.08 (m, 4H). MS m/z: 334 (M+H⁺).

### EXAMPLE 206

### 6-fluoro-4-(piperazin-1-yl)-8-(trifluoromethyl)tetrazolo[1,5-a]quinoxaline hydrochloride

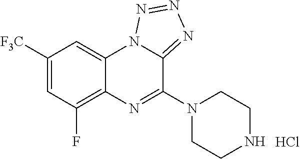

The title compound was prepared as described in Example 29, except that 3-fluoro-5-(trifluoromethyl)benzene-1,2-diamine (prepared as described in Example 48 steps 1-4) was substituted for 4-(trifluoromethyl)benzene-1,2-diamine as the starting material of that route. ¹H NMR (300 MHz, DMSO-d₆) δ: 9.58 (br, 2H), 8.48 (s, 1H), 8.07 (dd, J=10.8, 1.8 Hz, 1H), 4.61 (br, 4H), 3.33 (t, J=5.1 Hz, 4H). MS m/z: 342 (M+H⁺).

### EXAMPLE 207

### 6-fluoro-4-(4-methylpiperazin-1-yl)-8-(trifluoromethyl)tetrazolo[1,5-a]quinoxaline hydrochloride

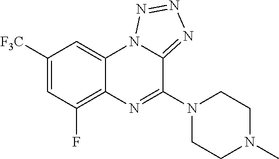

The title compound was prepared as described in Example 54, except that 6-fluoro-4-(piperazin-1-yl)-8-(trifluoromethyl)tetrazolo[1,5-a]quinoxaline hydrochloride (Example 206) was substituted for 8-bromo-4-piperazinyl-10-hydro-1,2,4-triazolo[4,3-a]quinoxaline HCl salt (Example 52). ¹H NMR (300 MHz, DMSO-d₆) δ: 8.38 (s, 1H), 7.97 (dd, J=10.8, 2.1 Hz, 1H), 4.37 (br, 4H), 2.54 (t, J=5.1 Hz, 4H), 2.25 (s, 3H). MS m/z: 356 (M+H⁺).

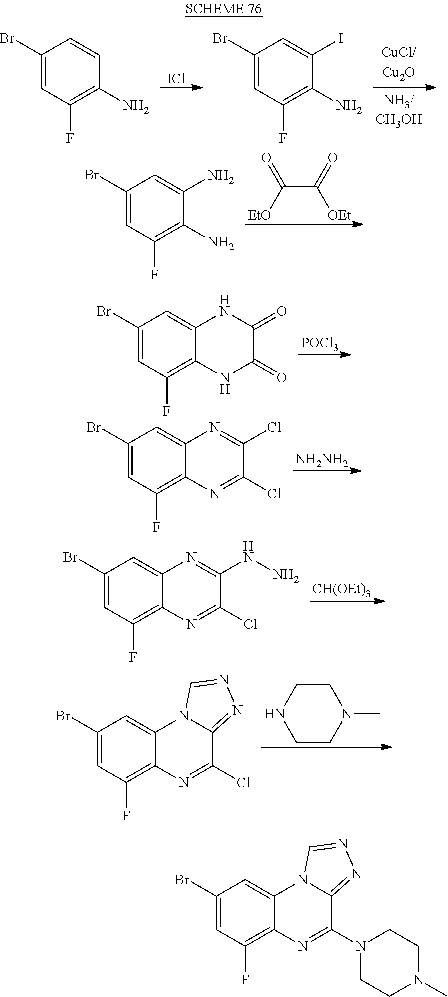

### EXAMPLE 208

### 8-bromo-6-fluoro-4-(4-methylpiperazin-1-yl)-[1,2,4]triazolo[4,3-a]quinoxaline

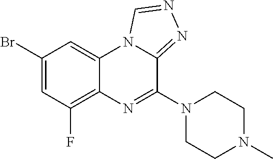

The title compound was prepared as described in Example 196, except that 4-bromo-2-fluoroaniline was substituted for 4-chloro-2-fluoroaniline in step 1, and N-methylpiperazine for N—BOC piperazine in step 7 of that route. ¹H NMR (300 MHz, CD₃OD) δ: 9.75 (s, 1H), 8.14 (d, J=1.5 Hz, 1H), 7.44 (dd, J=9.6, 1.8 Hz, 1H), 4.45 (br, 4H), 2.64 (t, J=5.1 Hz, 4H), 2.37 (s, 3H). MS m/z: 365 (M+H⁺).

### EXAMPLE 209

### 8-bromo-6-fluoro-4-(piperazin-1-yl)-[1,2,4]triazolo[4,3-a]quinoxaline

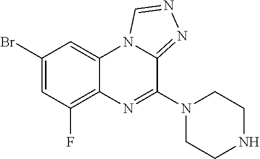

The title compound was prepared as described in Example 208, except that piperazine was substituted for N-methylpiperazine in the last step of that route. ¹H NMR (300 MHz, CD₃OD) δ: 9.75 (s, 1H), 8.13 (d, J=1.5 Hz, 1H), 7.44 (dd, J=9.6, 1.8 Hz, 1H), 4.41 (br, 4H), 3.01 (t, J=5.1 Hz, 4H). MS m/z: 351 (M+H⁺).

### EXAMPLE 210

### 8-bromo-6-fluoro-4-(4-methylpiperazin-1-yl)tetrazolo[1,5-a]quinoxaline

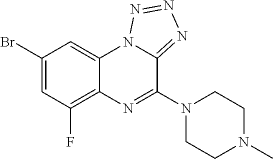

The title compound was prepared as described in Example 18, except that 5-bromo-3-fluorobenzene-1,2-diamine (prepared as described in Example 208, steps 1-2) was substituted for 4-methylbenzene-1,2-diamine as the starting material of that route. ¹H NMR (300 MHz, CD₃OD) δ: 8.34 (t, J=1.8 Hz, 1H), 7.50 (dd, J=9.9, 1.8 Hz, 1H), 4.47 (br, 4H), 2.63 (t, J=5.1 Hz, 4H), 2.39 (s, 3H). MS m/z: 366 (M+H⁺).

### EXAMPLE 211

### 8-bromo-7-fluoro-4-(piperazin-1-yl)tetrazolo[1,5-a]quinoxaline

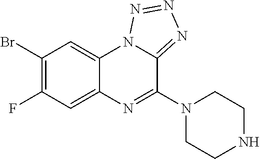

The HCl salt of the title compound was prepared as described in Example 29, except that 4-bromo-5-fluorobenzene-1,2-diamine (prepared as described in Example 184 steps 1-4) was substituted for 4-(trifluoromethyl)benzene-1,2-diamine as the starting material of that route. ¹H NMR (300 MHz, DMSO-d₆) δ: 9.45 (br, 2H), 8.70 (d, J=6.6 Hz, 1H), 7.75 (d, J=9.9 Hz, 1H), 4.52 (br, 4H), 3.30 (t, J=5.7 Hz, 4H). MS m/z: 352 (M+H⁺).

### EXAMPLE 212

### 8-bromo-7-fluoro-4-(4-methylpiperazin-1-yl)tetrazolo[1,5-a]quinoxaline

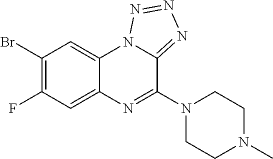

The title compound was prepared as described in Example 54, except that 8-bromo-7-fluoro-4-(piperazin-1-yl)tetrazolo[1,5-a]quinoxaline HCl salt (Example 211) was substituted for 8-bromo-4-piperazinyl-10-hydro-1,2,4-triazolo[4,3-a]quinoxaline HCl salt (Example 52). ¹H NMR (300 MHz, CDCl₃) δ: 8.57 (d, J=6.9 Hz, 1H), 7.46 (d, J=9.3 Hz, 1H), 4.46 (br, 4H), 2.61 (t, J=5.1 Hz, 4H), 2.38 (s, 3H). MS m/z: 366 (M+H⁺).

### EXAMPLE 213

### 8-chloro-4-(hexahydropyrrolo[1,2-a]pyrazin-2(1H)-yl)tetrazolo[1,5-a]quinoxaline

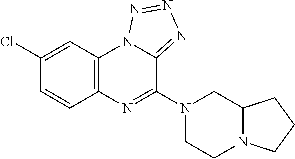

### Step 1

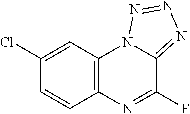

### 4,8-dichlorotetrazolo[1,5-a]quinoxaline

A 25 mL round bottom flask was charged with 2,6-dichloro-3-hydrazinylquinoxaline (prepared as described in Example 1, steps 1-3, 0.1 g, 0.44 mmol) and 1N aqueous HCl solution (2 mL). To the suspension was added dropwise a solution of sodium nitrite (45 mg, 0.44 mmol) in water (0.5 mL) at 0° C. The resulting mixture was stirred at 0-5° C. for further 0.5 h. Reaction progress was monitored by TLC (EtOAc/Petroleum ether=1:1). Work-up: the precipitate was collected by filtration and washed with water to afford 100 mg (95%) of the product as light yellow solids. ¹H NMR (300 MHz, DMSO-d₆) δ: 8.70 (d, J=2.1 Hz, 1H), 8.27 (d, J=8.7 Hz, 1H), 8.00 (dd, J=8.7, 2.1 Hz, 1H).

### Step 2

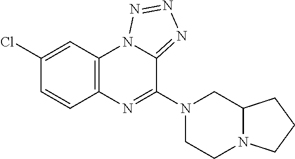

### 8-chloro-4-(hexahydropyrrolo[1,2-a]pyrazin-2(1H)-yl)tetrazolo[1,5-a]quinoxaline

The title compound was prepared as described in Example 19, except that octahydropyrrolo[1,2-a]pyrazine was substituted for piperazine in that route. ¹H NMR (300 MHz, DMSO-d₆) δ: 8.33 (d, J=2.1 Hz, 1H), 7.72 (d, J=8.7 Hz, 1H), 7.66 (dd, J=8.7, 2.1 Hz, 1H), 5.44-5.37 (m, 2H), 3.33-3.16 (m, 2H), 3.08-2.92 (m, 2H), 2.29-2.21 (m, 1H), 2.14-2.05 (m, 2H), 1.90-1.66 (m, 3H), 1.50-1.41 (m, 1H). MS m/z: 330 (M+H⁺).

### EXAMPLE 214

### 2-methyl-4-(4-methylpiperazin-1-yl)-8-(trifluoromethyl)oxazolo[4,5-c]quinoline

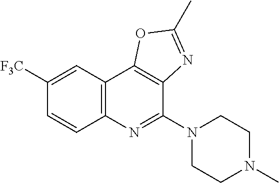

The title compound was prepared as described in Example 141, except that 2-amino-5-(trifluoromethyl)benzoic acid was substituted for 2-amino-5-chlorobenzoic acid as the starting material, and ethyl orthoacetate was substituted for ethyl orthoformate in step 4 of that route. ¹H NMR (300 MHz, DMSO-d₆) δ: 8.21 (s, 1H), 7.79 (m, 2H), 4.21 (br, 4H), 2.73 (s, 3H), 2.49 (m, 4H), 2.24 (s, 3H). MS m/z: 351 (M+H⁺).

### EXAMPLE 215

### 2-methyl-4-(piperazin-1-yl)-8-(trifluoromethyl)oxazolo[4,5-c]quinoline

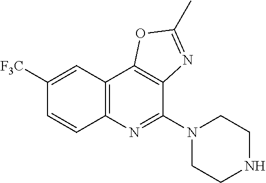

The title compound was prepared as described in Example 214, except that piperazine was substituted for N-methylpiperazine in the last step of that route. ¹H NMR (300 MHz, DMSO-d₆) δ: 8.19 (s, 1H), 7.77 (m, 2H), 4.15 (m, 4H), 2.85 (m, 4H), 2.72 (s, 3H). MS m/z: 337 (M+H⁺).

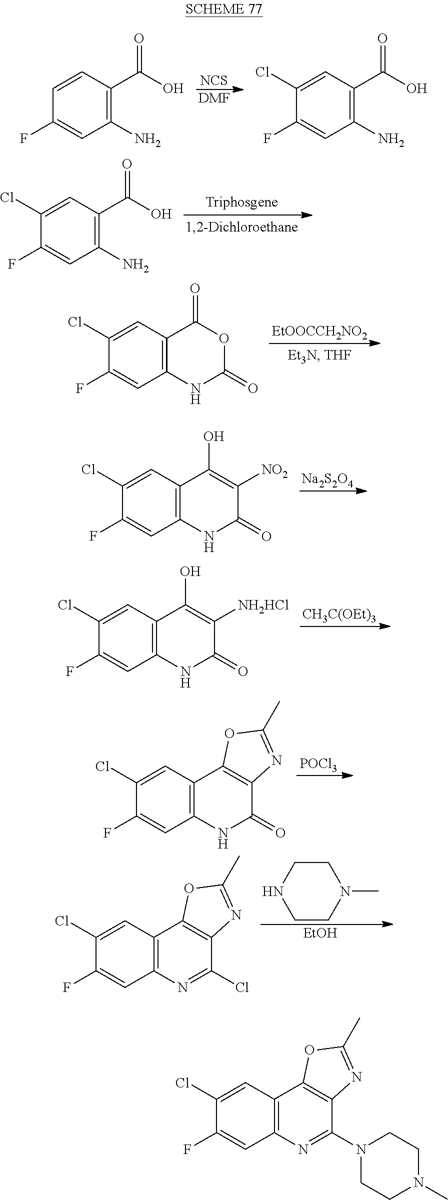

### EXAMPLE 216

### 8-chloro-7-fluoro-2-methyl-4-(4-methylpiperazin-1-yl)oxazolo[4,5-c]quinoline

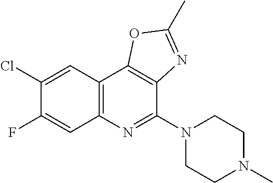

### Step 1

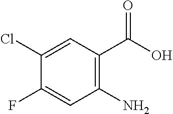

### 2-Amino-5-chloro-4-fluorobenzoic acid

A 500 mL 3-necked round bottom flask was charged with 2-amino-4-fluorobenzoic acid (5.0 g, 32.3 mmol) and anhydrous DMF (75 mL). To the above was added N-chlorosuccinimide (4.3 g, 32.3 mmol) in several portions at room temperature. The resulting mixture was heated at 50° C. for 2.5 h. Reaction progress was monitored by TLC (EtOAc/Petroleum ether=1:1, Rf=0.4). Work-up: the mixture was poured into water and filtered. The solid collected was washed with water and dried, to afford 4.53 g (74%) of the product, which was used in the next step without further purification.

### Steps 2-7

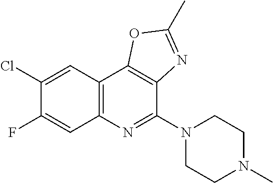

### 8-chloro-7-fluoro-2-methyl-4-(4-methylpiperazin-1-yl)oxazolo[4,5-c]quinoline

The title compound was prepared as described in Example 141, except that 2-amino-5-chloro-4-fluorobenzoic acid was substituted for 2-amino-5-chlorobenzoic acid in step 1, and ethyl orthoacetate was substituted for ethyl orthoformate in step 4 of that route. ¹H NMR (300 MHz, DMSO-d₆) δ: 8.07 (d, J=7.8 Hz, 1H), 7.55 (d, J=11.4 Hz, 1H), 4.15 (t, J=4.8 Hz, 4H), 2.70 (s, 3H), 2.46 (t, J=4.8 Hz, 4H), 2.23 (s, 3H). MS m/z: 335 (M+H⁺).

### EXAMPLE 217

### 8-chloro-7-fluoro-2-methyl-4-(piperazin-1-yl)oxazolo[4,5-c]quinoline

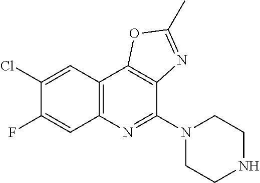

The title compound was prepared as described in Example 216, except that piperazine was substituted for N-methylpiperazine in the last step of that route. ¹H NMR (300 MHz, DMSO-d₆) δ: 8.02 (d, J=8.4 Hz, 1H), 7.50 (d, J=11.7 Hz, 1H), 4.08 (t, J=4.8 Hz, 4H), 2.83 (t, J=4.5 Hz, 4H), 2.69 (s, 3H). MS m/z: 321 (M+H⁺).

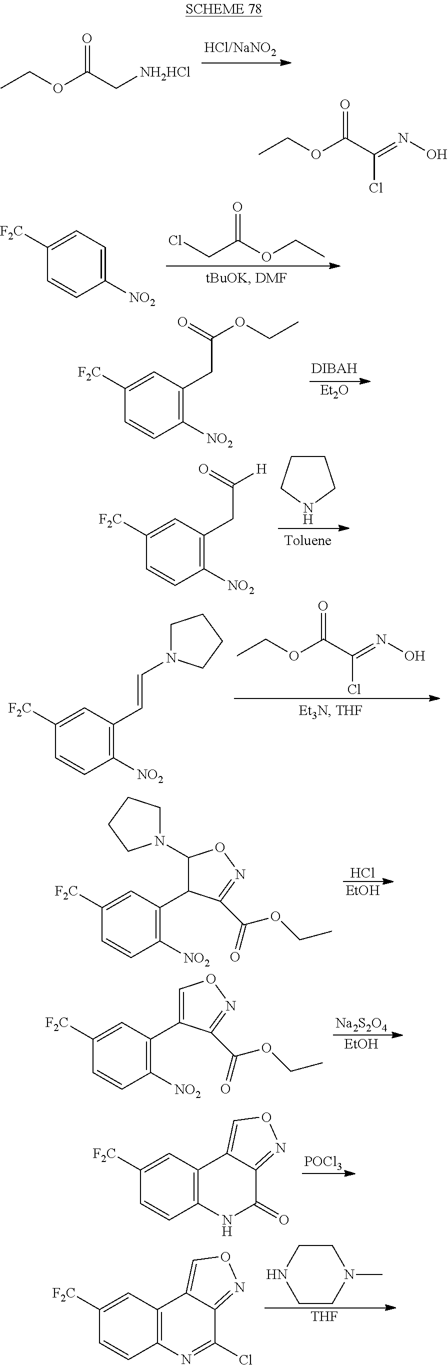

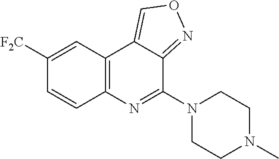

### EXAMPLE 218

### 4-(4-methylpiperazin-1-yl)-8-(trifluoromethyl)isoxazolo[3,4-c]quinoline

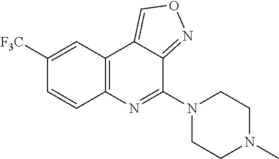

The title compound was prepared as described in Example 166, except that 4-nitrobenzotrifluoride was substituted for 1-chloro-4-nitrobenzene as the starting material. ¹H NMR (300 MHz, CDCl₃) δ: 9.33 (s, 1H), 8.02 (s, 1H), 7.66 (s, 2H), 4.35 (t, J=3.3 Hz, 4H), 2.61 (t, J=4.5 Hz, 4H), 2.38 (s, 3H). MS m/z: 337 (M+H⁺).

### EXAMPLE 219

### 4-piperazinyl-8-(trifluoromethyl)isoxazolo[3,4-c]quinoline

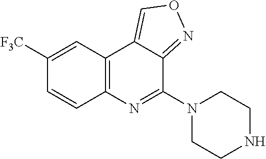

The title compound was prepared as described in Example 218, except that piperazine was substituted for N-methylpiperazine in the last step of that route. ¹H NMR (300 MHz, CDCl₃) δ: 9.33 (s, 1H), 8.02 (s, 1H), 7.66 (s, 2H), 4.33 (t, J=3.3 Hz, 4H), 3.09 (t, J=4.2 Hz, 4H). MS m/z: 323 (M+H⁺).

### EXAMPLE 220

### 4-(4-Methylpiperazinyl)-7-(trifluoromethyl)isoxazolo[3,4-c]quinoline

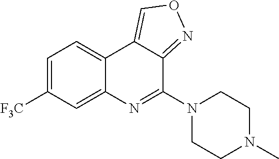

The title compound was prepared as described in Example 166, except that 3-nitrobenzotrifluoride was substituted for 1-chloro-4-nitrobenzene as the starting material. ¹H NMR (300 MHz, CDCl₃) δ: 9.32 (s, 1H), 7.85 (m, 2H), 7.42 (dd, J=8.1, 1.2 Hz, 1H), 4.32 (m, 4H), 2.60 (m, 4H), 2.37 (s, 3H). MS m/z: 337 (M+H⁺).

### EXAMPLE 221

### 4-(piperazin-1-yl)-7-(trifluoromethyl)isoxazolo[3,4-c]quinoline

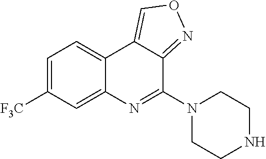

The title compound was prepared as described in Example 220, except that piperazine was substituted for N-methylpiperazine in the last step of that route. ¹H NMR (300 MHz, CDCl₃) δ: 9.33 (s, 1H), 7.86 (m, 2H), 7.42 (dd, J=7.8, 1.5 Hz, 1H), 4.27 (m, 4H), 3.06 (m, 4H). MS m/z: 323 (M+H⁺).

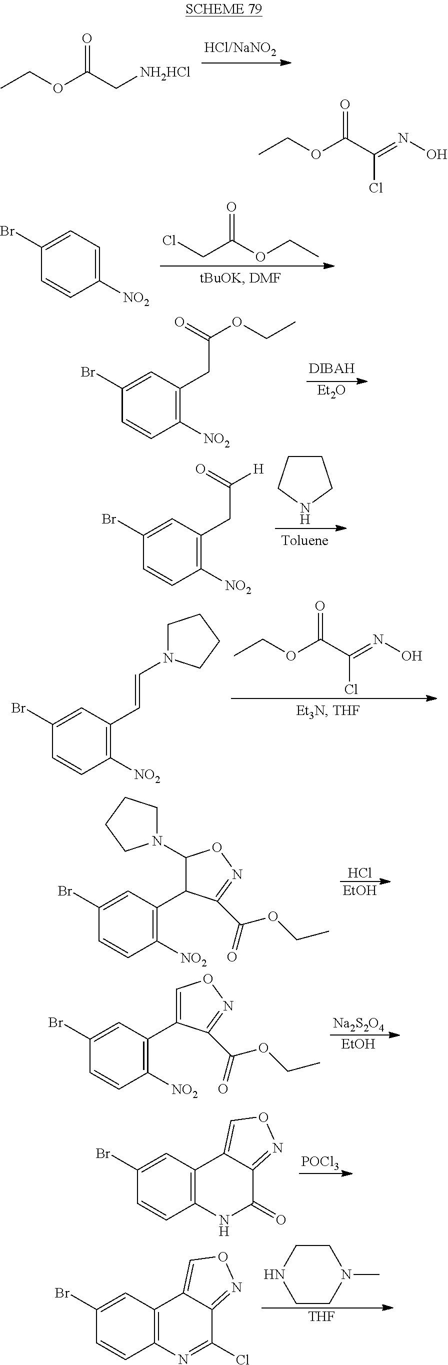

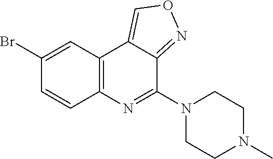

### EXAMPLE 222

### 8-bromo-4-(4-methylpiperazin-1-yl)isoxazolo[3,4-c]quinoline

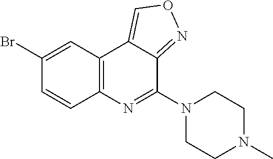

The title compound was prepared as described in Example 166, except that 1-bromo-4-nitrobenzene was substituted for 1-chloro-4-nitrobenzene as the starting material. ¹H NMR (300 MHz, CDCl₃) δ: 9.24 (s, 1H), 7.79 (d, J=1.8 Hz, 1H), 7.48 (m, 2H), 4.28 (t, J=4.8 Hz, 4H), 2.59 (t, J=5.1 Hz, 4H), 2.37 (s, 3H). MS m/z: 347 (M+H⁺).

### EXAMPLE 223

### 8-bromo-4-(piperazin-1-yl)isoxazolo[3,4-c]quinoline

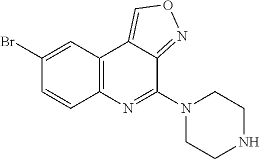

The title compound was prepared as described in Example 222, except that piperazine was substituted for N-methylpiperazine in the last step of that route. ¹H NMR (300 MHz, CDCl₃) δ: 9.24 (s, 1H), 7.89 (d, J=2.1, 1H), 7.48 (m, 2H), 4.24 (t, J=4.8 Hz, 4H), 3.06 (t, J=5.1 Hz, 4H). MS m/z: 333 (M+H⁺).

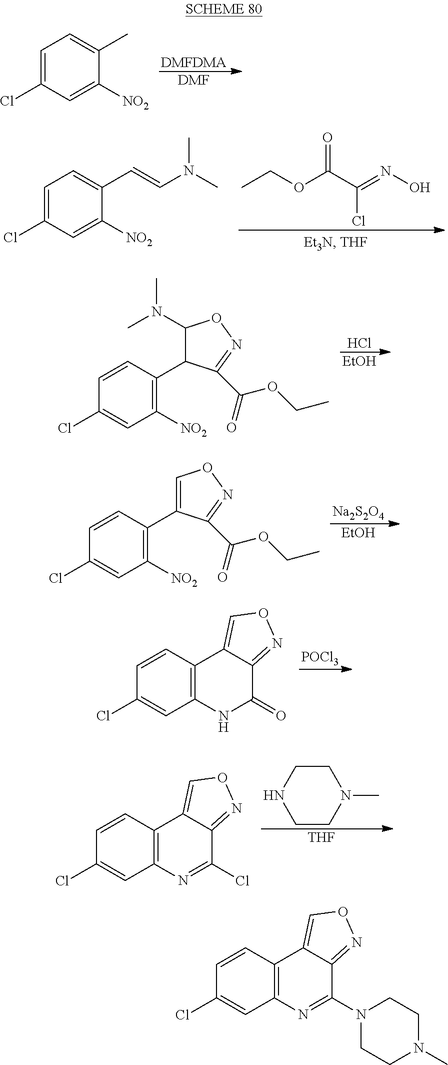

### EXAMPLE 224

### 7-chloro-4-(4-methylpiperazin-1-yl)isoxazolo[3,4-c]quinoline

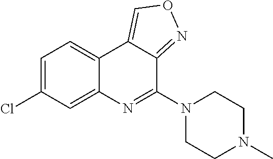

### Step 1

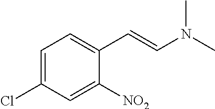

### (E)-2-(4-chloro-2-nitrophenyl)-N,N-dimethylethenamine

A 250 mL round bottom flask was charged with 4-chloro-2-nitrotoluene (10.0 g, 58.3 mmol), N,N-dimethylformamide dimethyl acetal (23 mL) and DMF (100 mL). The resulting mixture was stirred at reflux overnight. Work-up: the reaction mixture was concentrated in vacuo. The residue was used as such for the next step. ¹H NMR (300 MHz, CDCl₃) δ: 7.85 (d, J=2.1 Hz, 1H), 7.38 (d, J=8.7 Hz, 1H), 7.26 (m, 1H), 6.93 (d, J=16.2 Hz, 1H), 5.83 (d, J=13.5 Hz, 1H), 2.91 (s, 6H).

### Steps 2-6

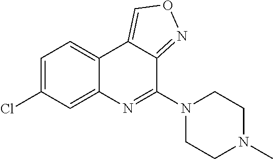

### 7-chloro-4-(4-methylpiperazin-1-yl)isoxazolo[3,4-c]quinoline

The title compound was prepared as described in Example 166, except that (E)-2-(4-chloro-2-nitrophenyl)-N,N-dimethylethenamine was substituted for [(1E)-2-(5-chloro-2-nitrophenyl)vinyl]pyrrolidine in step 4 of that route. ¹H NMR (300 MHz, CDCl₃) δ: 9.23 (s, 1H), 7.68 (d, J=8.4 Hz, 1H), 7.61 (d, J=1.8 Hz, 1H), 7.18 (dd, J=8.4, 2.1 Hz, 1H), 4.23 (t, J=4.8 Hz, 1H), 2.58 (t, J=5.1 Hz, 1H), 2.36 (s, 3H). MS m/z: 303 (M+H⁺).

### EXAMPLE 225

### 7-chloro-4-(piperazin-1-yl)isoxazolo[3,4-c]quinoline

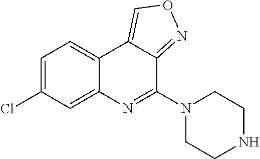

The title compound was prepared as described in Example 224, except that piperazine was substituted for N-methylpiperazine in the last step of that route. ¹H NMR (300 MHz, DMSO-d₆) δ: 10.15 (s, 1H), 7.99 (d, J=8.4 Hz, 1H), 7.46 (d, J=2.1 Hz, 1H), 7.26 (dd, J=8.1, 2.1 Hz, 1H), 4.09 (t, J=4.5 Hz, 1H), 2.83 (t, J=5.1 Hz, 1H). MS m/z: 289 (M+H⁺).

### EXAMPLE 226

### 7,8-difluoro-4-(4-methylpiperazin-1-yl)isoxazolo[3,4-c]quinoline

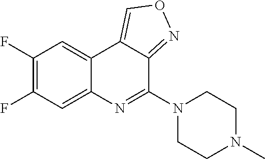

The title compound was prepared as described in Example 224, except that 4,5-difluoro-2-nitrotoluene was substituted for 4-chloro-2-nitrotoluene as the starting material. ¹H NMR (300 MHz, CDCl₃) δ: 9.19 (s, 1H), 7.51 (dd, J=10.0, 8.2 Hz, 1H), 7.37 (dd, J=12.0, 7.8 Hz, 1H), 4.26 (t, J=5.1 Hz, 4H), 2.59 (t, J=5.1 Hz, 4H), 2.36 (s, 3H). MS m/z: 305 (M+H⁺).

### EXAMPLE 227

### 7,8-difluoro-4-(piperazin-1-yl)isoxazolo[3,4-c]quinoline

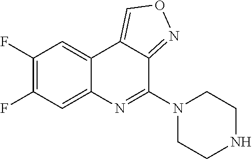

The title compound was prepared as described in Example 226, except that piperazine was substituted for N-methylpiperazine in the last step of that route. ¹H NMR (300 MHz, D₂O) δ: 9.70 (s, 1H), 7.76 (m, 1H), 7.53 (m, 1H), 4.55 (br, 4H), 3.55 (t, J=5.1 Hz, 4H). MS m/z: 291 (M+H⁺).

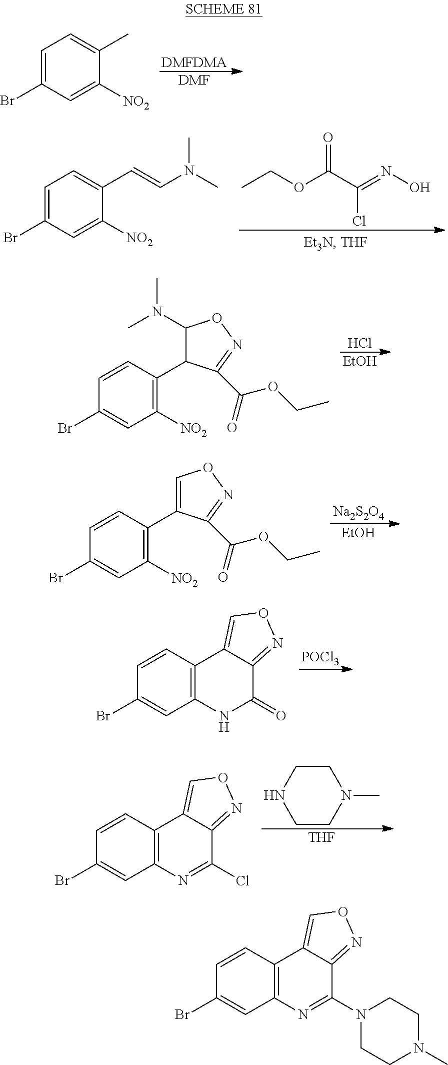

### EXAMPLE 228

### 7-bromo-4-(4-methylpiperazin-1-yl)isoxazolo[3,4-c]quinoline

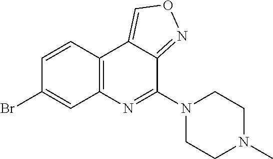

The title compound was prepared as described in Example 224, except that 4-bromo-2-nitrotoluene was substituted for 4-chloro-2-nitrotoluene as the starting material. ¹H NMR (300 MHz, CDCl₃) δ: 9.24 (s, 1H), 7.78 (d, J=1.8 Hz, 1H), 7.61 (d, J=8.1 Hz, 1H), 7.31 (dd, J=8.1, 1.8 Hz, 1H), 4.29 (t, J=4.8 Hz, 4H), 2.58 (t, J=5.1 Hz, 4H), 2.36 (s, 3H). MS m/z: 347 (M+H⁺).

### EXAMPLE 229

### 7-bromo-4-(piperazin-1-yl)isoxazolo[3,4-c]quinoline

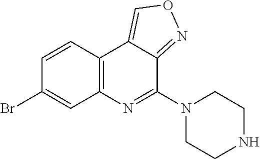

The title compound was prepared as described in Example 228, except that piperazine was substituted for N-methylpiperazine in the last step of that route. ¹H NMR (300 MHz, CDCl₃) δ: 9.25 (s, 1H), 7.78 (d, J=1.8 Hz, 1H), 7.61 (d, J=8.4 Hz, 1H), 7.31 (dd, J=8.1, 1.8 Hz, 1H), 4.26 (t, J=5.1 Hz, 4H), 3.06 (m, 4H). MS m/z: 333 (M+H⁺).

### EXAMPLE 230

### 8-chloro-4-(hexahydropyrrolo[1,2-a]pyrazin-2(1H)-yl)isoxazolo[3,4-c]quinoline

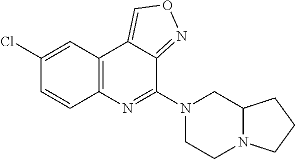

The HCl salt of the title compound was prepared as described in Example 166, except that 1,4-diazabicyclo[4.3.0]nonane was substituted for N-methylpiperazine in the last step. ¹H NMR (300 MHz, CD₃OD) δ: 9.85 (s, 1H), 8.04 (d, J=2.4 Hz, 1H), 7.59 (d, J=8.7 Hz, 1H), 7.45 (dd, J=9.0, 2.4 Hz, 1H), 5.52 (br, 1H), 4.57 (br 1H), 3.60 (br, 6H), 2.35-1.90 (m, 5H). MS: m/z 329 (M+H⁺).

### EXAMPLE 231

### 4-(piperazin-1-yl)-8-(trifluoromethyl)-3H-pyrazolo[3,4-c]quinoline

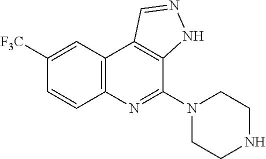

The HCl salt of the title compound was prepared as described in Example 157, except that piperazine was substituted for N-methylpiperazine in step 9 of that route. ¹H NMR (300 MHz, D₂O) δ: 8.60 (s, 1H), 8.20 (s, 1H), 7.79 (d, J=8.7 Hz, 1H), 7.69 (d, J=8.7 Hz, 1H), 4.60 (br, 4H), 3.54 (t, J=4.8 Hz, 4H). MS m/z: 322 (M+H⁺).

### EXAMPLE 232

### 8-bromo-4-(4-methylpiperazin-1-yl)-2H-pyrazolo[3,4-c]quinoline

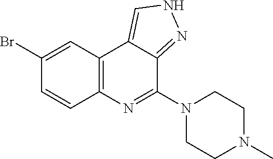

The title compound was prepared as described in Example 152, except that 5-bromoindole was substituted for 5-chloroindole as the starting material of that route. ¹H NMR (300 MHz, CD₃OD) δ: 8.56 (s, 1H), 8.13 (d, J=2.1 Hz, 1H), 7.54 (d, J=8.7 Hz, 1H), 7.45 (dd, J=8.7, 2.1 Hz, 1H), 4.22 (m, 4H), 2.64 (t, J=5.1 Hz, 4H), 2.36 (s, 3H). MS m/z: 346 (M+H⁺).

### EXAMPLE 233

### 8-bromo-4-(piperazin-1-yl)-2H-pyrazolo[3,4-c]quinoline

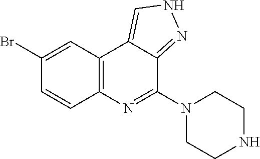

The HCl salt of the title compound was prepared as described in Example 153, except that 5-bromoindole was substituted for 5-chloroindole as the starting material of that route. ¹H NMR (300 MHz, D₂O) δ: 8.46 (s, 1H), 7.83 (s, 1H), 7.45 (s, 2H), 4.65 (br, 4H), 3.54 (m, 4H). MS m/z: 332 (M+H⁺).

### EXAMPLE 234

### 8-bromo-2-methyl-4-(4-methylpiperazin-1-yl)-2H-pyrazolo[3,4-c]quinoline

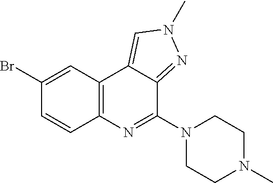

The title compound was prepared as described in Example 150, except that 5-bromoindole was substituted for 5-chloroindole as the starting material of that route. ¹H NMR (300 MHz, CD₃OD) δ: 8.44 (s, 1H), 8.00 (d, J=2.1 Hz, 1H), 7.46 (d, J=4.8 Hz, 1H), 7.43 (dd, J=4.8, 2.1 Hz, 1H), 4.27 (t, J=5.1 Hz, 4H), 4.18 (s, 3H), 2.61 (t, J=5.1 Hz, 4H), 2.35 (s, 3H). MS m/z: 360 (M+H⁺).

### EXAMPLE 235

### 8-bromo-2-methyl-4-(piperazin-1-yl)-2H-pyrazolo[3,4-c]quinoline

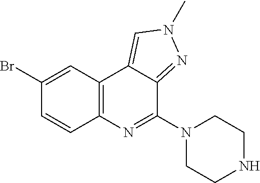

The title compound was prepared as described in Example 234, except that piperazine was substituted for N-methylpiperazine in the last step of that route. ¹H NMR (300 MHz, CD₃OD) δ: 8.42 (s, 1H), 7.98 (d, J=2.1 Hz, 1H), 7.44 (d, J=4.8 Hz, 1H), 7.43 (dd, J=4.8, 2.1 Hz, 1H), 4.22 (t, J=5.1 Hz, 4H), 4.17 (s, 3H), 2.98 (t, J=5.1 Hz, 4H). MS m/z: 346 (M+H⁺).

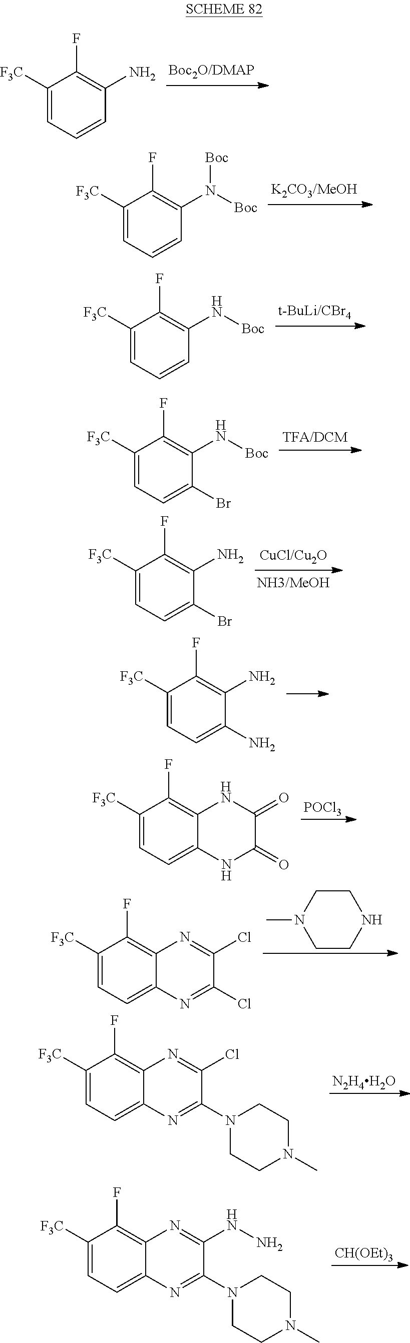

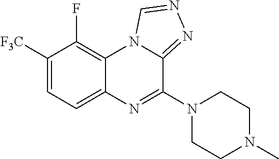

### EXAMPLE 236

### 9-fluoro-4-(4-methylpiperazin-1-yl)-8-(trifluoromethyl)-[1,2,4]triazolo[4,3-a]quinoxaline

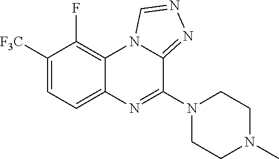

### Step 1

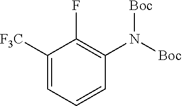

### tert-Butyl {(tert-butoxy)-N-[2-fluoro-3-(trifluoromethyl)phenyl]-carbonylamino}formate

A 1 L round bottom flask was charged with 2-fluoro-3-(trifluoromethyl)aniline (25 g, 0.14 mol), di-tert-butyl dicarbonate (91 g, 0.42 mol), 4-(dimethylamino)pyridine (1.7 g, 14 mmol) and THF (500 mL). The resulting mixture was stirred overnight at reflux. Reaction progress was monitored by TLC (EtOAc/Petroleum ether=1:10). Work-up: the reaction mixture was concentrated in vacuo. The residue was re-dissolved in EtOAc (500 mL) and washed with brine (100 mL). The organic layer was dried over anhydrous Na₂SO₄ and then concentrated in vacuo, to afford 43 g (81%) of the product as white oil.

### Step 2

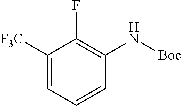

### tert-butyl 2-fluoro-3-(trifluoromethyl)phenylcarbamate

A 1 L round bottom flask was charged with tert-butyl {(tert-butoxy)-N-[2-fluoro-3-(trifluoromethyl)phenyl]carbonylamino}formate (43 g, 0.11 mol), K₂CO₃ (31 g, 0.22 mol) and MeOH (300 mL). The resulting mixture was stirred at reflux for 2 h. Reaction progress was monitored by TLC (EtOAc/Petroleum ether=1:30). Work-up: the reaction mixture was concentrated in vacuo. The residue was re-dissolved in EtOAc (200 mL) and washed with 0.5 N HCl (50 mL). The organic layer was dried over anhydrous Na₂SO₄ and then concentrated in vacuo. The residue was further purified by flash column chromatography on silica gel with 2% EtOAc in petroleum ether, to afford 16 g (52%) of the product as white oil. ¹H NMR (300 MHz, CDCl₃) δ: 8.34-8.29 (m, 1H), 7.25-7.16 (m, 2H), 6.78 (s, 1H), 1.53 (s, 9H).

### Step 3

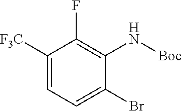

### tert-butyl 6-bromo-2-fluoro-3-(trifluoromethyl)phenylcarbamate

A 1 L 3-necked round bottom flask was charged with tert-butyl 2-fluoro-3-(trifluoromethyl)phenylcarbamate (10 g, 35.8 mmol) and dry THF (300 mL). To the above was added dropwise t-BuLi solution (1.3 M, 55.2 mL, 71.8 mmol) at −70° C. The resulting mixture was stirred at −50° C. for 1 h, followed by dropwise addition of a solution of CBr₄ (13.1 g, 39.5 mmol) in THF (50 mL) at −70° C. The reaction mixture was stirred at room temperature for further 1 h. It was then carefully mixed with ice water and extracted with Et₂O. The combined organic layers were dried over anhydrous Na₂SO₄ and concentrated in vacuo. The residue was further purified by flash column chromatography on silica gel with 2-5% EtOAc in petroleum ether, to afford 9.4 g (73%) of the product as yellow solid. ¹H NMR (300 MHz, CDCl₃) δ: 7.49 (d, J=8.4 Hz, 1H), 7.35 (t, J=8.4 Hz, 1H), 6.07 (s, 1H), 1.50 (s, 9H).

### Step 4

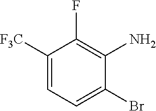

### 6-Bromo-2-fluoro-3-(trifluoromethyl)aniline

A 1 L 3-necked round bottom flask was charged with tert-butyl 6-bromo-2-fluoro-3-(trifluoromethyl)phenylcarbamate (9.4 g, 26 mmol), trifluoroacetic acid (40 mL) and CH₂Cl₂ (50 mL). The resulting mixture was stirred at room temperature for 1 h. Reaction progress was monitored by TLC (EtOAc/Petroleum ether=1:10). Work-up: the reaction mixture was concentrated in vacuo. The residue was re-dissolved in EtOAc (200 mL) and washed with brine (50 mL). The organic layer was dried over anhydrous Na₂SO₄ and then concentrated in vacuo. The residue was further purified by flash column chromatography on silica gel with 5% EtOAc in petroleum ether, to afford 6.2 g (91%) of the product.

### Step 5

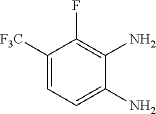

### 3-Fluoro-4-(trifluoromethyl)benzene-1,2-diamine

A 200 mL pressure tube was charged with 6-bromo-2-fluoro-3-(trifluoromethyl)aniline (7.0 g, 27 mmol), Cu₂O (1.0 g, 7.0 mmol), CuCl (1.0 g, 10 mmol) and saturated methanolic ammonia solution (100 mL). The tube was sealed and the resulting mixture was stirred at 150° C. overnight. Work-up: the reaction mixture was concentrated in vacuo. The residue was purified by flash column chromatography on silica gel with 30% EtOAc in petroleum ether, to afford 2.8 g (53%) of the product. ¹H NMR (300 MHz, CDCl₃) δ: 6.91 (t, J=7.5 Hz, 1H), 6.48 (d, J=8.4 Hz, 1H), 3.80 (s, 2H), 3.36 (s, 2H).

### Steps 6-10

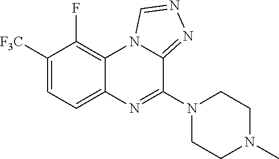

### 9-fluoro-4-(4-methylpiperazin-1-yl)-8-(trifluoromethyl)-[1,2,4]triazolo[4,3-a]quinoxaline

The title compound was prepared as described in Example 23, except that 3-fluoro-4-(trifluoromethyl)benzene-1,2-diamine was substituted for 4-(trifluoromethyl)benzene-1,2-diamine in step 1 of that route. ¹H NMR (300 MHz, CDCl₃) δ: 9.40 (d, J=2.4 Hz, 1H), 7.63 (t, J=8.1 Hz, 1H), 7.48 (d, J=10.2 Hz, 1H), 4.58 (br, 4H), 2.62 (t, J=4.8 Hz, 4H), 2.38 (s, 3H). MS m/z: 355 (M+H⁺).

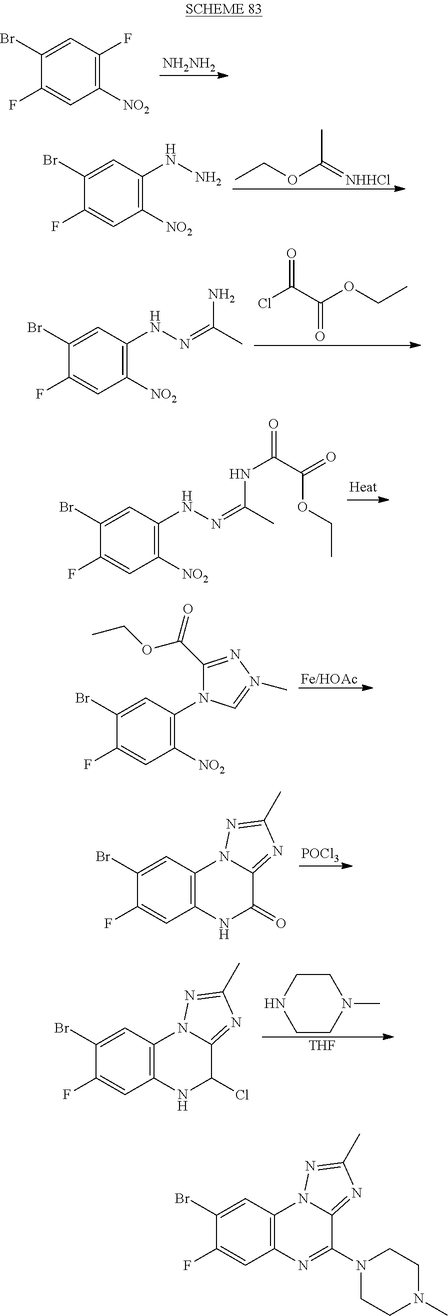

### EXAMPLE 237

### 8-bromo-7-fluoro-2-methyl-4-(4-methylpiperazin-1-yl)-[1,2,4]triazolo[1,5-a]quinoxaline

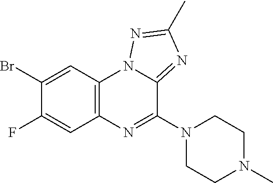

### Step 1

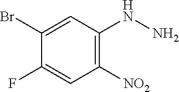

### (5-bromo-4-fluoro-2-nitrophenyl)hydrazine

A 250 mL round bottom flask was charged with 1-bromo-2,5-difluoro-4-nitrobenzene (5.0 g, 21 mmol) and ethanol (70 mL). To the solution was added dropwise hydrazine hydrate (2.1 mL, 42 mmol) at 0° C. The resulting mixture was stirred overnight at room temperature. Reaction progress was monitored by TLC (EtOAc/Petroleum ether=1:3). Work-up: the reaction mixture was partitioned between EtOAc (200 mL) and brine (100 mL). The organic layer was dried over anhydrous Na₂SO₄ then concentrated in vacuo to afford 5.2 g (quantitative yield) of the product, which was fairly pure and used in next step without further purification.

### Steps 2-7

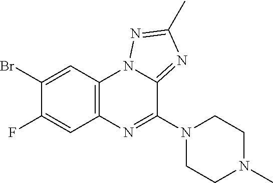

### 8-bromo-7-fluoro-2-methyl-4-(4-methylpiperazin-1-yl)-[1,2,4]triazolo[1,5-a]quinoxaline

The title compound was prepared as described in Example 165 steps 2-7, except that (5-bromo-4-fluoro-2-nitrophenyl)hydrazine was substituted for 5-chloro-2-nitrophenylhydrazine in step 2 of that route. ¹H NMR (300 MHz, DMSO-d₆) δ: 8.36 (d, J=7.2 Hz, 1H), 7.40 (d, J=9.6 Hz, 1H), 4.39 (br, 4H), 2.63 (s, 3H), 2.58 (t, J=5.1 Hz, 4H), 2.36 (s, 3H). MS m/z: 379 (M+H⁺).

### EXAMPLE 238

### 8-bromo-7-fluoro-2-methyl-4-(piperazin-1-yl)-[1,2,4]triazolo[1,5-a]quinoxaline

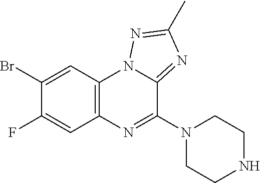

The title compound was prepared as described in Example 237, except that piperazine was substituted for N-methylpiperazine in the last step of that route. ¹H NMR (300 MHz, DMSO-d₆) δ: 8.35 (d, J=6.9 Hz, 1H), 7.39 (d, J=9.6 Hz, 1H), 4.34 (t, J=5.1 Hz, 4H), 3.05 (t, J=5.1 Hz, 4H), 2.63 (s, 3H). MS m/z: 365 (M+H⁺).

### EXAMPLE 239

### 7-fluoro-2,8-dimethyl-4-(4-methylpiperazin-1-yl)-[1,2,4]triazolo[1,5-a]quinoxaline

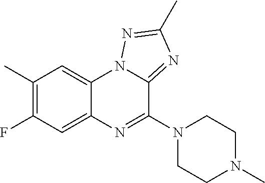

The title compound was prepared as described in Example 237, except that 2,5-difluoro-4-nitrotoluene was substituted for 1-bromo-2,5-difluoro-4-nitrobenzene as the starting material of that route. ¹H NMR (300 MHz, CD₃OD) δ: 7.93 (d, J=7.2 Hz, 1H), 7.26 (d, J=10.8 Hz, 1H), 4.30 (t, J=4.8 Hz, 4H), 2.62 (t, J=5.1 Hz, 4H), 2.58 (s, 3H), 2.39 (d, J=1.5 Hz, 3H), 2.36 (s, 3H). MS m/z: 315 (M+H⁺).

### EXAMPLE 240

### 7-fluoro-2,8-dimethyl-4-(piperazin-1-yl)-[1,2,4]triazolo[1,5-a]quinoxaline

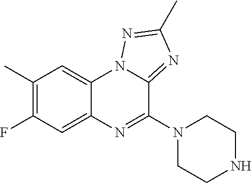

The title compound was prepared as described in Example 239, except that piperazine was substituted for N-methylpiperazine in the last step of that route. ¹H NMR (300 MHz, CD₃OD) δ: 7.89 (d, J=7.5 Hz, 1H), 7.23 (d, J=10.8 Hz, 1H), 4.31 (t, J=4.8 Hz, 4H), 3.09 (t, J=5.4 Hz, 4H), 2.57 (s, 3H), 2.38 (d, J=2.1 Hz, 3H). MS m/z: 301 (M+H⁺).

### EXAMPLE 241

### 8-bromo-2-methyl-4-(4-methylpiperazin-1-yl)-[1,2,4]triazolo[1,5-a]quinoxaline

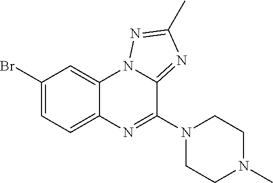

The title compound was prepared as described in Example 237, except that 1-bromo-3-fluoro-4-nitrobenzene was substituted for 1-bromo-2,5-difluoro-4-nitrobenzene as the starting material of that route. ¹H NMR (300 MHz, CDCl₃) δ: 8.33 (m, 1H), 7.55 (m, 2H), 4.37 (t, J=4.8 Hz, 4H), 2.64 (s, 3H), 2.60 (t, J=5.1 Hz, 4H), 2.37 (s, 3H). MS m/z: 361 (M+H⁺).

### EXAMPLE 242

### 8-bromo-2-methyl-4-(piperazin-1-yl)-[1,2,4]triazolo[1,5-a]quinoxaline

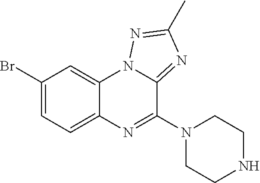

The title compound was prepared as described in Example 241, except that piperazine was substituted for N-methylpiperazine in the last step of that route. ¹H NMR (300 MHz, CDCl₃) δ: 8.33 (m, 1H), 7.54 (m, 2H), 4.32 (t, J=5.1 Hz, 4H), 3.06 (t, J=4.8 Hz, 4H), 2.64 (s, 3H). MS m/z: 347 (M+H⁺).

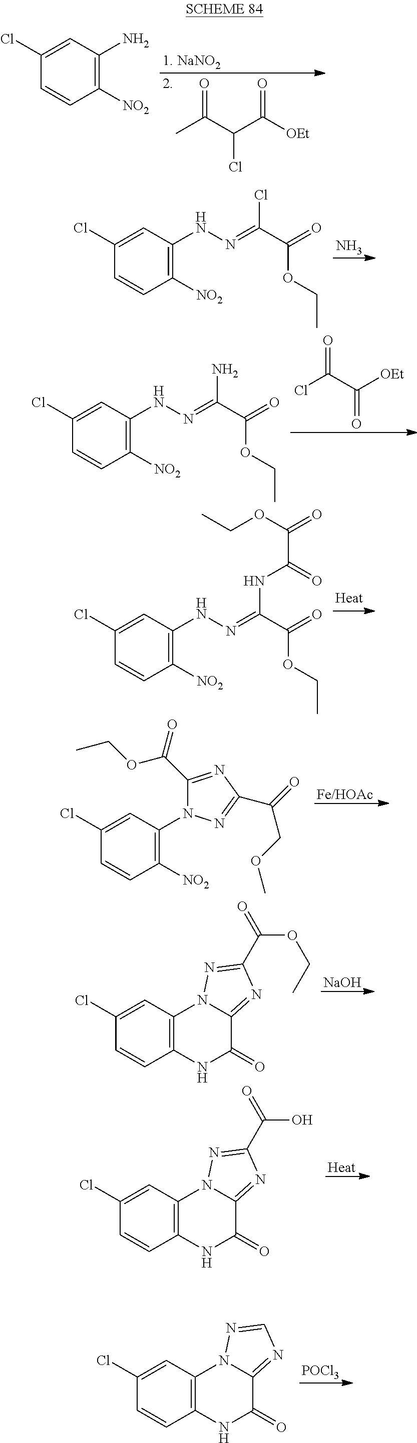

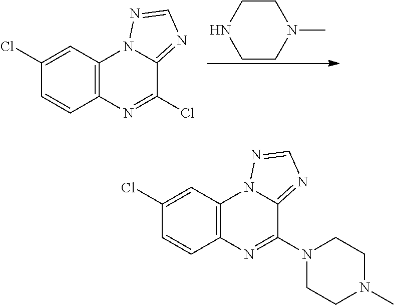

### EXAMPLE 243

### 8-chloro-4-(4-methylpiperazin-1-yl)-[1,2,4]triazolo[1,5-a]quinoxaline

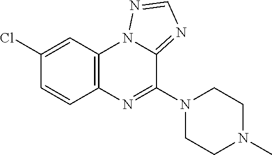

### Step 1

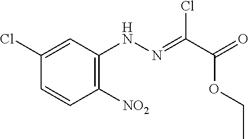

### (Z)-ethyl 2-chloro-2-(2-(5-chloro-2-nitrophenyl)hydrazono)acetate

A 500 mL round bottom flask was charged with 5-chloro-2-nitroaniline (14.8 g, 0.086 mol), concentrated HCl (40 mL), ethanol (20 mL) and water (20 mL). To the above was added dropwise a solution of NaNO₂ (6.5 g, 0.094 mol) in water (50 mL) at 0-5° C., followed by the addition of a cold solution of ethyl 2-chloroacetoacetate (12.7 g, 0.086 mol) and sodium acetate (8.08 g, 0.097 mol) in ethanol (370 mL) and water (40 mL). The reaction mixture was stirred at −5° C. for 4 h. Work up: The reaction was quenched with water (1.5 L) and stirred for further 2 h. The solid was collected and recrystallized from ethanol to give 20.5 g (78%) of the product. ¹H NMR (300 MHz, CDCl₃) δ: 11.39 (s, 1H), 8.20 (d, J=9.0 Hz, 1H), 7.95 (d, J=1.8 Hz, 1H), 7.06-7.02 (m, 1H), 4.48-4.40 (m, 2H), 1.46-1.41 (m, 3H).

### Step 2

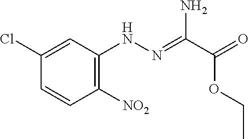

### (Z)-ethyl 2-amino-2-(2-(5-chloro-2-nitrophenyl)hydrazono)acetate

A 500 mL round bottom flask was charged with (Z)-ethyl 2-chloro-2-(2-(5-chloro-2-nitrophenyl)hydrazono)acetate (20.5 g, 0.067 mol) and THF (250 mL). Ammonia gas was introduced by bubbling through the reaction solution for 4 h. The reaction progress was monitored by TLC (EtOAc/Petroleum ether=1:4, Rf=0.5). Work up: The reaction solution was concentrated in vacuo to give 19.1 g (quantitative yield) of the product. MS m/z: 286 (M+H⁺).

### Steps 3-5

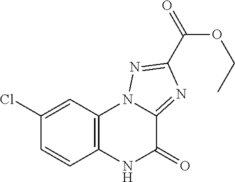

### Ethyl 8-chloro-4-oxo-4,5-dihydro-[1,2,4]triazolo[1,5-a]quinoxaline-2-carboxylate

The title compound was prepared as described in Example 164 steps 3-5, except that (Z)-ethyl 2-amino-2-(2-(5-chloro-2-nitrophenyl)hydrazono)acetate was substituted for ((1Z)-2-amino-1-azaprop-1-enyl)(5-chloro-2-nitrophenyl)amine in step 3 of that route. MS m/z: 293 (M+H⁺).

### Step 6

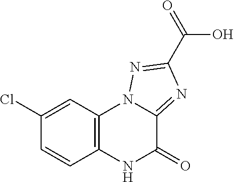

### 8-chloro-4-oxo-4,5-dihydro-[1,2,4]triazolo[1,5-a]quinoxaline-2-carboxylic acid

A 500 mL round bottom flask was charged with ethyl 8-chloro-4-oxo-4,5-dihydro-[1,2,4]triazolo[1,5-a]quinoxaline-2-carboxylate (1.5 g, 5.1 mmol), NaOH (4.0 g, 0.1 mol), water (85 mL) and ethanol (85 mL). The resulting mixture was heated at reflux for 3 h. The reaction progress was monitored by LC-MS. Work up: the solid was collected and dissolved in water (20 mL). To the aqueous solution was added dropwise 6N HCl (2 mL). The precipitate was collected by filtration, washed with water and dried, to afford 1.35 g (99%) of the product. ¹H NMR (300 MHz, DMSO-d₆) δ: 12.52 (s, 1H), 8.07 (s, 1H), 7.60 (d, J=8.1 Hz, 1H), 7.46 (d, J=8.1 Hz, 1H). MS m/z: 263 (M−H⁺).

### Step 7

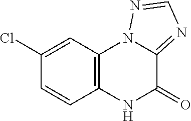

### 8-chloro-[1,2,4]triazolo[1,5-a]quinoxalin-4(5H)-one

A 50 mL round bottom flask was charged with 8-chloro-4-oxo-4,5-dihydro-[1,2,4]triazolo[1,5-a]quinoxaline-2-carboxylic acid (1.35 g, 5.1 mmol), Cu₂O (20 mg, 0.13 mmol) and HO(CH₂CH₂O)₂H (30 mL). The resulting mixture was heated at 135° C. overnight. The reaction progress was monitored by LC-MS. Work up: the solid was collected by filtration, washed with 0.5 M aqueous NaHCO₃ (10 mL) and then with a few drops of ammonia/ammonium chloride buffer (PH 9), and dried, to afford 0.84 g (75%) of the product. MS m/z: 219 (M−H⁺).

### Steps 8-9

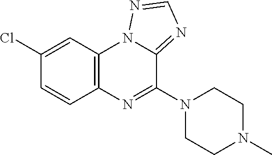

### 8-chloro-4-(4-methylpiperazin-1-yl)-[1,2,4]triazolo[1,5-a]quinoxaline

The title compound was prepared as described in Example 164 steps 6-7, except that 8-chloro-[1,2,4]triazolo[1,5-a]quinoxalin-4(5H)-one was substituted for 8-chloro-2-methyl-[1,2,4]triazolo[1,5-a]quinoxalin-4(5H)-one in step 6 and N-methylpiperazine for piperazine in step 7 of that route. ¹H NMR (300 MHz, CDCl₃) δ: 8.37 (s, 1H), 8.23 (d, J=2.1 Hz, 1H), 7.65 (d, J=9.0 Hz, 1H), 7.44 (dd, J=9.0, 2.1 Hz, 1H), 4.38 (t, J=5.0 Hz, 4H), 2.60 (t, J=5.1 Hz, 4H), 2.37 (s, 3H). MS m/z: 303 (M+H⁺).

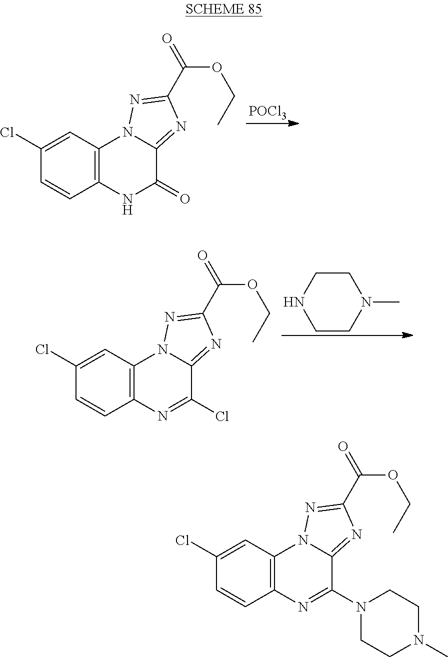

### EXAMPLE 244

### Ethyl 8-chloro-4-(4-methylpiperazin-1-yl)-[1,2,4]triazolo[1,5-a]quinoxaline-2-carboxylate

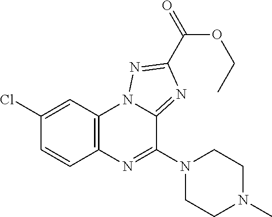

The title compound was prepared as described in Example 164 steps 6-7, except that ethyl 8-chloro-4-oxo-4,5-dihydro-[1,2,4]triazolo[1,5-a]quinoxaline-2-carboxylate (prepared as described in Example 243, steps 1-5) was substituted for 8-chloro-2-methyl-[1,2,4]triazolo[1,5-a]quinoxalin-4(5H)-one in step 6 and N-methylpiperazine for piperazine in step 7 of that route. ¹H NMR (300 MHz, CDCl₃) δ: 8.36 (d, J=2.1 Hz, 1H), 7.65 (d, J=9.0 Hz, 1H), 7.48 (dd, J=9.0, 2.1 Hz, 1H), 4.58 (q, J=7.2 Hz, 2H), 4.41 (br, 4H), 2.60 (t, J=5.1 Hz, 4H), 2.37 (s, 3H), 1.50 (t, J=7.2 Hz, 3H). MS m/z: 375 (M+H⁺).

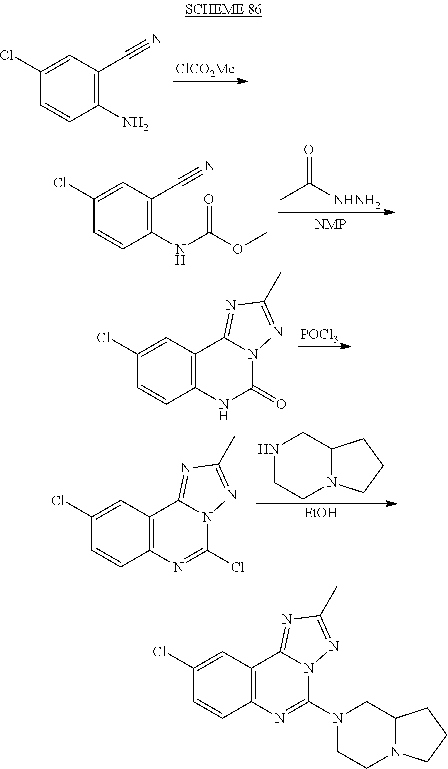

### EXAMPLE 245

### 9-chloro-5-(hexahydropyrrolo[1,2-a]pyrazin-2(1H)-yl)-2-methyl-[1,2,4]triazolo[1,5-c]quinazoline

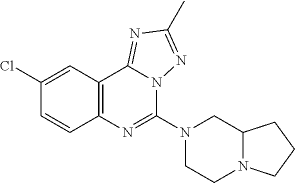

The title compound was prepared as described in Example 111, except that 1,4-diazabicyclo[4.3.0]nonane was substituted for N-methylpiperazine in the last step of that route. ¹H NMR (300 MHz, DMSO-d₆) δ: 8.12 (d, J=2.4 Hz, 1H), 7.70 (dd, J=8.7, 2.4 Hz, 1H), 7.63 (d, J=8.7 Hz, 1H), 4.90 (m, 2H), 3.28-2.99 (m, 3H), 2.85 (m, 1H), 2.52 (s, 3H), 2.34-2.27 (m, 1H), 2.14-2.05 (m, 2H), 1.83-1.64 (m, 3H), 1.40-1.35 (m, 1H). MS m/z: 343 (M+H⁺).

### EXAMPLE 246

### 9-chloro-7-fluoro-2-methyl-5-(4-methylpiperazin-1-yl)-[1,2,4]triazolo[1,5-c]quinazoline

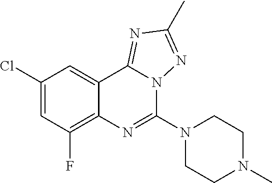

The title compound was prepared as described in Example 122, except that 4-chloro-2-fluoroaniline was substituted for 3-chloro-4-(trifluoromethyl)aniline as the starting material of that route. ¹H NMR (300 MHz, CDCl₃) δ: 8.08 (dd, J=2.1, 1.2 Hz, 1H), 7.37 (dd, J=10.2, 2.4 Hz, 1H), 4.17 (t, J=4.8 Hz, 4H), 2.64 (t, J=5.4 Hz, 4H), 2.64 (s, 3H), 2.38 (s, 3H). MS m/z: 335 (M+H⁺).

### EXAMPLE 247

### 9-chloro-7-fluoro-2-methyl-5-(piperazin-1-yl)-[1,2,4]triazolo[1,5-c]quinazoline

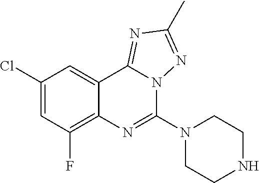

The title compound was prepared as described in Example 246, except that piperazine was substituted for N-methylpiperazine in the last step of that route. ¹H NMR (300 MHz, CD₃OD) δ: 7.92 (dd, J=2.4, 1.8 Hz, 1H), 7.46 (dd, J=10.2, 2.4 Hz, 1H), 4.12 (t, J=5.1 Hz, 4H), 3.09 (t, J=5.1 Hz, 4H), 2.59 (s, 3H). MS m/z: 321 (M+H⁺).

### EXAMPLE 248

### 9-bromo-7-fluoro-2-methyl-5-(4-methylpiperazin-1-yl)-[1,2,4]triazolo[1,5-c]quinazoline

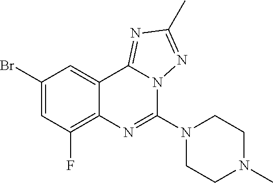

The title compound was prepared as described in Example 122, except that 4-bromo-2-fluoroaniline was substituted for 3-chloro-4-(trifluoromethyl)aniline as the starting material of that route. ¹H NMR (300 MHz, CDCl₃) δ: 8.25 (dd, J=2.1, 1.5 Hz, 1H), 7.50 (dd, J=9.9, 2.1 Hz, 1H), 4.23 (t, J=4.8 Hz, 4H), 2.72 (t, J=5.1 Hz, 4H), 2.63 (s, 3H), 2.43 (s, 3H). MS m/z: 379 (M+H⁺).

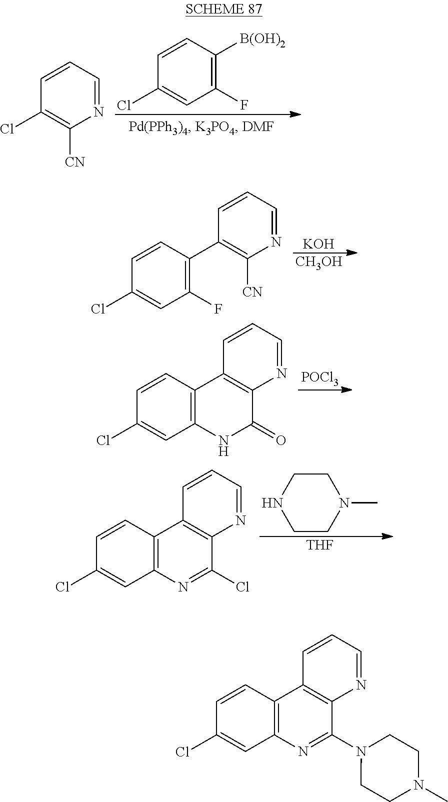

### EXAMPLE 249

### 8-chloro-5-(4-methylpiperazin-1-yl)benzo[f][1,7]naphthyridine

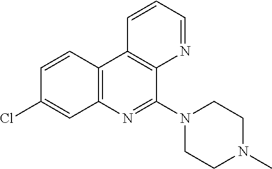

### Step 1

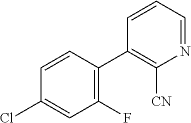

### 3-(4-chloro-2-fluorophenyl)picolinonitrile

A 20 mL microwave reaction tube was charged with 3-chloro-2-cyanopyridine (1.00 g, 7.2 mmol), 4-chloro-2-fluorophenylboronic acid (1.51 g, 8.7 mmol), Pd(PPh₃)₄ (417 mg, 0.36 mmol), K₃PO₄ (3.8 g, 18 mmol) and DMF (15 mL). After O₂ was purged by bubbling N₂ into the reaction solution, the tube was sealed and heated at 150° C. for 0.5 h in a Biotage microwave reactor. Work-up: the reaction mixture was poured into water (150 mL) and extracted with EtOAc (100 mL×3). The combined organic layers were dried over anhydrous Na₂SO₄ and then concentrated in vacuo. The residue was further purified by flash column chromatography on silica gel with 50% CH₂Cl₂ in petroleum ether, to afford 0.53 g (32%) of the product as white solids. ¹H NMR (300 MHz, CDCl₃) δ: 8.73 (dd, J=4.8, 1.6 Hz, 1H), 7.85 (dt, J=8.0, 1.4 Hz, 1H), 7.60 (dd, J=8.0, 4.7 Hz, 1H), 7.41 (t, J=8.2 Hz, 1H), 7.33-7.26 (m, 2H).

### Step 2

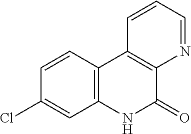

### 8-chlorobenzo[f][1,7]naphthyridin-5(6H)-one

A 20 mL microwave reaction tube was charged with 3-(4-chloro-2-fluorophenyl)picolinonitrile (0.44 g, 1.9 mmol), KOH (0.53 g, 9.5 mol) and methanol (10 mL). The tube was sealed and heated at 120° C. for 1 h in a Biotage microwave reactor. Work-up: the reaction mixture was poured into water (100 mL) and extracted with EtOAc (100 mL×4). The combined organic layers were dried over anhydrous Na₂SO₄ and then concentrated in vacuo, to afford 0.24 g (55%) of the product as white solids. ¹H NMR (300 MHz, DMSO-d₆) δ: 11.94 (br, 1H), 8.93-8.87 (m, 2H), 8.41 (d, J=8.8 Hz, 1H), 7.83 (dd, J=8.2, 4.4 Hz, 1H), 7.39 (d, J=2.0 Hz, 1H), 7.30 (dd, J=8.8, 2.0 Hz, 1H).

### Step 3

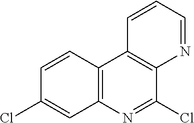

### 5,8-dichlorobenzo[f][1,7]naphthyridine

A 100 mL round bottom flask was charged with 8-chlorobenzo[f][1,7]naphthyridin-5(6H)-one (0.24 g, 1.0 mmol) and POCl₃ (50 mL). The resulting mixture was refluxed for 3 h and then concentrated in vacuo. The residue was carefully diluted with saturated aqueous NaHCO₃ (150 mL) and extracted with EtOAc (100 mL×3). The combined organic layers were dried over anhydrous Na₂SO₄ then concentrated in vacuo. The residue was further purified by flash column chromatography on silica gel with 0-2% CH₃OH in CH₂Cl₂, to afford 0.20 g (77%) of the product as white solids. ¹H NMR (300 MHz, CDCl₃) δ: 9.16 (dd, J=4.4, 1.5 Hz, 1H), 8.86 (dd, J=8.5, 1.5 Hz, 1H), 8.40 (d, J=8.8 Hz, 1H), 8.13 (d, J=2.2 Hz, 1H), 7.85 (dd, J=8.5, 4.4 Hz, 1H), 7.69 (dd, J=8.8, 2.2 Hz, 1H).

### Step 4

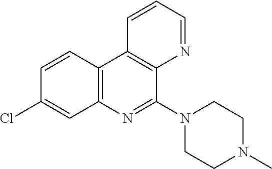

### 8-chloro-5-(4-methylpiperazin-1-yl)benzo[f][1,7]naphthyridine

A 20 mL microwave reaction tube was charged with 5,8-dichlorobenzo[f][1,7]naphthyridine (0.24 g, 0.96 mmol), N-methylpiperazine (0.33 mL, 3.0 mmol) and THF (10 mL). The tube was sealed and heated at 90° C. for 1 h in a Biotage microwave reactor. Work-up: the reaction mixture was poured into saturated aqueous NaHCO₃ (60 mL) and extracted with CH₂Cl₂ (50 mL×3). The combined organic layers were dried over anhydrous Na₂SO₄ and concentrated in vacuo. The residue was further purified by flash column chromatography on silica gel with CH₂Cl₂ (saturated with NH₃), to afford 0.26 g (86%) of the product as off-white solids. ¹H NMR (300 MHz, CDCl₃) δ: 8.91 (dd, J=4.3, 1.7 Hz, 1H), 8.74 (dd, J=8.4, 1.7 Hz, 1H), 8.19 (d, J=8.7 Hz, 1H), 7.84 (d, J=2.1 Hz, 1H), 7.66 (dd, J=8.4, 4.3 Hz, 1H), 7.36 (dd, J=8.7, 2.1 Hz, 1H), 4.13 (t, J=4.7 Hz, 4H), 2.70 (t, J=5.0 Hz, 4H), 2.39 (s, 3H). MS: m/z 313 (M+H⁺).

### EXAMPLE 250

### 8-chloro-5-(4-methylpiperazin-1-yl)pyrazino[2,3-c]quinoline

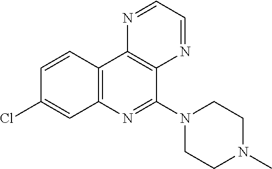

The title compound was prepared as described in Example 229, except that 2-chloro-3-cyanopyrazine was substituted for 3-chloro-2-cyanopyridine as the starting material. ¹H NMR (300 MHz, CDCl₃) δ: 8.96 (d, J=1.9 Hz, 1H), 8.82 (d, J=1.9 Hz, 1H), 8.71 (d, J=8.7 Hz, 1H), 7.80 (d, J=2.1 Hz, 1H), 7.39 (dd, J=8.7, 2.1 Hz, 1H), 4.14 (t, J=5.0 Hz, 4H), 2.68 (t, J=5.0 Hz, 4H), 2.39 (s, 3H). MS: m/z 314 (M+H⁺).

The following compounds can generally be made using the methods known in the art and/or as shown above. It is expected that these compounds when made will have activity similar to those that have been made in the examples above.

The following compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36. All SMILES strings used herein, as well as numerous IUPAC names, were generated using CambridgeSoft's ChemDraw ChemBioDraw Ultra 11.0.

C1CN(CCN1)C3=NC2=CC(═CC═C2N4N═NN═C34)Cl FC(F)(F)C=1C═CC=2N═C(C3=NN═NN3(C=2(C=1)))N4CCNCC4 CC2=NC=3C(═NC=1C═C(F)C(═CC=1C=3(O2))Br)N4CCN(C)CC4 CC2=NC=3C(═NC=1C═C(F)C(═CC=1C=3(O2))Br)N4CCNCC4 C1CN(CCN1)C3=NC2=CC═C(C═C2N4N═CN═C34)Cl FC4=CC(═CC1=C4(N═C(C2=NN═NN12)N3CCNCC3))Br CN1CCN(CC1)C3=NC=2C(F)═CC(═CC=2N4N═NN═C34)Cl FC4=CC(═CC1=C4(N═C(C2=NN═NN12)N3CCNCC3))Cl CC=2N═C3C=4C═C(C═C(F)C=4(N═C(N1CCNCC1)N3(N=2)))Br CC2=NC=3C(═NC1=CC═C(C═C1C=3(O2))Br)N4CCN(C)CC4 CC2=NC=3C(═NC1=CC═C(C═C1C=3(O2))Br)N4CCNCC4 CCOC(═O)C=2N═C3C(═NC1=CC═C(C═C1N3(N=2))Cl)N4CCNCC4 CN1CCN(CC1)C4=NC=2C═C(F)C(═CC=2C3=CON═C34)Cl FC1=CC=2N═C(C3=NOC═C3(C=2(C═C1Cl)))N4CCNCC4 C=1C═NC2=C(C=1)C=4C═C(C═CC=4(N═C2N3CCNCC3))Cl CN1CCN(CC1)C4=NC=2C═CC(═CC=2C=3N═CC═NC=34)Cl CN1CCN(CC1)C4=NC=2C═CC(═CC=2C=3C═CC═NC=34)Cl C=1C═NC2=C(N=1)C=4C═C(C═CC=4(N═C2N3CCNCC3))Cl CN1CCN(CC1)C4=NC=2C═CC(═CC=2C=3C═NC═NC=34)Cl C1CN(CCN1)C4=NC=2C═CC(═CC=2C=3C═NC═NC=34)Cl CC=2N═C3C(═NC1=CC(F)═C(C═C1N3(N=2))Cl)N4CCN(C)CC4 CC=2N═C3C(═NC1=CC(F)═C(C═C1N3(N=2))Cl)N4CCNCC4 CN1CCN(CC1)C4=NC=2C═C(F)C(═CC=2C3=CON═C34)Br FC1=CC=2N═C(C3=NOC═C3(C=2(C═C1Br)))N4CCNCC4 CC=2N═C3C=4C═C(C(F)═CC=4(N═C(N1CCN(C)CC1)N3(N=2)))Br CC=2N═C3C=4C═C(C(F)═CC=4(N═C(N1CCNCC1)N3(N=2)))Br CC=2N═C3C(═NC1=C(F)C═C(C═C1N3(N=2))Br)N4CCN(C)CC4 CC=2N═C3C(═NC1=C(F)C═C(C═C1N3(N=2))Br)N4CCNCC4 CN1CCN(CC1)C3=NC4=C(F)C═C(C═C4(C2=CON═C23))Br FC=2C═C(C═C3C1=CON═C1C(═NC=23)N4CCNCC4)Br CN1CCN(CC1)C3=NC2=CC═C(C═C2N4N═C(N═C34)C(═O)O)Cl O═C(O)C=2N═C3C(═NC1=CC═C(C═C1N3(N=2))Cl)N4CCNCC4 CN(CC4)CCN4C(C2=NN═CN23)=NC1=C3C═C(C(F)(F)C(F)(F)F)C═C1 FC(C(F)(F)F)(F)C1=CC3=C(N═C(N4CCNCC4)C2=NN═CN23)C═C1 CC=2N═C3C(═NC1=CC═C(C═C1N3(N=2))C(F)(F)F)N4CCN(C)CC4 CC=2N═C3C(═NC1=CC═C(C═C1N3(N=2))C(F)(F)F)N4CCNCC4 CN1CCN(CC1)C4=NC=2C═CC(═CC=2C3=C4(N═CS3))Br C1CN(CCN1)C4=NC=2C═CC(═CC=2C3=C4(N═CS3))Br C1CC2CN(CCN2(C1))C5=NC=3C═CC(═CC=3C4=C5(N═CS4))Br CN1CCN(CC1)C4=NC=2C═C(F)C(═CC=2C3=C4(N═CS3))Br FC1=CC=2N═C(C=3N═CSC=3(C=2(C═C1Br)))N4CCNCC4 FC1=CC=2N═C(C=3N═CSC=3 (C=2(C═C1Br)))N4CCN5CCCC5 (C4) CN1CCN(CC1)C4=NC=2C(F)═CC(═CC=2C3=C4(N═CS3))Br FC4=CC(═CC1=C4(N═C(C=2N═CSC1=2)N3CCNCC3))Br FC5=CC(═CC1=C5(N═C(C=2N═CSC1=2)N3CCN4CCCC4(C3)))Br CN1CCN(CC1)C4=NC=2C(F)═C(F)C(═CC=2C3=C4(N═CS3))Br FC4=C(F)C(═CC1=C4(N═C(C=2N═CSC1=2)N3CCNCC3))Br FC5=C(F)C(═CC1=C5(N═C(C=2N═CSC1=2)N3CCN4CCCC4(C3)))Br CN1CCN(CC1)C4=NC=2C═CC(═CC=2C3=C4(N═CS3))Cl C1CN(CCN1)C4=NC=2C═CC(═CC=2C3=C4(N═CS3))Cl C1CC2CN(CCN2(C1))C5=NC=3C═CC(═CC=3C4=C5(N═CS4))Cl CN1CCN(CC1)C4=NC=2C═C(F)C(═CC=2C3=C4(N═CS3))Cl FC1=CC=2N═C(C=3N═CSC=3(C=2(C═C1Cl)))N4CCNCC4 FC1=CC=2N═C(C=3N═CSC=3 (C=2(C═C1Cl)))N4CCN5CCCC5 (C4) CN1CCN(CC1)C4=NC=2C(F)═CC(═CC=2C3=C4(N═CS3))Cl FC4=CC(═CC1=C4(N═C(C=2N═CSC1=2)N3CCNCC3))Cl FC5=CC(═CC1=C5(N═C(C=2N═CSC1=2)N3CCN4CCCC4(C3)))Cl CN1CCN(CC1)C4=NC=2C(F)═C(F)C(═CC=2C3=C4(N═CS3))Cl FC4=C(F)C(═CC1=C4(N═C(C=2N═CSC1=2)N3CCNCC3))Cl FC5=C(F)C(═CC1=C5(N═C(C=2N═CSC1=2)N3CCN4CCCC4(C3)))Cl CN1CCN(CC1)C4=NC=2C═CC(═CC=2C3=C4(N═CS3))C(F)(F)F FC(F)(F)C=1C═CC=2N═C(C=3N═CSC=3(C=2(C=1)))N4CCNCC4 FC(F)(F)C=1C═CC=2N═C(C=3N═CSC=3 (C=2(C=1)))N4CCN5CCCC5 (C4) CN1CCN(CC1)C4=NC=2C═C(F)C(═CC=2C3=C4(N═CS3))C(F)(F)F FC1=CC=2N═C(C=3N═CSC=3(C=2(C═C1C(F)(F)F)))N4CCNCC4 FC1=CC=2N═C(C=3N═CSC=3 (C=2(C═C1C(F)(F)F)))N4CCN5CCCC5 (C4) CN1CCN(CC1)C4=NC=2C(F)═CC(═CC=2C3=C4(N═CS3))C(F)(F)F FC4=CC(═CC1=C4(N═C(C=2N═CSC1=2)N3CCNCC3))C(F)(F)F FC5=CC(═CC1=C5(N═C(C=2N═CSC1=2)N3CCN4CCCC4(C3)))C(F)(F)F CN1CCN(CC1)C4=NC=2C(F)═C(F)C(═CC=2C3=C4(N═CS3))C(F)(F)F FC4=C(F)C(═CC1=C4(N═C(C=2N═CSC1=2)N3CCNCC3))C(F)(F)F FC5=C(F)C(═CC1=C5(N═C(C=2N═CSC1=2)N3CCN4CCCC4(C3)))C(F)(F)F CN1CCN(CC1)C4=NC=2C═C(C(═CC=2C3=C4(N═CS3))C(F)(F)F)Cl FC(F)(F)C1=CC2=C(C═C1Cl)N═C(C=3N═CSC2=3)N4CCNCC4 FC(F)(F)C1=CC2=C(C═C1Cl)N═C(C=3N═CSC2=3)N4CCN5CCCC5 (C4) CN1CCN(CC1)C4=NC=2C═C(F)C(═CC=2C3=CC═NN34)Br FC2=CC=3N═C(N1CCNCC1)N4N═CC═C4(C=3(C═C2Br)) FC3=CC=4N═C(N1CCN2CCCC2(C1))N5N═CC═C5(C=4(C═C3Br)) CN1CCN(CC1)C4=NC=2C═CC(═CC=2C3=CC═NN34)Br C=2C═C3C=4C═C(C═CC=4(N═C(N1CCNCC1)N3(N=2)))Br C1CC2CN(CCN2(C1))C5=NC=3C═CC(═CC=3C4=CC═NN45)Br CN1CCN(CC1)C3=NC=4C(F)═CC(═CC=4(C2=CC═NN23))Br FC2=CC(═CC=3C1=CC═NN1C(═NC2=3)N4CCNCC4)Br FC2=CC(═CC=3C1=CC═NN1C(═NC2=3)N4CCN5CCCC5(C4))Br CN1CCN(CC1)C3=NC=4C(F)═C(F)C(═CC=4(C2=CC═NN23))Br FC2=C(F)C(═CC=3C1=CC═NN1C(═NC2=3)N4CCNCC4)Br FC2=C(F)C(═CC=3C1=CC═NN1C(═NC2=3)N4CCN5CCCC5(C4))Br CN1CCN(CC1)C4=NC=2C═C(F)C(═CC=2C3=CC═NN34)Cl FC2=CC=3N═C(N1CCNCC1)N4N═CC═C4(C=3(C═C2Cl)) FC3=CC=4N═C(N1CCN2CCCC2(C1))N5N═CC═C5(C=4(C═C3Cl)) CN1CCN(CC1)C4=NC=2C═CC(═CC=2C3=CC═NN34)Cl C=2C═C3C=4C═C(C═CC=4(N═C(N1CCNCC1)N3(N=2)))Cl C1CC2CN(CCN2(C1))C5=NC=3C═CC(═CC=3C4=CC═NN45)Cl CN1CCN(CC1)C3=NC=4C(F)═CC(═CC=4(C2=CC═NN23))Cl FC2=CC(═CC=3C1=CC═NN1C(═NC2=3)N4CCNCC4)Cl FC2=CC(═CC=3C1=CC═NN1C(═NC2=3)N4CCN5CCCC5(C4))Cl CN1CCN(CC1)C3=NC=4C(F)═C(F)C(═CC=4(C2=CC═NN23))Cl FC2=C(F)C(═CC=3C1=CC═NN1C(═NC2=3)N4CCNCC4)Cl FC2=C(F)C(═CC=3C1=CC═NN1C(═NC2=3)N4CCN5CCCC5(C4))Cl CN1CCN(CC1)C4=NC=2C═C(F)C(═CC=2C3=CC═NN34)C(F)(F)F FC2=CC=3N═C(N1CCNCC1)N4N═CC═C4(C=3(C═C2C(F)(F)F)) FC3=CC=4N═C(N1CCN2CCCC2(C1))N5N═CC═C5(C=4(C═C3C(F)(F)F)) CN1CCN(CC1)C4=NC=2C═CC(═CC=2C3=CC═NN34)C(F)(F)F FC(F)(F)C=2C═CC=3N═C(N1CCNCC1)N4N═CC═C4(C=3 (C=2)) FC(F)(F)C=3C═CC=4N═C(N1CCN2CCCC2(C1))N5N═CC═C5(C=4(C=3)) CN1CCN(CC1)C3=NC=4C(F)═C(F)C(═CC=4(C2=CC═NN23))C(F)(F)F FC2=C(F)C(═CC=3C1=CC═NN1C(═NC2=3)N4CCNCC4)C(F)(F)F FC1=C(F)C3=C(C2=CC═NN2C(N4CCN(CCC5)C5C4)=N3)C═C1C(F)(F)F CN1CCN(CC1)C3=NC=4C(F)═CC(═CC=4(C2=CC═NN23))C(F)(F)F FC2=CC(═CC=3C1=CC═NN1C(═NC2=3)N4CCNCC4)C(F)(F)F FC2=CC(═CC=3C1=CC═NN1C(═NC2=3)N4CCN5CCCC5(C4))C(F)(F)F CN1CCN(CC1)C4=NC=2C═C(C(═CC=2C3=CC═NN34)C(F)(F)F)Cl FC(F)(F)C1=CC3=C(C═C1Cl)N═C(N2CCNCC2)N4N═CC═C34 FC(F)(F)C1=CC4=C(C═C1Cl)N═C(N2CCN3CCCC3(C2))N5N═CC═C45 C1=CC=2C=4C═C(C═CC=4(N═C(C=2(N═N1))N3CCNCC3))Br CN1CCN(CC1)C4=NC=2C═CC(═CC=2C=3C═CN═NC=34)Br C1CC2CN(CCN2(C1))C5=NC=3C═CC(═CC=3C=4C═CN═NC=45)Br FC1=CC=2N═C(C=3N═NC═CC=3(C=2(C═C1Br)))N4CCNCC4 CN1CCN(CC1)C4=NC=2C═C(F)C(═CC=2C=3C═CN═NC=34)Br FC1=CC=2N═C(C=3N═NC═CC=3 (C=2(C═C1Br)))N4CCN5CCCC5 (C4) FC2=CC(═CC=3C=1C═CN═NC=1C(═NC2=3)N4CCNCC4)Br CN1CCN(CC1)C3=NC=4C(F)═CC(═CC=4(C=2C═CN═NC=23))Br FC2=CC(═CC=3C=1C═CN═NC=1C(═NC2=3)N4CCN5CCCC5(C4))Br FC2=C(F)C(═CC=3C=1C═CN═NC=1C(═NC2=3)N4CCNCC4)Br CN1CCN(CC1)C3=NC=4C(F)═C(F)C(═CC=4(C=2C═CN═NC=23))Br FC2=C(F)C(═CC=3C=1C═CN═NC=1C(═NC2=3)N4CCN5CCCC5(C4))Br C1=CC=2C=4C═C(C═CC=4(N═C(C=2(N═N1))N3CCNCC3))Cl CN1CCN(CC1)C4=NC=2C═CC(═CC=2C=3C═CN═NC=34)Cl C1CC2CN(CCN2(C1))C5=NC=3C═CC(═CC=3C=4C═CN═NC=45)Cl FC1=CC=2N═C(C=3N═NC═CC=3(C=2(C═C1Cl)))N4CCNCC4 CN1CCN(CC1)C4=NC=2C═C(F)C(═CC=2C=3C═CN═NC=34)Cl FC1=CC=2N═C(C=3N═NC═CC=3 (C=2(C═C1Cl)))N4CCN5CCCC5 (C4) FC2=CC(═CC=3C=1C═CN═NC=1C(═NC2=3)N4CCNCC4)Cl CN1CCN(CC1)C3=NC=4C(F)═CC(═CC=4(C=2C═CN═NC=23))Cl FC2=CC(═CC=3C=1C═CN═NC=1C(═NC2=3)N4CCN5CCCC5(C4))Cl FC2=C(F)C(═CC=3C=1C═CN═NC=1C(═NC2=3)N4CCNCC4)Cl CN1CCN(CC1)C3=NC=4C(F)═C(F)C(═CC=4(C=2C═CN═NC=23))Cl FC2=C(F)C(═CC=3C=1C═CN═NC=1C(═NC2=3)N4CCN5CCCC5(C4))Cl FC(F)(F)C1=CC2=C(C═C1Cl)N═C(C=3N═NC═CC2=3)N4CCNCC4 CN1CCN(CC1)C4=NC=2C═C(C(═CC=2C=3C═CN═NC=34)C(F)(F)F)Cl FC(F)(F)C1=CC2=C(C═C1Cl)N═C(C=3N═NC═CC2=3)N4CCN5CCCC5(C4) FC(F)(F)C(F)(F)C1=CC═C2N═C(C3=NN═CN3(C2(═C1)))N4CCNCC4 CN1CCN(CC1)C3=NC2=CC═C(C═C2N4C═NN═C34)C(F)(F)C(F)(F)F FC(F)(F)C(F)(F)C1=CC═C2N═C(C3=NN═CN3 (C2(═C1)))N4CCN5CCCC5 (C4) FC=1C═C2N═C(C3=NN═CN3(C2(═CC=1C(F)(F)C(F)(F)F)))N4CCNCC4 CN1CCN(CC1)C3=NC2=CC(F)═C(C═C2N4C═NN═C34)C(F)(F)C(F)(F)F FC=1C═C2N═C(C3=NN═CN3 (C2(═CC=1C(F)(F)C(F)(F)F)))N4CCN5CCCC5 (C4) FC=4C═C(C═C1C=4(N═C(C2=NN═CN12)N3CCNCC3))C(F)(F)C(F)(F)F CN1CCN(CC1)C3=NC2=C(F)C═C(C═C2N4C═NN═C34)C(F)(F)C(F)(F)F FC=5C═C(C═C1C=5(N═C(C2=NN═CN12)N3CCN4CCCC4(C3)))C(F)(F)C(F)(F)F FC=4C(F)═C(C═C1C=4(N═C(C2=NN═CN12)N3CCNCC3))C(F)(F)C(F)(F)F CN1CCN(CC1)C3=NC2=C(F)C(F)═C(C═C2N4C═NN═C34)C(F)(F)C(F)(F)F FC=5C(F)═C(C═C1C=5(N═C(C2=NN═CN12)N3CCN4CCCC4(C3)))C(F)(F)C(F)(F)F N#CC1=CC═C2N═C(C3=NN═CN3(C2(═C1)))N4CCNCC4 CN1CCN(CC1)C3=NC2=CC═C(C#N)C═C2N4C═NN═C34 N#CC1=CC═C2N═C(C3=NN═CN3(C2(═C1)))N4CCN5CCCC5(C4) N#CC=1C═C2C(═CC=1(F))N═C(C3=NN═CN23)N4CCNCC4 CN1CCN(CC1)C3=NC2=CC(F)═C(C#N)C═C2N4C═NN═C34 N#CC=1C═C2C(═CC=1(F))N═C(C3=NN═CN23)N4CCN5CCCC5(C4) N#CC=4C═C1C(N═C(C2=NN═CN12)N3CCNCC3)=C(F)C=4(F) CN1CCN(CC1)C3=NC2=C(F)C(F)═C(C#N)C═C2N4C═NN═C34 N#CC=5C═C1C(N═C(C2=NN═CN12)N3CCN4CCCC4(C3))═C(F)C=5(F) FC=4C(F)═C(C═C1C=4(N═C(C2=NN═CN12)N3CCNCC3))Br CN1CCN(CC1)C3=NC2=C(F)C(F)═C(C═C2N4C═NN═C34)Br FC=5C(F)═C(C═C1C=5(N═C(C2=NN═CN12)N3CCN4CCCC4(C3)))Br FC=4C(F)═C(C═C1C=4(N═C(C2=NN═CN12)N3CCNCC3))Cl CN1CCN(CC1)C3=NC2=C(F)C(F)═C(C═C2N4C═NN═C34)Cl FC=5C(F)═C(C═C1C=5(N═C(C2=NN═CN12)N3CCN4CCCC4(C3)))Cl FC=4C(F)═C(C═C1C=4(N═C(C2=NN═CN12)N3CCNCC3))C(F)(F)F CN1CCN(CC1)C3=NC2=C(F)C(F)═C(C═C2N4C═NN═C34)C(F)(F)F FC=5C(F)═C(C═C1C=5(N═C(C2=NN═CN12)N3CCN4CCCC4(C3)))C(F)(F)F CC1=CC(F)═C2N═C(C3=NN═CN3(C2(═C1)))N4CCNCC4 CC1=CC(F)═C2N═C(C3=NN═CN3(C2(═C1)))N4CCN(C)CC4 CC1=CC(F)═C2N═C(C3=NN═CN3(C2(═C1)))N4CCN5CCCC5(C4) CC=4C═C1C(N═C(C2=NN═CN12)N3CCNCC3)=C(F)C=4(F) CC=4C═C1C(N═C(C2=NN═CN12)N3CCN(C)CC3)=C(F)C=4(F) CC=5C═C1C(N═C(C2=NN═CN12)N3CCN4CCCC4(C3))=C(F)C=5(F) FC=4C═C1C(N═C(C2=NN═CN12)N3CCNCC3)=C(F)C=4(F) CN1CCN(CC1)C3=NC2=C(F)C(F)═C(F)C═C2N4C═NN═C34 FC=5C═C1C(N═C(C2=NN═CN12)N3CCN4CCCC4(C3))=C(F)C=5(F) FC(F)(F)C=1C═C2N═C(C3=NN═CN3(C2(═CC=1C(F)(F)F)))N4CCNCC4 CN1CCN(CC1)C3=NC2=CC(═C(C═C2N4C═NN═C34)C(F)(F)F)C(F)(F)F FC(F)(F)C=1C═C2N═C(C3=NN═CN3(C2(═CC=1C(F)(F)F)))N4CCN5CCCC5(C4) FC2(F)(C=1C═C3N═C(C4=NN═CN4(C3(═CC=1C(F)(F)C2(F)(F))))N5CCNCC5) CN1CCN(CC1)C3=NC2=CC5=C(C═C2N4C═NN═C34)C(F)(F)C(F)(F)C5(F)(F) FC2(F)(C=1C═C3N═C(C4=NN═CN4(C3(═CC=1C(F)(F)C2(F)(F))))N5CCN6CCCC6 (C5)) CC=2N═C3C4=CC(═CC═C4(N═C(N1CCNCC1)N3(N=2)))C(F)(F)C(F)(F)F CC=2N═C3C4=CC(═CC═C4(N═C(N1CCN(C)CC1)N3(N=2)))C(F)(F)C(F)(F)F CC=3N═C4C5=CC(═CC═C5(N═C(N1CCN2CCCC2(C1))N4(N=3)))C(F)(F)C(F)(F)F CC=2N═C3C4=CC(═C(F)C═C4(N═C(N1CCNCC1)N3(N=2)))C(F)(F)C(F)(F)F CC=2N═C3C4=CC(═C(F)C═C4(N═C(N1CCN(C)CC1)N3(N=2)))C(F)(F)C(F)(F)F CC=3N═C4C5=CC(═C(F)C═C5(N═C(N1CCN2CCCC2(C1))N4(N=3)))C(F)(F)C(F)(F)F CC=2N═C3C4=CC(═CC(F)═C4(N═C(N1CCNCC1)N3(N=2)))C(F)(F)C(F)(F)F CC=2N═C3C4=CC(═CC(F)═C4(N═C(N1CCN(C)CC1)N3(N=2)))C(F)(F)C(F)(F)F CC=3N═C4C5=CC(═CC(F)═C5(N═C(N1CCN2CCCC2(C1))N4(N=3)))C(F)(F)C(F)(F)F CC=2N═C3C4=CC(═C(F)C(F)═C4(N═C(N1CCNCC1)N3(N=2)))C(F)(F)C(F)(F)F CC=2N═C3C4=CC(═C(F)C(F)═C4(N═C(N1CCN(C)CC1)N3(N=2)))C(F)(F)C(F)(F)F CC=3N═C4C5=CC(═C(F)C(F)═C5(N═C(N1CCN2CCCC2(C1))N4(N=3)))C(F)(F)C(F)(F)F CC=2N═C3C4=CC(C#N)═C(F)C═C4(N═C(N1CCNCC1)N3(N=2)) CC=2N═C3C4=CC(C#N)═C(F)C═C4(N═C(N1CCN(C)CC1)N3(N=2)) CC=3N═C4C5=CC(C#N)═C(F)C═C5(N═C(N1CCN2CCCC2(C1))N4(N=3)) CC=2N═C3C4=CC(C#N)═CC═C4(N═C(N1CCNCC1)N3(N=2)) CC=2N═C3C4=CC(C#N)═CC═C4(N═C(N1CCN(C)CC1)N3(N=2)) CC=3N═C4C5=CC(C#N)═CC═C5(N═C(N1CCN2CCCC2(C1))N4(N=3)) CC=2N═C3C4=CC(C#N)═CC(F)═C4(N═C(N1CCNCC1)N3(N=2)) CC=2N═C3C4=CC(C#N)═CC(F)═C4(N═C(N1CCN(C)CC1)N3 (N=2)) CC=3N═C4C5=CC(C#N)═CC(F)═C5(N═C(N1CCN2CCCC2(C1))N4(N=3)) CC=2N═C3C4=CC(C#N)═C(F)C(F)═C4(N═C(N1CCNCC1)N3 (N=2)) CC=2N═C3C4=CC(C#N)═C(F)C(F)═C4(N═C(N1CCN(C)CC1)N3 (N=2)) CC=3N═C4C5=CC(C#N)═C(F)C(F)═C5(N═C(N1CCN2CCCC2(C1))N4(N=3)) CC=2N═C3C4=CC(═C(F)C(F)═C4(N═C(N1CCNCC1)N3(N=2)))Br CC=2N═C3C4=CC(═C(F)C(F)═C4(N═C(N1CCN(C)CC1)N3(N=2)))Br CC=3N═C4C5=CC(═C(F)C(F)═C5(N═C(N1CCN2CCCC2(C1))N4(N=3)))Br CC=2N═C3C4=CC(═C(F)C(F)═C4(N═C(N1CCNCC1)N3(N=2)))Cl CC=2N═C3C4=CC(═C(F)C(F)═C4(N═C(N1CCN(C)CC1)N3(N=2)))Cl CC=3N═C4C5=CC(═C(F)C(F)═C5(N═C(N1CCN2CCCC2(C1))N4(N=3)))Cl CC=2N═C3C4=CC(═C(F)C(F)═C4(N═C(N1CCNCC1)N3(N=2)))C(F)(F)F CC=2N═C3C4=CC(═C(F)C(F)═C4(N═C(N1CCN(C)CC1)N3(N=2)))C(F)(F)F CC=3N═C4C5=CC(═C(F)C(F)═C5(N═C(N1CCN2CCCC2(C1))N4(N=3)))C(F)(F)F CC=2N═C3C4=CC(C)═C(F)C═C4(N═C(N1CCNCC1)N3 (N=2)) CC=2N═C3C4=CC(C)═C(F)C═C4(N═C(N1CCN(C)CC1)N3 (N=2)) CC=3N═C4C5=CC(C)═C(F)C═C5(N═C(N1CCN2CCCC2(C1))N4(N=3)) CC2=CC(F)═C3N═C(N1CCNCC1)N4N═C(C)N═C4(C3(═C2)) CC2=CC(F)═C3N═C(N1CCN(C)CC1)N4N═C(C)N═C4(C3(═C2)) CC3=CC(F)═C4N═C(N1CCN2CCCC2(C1))N5N═C(C)N═C5(C4(═C3)) CC=2N═C3C4=CC(C)═C(F)C(F)═C4(N═C(N1CCNCC1)N3 (N=2)) CC=2N═C3C4=CC(C)═C(F)C(F)═C4(N═C(N1CCN(C)CC1)N3 (N=2)) CC=3N═C4C5=CC(C)═C(F)C(F)═C5(N═C(N1CCN2CCCC2(C1))N4(N=3)) CC=2N═C3C4=CC(F)═C(F)C(F)═C4(N═C(N1CCNCC1)N3 (N=2)) CC=2N═C3C4=CC(F)═C(F)C(F)═C4(N═C(N1CCN(C)CC1)N3 (N=2)) CC=3N═C4C5=CC(F)═C(F)C(F)═C5(N═C(N1CCN2CCCC2(C1))N4(N=3)) CC=2N═C3C4=CC(═C(C═C4(N═C(N1CCNCC1)N3(N=2)))C(F)(F)F)C(F)(F)F CC=2N═C3C4=CC(═C(C═C4(N═C(N1CCN(C)CC1)N3(N=2)))C(F)(F)F)C(F)(F)F CC=3N═C4C5=CC(═C(C═C5(N═C(N1CCN2CCCC2(C1))N4(N=3)))C(F)(F)F)C(F)(F)F CC=2N═C3C4=CC5=C(C═C4(N═C(N1CCNCC1)N3(N=2)))C(F)(F)C(F)(F)C5(F)(F) CC=2N═C3C4=CC5=C(C═C4(N═C(N1CCN(C)CC1)N3(N=2)))C(F)(F)C(F)(F)C5(F)(F) CC=3N═C4C5=CC6=C(C═C5(N═C(N1CCN2CCCC2(C1))N4(N=3)))C(F)(F)C(F)(F)C6(F)(F) FC(F)(F)C(F)(F)C1=CC═C2N═C(C3=NN═NN3(C2(═C1)))N4CCNCC4 FC=1C═C2N═C(C3=NN═NN3(C2(═CC=1C(F)(F)C(F)(F)F)))N4CCNCC4 FC=4C═C(C═C1C=4(N═C(C2=NN═NN12)N3CCNCC3))C(F)(F)C(F)(F)F CN1CCN(CC1)C3=NC2=CC═C(C═C2N4N═NN═C34)C(F)(F)C(F)(F)F CN1CCN(CC1)C3=NC2=CC(F)═C(C═C2N4N═NN═C34)C(F)(F)C(F)(F)F CN1CCN(CC1)C3=NC2=C(F)C═C(C═C2N4N═NN═C34)C(F)(F)C(F)(F)F FC(F)(F)C(F)(F)C1=CC═C2N═C(C3=NN═NN3 (C2(═C1)))N4CCN5CCCC5 (C4) FC=1C═C2N═C(C3=NN═NN3 (C2(═CC=1C(F)(F)C(F)(F)F)))N4CCN5CCCC5 (C4) FC=5C═C(C═C1C=5(N═C(C2=NN═NN12)N3CCN4CCCC4(C3)))C(F)(F)C(F)(F)F FC=4C(F)═C(C═C1C=4(N═C(C2=NN═NN12)N3CCNCC3))C(F)(F)C(F)(F)F CN1CCN(CC1)C3=NC2=C(F)C(F)═C(C═C2N4N═NN═C34)C(F)(F)C(F)(F)F FC=5C(F)═C(C═C1C=5(N═C(C2=NN═NN12)N3CCN4CCCC4(C3)))C(F)(F)C(F)(F)F N#CC1=CC═C2N═C(C3=NN═NN3(C2(═C1)))N4CCNCC4 CN1CCN(CC1)C3=NC2=CC═C(C#N)C═C2N4N═NN═C34 N#CC1=CC═C2N═C(C3=NN═NN3 (C2(═C1)))N4CCN5CCCC5 (C4) N#CC=1C═C2C(═CC=1(F))N═C(C3=NN═NN23)N4CCNCC4 CN1CCN(CC1)C3=NC2=CC(F)═C(C#N)C═C2N4N═NN═C34 N#CC=1C═C2C(═CC=1(F))N═C(C3=NN═NN23)N4CCN5CCCC5 (C4) N#CC1=CC(F)═C2N═C(C3=NN═NN3(C2(═C1)))N4CCNCC4 CN1CCN(CC1)C3=NC2=C(F)C═C(C#N)C═C2N4N═NN═C34 N#CC1=CC(F)═C2N═C(C3=NN═NN3 (C2(═C1)))N4CCN5CCCC5 (C4) N#CC=4C═C1C(N═C(C2=NN═NN12)N3CCNCC3)=C(F)C=4(F) CN1CCN(CC1)C3=NC2=C(F)C(F)═C(C#N)C═C2N4N═NN═C34 N#CC=5C═C1C(N═C(C2=NN═NN12)N3CCN4CCCC4(C3))=C(F)C=5(F) FC=4C(F)═C(C═C1C=4(N═C(C2=NN═NN12)N3CCNCC3))Br CN1CCN(CC1)C3=NC2=C(F)C(F)═C(C═C2N4N═NN═C34)Br FC=5C(F)═C(C═C1C=5(N═C(C2=NN═NN12)N3CCN4CCCC4(C3)))Br CC1=CC(F)═C2N═C(C3=NN═NN3(C2(═C1)))N4CCNCC4 CC=4C═C1C(N═C(C2=NN═NN12)N3CCNCC3)=C(F)C=4(F) FC=4C═C1C(N═C(C2=NN═NN12)N3CCNCC3)=C(F)C=4(F) FC2(F)(C=1C═C3N═C(C4=NN═NN4(C3(═CC=1C(F)(F)C2(F)(F))))N5CCNCC5) FC(F)(F)OC1=CC═C2N═C(C3=NN═NN3(C2(═C1)))N4CCNCC4 CN1CCN(CC1)C3=NC2=CC═C(C═C2N4N═NN═C34)OC(F)(F)F FC(F)(F)OC1=CC═C2N═C(C3=NN═NN3 (C2(═C1)))N4CCN5CCCC5 (C4) FC(F)(F)C1=CC═C2N═C(C3=NN═NN3(C2(═C1)))N4CCNCC4 CN1CCN(CC1)C3=NC2=CC═C(C═C2N4N═NN═C34)C(F)(F)F FC(F)(F)C1=CC═C2N═C(C3=NN═NN3 (C2(═C1)))N4CCN5CCCC5 (C4) CN1CCN(CC1)C3=NC2=CC5=C(C═C2N4N═NN═C34)C(F)(F)C(F)(F)C5(F)(F) FC2(F)(C=1C═C3N═C(C4=NN═NN4(C3(═CC=1C(F)(F)C2(F)(F))))N5CCN6CCCC6(C5)) CC1=CC(F)═C2N═C(C3=NN═NN3(C2(═C1)))N4CCN(C)CC4 CC=4C═C1C(N═C(C2=NN═NN12)N3CCN(C)CC3)=C(F)C=4(F) CN1CCN(CC1)C3=NC2=C(F)C(F)═C(F)C═C2N4N═NN═C34 CC1=CC(F)═C2N═C(C3=NN═NN3 (C2(═C1)))N4CCN5CCCC5 (C4) CC=5C═C1C(N═C(C2=NN═NN12)N3CCN4CCCC4(C3))=C(F)C=5(F) FC=5C═C1C(N═C(C2=NN═NN12)N3CCN4CCCC4(C3))=C(F)C=5(F) FC=4C(F)═C(C═C1C=4(N═C(C2=NN═NN12)N3CCNCC3))Cl CN1CCN(CC1)C3=NC2=C(F)C(F)═C(C═C2N4N═NN═C34)Cl FC=5C(F)═C(C═C1C=5(N═C(C2=NN═NN12)N3CCN4CCCC4(C3)))Cl FC=4C(F)═C(C═C1C=4(N═C(C2=NN═NN12)N3CCNCC3))C(F)(F)F CN1CCN(CC1)C3=NC2=C(F)C(F)═C(C═C2N4N═NN═C34)C(F)(F)F FC=5C(F)═C(C═C1C=5(N═C(C2=NN═NN12)N3CCN4CCCC4(C3)))C(F)(F)F CC=1C═C2C(═CC=1(F))N═C(C3=NN═NN23)N4CCNCC4 CC=1C═C2C(═CC=1(F))N═C(C3=NN═NN23)N4CCN(C)CC4 CC=1C═C2C(═CC=1(F))N═C(C3=NN═NN23)N4CCN5CCCC5 (C4) CC=2N═C3C(═NC1=CC═C(C═C1N3(N=2))C(F)(F)C(F)(F)F)N4CCNCC4 CC=2N═C3C(═NC1=CC═C(C═C1N3(N=2))C(F)(F)C(F)(F)F)N4CCN(C)CC4 CC=2N═C3C(═NC1=CC═C(C═C1N3 (N=2))C(F)(F)C(F)(F)F)N4CCN5CCCC5 (C4) CC=2N═C3C(═NC1=CC(F)═C(C═C1N3(N=2))C(F)(F)C(F)(F)F)N4CCNCC4 CC=2N═C3C(═NC1=CC(F)═C(C═C1N3(N=2))C(F)(F)C(F)(F)F)N4CCN(C)CC4 CC=2N═C3C(═NC1=CC(F)═C(C═C1N3 (N=2))C(F)(F)C(F)(F)F)N4CCN5CCCC5 (C4) CC=2N═C3C(═NC1=C(F)C═C(C═C1N3(N=2))C(F)(F)C(F)(F)F)N4CCNCC4 CC=2N═C3C(═NC1=C(F)C═C(C═C1N3(N=2))C(F)(F)C(F)(F)F)N4CCN(C)CC4 CC=2N═C3C(═NC1=C(F)C═C(C═C1N3 (N=2))C(F)(F)C(F)(F)F)N4CCN5CCCC5 (C4) CC=2N═C3C(═NC1=C(F)C(F)═C(C═C1N3(N=2))C(F)(F)C(F)(F)F)N4CCNCC4 CC=2N═C3C(═NC1=C(F)C(F)═C(C═C1N3(N=2))C(F)(F)C(F)(F)F)N4CCN(C)CC4 CC=2N═C3C(═NC1=CC═C(C#N)C═C1N3(N=2))N4CCNCC4 CC=2N═C3C(═NC1=C(F)C═C(C#N)C═C1N3(N=2))N4CCNCC4 CC=2N═C3C(═NC1=C(F)C(F)═C(C═C1N3(N=2))Br)N4CCNCC4 CC=2N═C3C(═NC1=C(F)C(F)═C(C═C1N3(N=2))Br)N4CCN(C)CC4 CC=2N═C3C(═NC1=C(F)C(F)═C(C═C1N3 (N=2))Br)N4CCN5CCCC5 (C4) CC=2N═C3C(═NC1=C(F)C(F)═C(C═C1N3(N=2))C1)N4CCNCC4 CC=2N═C3C(═NC1=C(F)C(F)═C(C═C1N3(N=2))C1)N4CCN(C)CC4 CC=2N═C3C(═NC1=C(F)C(F)═C(C═C1N3(N=2))C1)N4CCN5CCCC5(C4) CC=2N═C3C(═NC1=C(F)C(F)═C(C═C1N3(N=2))C(F)(F)F)N4CCNCC4 CC=2N═C3C(═NC1=C(F)C(F)═C(C═C1N3(N=2))C(F)(F)F)N4CCN(C)CC4 CC=2N═C3C(═NC1=C(F)C(F)═C(C═C1N3 (N=2))C(F)(F)F)N4CCN5CCCC5 (C4) CC=2N═C3C(═NC1=CC(F)═C(C═C1N3(N=2))C(F)(F)F)N4CCNCC4 CC=2N═C3C(═NC1=CC(F)═C(C═C1N3(N=2))C(F)(F)F)N4CCN(C)CC4 CC=2N═C3C(═NC1=CC(F)═C(C═C1N3 (N=2))C(F)(F)F)N4CCN5CCCC5 (C4) FC(F)(F)C(F)(F)C1=CC═C2N═C(C3=NC═NN3(C2(═C1)))N4CCNCC4 CN1CCN(CC1)C3=NC2=CC═C(C═C2N4N═CN═C34)C(F)(F)C(F)(F)F FC(F)(F)C(F)(F)C1=CC═C2N═C(C3=NC═NN3 (C2(═C1)))N4CCN5CCCC5 (C4) FC=1C═C2N═C(C3=NC═NN3(C2(═CC=1C(F)(F)C(F)(F)F)))N4CCNCC4 CN1CCN(CC1)C3=NC2=CC(F)═C(C═C2N4N═CN═C34)C(F)(F)C(F)(F)F FC=1C═C2N═C(C3=NC═NN3 (C2(═CC=1C(F)(F)C(F)(F)F)))N4CCN5CCCC5 (C4) FC=4C═C(C═C1C=4(N═C(C2=NC═NN12)N3CCNCC3))C(F)(F)C(F)(F)F CN1CCN(CC1)C3=NC2=C(F)C═C(C═C2N4N═CN═C34)C(F)(F)C(F)(F)F FC=5C═C(C═C1C=5(N═C(C2=NC═NN12)N3CCN4CCCC4(C3)))C(F)(F)C(F)(F)F N#CC1=CC═C2N═C(C3=NC═NN3(C2(═C1)))N4CCNCC4 N#CC=1C═C2C(═CC=1(F))N═C(C3=NC═NN23)N4CCNCC4 CN1CCN(CC1)C3=NC2=CC(F)═C(C#N)C═C2N4N═CN═C34 N#CC=1C═C2C(═CC=1(F))N═C(C3=NC═NN23)N4CCN5CCCC5 (C4) N#CC1=CC(F)═C2N═C(C3=NC═NN3(C2(═C1)))N4CCNCC4 CN1CCN(CC1)C3=NC2=C(F)C═C(C#N)C═C2N4N═CN═C34 N#CC1=CC(F)═C2N═C(C3=NC═NN3 (C2(═C1)))N4CCN5CCCC5 (C4) N#CC=4C═C1C(N═C(C2=NC═NN12)N3CCNCC3)=C(F)C=4(F) CN1CCN(CC1)C3=NC2=C(F)C(F)═C(C#N)C═C2N4N═CN═C34 N#CC=5C═C1C(N═C(C2=NC═NN12)N3CCN4CCCC4(C3))=C(F)C=5(F) FC=4C(F)═C(C═C1C=4(N═C(C2=NC═NN12)N3CCNCC3))Br CN1CCN(CC1)C3=NC2=C(F)C(F)═C(C═C2N4N═CN═C34)Br FC=5C(F)═C(C═C1C=5(N═C(C2=NC═NN12)N3CCN4CCCC4(C3)))Br FC=1C═C2N═C(C3=NC═NN3(C2(═CC=1Br)))N4CCNCC4 CN1CCN(CC1)C3=NC2=CC(F)═C(C═C2N4N═CN═C34)Br FC=1C═C2N═C(C3=NC═NN3 (C2(═CC=1Br)))N4CCN5CCCC5 (C4) FC=4C═C(C═C1C=4(N═C(C2=NC═NN12)N3CCNCC3))Br CN1CCN(CC1)C3=NC2=C(F)C═C(C═C2N4N═CN═C34)Br FC=5C═C(C═C1C=5(N═C(C2=NC═NN12)N3CCN4CCCC4(C3)))Br FC=4C(F)═C(C═C1C=4(N═C(C2=NC═NN12)N3CCNCC3))Br CN1CCN(CC1)C3=NC2=C(F)C(F)═C(C═C2N4N═CN═C34)Br FC=5C(F)═C(C═C1C=5(N═C(C2=NC═NN12)N3CCN4CCCC4(C3)))Br FC=1C═C2N═C(C3=NC═NN3(C2(═CC=1Cl)))N4CCNCC4 CN1CCN(CC1)C3=NC2=CC(F)═C(C═C2N4N═CN═C34)Cl FC=1C═C2N═C(C3=NC═NN3 (C2(═CC=1Cl)))N4CCN5CCCC5 (C4) FC=4C═C(C═C1C=4(N═C(C2=NC═NN12)N3CCNCC3))Cl CN1CCN(CC1)C3=NC2=C(F)C═C(C═C2N4N═CN═C34)Cl FC=5C═C(C═C1C=5(N═C(C2=NC═NN12)N3CCN4CCCC4(C3)))Cl FC=4C(F)═C(C═C1C=4(N═C(C2=NC═NN12)N3CCNCC3))Cl CN1CCN(CC1)C3=NC2=C(F)C(F)═C(C═C2N4N═CN═C34)Cl FC=5C(F)═C(C═C1C=5(N═C(C2=NC═NN12)N3CCN4CCCC4(C3)))Cl FC(F)(F)C1=CC═C2N═C(C3=NC═NN3(C2(═C1)))N4CCNCC4 CN1CCN(CC1)C3=NC2=CC═C(C═C2N4N═CN═C34)C(F)(F)F FC(F)(F)C1=CC═C2N═C(C3=NC═NN3 (C2(═C1)))N4CCN5CCCC5 (C4) FC=1C═C2N═C(C3=NC═NN3(C2(═CC=1C(F)(F)F)))N4CCNCC4 CN1CCN(CC1)C3=NC2=CC(F)═C(C═C2N4N═CN═C34)C(F)(F)F FC=1C═C2N═C(C3=NC═NN3(C2(═CC=1C(F)(F)F)))N4CCN5CCCC5(C4) FC=4C═C(C═C1C=4(N═C(C2=NC═NN12)N3CCNCC3))C(F)(F)F CN1CCN(CC1)C3=NC2=C(F)C═C(C═C2N4N═CN═C34)C(F)(F)F FC=5C═C(C═C1C=5(N═C(C2=NC═NN12)N3CCN4CCCC4(C3)))C(F)(F)F FC=4C(F)═C(C═C1C=4(N═C(C2=NC═NN12)N3CCNCC3))C(F)(F)F CN1CCN(CC1)C3=NC2=C(F)C(F)═C(C═C2N4N═CN═C34)C(F)(F)F FC=5C(F)═C(C═C1C=5(N═C(C2=NC═NN12)N3CCN4CCCC4(C3)))C(F)(F)F FC(F)(F)C=1C═C2C(═CC=1Cl)N═C(C3=NC═NN23)N4CCNCC4 CN1CCN(CC1)C3=NC2=CC(═C(C═C2N4N═CN═C34)C(F)(F)F)Cl FC(F)(F)C=1C═C2C(═CC=1Cl)N═C(C3=NC═NN23)N4CCN5CCCC5 (C4) FC=1C═C2N═C(C3=NOC═C3(C2(═CC=1Br)))N4CCNCC4 CN1CCN(CC1)C4=NC2=CC(F)═C(C═C2C3=CON═C34)Br FC=1C═C2N═C(C3=NOC═C3 (C2(═CC=1Br)))N4CCN5CCCC5 (C4) FC=2C═C(C═C3C1=CON═C1C(═NC=23)N4CCNCC4)Br CN1CCN(CC1)C3=NC4=C(F)C═C(C═C4(C2=CON═C23))Br FC=2C═C(C═C3C1=CON═C1C(═NC=23)N4CCN5CCCC5(C4))Br FC=1C(F)═C2N═C(C3=NOC═C3(C2(═CC=1Br)))N4CCNCC4 CN1CCN(CC1)C3=NC4=C(F)C(F)═C(C═C4(C2=CON═C23))Br FC=1C(F)═C2N═C(C3=NOC═C3 (C2(═CC=1Br)))N4CCN5CCCC5 (C4) FC=1C═C2N═C(C3=NOC═C3(C2(═CC=1Cl)))N4CCNCC4 FC=1C═C2N═C(C3=NOC═C3 (C2(═CC=1Cl)))N4CCN5CCCC5 (C4) FC=2C═C(C═C3C1=CON═C1C(═NC=23)N4CCNCC4)Cl CN1CCN(CC1)C3=NC4=C(F)C═C(C═C4(C2=CON═C23))Cl FC=2C═C(C═C3C1=CON═C1C(═NC=23)N4CCN5CCCC5(C4))Cl FC=1C(F)═C2N═C(C3=NOC═C3(C2(═CC=1Cl)))N4CCNCC4 CN1CCN(CC1)C3=NC4=C(F)C(F)═C(C═C4(C2=CON═C23))Cl FC=1C(F)═C2N═C(C3=NOC═C3 (C2(═CC=1Cl)))N4CCN5CCCC5 (C4) FC=1C═C2N═C(C3=NOC═C3(C2(═CC=1C(F)(F)F)))N4CCNCC4 CN1CCN(CC1)C4=NC2=CC(F)═C(C═C2C3=CON═C34)C(F)(F)F FC=1C═C2N═C(C3=NOC═C3 (C2(═CC=1C(F)(F)F)))N4CCN5CCCC5 (C4) FC=2C═C(C═C3C1=CON═C1C(═NC=23)N4CCNCC4)C(F)(F)F CN1CCN(CC1)C3=NC4=C(F)C═C(C═C4(C2=CON═C23))C(F)(F)F FC=2C═C(C═C3C1=CON═C1C(═NC=23)N4CCN5CCCC5(C4))C(F)(F)F FC=1C(F)═C2N═C(C3=NOC═C3(C2(═CC=1C(F)(F)F)))N4CCNCC4 CN1CCN(CC1)C3=NC4=C(F)C(F)═C(C═C4(C2=CON═C23))C(F)(F)F FC=1C(F)═C2N═C(C3=NOC═C3 (C2(═CC=1C(F)(F)F)))N4CCN5CCCC5 (C4) FC(F)(F)C=1C═C2C(═CC=1Cl)N═C(C3=NOC═C23)N4CCNCC4 CN1CCN(CC1)C4=NC2=CC(═C(C═C2C3=CON═C34)C(F)(F)F)Cl FC(F)(F)C=1C═C2C(═CC=1Cl)N═C(C3=NOC═C23)N4CCN5CCCC5 (C4) FC(F)(F)C(F)(F)C2=CC═C3N═C(N1CCNCC1)N4N═CN═C4(C3(═C2)) CN1CCN(CC1)C4=NC2=CC═C(C═C2C3=NC═NN34)C(F)(F)C(F)(F)F FC(F)(F)C(F)(F)C3=CC═C4N═C(N1CCN2CCCC2(C1))N5N═CN═C5(C4(═C3)) FC=2C═C3N═C(N1CCNCC1)N4N═CN═C4(C3(═CC=2C(F)(F)C(F)(F)F)) CN1CCN(CC1)C4=NC2=CC(F)═C(C═C2C3=NC═NN34)C(F)(F)C(F)(F)F FC=3C═C4N═C(N1CCN2CCCC2(C1))N5N═CN═C5(C4(═CC=3C(F)(F)C(F)(F)F)) FC=4C═C(C═C2C=4(N═C(N1CCNCC1)N3N═CN═C23))C(F)(F)C(F)(F)F CN1CCN(CC1)C4=NC2=C(F)C═C(C═C2C3=NC═NN34)C(F)(F)C(F)(F)F FC=5C═C(C═C3C=5(N═C(N1CCN2CCCC2(C1))N4N═CN═C34))C(F)(F)C(F)(F)F FC=4C(F)═C(C═C2C=4(N═C(N1CCNCC1)N3N═CN═C23))C(F)(F)C(F)(F)F CN1CCN(CC1)C4=NC2=C(F)C(F)═C(C═C2C3=NC═NN34)C(F)(F)C(F)(F)F FC=5C(F)═C(C═C3C=5(N═C(N1CCN2CCCC2(C1))N4N═CN═C34))C(F)(F)C(F)(F)F N#CC2=CC═C3N═C(N1CCNCC1)N4N═CN═C4(C3(═C2)) N#CC=1C═C3C(═CC=1(F))N═C(N2CCNCC2)N4N═CN═C34 N#CC2=CC(F)═C3N═C(N1CCNCC1)N4N═CN═C4(C3(═C2)) C1CN(CCN1)C4=NC2=CC═C(C═C2C3=NC═NN34)Br CN1CCN(CC1)C4=NC2=CC═C(C═C2C3=NC═NN34)Br C1CC2CN(CCN2(C1))C5=NC3=CC═C(C═C3C4=NC═NN45)Br FC=2C═C3N═C(N1CCNCC1)N4N═CN═C4(C3(═CC=2Br)) CN1CCN(CC1)C4=NC2=CC(F)═C(C═C2C3=NC═NN34)Br FC=3C═C4N═C(N1CCN2CCCC2(C1))N5N═CN═C5(C4(═CC=3Br)) FC=4C═C(C═C2C=4(N═C(N1CCNCC1)N3N═CN═C23))Br CN1CCN(CC1)C4=NC2=C(F)C═C(C═C2C3=NC═NN34)Br FC=5C═C(C═C3C=5(N═C(N1CCN2CCCC2(C1))N4N═CN═C34))Br FC=4C(F)═C(C═C2C=4(N═C(N1CCNCC1)N3N═CN═C23))Br CN1CCN(CC1)C4=NC2=C(F)C(F)═C(C═C2C3=NC═NN34)Br FC=5C(F)═C(C═C3C=5(N═C(N1CCN2CCCC2(C1))N4N═CN═C34))Br C1CN(CCN1)C4=NC2=CC═C(C═C2C3=NC═NN34)Cl C1CC2CN(CCN2(C1))C5=NC3=CC═C(C═C3C4=NC═NN45)Cl FC=2C═C3N═C(N1CCNCC1)N4N═CN═C4(C3(═CC=2Cl)) CN1CCN(CC1)C4=NC2=CC(F)═C(C═C2C3=NC═NN34)Cl FC=3C═C4N═C(N1CCN2CCCC2(C1))N5N═CN═C5(C4(═CC=3Cl)) FC=4C═C(C═C2C=4(N═C(N1CCNCC1)N3N═CN═C23))Cl CN1CCN(CC1)C4=NC2=C(F)C═C(C═C2C3=NC═NN34)Cl FC=5C═C(C═C3C=5(N═C(N1CCN2CCCC2(C1))N4N═CN═C34))Cl FC=4C(F)═C(C═C2C=4(N═C(N1CCNCC1)N3N═CN═C23))Cl CN1CCN(CC1)C4=NC2=C(F)C(F)═C(C═C2C3=NC═NN34)Cl FC=5C(F)═C(C═C3C=5(N═C(N1CCN2CCCC2(C1))N4N═CN═C34))Cl FC(F)(F)C2=CC═C3N═C(N1CCNCC1)N4N═CN═C4(C3(═C2)) CN1CCN(CC1)C4=NC2=CC═C(C═C2C3=NC═NN34)C(F)(F)F FC(F)(F)C3=CC═C4N═C(N1CCN2CCCC2(C1))N5N═CN═C5(C4(═C3)) FC=2C═C3N═C(N1CCNCC1)N4N═CN═C4(C3(═CC=2C(F)(F)F)) CN1CCN(CC1)C4=NC2=CC(F)═C(C═C2C3=NC═NN34)C(F)(F)F FC=3C═C4N═C(N1CCN2CCCC2(C1))N5N═CN═C5(C4(═CC=3C(F)(F)F)) FC=4C═C(C═C2C=4(N═C(N1CCNCC1)N3N═CN═C23))C(F)(F)F CN1CCN(CC1)C4=NC2=C(F)C═C(C═C2C3=NC═NN34)C(F)(F)F FC=5C═C(C═C3C=5(N═C(N1CCN2CCCC2(C1))N4N═CN═C34))C(F)(F)F FC=4C(F)═C(C═C2C=4(N═C(N1CCNCC1)N3N═CN═C23))C(F)(F)F CN1CCN(CC1)C4=NC2=C(F)C(F)═C(C═C2C3=NC═NN34)C(F)(F)F FC=5C(F)═C(C═C3C=5(N═C(N1CCN2CCCC2(C1))N4N═CN═C34))C(F)(F)F CC2=CC═C3N═C(N1CCNCC1)N4N═CN═C4(C3(═C2)) CC2=CC═C3N═C(N1CCN(C)CC1)N4N═CN═C4(C3(═C2)) CC3=CC═C4N═C(N1CCN2CCCC2(C1))N5N═CN═C5(C4(═C3)) CC=1C═C3C(═CC=1(F))N═C(N2CCNCC2)N4N═CN═C34 CC=1C═C3C(═CC=1(F))N═C(N2CCN(C)CC2)N4N═CN═C34 CC=1C═C4C(═CC=1(F))N═C(N2CCN3CCCC3(C2))N5N═CN═C45 CC2=CC(F)═C3N═C(N1CCNCC1)N4N═CN═C4(C3(═C2)) CC2=CC(F)═C3N═C(N1CCN(C)CC1)N4N═CN═C4(C3(═C2)) CC3=CC(F)═C4N═C(N1CCN2CCCC2(C1))N5N═CN═C5(C4(═C3)) FC(F)(F)C=1C═C3C(═CC=1Cl)N═C(N2CCNCC2)N4N═CN═C34 CN1CCN(CC1)C4=NC2=CC(═C(C═C2C3=NC═NN34)C(F)(F)F)Cl FC(F)(F)C=1C═C4C(═CC=1Cl)N═C(N2CCN3CCCC3(C2))N5N═CN═C45 C1CN(CCN1)C4=NC2=CC═C(C═C2C3=NN═NN34)Br CN1CCN(CC1)C4=NC2=CC═C(C═C2C3=NN═NN34)Br C1CC2CN(CCN2(C1))C5=NC3=CC═C(C═C3C4=NN═NN45)Br FC=2C═C3N═C(N1CCNCC1)N4N═NN═C4(C3(═CC=2Br)) CN1CCN(CC1)C4=NC2=CC(F)═C(C═C2C3=NN═NN34)Br FC=3C═C4N═C(N1CCN2CCCC2(C1))N5N═NN═C5(C4(═CC=3Br)) FC=4C═C(C═C2C=4(N═C(N1CCNCC1)N3N═NN═C23))Br CN1CCN(CC1)C4=NC2=C(F)C═C(C═C2C3=NN═NN34)Br FC=5C═C(C═C3C=5(N═C(N1CCN2CCCC2(C1))N4N═NN═C34))Br FC=4C(F)═C(C═C2C=4(N═C(N1CCNCC1)N3N═NN═C23))Br CN1CCN(CC1)C4=NC2=C(F)C(F)═C(C═C2C3=NN═NN34)Br FC=5C(F)═C(C═C3C=5(N═C(N1CCN2CCCC2(C1))N4N═NN═C34))Br FC=2C═C3N═C(N1CCNCC1)N4N═NN═C4(C3(═CC=2Cl)) CN1CCN(CC1)C4=NC2=CC(F)═C(C═C2C3=NN═NN34)Cl FC=3C═C4N═C(N1CCN2CCCC2(C1))N5N═NN═C5(C4(═CC=3Cl)) FC=4C═C(C═C2C=4(N═C(N1CCNCC1)N3N═NN═C23))Cl CN1CCN(CC1)C4=NC2=C(F)C═C(C═C2C3=NN═NN34)Cl FC=5C═C(C═C3C=5(N═C(N1CCN2CCCC2(C1))N4N═NN═C34))Cl FC=4C(F)═C(C═C2C=4(N═C(N1CCNCC1)N3N═NN═C23))Cl CN1CCN(CC1)C4=NC2=C(F)C(F)═C(C═C2C3=NN═NN34)Cl FC=5C(F)═C(C═C3C=5(N═C(N1CCN2CCCC2(C1))N4N═NN═C34))Cl FC(F)(F)C2=CC═C3N═C(N1CCNCC1)N4N═NN═C4(C3(═C2)) CN1CCN(CC1)C4=NC2=CC═C(C═C2C3=NN═NN34)C(F)(F)F FC(F)(F)C3=CC═C4N═C(N1CCN2CCCC2(C1))N5N═NN═C5(C4(═C3)) FC=2C═C3N═C(N1CCNCC1)N4N═NN═C4(C3(═CC=2C(F)(F)F)) CN1CCN(CC1)C4=NC2=CC(F)═C(C═C2C3=NN═NN34)C(F)(F)F FC=3C═C4N═C(N1CCN2CCCC2(C1))N5N═NN═C5(C4(═CC=3C(F)(F)F)) FC=4C═C(C═C2C=4(N═C(N1CCNCC1)N3N═NN═C23))C(F)(F)F CN1CCN(CC1)C4=NC2=C(F)C═C(C═C2C3=NN═NN34)C(F)(F)F FC=5C═C(C═C3C=5(N═C(N1CCN2CCCC2(C1))N4N═NN═C34))C(F)(F)F FC=4C(F)═C(C═C2C=4(N═C(N1CCNCC1)N3N═NN═C23))C(F)(F)F CN1CCN(CC1)C4=NC2=C(F)C(F)═C(C═C2C3=NN═NN34)C(F)(F)F FC=5C(F)═C(C═C3C=5(N═C(N1CCN2CCCC2(C1))N4N═NN═C34))C(F)(F)F CC=1C═C3C(═CC=1(F))N═C(N2CCNCC2)N4N═NN═C34 CC=1C═C3C(═CC=1(F))N═C(N2CCN(C)CC2)N4N═NN═C34 CC=1C═C4C(═CC=1(F))N═C(N2CCN3CCCC3(C2))N5N═NN═C45 CC2=CC(F)═C3N═C(N1CCNCC1)N4N═NN═C4(C3(═C2)) CC2=CC(F)═C3N═C(N1CCN(C)CC1)N4N═NN═C4(C3(═C2)) CC3=CC(F)═C4N═C(N1CCN2CCCC2(C1))N5N═NN═C5(C4(═C3)) CC2=NC=3C(═NC1=CC(F)═C(C═C1C=3(O2))Br)N4CCNCC4 CC2=NC=3C(═NC1=CC(F)═C(C═C1C=3(O2))Br)N4CCN(C)CC4 CC2=NC=3C(═NC1=CC(F)═C(C═C1C=3(O2))Br)N4CCN5CCCC5(C4) CC2=NC=3C(═NC1=C(F)C═C(C═C1C=3(O2))Br)N4CCNCC4 CC2=NC=3C(═NC1=C(F)C═C(C═C1C=3(O2))Br)N4CCN(C)CC4 CC2=NC=3C(═NC1=C(F)C═C(C═C1C=3(O2))Br)N4CCN5CCCC5(C4) CC2=NC=3C(═NC1=C(F)C(F)═C(C═C1C=3(O2))Br)N4CCNCC4 CC2=NC=3C(═NC1=C(F)C(F)═C(C═C1C=3(O2))Br)N4CCN(C)CC4 CC2=NC=3C(═NC1=C(F)C(F)═C(C═C1C=3(O2))Br)N4CCN5CCCC5(C4) CC2=NC=3C(═NC1=CC(F)═C(C═C1C=3(O2))Cl)N4CCNCC4 CC2=NC=3C(═NC1=CC(F)═C(C═C1C=3(O2))Cl)N4CCN(C)CC4 CC2=NC=3C(═NC1=CC(F)═C(C═C1C=3 (O2))Cl)N4CCN5CCCC5 (C4) CC2=NC=3C(═NC1=C(F)C═C(C═C1C=3(O2))Cl)N4CCNCC4 CC2=NC=3C(═NC1=C(F)C═C(C═C1C=3(O2))Cl)N4CCN(C)CC4 CC2=NC=3C(═NC1=C(F)C═C(C═C1C=3 (O2))Cl)N4CCN5CCCC5 (C4) CC2=NC=3C(═NC1=C(F)C(F)═C(C═C1C=3(O2))Cl)N4CCNCC4 CC2=NC=3C(═NC1=C(F)C(F)═C(C═C1C=3(O2))Cl)N4CCN(C)CC4 CC2=NC=3C(═NC1=C(F)C(F)═C(C═C1C=3 (O2))Cl)N4CCN5CCCC5 (C4) FC(F)(F)C(F)(F)C1=CC═C2N═C(C=3N═COC=3(C2(═C1)))N4CCNCC4 CN1CCN(CC1)C3=NC2=CC═C(C═C2C=4OC═NC3=4)C(F)(F)C(F)(F)F FC(F)(F)C(F)(F)C1=CC═C2N═C(C=3N═COC=3 (C2(═C1)))N4CCN5CCCC5 (C4) FC=1C═C2N═C(C=3N═COC=3(C2(═CC=1C(F)(F)C(F)(F)F)))N4CCNCC4 CN1CCN(CC1)C3=NC2=CC(F)═C(C═C2C=40C═NC3=4)C(F)(F)C(F)(F)F FC=1C═C2N═C(C=3N═COC=3 (C2(═CC=1C(F)(F)C(F)(F)F)))N4CCN5CCCC5 (C4) FC=4C═C(C═C1C=4(N═C(C=2N═C0C1=2)N3CCNCC3))C(F)(F)C(F)(F)F CN1CCN(CC1)C3=NC2=C(F)C═C(C═C2C=40C═NC3=4)C(F)(F)C(F)(F)F FC=5C═C(C═C1C=5(N═C(C=2N═COC1=2)N3CCN4CCCC4(C3)))C(F)(F)C(F)(F)F FC=1C═C2N═C(C=3N═COC=3(C2(═CC=1Br)))N4CCNCC4 CN1CCN(CC1)C3=NC2=CC(F)═C(C═C2C=40C═NC3=4)Br FC=1C═C2N═C(C=3N═COC=3 (C2(═CC=1Br)))N4CCN5CCCC5 (C4) FC=4C═C(C═C1C=4(N═C(C=2N═C0C1=2)N3CCNCC3))Br CN1CCN(CC1)C3=NC2=C(F)C═C(C═C2C=40C═NC3=4)Br FC=5C═C(C═C1C=5(N═C(C=2N═COC1=2)N3CCN4CCCC4(C3)))Br FC=1C(F)═C2N═C(C=3N═COC=3(C2(═CC=1Br)))N4CCNCC4 CN1CCN(CC1)C3=NC2=C(F)C(F)═C(C═C2C=40C═NC3=4)Br FC=1C(F)═C2N═C(C=3N═COC=3 (C2(═CC=1Br)))N4CCN5CCCC5 (C4) FC=1C═C2N═C(C=3N═COC=3(C2(═CC=1Cl)))N4CCNCC4 CN1CCN(CC1)C3=NC2=CC(F)═C(C═C2C=40C═NC3=4)Cl FC=1C═C2N═C(C=3N═COC=3 (C2(═CC=1Cl)))N4CCN5CCCC5 (C4) FC=4C═C(C═C1C=4(N═C(C=2N═C0C1=2)N3CCNCC3))Cl CN1CCN(CC1)C3=NC2=C(F)C═C(C═C2C=40C═NC3=4)Cl FC=5C═C(C═C1C=5(N═C(C=2N═COC1=2)N3CCN4CCCC4(C3)))Cl FC=1C(F)═C2N═C(C=3N═COC=3(C2(═CC=1Cl)))N4CCNCC4 CN1CCN(CC1)C3=NC2=C(F)C(F)═C(C═C2C=40C═NC3=4)Cl FC=1C(F)═C2N═C(C=3N═COC=3 (C2(═CC=1Cl)))N4CCN5CCCC5 (C4) C1CN(CCN1)C3=NC2=CC═C(C═C2N4C═CN═C34)Br CN1CCN(CC1)C3=NC2=CC═C(C═C2N4C═CN═C34)Br C1CC2CN(CCN2(C1))C4=NC3=CC═C(C═C3N5C═CN═C45)Br FC=1C═C2N═C(C3=NC═CN3(C2(═CC=1Br)))N4CCNCC4 CN1CCN(CC1)C3=NC2=CC(F)═C(C═C2N4C═CN═C34)Br FC=1C═C2N═C(C3=NC═CN3 (C2(═CC=1Br)))N4CCN5CCCC5 (C4) FC=4C═C(C═C1C=4(N═C(C2=NC═CN12)N3CCNCC3))Br CN1CCN(CC1)C3=NC2=C(F)C═C(C═C2N4C═CN═C34)Br FC=5C═C(C═C1C=5(N═C(C2=NC═CN12)N3CCN4CCCC4(C3)))Br FC=4C(F)═C(C═C1C=4(N═C(C2=NC═CN12)N3CCNCC3))Br CN1CCN(CC1)C3=NC2=C(F)C(F)═C(C═C2N4C═CN═C34)Br FC=5C(F)═C(C═C1C=5(N═C(C2=NC═CN12)N3CCN4CCCC4(C3)))Br FC=4C═C(C═C1C=4(N═C(C2=NC═CN12)N3CCNCC3))Cl CN1CCN(CC1)C3=NC2=C(F)C═C(C═C2N4C═CN═C34)Cl FC=5C═C(C═C1C=5(N═C(C2=NC═CN12)N3CCN4CCCC4(C3)))Cl FC=4C(F)═C(C═C1C=4(N═C(C2=NC═CN12)N3CCNCC3))Cl CN1CCN(CC1)C3=NC2=C(F)C(F)═C(C═C2N4C═CN═C34)Cl FC=5C(F)═C(C═C1C=5(N═C(C2=NC═CN12)N3CCN4CCCC4(C3)))Cl FC(F)(F)C1=CC═C2N═C(C3=NC═CN3 (C2(═C1)))N4CCN5CCCC5 (C4) FC=1C═C2N═C(C3=NC═CN3(C2(═CC=1C(F)(F)F)))N4CCNCC4 CN1CCN(CC1)C3=NC2=CC(F)═C(C═C2N4C═CN═C34)C(F)(F)F FC=1C═C2N═C(C3=NC═CN3 (C2(═CC=1C(F)(F)F)))N4CCN5CCCC5 (C4) FC=4C═C(C═C1C=4(N═C(C2=NC═CN12)N3CCNCC3))C(F)(F)F CN1CCN(CC1)C3=NC2=C(F)C═C(C═C2N4C═CN═C34)C(F)(F)F FC=5C═C(C═C1C=5(N═C(C2=NC═CN12)N3CCN4CCCC4(C3)))C(F)(F)F FC=4C(F)═C(C═C1C=4(N═C(C2=NC═CN12)N3CCNCC3))C(F)(F)F CN1CCN(CC1)C3=NC2=C(F)C(F)═C(C═C2N4C═CN═C34)C(F)(F)F FC=5C(F)═C(C═C1C=5(N═C(C2=NC═CN12)N3CCN4CCCC4(C3)))C(F)(F)F CC1=CC═C2N═C(C3=NC═CN3(C2(═C1)))N4CCNCC4 CC1=CC═C2N═C(C3=NC═CN3(C2(═C1)))N4CCN(C)CC4 CC1=CC═C2N═C(C3=NC═CN3 (C2(═C1)))N4CCN5CCCC5 (C4) CC=1C═C2C(═CC=1(F))N═C(C3=NC═CN23)N4CCNCC4 CC=1C═C2C(═CC=1(F))N═C(C3=NC═CN23)N4CCN(C)CC4 CC=1C═C2C(═CC=1(F))N═C(C3=NC═CN23)N4CCN5CCCC5 (C4) FC(F)(F)C=1C═C2C(═CC=1Cl)N═C(C3=NC═CN23)N4CCNCC4 CN1CCN(CC1)C3=NC2=CC(═C(C═C2N4C═CN═C34)C(F)(F)F)Cl FC(F)(F)C=1C═C2C(═CC=1Cl)N═C(C3=NC═CN23)N4CCN5CCCC5 (C4) FC=1C═C2C(═CC=1C(F)(F)F)N═C(C3=NC═CN23)N4CCNCC4 FC2(F)(C=1C═C3N═C(C4=NC═CN4(C3(═CC=1C(F)(F)C2(F)(F))))N5CCNCC5) FC(F)(F)OC1=CC═C2N═C(C3=NC═CN3(C2(═C1)))N4CCNCC4 FC(F)(F)C(F)(F)C1=CC═C2N═C(C3=NNC═C3(C2(═C1)))N4CCNCC4 CN1CCN(CC1)C4=NC2=CC═C(C═C2C3=CNN═C34)C(F)(F)C(F)(F)F FC(F)(F)C(F)(F)C1=CC═C2N═C(C3=NNC═C3 (C2(═C1)))N4CCN5CCCC5 (C4) FC=1C═C2N═C(C3=NNC═C3(C2(═CC=1C(F)(F)C(F)(F)F)))N4CCNCC4 CN1CCN(CC1)C4=NC2=CC(F)═C(C═C2C3=CNN═C34)C(F)(F)C(F)(F)F FC=1C═C2N═C(C3=NNC═C3 (C2(═CC=1C(F)(F)C(F)(F)F)))N4CCN5CCCC5 (C4) FC=2C═C(C═C3C1=CNN═C1C(═NC=23)N4CCNCC4)C(F)(F)C(F)(F)F CN1CCN(CC1)C3=NC4=C(F)C═C(C═C4(C2=CNN═C23))C(F)(F)C(F)(F)F FC=2C═C(C═C3C1=CNN═C1C(═NC=23)N4CCN5CCCC5(C4))C(F)(F)C(F)(F)F N#CC1=CC(F)═C2N═C(C3=NNC═C3(C2(═C1)))N4CCNCC4 CN1CCN(CC1)C3=NC4=C(F)C═C(C#N)C═C4(C2=CNN═C23) N#CC1=CC(F)═C2N═C(C3=NNC═C3 (C2(═C1)))N4CCN5CCCC5 (C4) FC=1C═C2N═C(C3=NNC═C3(C2(═CC=1Br)))N4CCNCC4 CN1CCN(CC1)C4=NC2=CC(F)═C(C═C2C3=CNN═C34)Br FC=1C═C2N═C(C3=NNC═C3 (C2(═CC=1Br)))N4CCN5CCCC5 (C4) FC=2C(F)═C(C═C3C1=CNN═C1C(═NC=23)N4CCNCC4)Br CN1CCN(CC1)C3=NC4=C(F)C(F)═C(C═C4(C2=CNN═C23))Br FC=2C(F)═C(C═C3C1=CNN═C1C(═NC=23)N4CCN5CCCC5(C4))Br FC=1C═C2N═C(C3=NNC═C3(C2(═CC=1Cl)))N4CCNCC4 CN1CCN(CC1)C4=NC2=CC(F)═C(C═C2C3=CNN═C34)Cl FC=1C═C2N═C(C3=NNC═C3 (C2(═CC=1Cl)))N4CCN5CCCC5 (C4) FC=2C═C(C═C3C1=CNN═C1C(═NC=23)N4CCNCC4)Cl CN1CCN(CC1)C3=NC4=C(F)C═C(C═C4(C2=CNN═C23))Cl FC=2C═C(C═C3C1=CNN═C1C(═NC=23)N4CCN5CCCC5(C4))Cl FC=2C(F)═C(C═C3C1=CNN═C1C(═NC=23)N4CCNCC4)Cl CN1CCN(CC1)C3=NC4=C(F)C(F)═C(C═C4(C2=CNN═C23))Cl FC=2C(F)═C(C═C3C1=CNN═C1C(═NC=23)N4CCN5CCCC5(C4))Cl FC=1C═C2N═C(C3=NNC═C3(C2(═CC=1C(F)(F)F)))N4CCNCC4 CN1CCN(CC1)C4=NC2=CC(F)═C(C═C2C3=CNN═C34)C(F)(F)F FC=1C═C2N═C(C3=NNC═C3 (C2(═CC=1C(F)(F)F)))N4CCN5CCCC5 (C4) FC=2C═C(C═C3C1=CNN═C1C(═NC=23)N4CCNCC4)C(F)(F)F CN1CCN(CC1)C3=NC4=C(F)C═C(C═C4(C2=CNN═C23))C(F)(F)F FC=2C═C(C═C3C1=CNN═C1C(═NC=23)N4CCN5CCCC5(C4))C(F)(F)F FC=2C(F)═C(C═C3C1=CNN═C1C(═NC=23)N4CCNCC4)C(F)(F)F CN1CCN(CC1)C3=NC4=C(F)C(F)═C(C═C4(C2=CNN═C23))C(F)(F)F FC=2C(F)═C(C═C3C1=CNN═C1C(═NC=23)N4CCN5CCCC5(C4))C(F)(F)F FC(F)(F)C=1C═C2C(═CC=1Cl)N═C(C3=NNC═C23)N4CCNCC4 CN1CCN(CC1)C4=NC2=CC(═C(C═C2C3=CNN═C34)C(F)(F)F)Cl FC(F)(F)C=1C═C2C(═CC=1Cl)N═C(C3=NNC═C23)N4CCN5CCCC5 (C4) CN1CCN(CC1)C4=NC2=CC═C(C═C2C3=CN(C)N═C34)C(F)(F)C(F)(F)F CN1C═C2C5=CC(═CC═C5(N═C(C2(═N1))N3CCN4CCCC4(C3)))C(F)(F)C(F)(F)F CN1CCN(CC1)C4=NC2=CC(F)═C(C═C2C3=CN(C)N═C34)C(F)(F)C(F)(F)F CN1C═C2C5=CC(═C(F)C═C5(N═C(C2(═N1))N3CCN4CCCC4(C3)))C(F)(F)C(F)(F)F CN1CCN(CC1)C3=NC4=C(F)C═C(C═C4(C2=CN(C)N═C23))C(F)(F)C(F)(F)F CN1C═C2C5=CC(═CC(F)═C5(N═C(C2(═N1))N3CCN4CCCC4(C3)))C(F)(F)C(F)(F)F CN1CCN(CC1)C3=NC4=C(F)C(F)═C(C═C4(C2=CN(C)N═C23))C(F)(F)C(F)(F)F CN1C═C2C5=CC(═C(F)C(F)═C5(N═C(C2(═N1))N3CCN4CCCC4(C3)))C(F)(F)C(F)(F)F CN1CCN(CC1)C4=NC2=CC═C(C#N)C═C2C3=CN(C)N═C34 CN1C═C2C5=CC(C#N)═CC═C5(N═C(C2(═N1))N3CCN4CCCC4(C3)) CN1CCN(CC1)C4=NC2=CC(F)═C(C#N)C═C2C3=CN(C)N═C34 CN1C═C2C5=CC(C#N)═C(F)C═C5(N═C(C2(═N1))N3CCN4CCCC4(C3)) CN1CCN(CC1)C3=NC4=C(F)C═C(C#N)C═C4(C2=CN(C)N═C23) CN1C═C2C5=CC(C#N)═CC(F)═C5(N═C(C2(═N1))N3CCN4CCCC4(C3)) CN1CCN(CC1)C4=NC2=CC(F)═C(C═C2C3=CN(C)N═C34)Br CN1C═C2C5=CC(═C(F)C═C5(N═C(C2(═N1))N3CCN4CCCC4(C3)))Br CN1CCN(CC1)C3=NC4=C(F)C═C(C═C4(C2=CN(C)N═C23))Br CN1C═C2C5=CC(═CC(F)═C5(N═C(C2(═N1))N3CCN4CCCC4(C3)))Br CN1CCN(CC1)C3=NC4=C(F)C(F)═C(C═C4(C2=CN(C)N═C23))Br CN1C═C2C5=CC(═C(F)C(F)═C5(N═C(C2(═N1))N3CCN4CCCC4(C3)))Br CN1CCN(CC1)C4=NC2=CC(F)═C(C═C2C3=CN(C)N═C34)Cl CN1C═C2C5=CC(═C(F)C═C5(N═C(C2(═N1))N3CCN4CCCC4(C3)))Cl CN1CCN(CC1)C3=NC4=C(F)C═C(C═C4(C2=CN(C)N═C23))Cl CN1C═C2C5=CC(═CC(F)═C5(N═C(C2(═N1))N3CCN4CCCC4(C3)))Cl CN1CCN(CC1)C3=NC4=C(F)C(F)═C(C═C4(C2=CN(C)N═C23))Cl CN1C═C2C5=CC(═C(F)C(F)═C5(N═C(C2(═N1))N3CCN4CCCC4(C3)))Cl CN1CCN(CC1)C4=NC2=CC(F)═C(C═C2C3=CN(C)N═C34)C(F)(F)F CN1C═C2C5=CC(═C(F)C═C5(N═C(C2(═N1))N3CCN4CCCC4(C3)))C(F)(F)F CN1CCN(CC1)C3=NC4=C(F)C═C(C═C4(C2=CN(C)N═C23))C(F)(F)F CN1C═C2C5=CC(═CC(F)═C5(N═C(C2(═N1))N3CCN4CCCC4(C3)))C(F)(F)F CN1CCN(CC1)C3=NC4=C(F)C(F)═C(C═C4(C2=CN(C)N═C23))C(F)(F)F CN1C═C2C5=CC(═C(F)C(F)═C5(N═C(C2(═N1))N3CCN4CCCC4(C3)))C(F)(F)F CC1=CC═C2N═C(C3=NN(C)C═C3(C2(═C1)))N4CCN(C)CC4 CC1=CC═C2N═C(C3=NN(C)C═C3 (C2(═C1)))N4CCN5CCCC5 (C4) CC=1C═C2C(═CC=1(F))N═C(C3=NN(C)C═C23)N4CCN(C)CC4 CC=1C═C2C(═CC=1(F))N═C(C3=NN(C)C═C23)N4CCN5CCCC5 (C4) CC1=CC(F)═C2N═C(C3=NN(C)C═C3(C2(═C1)))N4CCN(C)CC4 CC1=CC(F)═C2N═C(C3=NN(C)C═C3 (C2(═C1)))N4CCN5CCCC5 (C4) C1CN(CCN1)C4=NC2=CC═C(C═C2C=3NC═NC=34)Br CN1CCN(CC1)C4=NC2=CC═C(C═C2C=3NC═NC=34)Br C1CC2CN(CCN2(C1))C5=NC3=CC═C(C═C3C=4NC═NC=45)Br FC=1C═C2N═C(C=3N═CNC=3(C2(═CC=1Br)))N4CCNCC4 CN1CCN(CC1)C4=NC2=CC(F)═C(C═C2C=3NC═NC=34)Br FC=1C═C2N═C(C=3N═CNC=3 (C2(═CC=1Br)))N4CCN5CCCC5 (C4) FC=4C═C(C═C1C=4(N═C(C=2N═CNC1=2)N3CCNCC3))Br CN1CCN(CC1)C4=NC2=C(F)C═C(C═C2C=3NC═NC=34)Br FC=5C═C(C═C1C=5(N═C(C=2N═CNC1=2)N3CCN4CCCC4(C3)))Br FC=1C═C2N═C(C=3N═CNC=3(C2(═CC=1Cl)))N4CCNCC4 CN1CCN(CC1)C4=NC2=CC(F)═C(C═C2C=3NC═NC=34)Cl FC=1C═C2N═C(C=3N═CNC=3 (C2(═CC=1Cl)))N4CCN5CCCC5 (C4) FC=4C═C(C═C1C=4(N═C(C=2N═CNC1=2)N3CCNCC3))Cl CN1CCN(CC1)C4=NC2=C(F)C═C(C═C2C=3NC═NC=34)Cl FC=5C═C(C═C1C=5(N═C(C=2N═CNC1=2)N3CCN4CCCC4(C3)))Cl CN1CCN(CC1)C4=NC2=CC═C(C═C2C=3NC═NC=34)C(F)(F)F FC(F)(F)C1=CC═C2N═C(C=3N═CNC=3 (C2(═C1)))N4CCN5CCCC5 (C4) CN1CCN(CC1)C4=NC2=CC(F)═C(C═C2C=3NC═NC=34)C(F)(F)F FC=1C═C2N═C(C=3N═CNC=3 (C2(═CC=1C(F)(F)F)))N4CCN5CCCC5 (C4) CN1CCN(CC1)C4=NC2=C(F)C═C(C═C2C=3NC═NC=34)C(F)(F)F FC=5C═C(C═C1C=5(N═C(C=2N═CNC1=2)N3CCN4CCCC4(C3)))C(F)(F)F FC(F)(C(C1=CC═C(N═C(N4CCNCC4)C3=C2C═NC═N3)C2=C1)(F)F)F FC(F)(F)C(F)(F)C1=CC═C2N═C(C=3N═CC═NC=3(C2(═C1)))N4CCNCC4 FC(F)(F)C(F)(F)C1=CC═C2N═C(C=3N═CC═CC=3(C2(═C1)))N4CCNCC4 CN(CC4)CCN4C2=NC1=CC═C(C(F)(C(F)(F)F)F)C═C1C3=C2N═CN═C3 CN1CCN(CC1)C4=NC2=CC═C(C═C2C=3N═CC═NC=34)C(F)(F)C(F)(F)F CN1CCN(CC1)C4=NC2=CC═C(C═C2C=3C═CC═NC=34)C(F)(F)C(F)(F)F FC(F)(C(C1=CC═C(N═C(N4CCN(CCC5)C5C4)C3=C2C═NC═N3)C2=C1)(F)F)F FC(F)(F)C(F)(F)C1=CC═C2N═C(C=3N═CC═NC=3(C2(═C1)))N4CCN5CCCC5(C4) FC(F)(F)C(F)(F)C1=CC═C2N═C(C=3N═CC═CC=3(C2(═C1)))N4CCN5CCCC5(C4) FC1=C(C(F)(C(F)(F)F)F)C═C2C(N═C(N4CCNCC4)C3=C2C═NC═N3)=C1 FC=1C═C2N═C(C=3N═CC═NC=3(C2(═CC=1C(F)(F)C(F)(F)F)))N4CCNCC4 FC=1C═C2N═C(C=3N═CC═CC=3(C2(═CC=1C(F)(F)C(F)(F)F)))N4CCNCC4 CN(CC4)CCN4C2=NC1=CC(F)═C(C(F)(C(F)(F)F)F)C═C1C3=C2N═CN═C3 CN1CCN(CC1)C4=NC2=CC(F)═C(C═C2C=3N═CC═NC=34)C(F)(F)C(F)(F)F CN1CCN(CC1)C4=NC2=CC(F)═C(C═C2C=3C═CC═NC=34)C(F)(F)C(F)(F)F FC1=C(C(F)(C(F)(F)F)F)C═C2C(N═C(N4CCN(CCC5)C5C4)C3=C2C═NC═N3)=C1 FC=1C═C2N═C(C=3N═CC═NC=3(C2(═CC=1C(F)(F)C(F)(F)F)))N4CCN5CCCC5(C4) FC=1C═C2N═C(C=3N═CC═CC=3(C2(═CC=1C(F)(F)C(F)(F)F)))N4CCN5CCCC5(C4) FC1=C(N═C(N4CCNCC4)C3=C2C═NC═N3)C2=CC(C(C(F)(F)F)(F)F)═C1 FC=4C═C(C═C1C=4(N═C(C=2N═CC═NC1=2)N3CCNCC3))C(F)(F)C(F)(F)F FC=2C═C(C═C3C=1C═CC═NC=1C(═NC=23)N4CCNCC4)C(F)(F)C(F)(F)F CN(CC4)CCN4C2=NC1=C(F)C═C(C(C(F)(F)F)(F)F)C═C1C3=C2N═CN═C3 CN1CCN(CC1)C4=NC2=C(F)C═C(C═C2C=3N═CC═NC=34)C(F)(F)C(F)(F)F CN1CCN(CC1)C3=NC4=C(F)C═C(C═C4(C=2C═CC═NC=23))C(F)(F)C(F)(F)F FC1=C(N═C(N4CCN(CCC5)C5C4)C3=C2C═NC═N3)C2=CC(C(F)(C(F)(F)F)F)═C1 FC=5C═C(C═C1C=5(N═C(C=2N═CC═NC1=2)N3CCN4CCCC4(C3)))C(F)(F)C(F)(F)F FC=2C═C(C═C3C=1C═CC═NC=1C(═NC=23)N4CCN5CCCC5(C4))C(F)(F)C(F)(F)F FC1=C(C(C(F)(F)F)(F)F)C═C2C(N═C(N4CCNCC4)C3=C2C═NC═N3)=C1F FC=4C(F)═C(C═C1C=4(N═C(C=2N═CC═NC1=2)N3CCNCC3))C(F)(F)C(F)(F)F FC=2C(F)═C(C═C3C=1C═CC═NC=1C(═NC=23)N4CCNCC4)C(F)(F)C(F)(F)F CN(CC4)CCN4C2=NC1=C(F)C(F)═C(C(F)(C(F)(F)F)F)C═C1C3=C2N═CN═C3 CN1CCN(CC1)C4=NC2=C(F)C(F)═C(C═C2C=3N═CC═NC=34)C(F)(F)C(F)(F)F CN1CCN(CC1)C3=NC4=C(F)C(F)═C(C═C4(C=2C═CC═NC=23))C(F)(F)C(F)(F)F FC1=C(C(F)(C(F)(F)F)F)C═C2C(N═C(N4CCN(CCC5)C5C4)C3=C2C═NC═N3)=C1F FC=5C(F)═C(C═C1C=5(N═C(C=2N═CC═NC1=2)N3CCN4CCCC4(C3)))C(F)(F)C(F)(F)F FC=2C(F)═C(C═C3C=1C═CC═NC=1C(═NC=23)N4CCN5CCCC5(C4))C(F)(F)C(F)(F)F N#CC1=CC═C2N═C(C=3N═CN═CC=3(C2(═C1)))N4CCNCC4 N#CC1=CC═C2N═C(C=3N═CC═NC=3(C2(═C1)))N4CCNCC4 N#CC1=CC═C2N═C(C=3N═CC═CC=3(C2(═C1)))N4CCNCC4 CN1CCN(CC1)C4=NC2=CC═C(C#N)C═C2C=3C═NC═NC=34 CN1CCN(CC1)C4=NC2=CC═C(C#N)C═C2C=3N═CC═NC=34 CN1CCN(CC1)C4=NC2=CC═C(C#N)C═C2C=3C═CC═NC=34 N#CC1=CC═C2N═C(C=3N═CN═CC=3 (C2(═C1)))N4CCN5CCCC5 (C4) N#CC1=CC═C2N═C(C=3N═CC═NC=3 (C2(═C1)))N4CCN5CCCC5 (C4) N#CC1=CC═C2N═C(C=3N═CC═CC=3 (C2(═C1)))N4CCN5CCCC5 (C4) N#CC=1C═C2C(═CC=1(F))N═C(C=3N═CN═CC2=3)N4CCNCC4 N#CC=1C═C2C(═CC=1(F))N═C(C=3N═CC═NC2=3)N4CCNCC4 N#CC=1C═C2C(═CC=1(F))N═C(C=3N═CC═CC2=3)N4CCNCC4 CN1CCN(CC1)C4=NC2=CC(F)═C(C#N)C═C2C=3C═NC═NC=34 CN1CCN(CC1)C4=NC2=CC(F)═C(C#N)C═C2C=3N═CC═NC=34 CN1CCN(CC1)C4=NC2=CC(F)═C(C#N)C═C2C=3C═CC═NC=34 N#CC=1C═C2C(═CC=1(F))N═C(C=3N═CN═CC2=3)N4CCN5CCCC5 (C4) N#CC=1C═C2C(═CC=1(F))N═C(C=3N═CC═NC2=3)N4CCN5CCCC5 (C4) N#CC=1C═C2C(═CC=1(F))N═C(C=3N═CC═CC2=3)N4CCN5CCCC5 (C4) FC=2C═C(C═C3C=1C═NC═NC=1C(═NC=23)N4CCNCC4)Cl FC=4C═C(C═C1C=4(N═C(C=2N═CC═NC1=2)N3CCNCC3))Cl FC=2C═C(C═C3C=1C═CC═NC=1C(═NC=23)N4CCNCC4)Cl CN1CCN(CC1)C3=NC4=C(F)C═C(C═C4(C=2C═NC═NC=23))Cl CN1CCN(CC1)C4=NC2=C(F)C═C(C═C2C=3N═CC═NC=34)Cl CN1CCN(CC1)C3=NC4=C(F)C═C(C═C4(C=2C═CC═NC=23))Cl FC=2C═C(C═C3C=1C═NC═NC=1C(═NC=23)N4CCN5CCCC5(C4))Cl FC=5C═C(C═C1C=5(N═C(C=2N═CC═NC1=2)N3CCN4CCCC4(C3)))Cl FC=2C═C(C═C3C=1C═CC═NC=1C(═NC=23)N4CCN5CCCC5(C4))Cl FC=2C(F)═C(C═C3C=1C═NC═NC=1C(═NC=23)N4CCNCC4)Cl FC=4C(F)═C(C═C1C=4(N═C(C=2N═CC═NC1=2)N3CCNCC3))Cl FC=2C(F)═C(C═C3C=1C═CC═NC=1C(═NC=23)N4CCNCC4)Cl CN1CCN(CC1)C3=NC4=C(F)C(F)═C(C═C4(C=2C═NC═NC=23))Cl CN1CCN(CC1)C4=NC2=C(F)C(F)═C(C═C2C=3N═CC═NC=34)Cl CN1CCN(CC1)C3=NC4=C(F)C(F)═C(C═C4(C=2C═CC═NC=23))Cl FC=2C(F)═C(C═C3C=1C═NC═NC=1C(═NC=23)N4CCN5CCCC5(C4))Cl FC=5C(F)═C(C═C1C=5(N═C(C=2N═CC═NC1=2)N3CCN4CCCC4(C3)))Cl FC=2C(F)═C(C═C3C=1C═CC═NC=1C(═NC=23)N4CCN5CCCC5(C4))Cl FC(F)(F)C1=CC═C2N═C(C=3N═CN═CC=3(C2(═C1)))N4CCNCC4 FC(F)(F)C1=CC═C2N═C(C=3N═CC═NC=3(C2(═C1)))N4CCNCC4 FC(F)(F)C1=CC═C2N═C(C=3N═CC═CC=3(C2(═C1)))N4CCNCC4 CN1CCN(CC1)C4=NC2=CC═C(C═C2C=3C═NC═NC=34)C(F)(F)F CN1CCN(CC1)C4=NC2=CC═C(C═C2C=3N═CC═NC=34)C(F)(F)F CN1CCN(CC1)C4=NC2=CC═C(C═C2C=3C═CC═NC=34)C(F)(F)F FC(F)(F)C1=CC═C2N═C(C=3N═CN═CC=3(C2(═C1)))N4CCN5CCCC5(C4) FC(F)(F)C1=CC═C2N═C(C=3N═CC═NC=3(C2(═C1)))N4CCN5CCCC5(C4) FC(F)(F)C1=CC═C2N═C(C=3N═CC═CC=3 (C2(═C1)))N4CCN5CCCC5 (C4) FC=1C═C2N═C(C=3N═CN═CC=3(C2(═CC=1C(F)(F)F)))N4CCNCC4 FC=1C═C2N═C(C=3N═CC═NC=3(C2(═CC=1C(F)(F)F)))N4CCNCC4 FC=1C═C2N═C(C=3N═CC═CC=3(C2(═CC=1C(F)(F)F)))N4CCNCC4 CN1CCN(CC1)C4=NC2=CC(F)═C(C═C2C=3C═NC═NC=34)C(F)(F)F CN1CCN(CC1)C4=NC2=CC(F)═C(C═C2C=3N═CC═NC=34)C(F)(F)F CN1CCN(CC1)C4=NC2=CC(F)═C(C═C2C=3C═CC═NC=34)C(F)(F)F FC=1C═C2N═C(C=3N═CN═CC=3 (C2(═CC=1C(F)(F)F)))N4CCN5CCCC5 (C4) FC=1C═C2N═C(C=3N═CC═NC=3 (C2(═CC=1C(F)(F)F)))N4CCN5CCCC5 (C4) FC=1C═C2N═C(C=3N═CC═CC=3 (C2(═CC=1C(F)(F)F)))N4CCN5CCCC5 (C4) FC=2C═C(C═C3C=1C═NC═NC=1C(═NC=23)N4CCNCC4)C(F)(F)F FC=4C═C(C═C1C=4(N═C(C=2N═CC═NC1=2)N3CCNCC3))C(F)(F)F FC=2C═C(C═C3C=1C═CC═NC=1C(═NC=23)N4CCNCC4)C(F)(F)F CN1CCN(CC1)C3=NC4=C(F)C═C(C═C4(C=2C═NC═NC=23))C(F)(F)F CN1CCN(CC1)C4=NC2=C(F)C═C(C═C2C=3N═CC═NC=34)C(F)(F)F CN1CCN(CC1)C3=NC4=C(F)C═C(C═C4(C=2C═CC═NC=23))C(F)(F)F FC=2C═C(C═C3C=1C═NC═NC=1C(═NC=23)N4CCN5CCCC5(C4))C(F)(F)F FC=5C═C(C═C1C=5(N═C(C=2N═CC═NC1=2)N3CCN4CCCC4(C3)))C(F)(F)F FC=2C═C(C═C3C=1C═CC═NC=1C(═NC=23)N4CCN5CCCC5(C4))C(F)(F)F FC=2C(F)═C(C═C3C=1C═NC═NC=1C(═NC=23)N4CCNCC4)C(F)(F)F FC=4C(F)═C(C═C1C=4(N═C(C=2N═CC═NC1=2)N3CCNCC3))C(F)(F)F FC=2C(F)═C(C═C3C=1C═CC═NC=1C(═NC=23)N4CCNCC4)C(F)(F)F CN1CCN(CC1)C3=NC4=C(F)C(F)═C(C═C4(C=2C═NC═NC=23))C(F)(F)F CN1CCN(CC1)C4=NC2=C(F)C(F)═C(C═C2C=3N═CC═NC=34)C(F)(F)F CN1CCN(CC1)C3=NC4=C(F)C(F)═C(C═C4(C=2C═CC═NC=23))C(F)(F)F FC=2C(F)═C(C═C3C=1C═NC═NC=1C(═NC=23)N4CCN5CCCC5(C4))C(F)(F)F FC=5C(F)═C(C═C1C=5(N═C(C=2N═CC═NC1=2)N3CCN4CCCC4(C3)))C(F)(F)F FC=2C(F)═C(C═C3C=1C═CC═NC=1C(═NC=23)N4CCN5CCCC5(C4))C(F)(F)F FC(F)(F)C=1C═C2C(═CC=1Cl)N═C(C=3N═CN═CC2=3)N4CCNCC4 FC(F)(F)C=1C═C2C(═CC=1Cl)N═C(C=3N═CC═NC2=3)N4CCNCC4 FC(F)(F)C=1C═C2C(═CC=1Cl)N═C(C=3N═CC═CC2=3)N4CCNCC4 CN1CCN(CC1)C4=NC2=CC(═C(C═C2C=3C═NC═NC=34)C(F)(F)F)Cl CN1CCN(CC1)C4=NC2=CC(═C(C═C2C=3N═CC═NC=34)C(F)(F)F)Cl CN1CCN(CC1)C4=NC2=CC(═C(C═C2C=3C═CC═NC=34)C(F)(F)F)Cl FC(F)(F)C=1C═C2C(═CC=1Cl)N═C(C=3N═CN═CC2=3)N4CCN5CCCC5(C4) FC(F)(F)C=1C═C2C(═CC=1Cl)N═C(C=3N═CC═NC2=3)N4CCN5CCCC5(C4) FC(F)(F)C=1C═C2C(═CC=1Cl)N═C(C=3N═CC═CC2=3)N4CCN5CCCC5(C4) CN1CCN(C(C3=NN═CN34)=NC2=C4C═C(C#N)C═C2)CC1

The activity of the compounds in Examples 1-250 as H₁R and/or H₄R inhibitors is illustrated in the following assay. The other compounds listed above, which have not yet been made and/or tested, are predicted to have activity in these assays as well.

### Biological Activity Assay

### In Vitro Histamine Receptor Cell-Based Assays

The cell-based assays utilize an aequorin dependent bioluminescence signal. Doubly-transfected, stable CHO-K1 cell lines expressing human H₁ or H₄, mitochondrion-targeted aequorin, and (H₄ only) human G protein Gα16 are obtained from Perkin-Elmer. Cells are maintained in F12 (Ham's) growth medium, containing 10% (vol./vol.) fetal bovine serum, penicillin (100 IU/mL), streptomycin (0.1 mg/mL), zeocin (0.25 mg/mL) and geneticin (0.40 mg/mL). Cell media components are from Invitrogen, Inc. One day prior to assay, the growth medium is replaced with the same, excluding zeocin and geneticin.

For assay preparation, growth medium is aspirated, and cells are rinsed with calcium-free, magnesium-free phosphate-buffered saline, followed by two to three minute incubation in Versene (Invitrogen, Inc.) at 37° C. Assay medium (DMEM:F12 [50:50], phenol-red free, containing 1 mg/mL protease-free bovine serum albumin) is added to collect the released cells, which are then centrifuged. The cell pellet is re-suspended in assay medium, centrifuged once more, and re-suspended in assay medium to a final density of 5×10⁶ cells/mL. Coelenterazine-h dye (500 μM in ethanol) is added to a final concentration of 5 μM, and mixed immediately. The conical tube containing the cells is then wrapped with foil to protect the light-sensitive dye. The cells are incubated for four hours further at room temperature (approximately 21° C.) with end-over-end rotation to keep them in suspension.

Just before assay, the dye-loaded cells are diluted to 0.75×10⁶ cells/mL (H₁ receptor) or 1.5×10⁶ cells/mL (H₄ receptor) with additional assay medium. Cells are dispensed to 1536 well micro-titer plates at 3 μL/well. To assay receptor antagonism, 60 nl of 100× concentration test compounds in 100% dimethyl sulfoxide (DMSO) are dispensed to the wells, one compound per well, by passive pin transfer, and the plates are incubated for 15 minutes at room temperature. Assay plates are then transferred to a Lumilux bioluminescence plate reader (Perkin-Elmer) equipped with an automated 1536 disposable tip pipette. The pipette dispenses 3 μL/well of agonist (histamine, at twice the final concentration, where final concentration is a previously determined EC₈₀) in assay medium, with concurrent bioluminescence detection. Agonist activity of test compounds is excluded by separate assays that measure response to test compounds immediately, without added histamine agonist.

CCD image capture on the Lumilux includes a 5 second baseline read prior to agonist addition, and generally a 40 second read per plate after agonist addition. A decrease in bioluminescence signal (measured either as area-under-the-curve, or maximum signal amplitude minus minimum signal amplitude) correlates with receptor antagonism in a dose dependent manner. The negative control is DMSO lacking any test compound. For antagonist assays, the positive controls are diphenhydramine (2-Diphenylmethoxy-N,N-dimethylethylamine, 10 μM final concentration, H₁ receptor) or JNJ7777120 (1-[(5-Chloro-1H-indol-2-yl)carbonyl]-4-methyl-piperazine, 10 μM final concentration, H₄ receptor). Efficacy is measured as a percentage of positive control activity.

Data reported as NT refers to the example having been not tested. It is expected that these compounds when tested will be active and will have utility similar to those that have been tested.

TABLE 1
Biological Activity

| | H₄ Antagonist EC₅₀, | H₁ Antagonist EC₅₀, |
|---|---|---|
| | “+” indicates ≦ 10 μM, | “+” indicates ≦ 10 μM, |
| Example # | “−” indicates > 10 μM | “−” indicates > 10 μM |
| 1 | − | − |
| 2 | + | − |
| 3 | − | − |
| 4 | + | − |
| 5 | + | − |
| 6 | + | − |
| 7 | + | − |
| 8 | + | − |
| 9 | + | − |
| 10 | − | − |
| 11 | + | − |
| 12 | + | − |
| 13 | + | − |
| 14 | − | + |
| 15 | + | − |
| 16 | + | − |
| 17 | + | − |
| 18 | + | − |
| 19 | + | − |
| 20 | + | − |
| 21 | + | − |
| 22 | + | − |
| 23 | + | − |
| 24 | + | − |
| 25 | + | − |
| 26 | − | − |
| 27 | + | − |
| 28 | + | − |
| 29 | + | − |
| 30 | + | − |
| 31 | + | − |
| 32 | + | − |
| 33 | + | − |
| 34 | + | − |
| 35 | − | − |
| 36 | + | − |
| 37 | + | − |
| 38 | + | − |
| 39 | + | − |
| 40 | + | − |
| 41 | + | − |
| 42 | + | − |
| 43 | + | − |
| 44 | + | − |
| 45 | + | − |
| 46 | + | − |
| 47 | + | − |
| 48 | + | − |
| 49 | + | − |
| 50 | + | − |
| 51 | + | − |
| 52 | + | − |
| 53 | − | − |
| 54 | + | − |
| 55 | + | − |
| 56 | − | − |
| 57 | + | − |
| 58 | + | − |
| 59 | + | − |
| 60 | + | − |
| 61 | + | − |
| 62 | + | − |
| 63 | + | − |
| 64 | + | − |
| 65 | + | − |
| 66 | + | − |
| 67 | + | − |
| 68 | + | − |
| 69 | − | − |
| 70 | − | − |
| 71 | − | − |
| 72 | − | − |
| 73 | − | − |
| 74 | − | − |
| 75 | − | − |
| 76 | − | − |
| 77 | − | − |
| 78 | − | − |
| 79 | − | − |
| 80 | − | − |
| 81 | − | − |
| 82 | + | − |
| 83 | − | − |
| 84 | + | − |
| 85 | − | − |
| 86 | + | − |
| 87 | + | − |
| 88 | + | − |
| 89 | + | − |
| 90 | + | − |
| 91 | + | − |
| 92 | + | − |
| 93 | + | − |
| 94 | + | − |
| 95 | + | − |
| 96 | + | − |
| 97 | + | − |
| 98 | + | − |
| 99 | + | − |
| 100 | − | − |
| 101 | + | − |
| 102 | − | − |
| 103 | + | − |
| 104 | + | − |
| 105 | + | − |
| 106 | + | − |
| 107 | + | − |
| 108 | − | − |
| 109 | + | − |
| 110 | + | − |
| 111 | + | − |
| 112 | + | − |
| 113 | + | − |
| 114 | + | − |
| 115 | + | − |
| 116 | − | − |
| 117 | − | − |
| 118 | − | − |
| 119 | − | − |
| 120 | + | − |
| 121 | − | − |
| 122 | + | − |
| 123 | + | − |
| 124 | + | − |
| 125 | + | − |
| 126 | + | − |
| 127 | + | − |
| 128 | + | − |
| 129 | + | − |
| 130 | + | − |
| 131 | + | − |
| 132 | + | − |
| 133 | + | − |
| 134 | + | − |
| 135 | + | + |
| 136 | + | − |
| 137 | + | − |
| 138 | + | − |
| 139 | + | − |
| 140 | + | − |
| 141 | + | + |
| 142 | + | − |
| 143 | + | + |
| 144 | + | − |
| 145 | + | − |
| 146 | + | + |
| 147 | + | − |
| 148 | + | − |
| 149 | + | + |
| 150 | + | − |
| 151 | + | + |
| 152 | + | − |
| 153 | + | + |
| 154 | − | − |
| 155 | + | − |
| 156 | + | + |
| 157 | + | − |
| 158 | − | − |
| 159 | + | − |
| 160 | + | − |
| 161 | + | + |
| 162 | − | − |
| 163 | − | − |
| 164 | + | − |
| 165 | + | + |
| 166 | + | − |
| 167 | + | + |
| 168 | + | − |
| 169 | + | − |
| 170 | + | − |
| 171 | + | NT |
| 172 | + | − |
| 173 | + | − |
| 174 | + | − |
| 175 | + | − |
| 176 | − | − |
| 177 | + | − |
| 178 | + | − |
| 179 | + | − |
| 180 | + | + |
| 181 | + | − |
| 182 | + | + |
| 183 | − | − |
| 184 | + | − |
| 185 | + | − |
| 186 | + | − |
| 187 | + | − |
| 188 | + | − |
| 189 | + | − |
| 190 | + | − |
| 191 | + | − |
| 192 | − | − |
| 193 | + | − |
| 194 | + | − |
| 195 | + | − |
| 196 | + | − |
| 197 | + | − |
| 198 | + | − |
| 199 | + | − |
| 200 | + | − |
| 201 | + | − |
| 202 | + | NT |
| 203 | + | NT |
| 204 | + | NT |
| 205 | + | NT |
| 206 | + | NT |
| 207 | + | NT |
| 208 | + | NT |
| 209 | + | NT |
| 210 | + | NT |
| 211 | + | NT |
| 212 | + | NT |
| 213 | + | NT |
| 214 | + | − |
| 215 | + | − |
| 216 | + | − |
| 217 | + | − |
| 218 | + | − |
| 219 | + | − |
| 220 | + | − |
| 221 | − | NT |
| 222 | + | − |
| 223 | + | + |
| 224 | + | NT |
| 225 | + | NT |
| 226 | + | NT |
| 227 | + | NT |
| 228 | + | NT |
| 229 | − | NT |
| 230 | + | NT |
| 231 | + | NT |
| 232 | + | NT |
| 233 | + | NT |
| 234 | + | NT |
| 235 | + | NT |
| 236 | + | − |
| 237 | + | NT |
| 238 | + | NT |
| 239 | + | NT |
| 240 | + | NT |
| 241 | + | NT |
| 242 | + | NT |
| 243 | + | NT |
| 244 | − | NT |
| 245 | + | + |
| 246 | + | NT |
| 247 | + | NT |
| 248 | + | NT |
| 249 | + | + |
| 250 | + | + |

### In Vivo Assay Number One

Assessment of H₄ Antagonism—Model of Scratching Induced by Histamine in CD-1 mice.

### Animals

Female CD-1 mice (Charles River, Hollister, Calif.), approximately 10 weeks old were housed under controlled conditions (12 h light: 12 h dark, 21° C.) and allowed ad libitum access to food (Purina LabDiet 5P14) and water. Animals were deprived of access to food and water for 1 hour during the experimental itch protocol. All studies were performed under the guidelines of the Institutional Animal Care and Use Committee of Kalypsys, Inc.

### Induction and Measurement of Itch

At least 24 hours prior to study initiation, the hair on the rostral dorsum of the animal was clipped to clear a location for intradermal (i.d.) injection of pruritogen (histamine, dissolved in Dulbecco's PBS [pH 7.4] at a concentration of 10 μmol per 20 μL). Animals were dosed by oral gavage with vehicle (9/0.5/0.5/90 PEG-400/Tween-80/PVP-K30/1% carboxymethylcellulose in water) or test compounds (formulated as suspensions in vehicle) at 30 mg/kg in 200 μL by means of a 20 gauge 1.5″ feeding needle affixed to a 1 mL syringe. There were 8 mice per study group. Thirty minutes after oral dosing, animals were injected i.d. with 20 μL of histamine. Immediately afterward animals were placed into individual sections of a standard acrylic cage for observation, which was recorded digitally for a 20 minute period by video cameras (Panasonic SDR-S70/PC) for later review.

Quantitation of induced itch was measured as described previously (Bell, J. K. et al., British Journal of Pharmacology, 142:374-380, 2004) by counting the number of scratching bouts per animal in the 20 minute period after i.d. injection. A scratching bout was defined as three rapid scratch movements of the hind paw in the area of the injection site. Activity with the fore paws was deemed to be grooming and not scratching, and thus was not counted. All data were analyzed using GraphPad Prism (San Diego, Calif.) software, and reported as mean percentage reduction in scratching bouts versus vehicle control. The significance of antagonist effect on agonist-induced itching was analyzed using the nonparametric Mann-Whitney test with P values <0.05 being designated as statistically significant.

Data reported as NT refers to the example having been not tested. It is expected that these compounds when tested will be active and will have utility similar to those that have been tested. In Table 2 below, entries with superscript “1” are statistically significant according to the criteria outlined in the protocol above. Entries with superscript “2” are examples that have been tested on two separate days and the results reported below are the mean of the two experiments

TABLE 2
In Vivo Activity

| | Scratching Bouts (% change from |
|---|---|
| Example # | vehicle control) |
| 20 | −1 |
| 55 | −65^{1,2} |
| 57 | −66¹ |
| 103 | −37 |
| 112 | −73¹ |
| 113 | −66^{1,2} |
| 152 | −13 |

### In Vivo Assay Number Two

### Allergic Conjunctivitis in Passively Sensitized Guinea Pigs

Male Hartley VAF outbred guinea pigs were passively sensitized to ovalbumin by a single OD subconjunctival injection of undiluted guinea pig anti-ovalbumin antiserum 24 hours before OD topical challenge with 500 μg ovalbumin in saline. Control animals were injected with saline only and challenged with ovalbumin. To determine acute phase drug efficacy, 30 min after challenge animals were clinically scored by a masked observer for severity of signs of conjunctivitis based on a standard scale. Test compounds were administered topically 1 hour prior to challenge (QD protocol), or 1 hour prior to challenge and again 8 hours after challenge (BID protocol). Twenty-four hours after challenge, animals were euthanized and conjunctivae were harvested for determination of tissue eosinophil peroxidase (EPO) concentration as a marker of allergic inflammation. Homogenates of freshly collected tissues were prepared by shaking the tissues in 2 mL round-bottom tubes containing 0.5 mL of homogenization buffer (50 mM Tris HCl, pH 8.0, 6 mM KBr) and one 5-mm stainless steel bead on a Qiagen TissueLyser at 30 Hz for 5 min. Homogenates were frozen and thawed once, then centrifuged at 10,000 rpm for 5 min. EPO activity in supernatants was measured by reacting diluted homogenates with a solution of 6 mM o-phenylenediamine substrate and 8.8 mM H₂O₂ in homogenization buffer for 3 min. The reaction was stopped with 4M H₂SO₄ and absorbances were measured at 490 nM on a spectrophotometric plate reader. Total EPO in samples was calculated from a standard curve of recombinant human EPO in each assay. EPO activity was normalized to total protein concentration (Pierce BCA assay) in supernatants. Background EPO activity was determined from the unsensitized, antigen-challenged control group. Percent inhibition was calculated from the sensitized, antigen-challenged, vehicle-treated control group in each experiment. Ovalbumin-injected animals dosed topically with 0.1% w/v dexamethasone (dex) served as positive control. Groups were compared by ANOVA with Dunnett's or Tukey's post-hoc tests where appropriate with significance assigned at the 95% confidence level.

The table below summarizes the results. In the column labeled “BID activity”, a test compound was assigned a “+” if a 0.01% bid dose was statistically equivalent to dexamethasone with respect to reduction of EPO activity, while a “−” was assigned if the compound was statistically inferior to dexamethasone and not different than vehicle. In the column labeled “QD activity”, a test compound was assigned a “+” if a ≦0.1% qd dose was statistically equivalent to dexamethasone with respect to reduction of EPO activity, while a “−” was assigned if the compound was statistically inferior to dexamethasone and not different than vehicle.

Data reported as NT refers to the example having been not tested. It is expected that these compounds when tested will be active and will have utility similar to those that have been tested.

TABLE 3
In Vivo Activity

| Example # | BID activity | QD activity |
|---|---|---|
| 7 | − | NT |
| 19 | + | + |
| 20 | − | NT |
| 21 | − | NT |
| 23 | + | NT |
| 24 | NT | + |
| 27 | NT | − |
| 29 | + | + |
| 31 | NT | − |
| 32 | + | NT |
| 37 | + | NT |
| 39 | + | NT |
| 40 | NT | − |
| 41 | NT | − |
| 45 | NT | + |
| 49 | NT | − |
| 52 | NT | − |
| 54 | NT | − |
| 55 | + | + |
| 57 | + | + |
| 66 | NT | − |
| 95 | − | NT |
| 99 | + | − |
| 103 | NT | + |
| 104 | NT | − |
| 109 | − | NT |
| 113 | − | NT |
| 124 | NT | − |
| 125 | NT | − |
| 126 | NT | + |
| 127 | NT | + |
| 129 | NT | − |
| 133 | NT | − |
| 136 | NT | − |
| 143 | + | − |
| 145 | NT | − |
| 150 | − | NT |
| 152 | − | NT |
| 153 | − | NT |
| 160 | − | NT |
| 161 | + | NT |
| 165 | + | + |
| 166 | + | + |

### Compositions

The following are examples of compositions which may be used to orally deliver compounds disclosed herein as a capsule.

A solid form of a compound of Formula (I) may be passed through one or more sieve screens to produce a consistent particle size. Excipients, too, may be passed through a sieve. Appropriate weights of compounds, sufficient to achieve the target dosage per capsule, may be measured and added to a mixing container or apparatus, and the blend is then mixed until uniform. Blend uniformity may be done by, for example, sampling 3 points within the container (top, middle, and bottom) and testing each sample for potency. A test result of 95-105% of target, with an RSD of 5%, would be considered ideal; optionally, additional blend time may be allowed to achieve a uniform blend. Upon acceptable blend uniformity results, a measured aliquot of this stock formulation may be separated to manufacture the lower strengths. Magnesium stearate may be passed through a sieve, collected, weighed, added to the blender as a lubricant, and mixed until dispersed. The final blend is weighed and reconciled. Capsules may then be opened and blended materials flood fed into the body of the capsules using a spatula. Capsules in trays may be tamped to settle the blend in each capsule to assure uniform target fill weight, and then sealed by combining the filled bodies with the caps.

### COMPOSITION EXAMPLE 1

10 mg Capsule: Total fill weight of capsule is 300 mg, not including capsule weight. Target compound dosage is 10 mg per capsule, but may be adjusted to account for the weight of counterions and/or solvates if given as a salt or solvated polymorph thereof. In such a case the weight of the other excipients, typically the filler, is reduced.

| Ingredient | Quantity per Capsule, mg |
|---|---|
| Compound of Formula (I) | 10.00 |
| Lactose monohydrate | 269.00 |
| Silicon dioxide | 3.00 |
| Crospovidone | 15.00 |
| Magnesium stearate (vegetable grade) | 3.00 |

### COMPOSITION EXAMPLE 2

20 mg Capsule: Total fill weight of capsule is 300 mg, not including capsule weight. Target compound dosage is 20 mg per capsule, but may be adjusted to account for the weight of counterions and/or solvates if given as a salt or solvated polymorph thereof. In such a case the weight of the other excipients, typically the filler, is reduced.

| Ingredient | Quantity per Capsule, mg |
|---|---|
| Compound of Formula (I) | 20.00 |
| Microcrystalline cellulose (MCC) | 277.00 |
| Magnesium stearate (vegetable grade) | 3.00 |

The following are examples of compositions which may be used to topically deliver compounds disclosed herein, for example to the eye or nasal passages.

### COMPOSITION EXAMPLE 3

| Ingredient | Concentration (w/v %) |
|---|---|
| Compound of Formula (I) | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### COMPOSITION EXAMPLE 4

| Ingredient | Concentration (w/v %) |
|---|---|
| Compound of Formula (I) | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula (I): 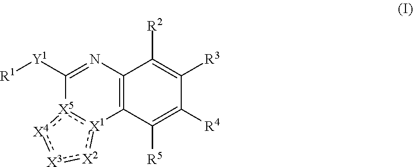
or a salt thereof, wherein:
the ring comprising X¹-X⁵ is aromatic;
X¹ and X⁵ are independently selected from the group consisting of C, CH and N;
X² is selected from the group consisting of [C(R⁶)(R⁷)]ₙ, NR⁸, O and S;
X³ is selected from the group consisting of [C(R⁹)(R¹⁰)]ₘ, NR¹¹, O, and S;
X⁴ is selected from the group consisting of [C(R¹²)(R¹³)], NR¹⁴, O and S;
n and m are each an integer from 1 to 2;
Y¹ is selected from the group consisting of a bond, lower alkyl, lower alkoxy, OR¹⁵, NR¹⁶R¹⁷, and lower aminoalkyl;
R¹ is selected from the group consisting of:
null, when Y¹ is selected from the group consisting of OR¹⁵, and NR¹⁶R¹⁷; and
aryl, heterocycloalkyl, cycloalkyl, and heteroaryl, any of which may be optionally substituted, when Y¹ is a bond;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁶, R⁷, R⁹, R¹⁰, R¹², and R¹³ are independently selected from the group consisting of null, hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁸, R¹¹, and R¹⁴ are independently selected from the group consisting of null, hydrogen, alkyl, heteroalkyl, alkoxy, haloalkyl, perhaloalkyl, aminoalkyl, C-amido, carboxyl, acyl, hydroxy, aryl, arylalkyl, cycloalkyl, cycloalkylalkyl, heterocycloalkyl, heterocycloalkylalkyl, heteroaryl, and heteroarylalkyl, any of which may be optionally substituted;
R¹⁵ and R¹⁶ are independently selected from the group consisting of aminoalkyl, alkylaminoalkyl, aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted; and
R¹⁷ is independently selected from the group consisting of hydrogen, aminoalkyl, alkylaminoalkyl aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted.

2. The compound as recited in claim 1, wherein:
X¹ and X⁵ are independently selected from the group consisting of C and N;
X² is selected from the group consisting of [C(R⁶)(R⁷)]ₙ, NR⁸, and O;
X³ is selected from the group consisting of [C(R⁹)(R¹⁰)]ₘ, NR¹¹, and O;
X⁴ is selected from the group consisting of NR¹⁴, O, and S; and
Y¹ is selected from the group consisting of bond, OR¹⁵, and NR¹⁶R¹⁷;
R¹ is selected from the group consisting of:
null, when Y¹ is selected from the group consisting of OR¹⁵ and NR¹⁶R¹⁷; and
optionally substituted heterocycloalkyl, when Y¹ is a bond.

3. The compound as recited in claim 2, wherein R⁸, R¹¹, and R¹⁴ are independently selected from the group consisting of null, hydrogen, and C₁-C₃ alkyl.

4. The compound as recited in claim 3, wherein:
Y¹ is bond;
X⁴ is NR¹⁴;
R¹ is heterocycloalkyl; and
R¹⁴ is null.

5. A compound as recited in claim 4, having structural Formula (II): 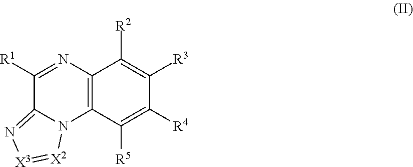
or a salt thereof, wherein:
X² is selected from the group consisting of:
CH and N;
X³ is selected from the group consisting of:
CR⁹ and N;
with the proviso that at least one of X² and X³ is N;
R¹ is selected from the group consisting of heterocycloalkyl, which may be optionally substituted;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; and
R⁹ is selected from the group consisting of hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
with the provisos that
when X³ is CR⁹; and R⁹ is 2-furanyl; and R¹ is selected from the group consisting of piperazin-1-yl and 4-(2-hydroxyethyl)piperazin-1-yl; then R², R³, R⁴, and R⁵ are not all hydrogen; and
when X³ is N; then R¹ is selected from the group consisting of 4-methylpiperazin-1-yl, piperazin-1-yl, and 4-(hexahydropyrrolo[1,2-a]pyrazin-2(1H)-yl); and
when compounds have structural Formula (IIIa), wherein: 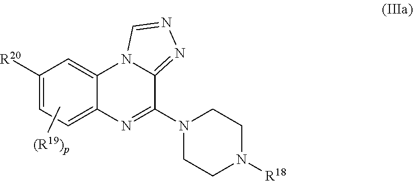
p is an integer from 0 to 3; and
R¹⁸ is selected from the group consisting of hydrogen and methyl; and
R²⁰ is selected from the group consisting of hydrogen and chlorine; and
R¹⁹ is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R¹⁹ are not all hydrogen; and
when compounds have structural Formula (IIIa), wherein:
p is an integer from 0 to 3; and
R¹⁸ is methyl; and
R²⁰ is nitro; and
R¹⁹ is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R¹⁹ are not all hydrogen; and
when compounds have structural Formula (IIIb), wherein: 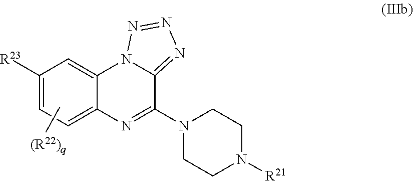
q is an integer from 0 to 3; and
R²¹ is methyl; and
R²³ is selected from the group consisting of hydrogen and methyl; and
R²² is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R²² are not all hydrogen; and
when compounds have structural Formula (IIIb), wherein:
R²¹ and R²³ are hydrogen; and
R²² is independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; then R²² are not all hydrogen.

6. The compound as recited in claim 5, wherein:
R², R³, R⁴, and R⁵ are independently selected from the group consisting of hydrogen, C₁-C₁₀ alkyl, C₁-C₁₀ alkenyl, alkoxy, halogen, haloalkyl, perhaloalkyl, perhaloalkoxy, cyano, and nitro; and
R⁹ is selected from the group consisting of hydrogen, C₁-C₁₀ alkyl, heteroalkyl, alkoxy, halogen, haloalkyl, perhaloalkyl, amino, carboxyl, cyano, nitro, aryl, heteroaryl, cycloalkyl, heterocycloalkyl, any of which may be optionally substituted.

7. The compound as recited in claim 6, wherein:
X² is CH;
X³ is N; and
R¹ is selected from the group consisting of 4-methylpiperazin-1-yl and piperazin-1-yl.

8. The compound as recited in claim 7, wherein:
R², R³, and R⁵ are independently selected from the group consisting of hydrogen, lower alkyl, lower alkenyl, halogen, perhaloalkyl, haloalkyl, and perhaloalkoxy; and
R⁴ is selected from the group consisting of lower alkyl, lower alkenyl, halogen, perhaloalkyl, haloalkyl, and perhaloalkoxy.

9. The compound as recited in claim 8, wherein R⁴ is selected from the group consisting of halogen, lower alkyl, lower alkenyl, perhaloalkoxy, and perhaloalkyl.

10. The compound as recited in claim 9, wherein R² and R⁵ are independently selected from the group consisting of hydrogen, lower alkyl, halogen, and perhaloalkyl.

11. The compound as recited in claim 10, wherein R³ is selected from the group consisting of hydrogen, C₁-C₃ alkyl, halogen, and perhaloalkyl.

12. The compound as recited in claim 6, wherein:
X² is N;
X³ is CR⁹; and
R⁹ is selected from the group consisting of hydrogen, lower alkyl, halogen, haloalkyl, perhaloalkyl, amino, carboxyl, cyano, nitro, aryl, cycloalkyl, heterocycloalkyl, any of which may be optionally substituted.

13. The compound as recited in claim 12, wherein R¹ is selected from the group consisting of 4-methylpiperazin-1-yl and piperazin-1-yl.

14. The compound as recited in claim 13, wherein R⁹ is selected from the group consisting of hydrogen and C₁-C₃ alkyl.

15. The compound as recited in claim 14, wherein R², R³, R⁴, and R⁵ are independently selected from the group consisting of hydrogen, lower alkyl, halogen, haloalkyl, perhaloalkyl, and perhaloalkoxy.

16. The compound as recited in claim 15, wherein R² and R³ are independently selected from the group consisting of hydrogen and halogen.

17. The compound as recited in claim 16, wherein R⁴ is selected from the group consisting of halogen and perhaloalkyl.

18. The compound as recited in claim 6, wherein:
X² and X³ are N;
R¹ is selected from the group consisting of 4-methylpiperazin-1-yl and piperazin-1-yl; and
R⁴ is selected from the group consisting of halogen, haloalkyl, perhaloalkyl, and perhaloalkoxy.

19. The compound as recited in claim 18, wherein R², R³, and R⁵ are independently selected from the group consisting of hydrogen, lower alkyl, halogen, haloalkyl, perhaloalkyl, and perhaloalkoxy.

20. The compound as recited in claim 19, wherein R⁴ is selected from the group consisting of halogen and perhaloalkyl.

21. The compound as recited in claim 20, wherein R² and R³ are independently selected from the group consisting of hydrogen and halogen.

22. A compound as recited in claim 4, having structural Formula (IV): 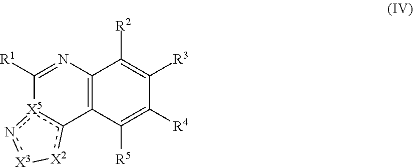
or a salt thereof, wherein:
the 5-membered ring comprising X², X³, and X⁵ is aromatic;
X⁵ is selected from the group consisting of C and N;
X² is selected from the group consisting of:
N, when X⁵ is N; and
O and CR⁶, when X⁵ is C;
X³ is selected from the group consisting of CR⁹ and O, when X⁵ is C; and
CR⁹, when X⁵ is N;
R¹ is heterocycloalkyl, which may be optionally substituted;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; and
R⁶ and R⁹ are independently selected from the group consisting of hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
with the provisos that
when X⁵ is N; then R¹ is selected from the group consisting of 4-methylpiperazin-1-yl, piperazin-1-yl, and bicyclic heterocycloalkyl;
when X² is O; and X³ is CR⁹; and X⁵ is C; then R¹ cannot be 4-morpholino, 4-piperidinyl, or 4-phenylpiperidin-4-ol;
when X² is N; and X³ is CR⁹; and X⁵ is N; and R¹ is 4-methylpiperazin-1-yl;
and R⁴ is hydrogen; then R², R³, R⁵, and R⁹ are not all hydrogen; and
when X² is N; and X³ is CR⁹; and X⁵ is N; and R¹ is piperazin-1-yl; and R⁴ is methyl; then R², R³, R⁵, and R⁹ are not all hydrogen; and
when X² is N; and X³ is CR⁹; and X⁵ is N; and R¹ is 4-methylpiperazin-1-yl; and R⁴ is methoxy; then R³ cannot be methoxy.

23. The compound as recited in claim 22, wherein X⁵ is N.

24. The compound as recited in claim 23, wherein:
X² is N;
X³ is CR⁹;
R⁴ is selected from the group consisting of halogen, haloalkyl, lower alkenyl, perhaloalkyl, and perhaloalkoxy; and
R⁹ is selected from the group consisting of hydrogen and lower alkyl.

25. The compound as recited in claim 24, wherein R¹ is selected from the group consisting of 4-methylpiperazin-1-yl and piperazin-1-yl.

26. The compound as recited in claim 25, wherein R², R³, and R⁵ are independently selected from the group consisting of hydrogen, halogen, haloalkyl, lower alkyl, lower alkenyl, alkoxy, perhaloalkyl, and perhaloalkoxy.

27. The compound as recited in claim 26, wherein R⁴ is selected from the group consisting of halogen and perhaloalkyl.

28. The compound as recited in claim 27, wherein R² and R³ are independently selected from the group consisting of hydrogen and halogen.

29. The compound as recited in claim 22, wherein X⁵ is C.

30. The compound as recited in claim 29, wherein:
X² is CR⁶; and
X³ is O.

31. The compound as recited in claim 30, wherein R¹ is selected from the group consisting of 4-methylpiperazin-1-yl and piperazin-1-yl.

32. The compound as recited in claim 31, wherein R², R³, R⁴, and R⁵ are independently selected from the group consisting of hydrogen, lower alkyl, halogen, haloalkyl, perhaloalkyl, and perhaloalkoxy.

33. The compound as recited in claim 32, wherein R⁴ is selected from the group consisting of halogen and perhaloalkyl.

34. The compound as recited in claim 33, wherein, R² and R³ are independently selected from the group consisting of hydrogen and halogen.

35. The compound as recited in claim 29, wherein:
X² is O;
X³ is CR⁹; and
R¹ is selected from the group consisting of a 5-membered heterocycloalkyl and a 6-membered heterocycloalkyl containing at least two nitrogens.

36. The compound as recited in claim 35, wherein R¹ is selected from the group consisting of 4-methylpiperazin-1-yl and piperazin-1-yl.

37. The compound as recited in claim 36, wherein R², R³, R⁴, and R⁵ are independently selected from the group consisting of hydrogen, lower alkyl, halogen, haloalkyl, perhaloalkyl, and perhaloalkoxy.

38. The compound as recited in claim 37, wherein R⁹ is selected from the group consisting of hydrogen and C₁-C₃ alkyl.

39. The compound as recited in claim 38, wherein R⁴ is selected from the group consisting of halogen and perhaloalkyl.

40. The compound as recited in claim 39, wherein R² and R³ are independently selected from the group consisting of hydrogen and halogen.

41. A compound selected from the group consisting of Examples 1 to 14, 16 to 54, 56, and 59 to 250.

42. A pharmaceutical composition comprising a compound as recited in claim 1 together with a pharmaceutically acceptable carrier.

43. A pharmaceutical composition comprising at least one compound selected from the group consisting of those recited in Examples 1 to 250, together with a pharmaceutically acceptable carrier.

44. A pharmaceutical composition comprising:
a. a compound as selected in claim 1;
b. a H₁R antagonist; and
c. one or more pharmaceutically acceptable carriers or adjuvants.

45. The pharmaceutical composition as recited in claim 44, wherein said H₁R antagonist is selected from the group consisting of acrivastine, alcaftadine, antazoline, azelastine, bromazine, brompheniramine, cetirizine, chlorpheniramine, clemastine, desloratidine, diphenhydramine, diphenylpyraline, ebastine, emedastine, epinastine, fexofenadine, hydroxyzine, ketotifen, levocabastine, levocetirizine, loratidine, methdilazine, mizolastine, promethazine, olopatadine, and triprolidine.

46. A pharmaceutical composition comprising:
a. a compound as selected in claim 1;
b. a H₃R antagonist; and
c. one or more pharmaceutically acceptable carriers or adjuvants.

47. A pharmaceutical composition comprising:
a. a compound as selected in claim 1;
b. a H₁R antagonist and a H₃R antagonist; and
c. one or more pharmaceutically acceptable carriers or adjuvants.

48. A method of treatment of an H₄R-mediated disease comprising the administration, to a patient in need thereof, of a therapeutically effective amount of a compound having structural Formula (I): 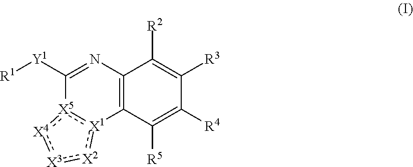
or a salt thereof, wherein:
the ring comprising X¹-X⁵ is aromatic;
X¹ and X⁵ are independently selected from the group consisting of C, CH and N;
X² is selected from the group consisting of [C(R⁶)(R⁷)]ₙ, NR⁸, O and S;
X³ is selected from the group consisting of [C(R⁹)(R¹⁰)]ₘ, NR¹¹, O, and S;
X⁴ is selected from the group consisting of [C(R¹²)(R¹³)], NR¹⁴, O and S;
n and m are each an integer from 1 to 2;
Y¹ is selected from the group consisting of a bond, lower alkyl, lower alkoxy, OR¹⁵, NR¹⁶R¹⁷, and lower aminoalkyl;
R¹ is selected from the group consisting of:
null, when Y¹ is selected from the group consisting of OR¹⁵, and NR¹⁶R¹⁷; and
aryl, heterocycloalkyl, cycloalkyl, and heteroaryl, any of which may be optionally substituted, when Y¹ is a bond;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁶, R⁷, R⁹, R¹⁰, R¹², and R¹³ are independently selected from the group consisting of null, hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁸, R¹¹, and R¹⁴ are independently selected from the group consisting of null, hydrogen, alkyl, heteroalkyl, alkoxy, haloalkyl, perhaloalkyl, aminoalkyl, C-amido, carboxyl, acyl, hydroxy, aryl, arylalkyl, cycloalkyl, cycloalkylalkyl, heterocycloalkyl, heterocycloalkylalkyl, heteroaryl, and heteroarylalkyl, any of which may be optionally substituted;
R¹⁵ and R¹⁶ are independently selected from the group consisting of aminoalkyl, alkylaminoalkyl, aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted; and
R¹⁷ is independently selected from the group consisting of hydrogen, aminoalkyl, alkylaminoalkyl aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted.

49. The method as recited in claim 48, wherein:
X¹ and X⁵ are independently selected from the group consisting of C and N;
X² is selected from the group consisting of [C(R⁶)(R⁷)]ₙ, NR⁸, and O;
X³ is selected from the group consisting of [C(R⁹)(R¹⁰)]ₘ, NR¹¹, and O;
X⁴ is selected from the group consisting of NR¹⁴, O, and S; and
Y¹ is selected from the group consisting of bond, OR¹⁵, and NR¹⁶R¹⁷.
R¹ is selected from the group consisting of:
null, when Y¹ is selected from the group consisting of OR¹⁵ and NR¹⁶R¹⁷; and
optionally substituted heterocycloalkyl, when Y¹ is a bond.

50. The method as recited in claim 49, wherein R⁸, R¹¹, and R¹⁴ are independently selected from the group consisting of null, hydrogen, and C₁-C₃ alkyl.

51. The method as recited in claim 50, wherein:
Y¹ is bond;
X⁴ is NR¹⁴;
R¹ is heterocycloalkyl; and
R¹⁴ is null.

52. A method as recited in claim 51, said compound having structural Formula (II): 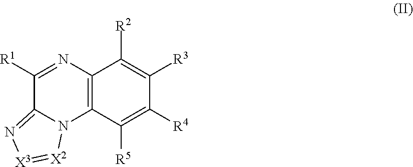
or a salt thereof, wherein:
X² is selected from the group consisting of:
CH and N;
X³ is selected from the group consisting of:
CR⁹ and N;
with the proviso that at least one of X² and X³ is N;
R¹ is selected from the group consisting of heterocycloalkyl, which may be optionally substituted;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; and
R⁹ is selected from the group consisting of hydrogen, lower alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted.

53. The method as recited in claim 52, wherein:
R², R³, R⁴, and R⁵ are independently selected from the group consisting of hydrogen, C₁-C₁₀ alkyl, C₁-C₁₀ alkenyl, alkoxy, halogen, haloalkyl, perhaloalkyl, perhaloalkoxy, cyano, and nitro; and
R⁹ is selected from the group consisting of hydrogen, C₁-C₁₀ alkyl, heteroalkyl, alkoxy, halogen, haloalkyl, perhaloalkyl, amino, carboxyl, cyano, nitro, heteroaryl, aryl, cycloalkyl, heterocycloalkyl, any of which may be optionally substituted.

54. The method as recited in claim 53, wherein:
X² is CH;
X³ is N; and
R¹ is selected from the group consisting of 4-methylpiperazin-1-yl and piperazin-1-yl.

55. The method as recited in claim 54, wherein:
R², R³, and R⁵ are independently selected from the group consisting of hydrogen, lower alkyl, lower alkenyl, halogen, perhaloalkyl, haloalkyl, and perhaloalkoxy; and
R⁴ is selected from the group consisting of lower alkyl, lower alkenyl, halogen, perhaloalkyl, haloalkyl, and perhaloalkoxy.

56. The method as recited in claim 55, wherein R⁴ is selected from the group consisting of halogen, lower alkyl, lower alkenyl, perhaloalkoxy, and perhaloalkyl.

57. The method as recited in claim 56, wherein R² and R⁵ are independently selected from the group consisting of hydrogen, lower alkyl, halogen, and perhaloalkyl.

58. The method as recited in claim 57, R³ is selected from the group consisting of hydrogen, C₁-C₃ alkyl, halogen, and perhaloalkyl.

59. The method as recited in claim 53, wherein:
X² is N;
X³ is CR⁹; and
R⁹ is selected from the group consisting of hydrogen, lower alkyl, halogen, haloalkyl, perhaloalkyl, amino, carboxyl, cyano, nitro, aryl, cycloalkyl, heterocycloalkyl, any of which may be optionally substituted.

60. The method as recited in claim 59, wherein R¹ is selected from the group consisting of 4-methylpiperazin-1-yl and piperazin-1-yl.

61. The method as recited in claim 60, wherein R², R³, R⁴, and R⁵ are independently selected from the group consisting of hydrogen, lower alkyl, halogen, haloalkyl, perhaloalkyl, and perhaloalkoxy.

62. The method as recited in claim 61, wherein R² and R³ are independently selected from the group consisting of hydrogen and halogen.

63. The method as recited in claim 62, wherein R⁴ is selected from the group consisting of halogen and perhaloalkyl.

64. The method as recited in claim 53, wherein:
X² and X³ are N;
R¹ is selected from the group consisting of 4-methylpiperazin-1-yl and piperazin-1-yl; and
R⁴ is selected from the group consisting of halogen, haloalkyl, perhaloalkyl, and perhaloalkoxy.

65. The method as recited in claim 64, wherein R², R³ and R⁵ are independently selected from the group consisting of hydrogen, lower alkyl, halogen, haloalkyl, perhaloalkyl, and perhaloalkoxy.

66. The method as recited in claim 65, wherein R⁴ is selected from the group consisting of halogen and perhaloalkyl.

67. The method as recited in claim 66, wherein R² and R³ are independently selected from the group consisting of hydrogen and halogen.

68. A method as recited in claim 51, said compound having structural Formula (IV): 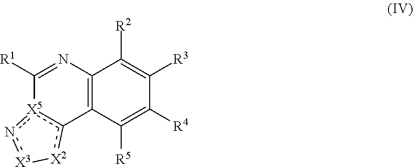
or a salt thereof, wherein:
the 5-membered ring comprising X², X³, and X⁵ is aromatic;
X⁵ is selected from the group consisting of C and N;
X² is selected from the group consisting of:
N, when X⁵ is N; and
O and CR⁶, when X⁵ is C;
X³ is selected from the group consisting of CR⁹ and O, when X⁵ is C; and
CR⁹, when X⁵ is N;
R¹ is heterocycloalkyl, which may be optionally substituted;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted; and
R⁶ and R⁹ are independently selected from the group consisting of hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted.

69. The method as recited in claim 68, wherein X⁵ is N.

70. The method as recited in claim 69, wherein:
X² is N;
X³ is CR⁹;
R⁴ is selected from the group consisting of halogen, haloalkyl, lower alkenyl, perhaloalkyl, and perhaloalkoxy; and
R⁹ is selected from the group consisting of hydrogen and lower alkyl.

71. The method as recited in claim 70, wherein R¹ is selected from the group consisting of 4-methylpiperazin-1-yl and piperazin-1-yl.

72. The method as recited in claim 71, wherein R², R³, and R⁵ are independently selected from the group consisting of hydrogen, halogen, haloalkyl, lower alkyl, lower alkenyl, alkoxy, perhaloalkyl, and perhaloalkoxy.

73. The method as recited in claim 72, wherein R⁴ is selected from the group consisting of halogen and perhaloalkyl.

74. The method as recited in claim 73, wherein R⁹ is selected from the group consisting of hydrogen and C₁-C₃ alkyl.

75. The method as recited in claim 74, wherein R² and R³ are independently selected from the group consisting of hydrogen and halogen.

76. The method as recited in claim 68, wherein X⁵ is C.

77. The method as recited in claim 76, wherein:
X² is CR⁶; and
X³ is O.

78. The method as recited in claim 77, wherein R¹ is selected from the group consisting of 4-methylpiperazin-1-yl and piperazin-1-yl.

79. The method as recited in claim 78, wherein R², R³, R⁴, and R⁵ are independently selected from the group consisting of hydrogen, lower alkyl, halogen, haloalkyl, perhaloalkyl, and perhaloalkoxy.

80. The method as recited in claim 79, wherein R⁴ is selected from the group consisting of halogen and perhaloalkyl.

81. The method as recited in claim 80, wherein, R² and R³ are independently selected from the group consisting of hydrogen and halogen.

82. The method as recited in claim 76, wherein:
X² is O;
X³ is CR⁹; and
R¹ is selected from the group consisting of a 5-membered heterocycloalkyl and a 6-membered heterocycloalkyl containing at least two nitrogens.

83. The method as recited in claim 82, wherein R¹ is selected from the group consisting of 4-methylpiperazin-1-yl and piperazin-1-yl.

84. The method as recited in claim 83, wherein R², R³, R⁴, and R⁵ are independently selected from the group consisting of hydrogen, lower alkyl, halogen, haloalkyl, perhaloalkyl, and perhaloalkoxy.

85. The method as recited in claim 84, wherein R⁹ is selected from the group consisting of hydrogen and C₁-C₃ alkyl.

86. The method as recited in claim 85, wherein R⁴ is selected from the group consisting of halogen and perhaloalkyl.

87. The method as recited in claim 86, wherein R² and R³ are independently selected from the group consisting of hydrogen and halogen.

88. The method as recited in claim 48, wherein said treatment is systemic.

89. The method as recited in claim 48, wherein said administration is topical.

90. The method as recited in claim 48, wherein said disease is selected from the group consisting of an inflammatory disease, an autoimmune disease, an allergic disorder, and an ocular disorder.

91. The method as recited in claim 90, wherein disease is selected from the group consisting of pruritus, eczema, atopic dermatitis, asthma, rhinitis, dry eye, ocular inflammation, allergic conjunctivitis, vernal conjunctivitis, vernal keratoconjunctivitis, and giant papillary conjunctivitis.

92. The method as recited in claim 89, wherein said topical administration is to the skin.

93. The method as recited in claim 89, wherein said topical administration is to the eye.

94. The method as recited in claim 89, wherein said topical administration is intranasal or by inhalation.

95. A method of inhibition of H₄R comprising contacting H₄R with a compound as recited in claim 1.

96. A method of treatment of the pain or inflammation resulting from cataract surgery, comprising delivering to a patient in need of such treatment with a therapeutically effective amount of a compound as recited in claim 1.

97. A method of treatment of an H₄R-mediated disease comprising the administration of:
a. a therapeutically effective amount of a compound as recited in any one of claim 1; and
b. another therapeutic agent.

98. A method for achieving an effect in a patient comprising the administration of a therapeutically effective amount of a compound as recited in claim 1 to a patient, wherein the effect is selected from the group consisting of reduction in the number of mast cells, inhibition of eosiniphil migration optionally to the nasal mucosa, the eye, or the wound site, reduction in inflammatory markers, reduction in inflammatory cytokines, reduction in scratching, decreased watering or redness of the eyes, and reduction in ocular pain.

99. A compound as recited in claim 1 for use as a medicament.

100. A compound as recited in claim 1 for use in the manufacture of a medicament for the prevention or treatment of a disease or condition ameliorated by the inhibition of H₁R and/or H₄R.
